SEQUENCE LISTING (31 sequences)

SEQ ID NO: 1 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 2 - DNA, 3106 bp, Homo sapiens
gcggccgcgctccggccgctttgtcgaaagccggcccgactggagcaggacgaagggggagggtctcgaggccgagtcctgttcttctgagggacggaccccagctggggtggaaaagcagtaccagagagcctccgaggcgcgcggtgccaaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgtccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttgggg

SEQ ID NO: 3 - DNA, 33 bp, Artificial Sequence
  Description of Artificial Sequence Primer
gtcgtatcatatgtcagcagcagaggaagaaac

SEQ ID NO: 4 - DNA, 32 bp, Artificial Sequence
  Description of Artificial Sequence Primer
tctgcagtcgacaggccactcagttccgcttg

SEQ ID NO: 5 - DNA, 391 bp, Artificial Sequence
  Description of Artificial Sequence Pet15S construct
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgggatccggaattcaaaggcctacgtcgactagagcctgcagtctcgaccatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttg

SEQ ID NO: 6 - protein, 21 aa, Artificial Sequence
  Description of Artificial Sequence Pet15S construct
MGSSHHHHHHSSGLVPRGSHM

SEQ ID NO: 7 - protein, 6 aa, Artificial Sequence
  Description of Artificial Sequence 6-His tag
HHHHHH

SEQ ID NO: 8 - protein, 366 aa, Homo sapiens
MGSSHHHHHHSSGLVPRGSHMSAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 9 - DNA, 1185 bp, Artificial Sequence
  Description of Artificial Sequence Pet15s construct
atataccatgggcagcagccatcatcatcatcatcacagcagcggcctggtgccgcgcggcagccatatgtcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctgtcgactagagcctgcagtctcgaccatcatcatcatcatcattaataaaagggcgaattccagcacact

SEQ ID NO: 10 - protein, 341 aa, Homo sapiens
LNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSP

SEQ ID NO: 11 - protein, 337 aa, Homo sapiens
SISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQAP

SEQ ID NO: 12 - protein, 341 aa, Homo sapiens
LTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFPQGDRERERGMEISPMCDKHNASVEKSQVGFIDYIVHPLWETWADLVHPDAQDILDTLEDNREWYQSTIPQSP

SEQ ID NO: 13 - protein, 341 aa, Homo sapiens
LNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSP

SEQ ID NO: 14 - protein, 341 aa, Homo sapiens
LSSATVPRFGVQTDQEEQLAKDVEDTNKWGLDVFKVAELSGNRPLTAIIFSIFQERDLLKTFQIPADTLATYLLMLEGHYHANVAYHNSLHAADVAQSTHVLLATPALEAVFTDLEILAALFASAIHDVDHPGVSNQFLINTNSDVALMYNDASVLENHHLAVGFKLLQAENCDIFQNLSAKQRLSLRRMVIDMVLATDMSKHMNLLADLKTMVETKKVTSLGVLLLDNYSDRIQVLQNLVHCADLSNPTKPLPLYRQWTDRIMAEFFQQGDRERESGLDISPMCDKHTASVEKSQVGFIDYIAHPLWETWADLVHPDAQDLLDTLEDNREWYQSKIPRSP

SEQ ID NO: 15 - protein, 392 aa, Homo sapiens
ERMYRKTYHMVGLAYPAAVIVTLKDVDKWSFDVFALNEASGEHSLKFMIYELFTRYDLINRFKIPVSCLITFAEALEVGYSKYKNPYHNLIHAADVTQTVHYIMLHTGIMHWLTELEILAMVFAAAIHDYEHTGTTNNFHIQTRSDVAILYNDRSVLENHHVSAAYRLMQEEEMNILINLSKDDWRDLRNLVIEMVLSTDMSGHFQQIKNIRNSLQQPEGIDRAKTMSLILHAADISHPAKSWKLHYRWTMALMEEFFLQGDKEAELGLPFSPLCDRKSTMVAQSQIGFIDFIVEPTFSLLTDSTEKIVIPLIEEASKAETSSYVASSSTTIVGLHIADALRRSNTKGSMSDGSYSPDYSLAAVDLKSFKNNLVDIIQQNKERWKELAAQRA

SEQ ID NO: 16 - protein, 389 aa, Homo sapiens
ERMYRRTSNMVGLSYPPAVIEALKDVDKWSFDVFSLNEASGDHALKFIFYELLTRYDLISRFKIPISALVSFVEALEVGYSKHKNPYHNLMHAADVTQTVHYLLYKTGVANWLTELEIFAIIFSAAIHDYEHTGTTNNFHIQTRSDPAILYNDRSVLENHHLSAAYRLLQDDEEMNILINLSKDDWREFRTLVIEMVMATDMSCHFQQIKAMKTALQQPEAIEKPKALSLMLHTADISHPAKAWDLHHRWTMSLLEEFFRQGDREAELGLPFSPLCDRKSTMVAQSQVGFIDFIVEPTFTVLTDMTEKIVSPLIDETSQTGGTGQRRSSLNSISSSDAKRSGVKTSGSEGSAPINNSVISVDYKSFKATWTEVVHINRERWRAKVPKEE

SEQ ID NO: 17 - protein, 369 aa, Homo sapiens
ERMFRRTYTSVGPTYSTAVLNCLKNLDLWCFDVFSLNQAADDHALRTIVFELLTRHNLISRFKIPTVFLMSFLDALETGYGKYKNPYHNQIHAADVTQTVHCFLLRTGMVHCLSEIELLAIIFAAAIHDYEHTGTTNSFHIQTKSECAIVYNDRSVLENHHISSVFRLMQDDEMNIFINLTKDEFVELRALVIEMVLATDMSCHFQQVKTMKTALQQLERIDKPKALSLLLHAADISHPTKQWLVHSRWTKALMEEFFRQGDKEAELGLPFSPLCDRTSTLVAQSQIGFIDFIVEPTFSVLTDVAEKSVQPLADEDSKSKNQPSFQWRQPSLDVEVGDPNPDVVSFRSTWVKRIQENKQKWKERAASGI

SEQ ID NO: 18 - protein, 430 aa, Homo sapiens
MFLDKPILAPEPLVMDNLDSIMEQLNTWNFPIFDLVENIGRKCGRILSQVSYRLFEDMGLFEAFKIPIREFMNYFHALEIGYRDIPYHNRIHATDVLHAVWYLTTQPIPGLSTVINDHGSTSDSDSDSGFTHGHMGYVFSKTYNVTDDKYGCLSGNIPALELMALYVAAAMHDYDHPGRTNAFLVATSAPQAVLYNDRSVLENHHAAAAWNLFMSRPEYNFLINLDHVEFKHFRFLVIEAILATDLKKHFDFVAKFNGKVNDDVGIDWTNENDRLLVCQMCIKLADINGPAKCKELHLQWTDGIVNEFYEQGDEEASLGLPISPFMDRSAPQLANLQESFISHIVGPLCNSYDSAGLMPGKWVEDSDESGDTDDPEEEEEEAPAPNEEETCENNESPKKKTFKRRKIYCQITQHLLQNHKMWKKVIEEEQ

SEQ ID NO: 19 - protein, 432 aa, Homo sapiens
QQTNIEQEVSLDLILVEEYDSLIEKMSNWNFPIFELVEKMGEKSGRILSQVMYTLFQDTGLLEIFKIPTQQFMNYFRALENGYRDIPYHNRIHATDVLHAVWYLTTRPVPGLQQIHNGCGTGNETDSDGRINHGRIAYISSKSCSNPDESYGCLSSNIPALELMALYVAAAMHDYDHPGRTNAFLVATNAPQAVLYNDRSVLENHHAASAWNLYLSRPEYNFLLHLDHVEFKRFRFLVIEAILATDLKKHFDFLAEFNAKANDVNSNGIEWSNENDRLLVCQVCIKLADINGPAKVRDLHLKWTEGIVNEFYEQGDEEANLGLPISPFMDRSSPQLAKLQESFITHIVGPLCNSYDAAGLLPGQWLEAEEDNDTESGDDEDGEELDTEDEEMENNLNPKPPRRKSRRRIFCQLMHHLTENHKIWKEIVEEEE

SEQ ID NO: 20 - protein, 352 aa, Homo sapiens
MVSSNIITPISLDDVPPRIARAMENEEYWDFDIFELEAATHNRPLIYLGLKMFARFGICEFLHCSESTLRSWLQIIEANYHSSNPYHNSTHSADVLHATAYFLSKERIKETLDPIDEVAALIAATIHDVDHPGRTNSFLCNAGSELAILYNDTAVLESHHAALAFQLTTGDDKCNIFKNMERNDYRTLRQGIIDMVLATEMTKHFEHVNKFVNSINKPLATLEENGETDKNQEVINTMLRTPENRTLIKRMLIKCADVSNPCRPLQYCIEWAARISEEYFSQTDEEKQQGLPVVMPVFDRNTCSIPKSQISFIDYFITDMFDAWDAFVDLPDLMQHLDNNFKYWKGLDEMKL

SEQ ID NO: 21 - protein, 341 aa, Homo sapiens
MPITINDVPPCISQLLDNEESWDFNIFELEAITHKRPLVYLGLKVFSRFGVCEFLNCSETTLRAWFQVIEANYHSSNAYHNSTHAADVLHATAFFLGKERVKGSLDQLDEVAALIAATVHDVDHPGRTNSFLCNAGSELAVLYNDTAVLESHHTALAFQLTVKDTKCNIFKNIDRNHYRTLRQAIIDMVLATEMTKHFEHVNKFVNSINKPMAAEIEGSDCECNPAGKNFPENQILIKRMMIKCADVANPCRPLDLCIEWAGRISEEYFAQTDEEKRQGLPVVMPVFDRNTCSIPKSQISFIDYFITDMFDAWDAFAHLPALMQHLADNYKHWKTLDDLEC

SEQ ID NO: 22 - protein, 334 aa, Homo sapiens
GTAVSNSLNILDDDYNGQAKCMLEKVGNWNFDIFLFDRLTNGNSLVSLTFHLFSLHGLIEYFHLDMMKLRRFLVMIQEDYHSQNPYHNAVHAADVTQAMHCYLKEPKLANSVTPWDILLSLIAAATHDLDHPGVNQPFLIKTNHYLATLYKNTSVLENHHWRSAVGLLRESGLFSHLPLESRQQMETQIGALILATDISRQNEYLSLFRSHLDRGDLCLEDTRHRHLVLQMALKCADICNPCRTWELSKQWSEKVTEEFFHQGDIEKKYHLGVSPLCDRHTESIANIQIGFMTYLVEPLFTEWARFSNTRLSQTMLGHVGLNKASWKGLQREQS

SEQ ID NO: 23 - protein, 335 aa, Homo sapiens
GIIPQAPLHLLDEDYLGQARHMLSKVGMWDFDIFLFDRLTNGNSLVTLLCHLFNTHGLIHHFKLDMVTLHRFLVMVQEDYHSQNPYHNAVHAADVTQAMHCYLKEPKLASFLTPLDIMLGLLAAAAHDVDHPGVNQPFLIKTNHHLANLYQNMSVLENHHWRSTIGMLRESRLLAHLPKEMTQDIEQQLGSLILATDINRQNEFLTRLKAHLHNKDLRLEDAQDRHFMLQIALKCADICNPCRIWEMSKQWSERVCEEFYRQGELEQKFELEISPLCNQQKDSIPSIQIGFMSYIVEPLFREWAHFTGNSTLSENMLGHLAHNKAQWKSLLPRQH

SEQ ID NO: 24 - protein, 336 aa, Homo sapiens
LSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQE

SEQ ID NO: 25 - protein, 336 aa, Homo sapiens
LDVLSYHATCSKAEVDKFKAANIPLVSELAIDDIHFDDFSLDVDAMITAALRMFMELGMVQKFKIDYETLCRWLLTVRKNYRMVLYHNWRHAFNVCQLMFAMLTTAGFQDILTEVEILAVIVGCLCHDLDHRGTNNAFQAKSGSALAQLYGTSATLEHHHFNHAVMILQSEGHNIFANLSSKEYSDLMQLLKQSILATDLTLYFERRTEFFELVSKGEYDWNIKNHRDIFRSMLMTACDLGAVTKPWEISRQVAELVTSEFFEQGDRERLELKLTPSAIFDRNRKDELPRLQLEWIDSICMPLYQALVKVNVKLKPMLDSVATNRSKWEELHQKRL

SEQ ID NO: 26 - protein, 329 aa, Homo sapiens
MEKLSYHSICTSEEWQGLMQFTLPVRLCKEIELFHFDIGPFENMWPGIFVYMVHRSCGTSCFELEKLCRFIMSVKKNYRRVPYHNWKHAVTVAHCMYAILQNNHTLFTDLERKGLLIACLCHDLDHRGFSNSYLQKFDHPLAALYSTSTMEQHHFSQTVSILQLEGHNIFSTLSSSEYEQVLEIIRKAIIATDLALYFGNRKQLEEMYQTGSLNLNNQSHRDRVIGLMMTACDLCSVTKLWPVTKLTANDIYAEFWAEGDEMKKLGIQPIPMMDRDKKDEVPQGQLGFYNAVAIPCYTTLTQILPPTEPLLKACRDNLSQWEKVIRGEE

SEQ ID NO: 27 - protein, 345 aa, Homo sapiens
EVYGKEPWECEEEELAEILQAELPDADKYEINKFHFSDLPLTELELVKCGIQMYYELKVVDKFHIPQEALVRFMYSLSKGYRKITYHNWRHGFNVGQTMFSLLVTGKLKRYFTDLEALAMVTAAFCHDIDHRGTNNLYQMKSQNPLAKLHGSSILERHHLEFGKTLLRDESLNIFQNLNRRQHEHAIHMMDIAIIATDLALYFKKRTMFQKIVDQSKTYESEQEWTQYMMLEQTRKEIVMAMMMTACDLSAITKPWEVQSQVALLVAAEFWEQGDLERTVLQQNPIPMMDRNKADELPKLQVGFIDFVCTFVYKEFSRFHEEITPMLDGITNNRKEWKALADEYD

SEQ ID NO: 28 - protein, 345 aa, Homo sapiens
ARLGKEPADCDEDELGEILKEELPGPTTFDIYEFHFSDLECTELDLVKCGIQMYYELGVVRKFQIPQEVLVRFLFSISKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRAMFQKIVDESKNYQDKKSWVEYLSLETTRKEIVMAMMMTACDLSAITKPWEVQSKVALLVAAEFWEQGDLERTVLDQQPIPMMDRNKAAELPKLQVGFIDFVCTFVYKEFSRFHEEILPMFDRLQNNRKEWKALADEYE

SEQ ID NO: 29 - protein, 345 aa, Homo sapiens
KLNVDVIDDCEEKQLVAILKEDLPDPRSAELYEFRFSDFPLTEHGLIKCGIRLFFEINVVEKFKVPVEVLTRWMYTVRKGYRAVTYHNWQHGFNVGQTMFTLLMTGRLKKYYTDLEAFAMLAAAFCHDIDHRGTNNLYQMKSTSPLARLHGSSILERHHLEYSKTLLQDESLNIFQNLNKRQFETVIHLFEVAIIATDLALYFKKRTMFQKIVDACEQMQTEEEAIKYVTVDPTKKEIIMAMMMTACDLSAITKPWEVQSQVALMVANEFWEQGDLERTVLQQQPIPMMDRNKRDELPKLQVGFIDFVCTFVYKEFSRFHKEITPMLSGLQNNRVEWKSLADEYD

SEQ ID NO: 30 - protein, 334 aa, Homo sapiens
MMMYHMKVSDDEYTKLLHDGIQPVAAIDSNFASFTYTPRSLPEDDTSMAILSMLQDMNFINNYKIDCPTLARFCLMVKKGYRDPPYHNWMHAFSVSHFCYLLYKNLELTNYLEDIEIFALFISCMCHDLDHRGTNNSFQVASKSVLAALYSSEGSVMERHHFAQAIAILNTHGCNIFDHFSRKDYQRMLDLMRDIILATDLAHHLRIFKDLQKMAEVGYDRNNKQHHRLLLCLLMTSCDLSDQTKGWKTTRKIAELIYKEFFSQGDLEKAMGNRPMEMMDREKAYIPELQISFMEHIAMPIYKLLQDLFPKAAELYERVASNREHWTKVSHKFT

SEQ ID NO: 31 - protein, 333 aa, Homo sapiens
SFLDNHKKLTPRRDVPTYPKYLLSPETIEALRKPTFDVWLWEPNEMLSCLEHMYHDLGLVRDFSINPVTLRRWLFCVHDNYRNNPFHNFRHCFCVAQMMYSMVWLCSLQEKFSQTDILILMTAAICHDLDHPGYNNTYQINARTELAVRYNDISPLENHHCAVAFQILAEPECNIFSNIPPDGFKQIRQGMITLILATDMARHAEIMDSFKEKMENFDYSNEEHMTLLKMILIKCCDISNEVRPMEVAEPWVDCLLEEYFMQSDREKSEGLPVAPFMDRDKVTKATAQIGFIKFVLIPMFETVTKLFPMVEEIMLQPLWESRDRYEELKRIDD

(19) United States
(12) Patent Application Publication
(10) Pub. No.: US 2005/0048573 A1
(43) Pub. Date: Mar. 3, 2005

(54) PDE5A crystal structure and uses

(75) Inventors: Dean R. Artis, Kensington, CA (US); Gideon Bollag, Orinda, CA (US); Graeme Card, Oakland, CA (US); Fernando Martin, Toronto (CA); Michael V. Milburn, Cary, NC (US); Kam Zhang, Walnut Creek, CA (US)

Correspondence Address: FOLEY & LARDNER, P.O. BOX 80278, SAN DIEGO, CA, 92138-0278, US

(73) Assignee: Plexxikon, Inc.

(21) Appl. No.: 10/771,833

(22) Filed: Feb. 3, 2004

Related U.S. Application Data
(60) Provisional application No. 60/485,627, filed on Jul. 7, 2003.
(60) Provisional application No. 60/444,734, filed on Feb. 3, 2003.

(51) Int. Cl. G01N 33/53; G01N 33/543

(52) U.S. Cl. 435/7.1; 436/518

(57) ABSTRACT

A crystal structure of PDE5A is described that was determined by X-ray crystallography. The use of PDE5A crystals and strucural information can, for example, be used for identifying molecular scaffolds and for developing ligands that bind to and modulate PDE5A.

### CROSS-REFERENCE TO RELATED PATENT APPLICATIONS

This application claims the benefit of Milburn, U.S. Provisional Application 60/444,734, filed Feb. 3, 2003 and Artis et al., U.S. Provisional Application 60/485,627, filed Jul. 7, 2003, all of which are incorporated herein by reference in their entireties, including drawings.

### BACKGROUND OF THE INVENTION

This invention relates to the field of development of ligands for phosphodiesterase 5A (PDE5A) and to the use of crystal structures of PDE5A. The information provided is intended solely to assist the understanding of the reader. None of the information provided nor references cited is admitted to be prior art to the present invention.

PDEs were first detected by Sutherland and co-workers (Rall, et al., J. Biol. Chem., 232:1065-1076 (1958), Butcher, et al., J. Biol. Chem., 237:1244-1250 (1962)). The superfamily of PDEs is subdivided into two major classes, class I and class II (Charbonneau, H., Cyclic Nucleotide Phosphodiesterases: Structure, Regulation and Drug Action, Beavo, J., and Houslay, M. D., eds) 267-296 John Wiley & Sons, Inc., New York (1990)), which have no recognizable sequence similarity. Class I includes all known mammalian PDEs and is comprised of 11 identified families that are products of separate genes (Beavo, et al., Mol. Pharmacol., 46:399-405 (1994); Conti, et al., Endocr. Rev., 16:370-389 (1995); Degerman, et al., J. Biol. Chem., 272:6823-6826 (1997); Houslay, M. D., Adv. Enzyme Regul., 35:303-338 (1995); Bolger, G. B., Cell Signal, 6:851-859 (1994); Thompson, et al, Adv. Second Messenger Phosphoprotein Res., 25:165-184 (1992); Underwood, et al., J. Pharmacol. Exp. Ther., 270:250-259 (1994); Michaeli, et al., J. Biol. Chem., 268:12925-12932 (1993); Soderling, et al., Proc. Natl. Acad. Sci. U.S.A., 95:8991-8996 (1998); Soderling, et al., J. Biol. Chem., 273:15553-15558 (1998); Fisher, et al., J. Biol. Chem., 273:15559-15564 (1998)). Some PDEs are highly specific for hydrolysis of cAMP (PDE4, PDE7, PDE8), some are highly cGMP-specific (PDE5, PDE6, PDE9), and some have mixed specificity (PDE1, PDE2, PDE3, PDE10).

All of the characterized mammalian PDEs are dimeric, but the importance of the dimeric structure for function in each of the PDEs is unknown. Each PDE has a conserved catalytic domain of ˜270 amino acids with a high degree of conservation (25-30%) of amino acid sequence among PDE families, which is located carboxyl-terminal to its regulatory domain. Activators of certain PDEs appear to relieve the influence of autoinhibitory domains located within the enzyme structures (Sonnenberg, et al., J. Biol. Chem., 270:30989-31000 (1995); Jin, et al., J. Biol. Chem., 267:18929-18939 (1992)).

PDEs cleave the cyclic nucleotide phosphodiester bond between the phosphorus and oxygen atoms at the 3′-position with inversion of configuration at the phosphorus atom (Goldberg, et al., J. Biol. Chem., 255:10344-10347 (1980); Burgers, et al., J. Biol. Chem., 254:9959-9961 (1979)). This apparently results from an in-line nucleophilic attack by the OH— of ionized H₂O. It has been proposed that metals bound in the conserved metal binding motifs within PDEs facilitate the production of the attacking OH— (Francis, et al., J. Biol. Chem., 269:22477-22480 (1994)). The kinetic properties of catalysis are consistent with a random order mechanism with respect to cyclic nucleotide and the divalent cations(s) that are required for catalysis (Srivastava, et al., Biochem. J, 308:653-658 (1995)). The catalytic domains of all known mammalian PDEs contain two sequences (HX₃ HXₙ(E/D)) arranged in tandem, each of which resembles the single Zn²⁺-binding site of metalloendoproteases such as thermolysin (Francis, et al., J. Biol. Chem., 269:22477-22480 (1994)). PDE5 specifically binds Zn²⁺, and the catalytic activities of PDE4, PDE5, and PDE6 are supported by submicromolar concentrations of Zn²⁺ (Francis, et al., J. Biol. Chem., 269:22477-22480 (1994); Percival, et al., Biochem. Biophys. Res. Commun., 241:175-180 (1997)). Whether each of the Zn²⁺-binding motifs binds Zn²⁺ independently or whether the two motifs interact to form a novel Zn²⁺-binding site is not known. The catalytic mechanism for cleaving phosphodiester bonds of cyclic nucleotides by PDEs may be similar to that of certain proteases for cleaving the amide ester of peptides, but the presence of two Zn²⁺ motifs arranged in tandem in PDEs is unprecedented.

The group of Sutherland and Rall (Berthet, et al., J. Biol. Chem., 229:351-361 (1957)), in the late 1950s, was the first to realize that at least part of the mechanism(s) whereby caffeine enhanced the effect of glucagon, a stimulator of adenylyl cyclase, on cAMP accumulation and glycogenolysis in liver involved inhibition of cAMP PDE activity. Since that time chemists have synthesized thousands of PDE inhibitors, including the widely used 3-isobutyl-1-methylxanthine (IBMX). Many of these compounds, as well as caffeine, are non-selective and inhibit many of the PDE families. One important advance in PDE research has been the discovery/design of family-specific inhibitors such as the PDE4 inhibitor, rolipram, and the PDE5 inhibitor, sildenafil.

Precise modulation of PDE function in cells is critical for maintaining cyclic nucleotide levels within a narrow rate-limiting range of concentrations. Increases in cGMP of 2-4-fold above the basal level will usually produce a maximum physiological response. There are three general schemes by which PDEs are regulated: (a) regulation by substrate availability, such as by stimulation of PDE activity by mass action after elevation of cyclic nucleotide levels or by alteration in the rate of hydrolysis of one cyclic nucleotide because of competition by another, which can occur with any of the dual specificity PDEs (e.g. PDE1, PDE2, PDE3); (b) regulation by extracellular signals that alter intracellular signaling (e.g. phosphorylation events, Ca²⁺, phosphatidic acid, inositol phosphates, protein-protein interactions, etc.) resulting, for example, in stimulation of PDE3 activity by insulin (Degerman, et al., J. Biol. Chem., 272:6823-6826 (1997)), stimulation of PDE6 activity by photons through the transducin system (Yamazaki, et al., J. Biol. Chem., 255:11619-11624 (1980)), which alters PDE6 interaction with this enzyme, or stimulation of PDE1 activity by increased interaction with Ca²⁺/calmodulin; (c) feedback regulation, such as by phosphorylation of PDE1, PDE3, or PDE4 catalyzed by PKA after cAmP elevation (Conti, et al., Endocr. Rev., 16:370-389 (1995); Degerman, et al., J. Biol. Chem., 272:6823-6826 (1997); Gettys, et al., J. Biol. Chem. 262:333-339 (1987); Florio, et al, Biochemistry, 33:8948-8954 (1994)), by allosteric cGMP binding to PDE2 to promote breakdown of cAMP or cGMP after cGMP elevation, or by modulation of PDE protein levels, such as the desensitization that occurs by increased concentrations of PDE3 or PDE4 following chronic exposure of cells to cAMP-elevating agents (Conti, et al., Endocr. Rev., 16:370-389 (1995), Sheth, et al., Throm. Haemostasis, 77:155-162 (1997)) or by developmentally related changes in PDE5 content. Other factors that could influence any of the three schemes outlined above are cellular compartmentalization of PDEs (Houslay, M. D., Adv. Enzyme Regul., 35:303-338 (1995)) effected by covalent modifications such as prenylation or by specific targeting sequences in the PDE primary structure and perhaps translocation of PDEs between compartments within a cell.

Within the PDE superfamily, four (PDE2, PDE5, PDE6, and PDE10) of the 10 families contain highly cGMP-specific allosteric (non-catalytic) cgMP-binding sites in addition to a catalytic site of varying substrate specificity. Each of the monomers of these dimeric cGMP-binding PDEs contains two homologous cGMP-binding sites of 1 10 amino acids arranged in tandem and located in the amino-terminal portion of the protein (Charbonneau, H., Cyclic Nucleotide Phosphodiesterases: Structure, Regulation and Drug Action, Beavo, J., and Houslay, M. D., eds) 267-296 (1990); McAllister-Lucas, et al., J. Biol. Chem., 270:30671-30679 (1995)). In PDE2, binding of the cGMP to these sites stimulates the hydrolysis of cAMP at the catalytic site (Beavo, et al., Mol. Pharmacol., 46:399-405 (1994)). PDE2 hydrolyzed cGMP as well as cAMP, and cGMP hydrolysis is stimulated by cGMP binding at the allosteric sites in accordance with positively cooperative kinetics (Manganiello, et al., Cyclic Nucleotide Phosphodiesterases. Structure, Regulation, and Drug Action, Beavo, J., and Houslay, M. D., eds, 61-85 John Wiley & Sons, Inc., New York (1990)). This could represent a negative feedback process for regulation of tissue cGMP levels (Manganiello, et al., Cyclic Nucleotide Phosphodiesterases: Structure, Regulation, and Drug Action, Beavo, J., and Houslay, M. D., eds, 61-85 John Wiley & Sons, Inc., New York (1990)), which occurs in addition to the cross-talk between cyclic nucleotide pathways represented by cGMP stimulation of cAMP breakdown. Binding of cGMP to the allosteric sites of PDE6 has not been shown to affect catalysis, but this binding may modulate the interaction of PDE6 with the regulatory protein, transducin, and the inhibitory γ subunit of PDE6 (Yamazaki, et al., Adv. Cyclic Nucleotide Protein Phosphorylation Res., 16:381-392 (1984)).

The first recognized cGMP-binding PDE was discovered as a cGMP-binding protein in lung tissue during a search for cyclic nucleotide-binding proteins other than cyclic nucleotide-dependent protein kinases (Lincoln, et al., Proc. Natl. Acad. Sci U.S.A., 73:2559-2563 (1976)). By DEAE-cellulose chromatography, this protein appeared as a “peak 1” cGMP-binding protein that was separated from a “peak 2” cGMP-binding protein, which was shown to be PKG. The peak 1 protein possessed both cGMP-binding as well as a distinct cGMP-specific PDE catalytic activity (Francis, et al., J. Biol. Chem., 255:620-626 (1980)), and it was subsequently named PDE5. Davis and Kuo (Davis, et al., J. Biol. Chem., 252:4078-4084 (1977)) also described a cGMP-specific PDE activity in lung tissue, and Hamet and Coquil (Hamet, et al., J. Cyclic Nucleotide Res., 4:281-290 (1978)) characterized a cGMP-binding, cGMP-specific PDE in platelets.

PDE5 has been purified and cloned (Francis, et al., J. Biol. Chem., 255:620-626 (1980); Francis, et al., Methods Enzymol., 159:722-729 (1988); Thomas, et al., J. Biol. Chem., 265:14964-14970 (1990); McAllister-Lucas, et al., J. Biol. Chem., 268:22863-22873 (1993)). Two alternatively spliced variants of PDE5 have recently been identified (Yanaka, et al., Eur. J. Biochem., 255:391-399 (1998); Loughney, et al., Gene (Amst.)., 216:137-147 (1998)). The tissue distribution of PDE5 (subunit Mᵣ˜100,000) commonly coincides with that of PKG. This is probably not fortuitous because both PDE5 and PKG are major intracellular receptors for cGMP, and PKG is an excellent catalyst in vitro for phosphorylation of PDE5 (Thomas, et al., J. Biol. Chem., 265:14971-14978 (1990)).

Evidence regarding the presence of conserved Zn²⁺-binding motifs (HX₃ HXₙ(E/D)) in PDEs and their involvement in catalysis was first demonstrated using PDE5 (Francis, et al., J. Biol. Chem., 269:22477-22480 (1994)). Site-directed mutagenesis confirms the catalytic importance of each residue of these motifs A and B (Turko, et al., J. Biol. Chem., 273:6460-6466 (1998)). Substitution of either of the invariant aspartic acid residues (Asp-714, Asp-754) further downstream in the sequence is also highly deleterious, and each of these residues may participate in the catalytic process perhaps as a catalytic base or as a coordinating ligand for a required metal. The most dramatic increases in Km for cGMP are caused by site-directed mutagenesis of Tyr-602 and Glu-775. These two residues could form part of the cGMP-binding pocket of the catalytic site of PDE5. Because some mutations affecting k_{cat} and Kₘ are juxtaposed in the primary sequence, the cGMP-binding pocket and catalytic machinery are likely to involve overlapping subdomains within the catalytic domain of PDE5. All of the components required for catalytic activity of PDE5 are contained within a single monomeric catalytic domain. (Furchgott & Vanhoutte, FASEB J. 3:2007-2018 (1997).)

Occupation of the allosteric cGMP-binding sites of PDE5 is required for specific phosphorylation of Ser-92 by PKG or PKA, and occupation of the binding sites is also associated with an increase in the Stokes radius of the enzyme, implying that a conformational change occurs (Francis, et al., Methods, 14:81-92 (1998)). A direct effect of cGMP binding to the allosteric sites on cGMP breakdown at the catalytic site has not been demonstrated, although the principle of reciprocity (binding of cGMP at the catalytic site stimulates binding at the allosteric sites) dictates that there should be an effect (Weber, G., Adv. Protein Chem., 29:1-83 (1975); Francis, et al., Cyclic Nucleotide Phosphodiesterases: Structure, Regulation and Drug Action, Beavo, J., and Houslay, M. D., eds, 117-140, John Wiley & Sons, Inc., New York (1990)). The stimulatory effect of cGMP analogs specific for the catalytic site on cGMP binding to the allosteric site(s) of PDE5 suggests that interaction of cGMP with the catalytic site precedes cGMP binding to the allosteric binding site(s) (Francis, et al., J. Biol. Chem., 255:620-626 (1980); Thomas, et al., J. Biol. Chem., 265:14971-14978 (1990)). This implies that upon cGMP elevation in cells, cGMP breakdown at the catalytic site would increase because of mass action (increased substrate availability). This increased cGMP interaction at the catalytic site would enhance cGMP binding at the allosteric sites, thus increasing phosphorylation of the enzyme to promote further increases in cGMP breakdown. Although experimental results are consistent with such a sequence of events, this pathway has not been proven unequivocally in broken cell systems. However, rapid phosphorylation of PDE5, which is associated with increased PDE activity, occurs in intact tissues in response to stimulation by atrial natriuretic factor and may be caused by PKG action (Wyatt, et al., Am. J. Physiol., 274:H448-H455 (1998)). This process could represent negative-feedback regulation of cGMP levels in cells. PKA can also phosphorylate PDE5 in vitro, albeit at about 10% the rate at which PKG catalyzes this reaction; whether or not this occurs in vivo is uncertain because the concomitant elevation of cGMP and cAMP would be required to expose Ser-92 and activate PKA, respectively. Burns et al. (Burns, et al., Biochem. J, 283:487-491 (1992)) have reported that a partially purified PDE5 from guinea pig lung is activated when phosphorylated by PKA. PDE5 may also be regulated by other low molecular weight factors, and these could alter the effects of phosphorylation (Lochhead, et al., J. Biol. Chem., 272:18397-18403 (1997)). As is the case for PDE4, PDE5 may also be subject to long term regulation through changes in enzyme concentration in some cell types (Sanchez, et al., Pediatr. Res., 43:163-168 (1998); Kotera, et al., Eur. J. Biochem., 249:434-442 (1997); Bakre, et al., FEBS Lett., 408:345-349 (1997)).

The K_{D} of PDE5 for binding cGMP in the allosteric sites is ˜0.2 μM (Thomas, et al., J. Biol. Chem., 265:14964-14970 (1990)). The presence of two kinetically distinct allosteric cGMP-binding sites in PDE5 was first suggested by the curvilinear pattern of cGMP dissociation from the enzyme. Studies using site-directed mutagenesis confirm the presence of two sites and indicate that the binding of cGMP to each allosteric site could involve a NK(X)ₙD motif (McAllister-Lucas, et al., J. Biol. Chem., 270:30671-30679 (1995); Turko, et al., J. Biol. Chem., 271:22240-22244 (1996)), which resembles that used by G proteins for binding GTP (Pai, et al, Nature, 341:209-214 (1989)). The conserved sequence of the allosteric cyclic nucleotide-binding sites in PDE2, PDE5, PDE6, and PDE10 is evolutionarily distinct from that of the family containing PKG, PKA, and cation channels (McAllister-Lucas, et al., J. Biol. Chem., 268:22863-22873 (1993)), indicating that the allosteric cGMP-binding sites of these PDEs represent a newly recognized class of cyclic nucleotide receptors. Another class may be represented by the catalytic sites of PDEs, the sequences of which contain a binding pocket for cyclic nucleotides in the catalytic domain in order to optimize the catalytic process. In PDE5, classical PDE inhibitors and selected cyclic nucleotide analogs compete with cGMP at the catalytic site but do not interact with the cGMP-binding allosteric sites (Francis, et al., Cyclic Nucleotide Phosphodiesterases: Structure, Regulation and Drug Action, Beavo, J., and Houslay, M. D., eds, 117-140, John Wiley & Sons, Inc., New York (1990)). The order of potency of some common PDE inhibitors for PDE5 is sildenafil>zaprinast>dipyridamole>IBMX>cilostamide>theophylline>caffeine>rolipram (FIG. 3) (Thomas, et al., J. Biol. Chem., 265:14964-14970 (1990); Ballard, et al., J. Urol., 159:2164-2171 (1998)). Many cyclic nucleotide analogs are also inhibitors of PDE5 (Francis, et al., Cyclic Nucleotide Phosphodiesterases: Structure, Regulation and Drug Action, Beavo, J., and Houslay, M. D., eds, 117-140, John Wiley & Sons, Inc., New York (1990)), which is to be expected based on the structural similarity of these compounds with cGMP. Some IBMX analogs modified at the 8-position, such as 8-(2-chlorobenzyl)-IBMX, are more potent inhibitors than are any of the cyclic nucleotide analogs (Sekhar, et al., Phosphodiesterase Inhibitors, Schudt, C., Dent, G., and Rabe, K. F., eds, 135-146, Academic Press, New York (1996)). Even though the IBMX analogs are generally better PDE5 inhibitors than are cyclic nucleotide analogs, many of the latter are more potent for relaxing intact vascular smooth muscle.

Because the PDE inhibitors show competitive kinetics with respect to cGMP in the catalytic site of PDE5, they would be expected to form molecular contacts like those formed by cGMP. However, results of mutagenesis of PDE5 indicate that, although both zaprinast, a potent PDE5 inhibitor, and cGMP appear to make contact with several of the same amino acids in the catalytic domain, some of the residues that are important for interaction with zaprinast, e.g. Asp-754 and Gly-780, are not critical for interaction with cGMP (Turko, et al., J. Biol. Chem., 273:6460-6466 (1998)). As noted above, Asp-754 is crucial for efficient catalysis, which is suggestive that inhibition by zaprinast could be due in part to interference with an important function of Asp-754.

The PDE5 subfamily has only one member: PDE5A (Corbin and Francis, “Cyclic GMP Phosphodiesterase-5: Target of Sildenafil,” The Journal of Biological Chemistry, 274(20):13729-13732 (1999)). PDE5 possesses a preference for cGMP over cAMP as a substrate. PDE5 is expressed in smooth muscle tissue (Table 1), importantly in the corpus cavemosum. This enzyme possesses two GAF domains in the N-terminal regulatory region. These GAF domains act in concert to bind cGMP and mediate dimerization and activation PDE activity. A recent crystal structure of the PDE2 GAF domain suggests possible mechanisms by which the GAF domains bind cGMP and mediate dimerization (Martinez et al., Proc Natl Acad Sci USA 99:13260-13265 (2002)). PDE5 has attracted considerable attention as a therapeutic target due to the tremendous commercial success of Viagra (Pfizer) (Rotella, 2002, Phosphodiesterase 5 inhibitors: Current status and potential applications, Nature Reviews 1:674-682). In addition to Viagra (sildenafil), two other drugs are quite far along in the approval process, namely vardenafil (Bayer) and tadalafil (Lilly/ICOS). One apparent drawback to these compounds is some cross-reactivity with the closely related PDE families PDE6 and PDE 11 (Gresser and Gleiter, Eur J Med Res 7:435-446 (2002)). The availability of PDE5 structural information may enable the discovery of PDE5 inhibitors with improved selectivity versus PDE6 and PDE 11. The crystal structure of PDE5 has not been reported in the literature.

### SUMMARY OF THE INVENTION

The present invention concerns structural information about PDE5A, crystals of PDE5A with and without binding compounds, and the use of the PDE5A crystals and structural information about the PDE5A to develop PDE5A ligands, which can be developed from new chemical classes, or can be developed from previously known PDE5A ligands.

Thus, in a first aspect, the invention concerns a method for developing ligands binding to a PDE5A, where the method includes identifying as molecular scaffolds one or more compounds that bind to a binding site of PDE5A; determining the orientation of at least one molecular scaffold in co-crystals with PDE5A; identifying chemical structures of one or more of the molecular scaffolds, that, when modified, alter the binding affinity or binding specificity or both between the molecular scaffold and the PDE5A; and synthesizing a ligand in which one or more of the chemical structures of the molecular scaffold is modified to provide a ligand that binds to the PDE5A with altered binding affinity or binding specificity or both.

The terms “PDE5A phosphodiesterase” and “PDE5A” mean an enzymatically active phosphodiesterase that contains a portion with greater than 90% amino acid sequence identity to amino acid residues 531-875 of native PDE5A as shown in Table 4, for a maximal alignment over an equal length segment; or that contains a portion with greater than 90% amino acid sequence identity to at least 200 contiguous amino acids from amino acid residues 531-875 of native PDE5A or the amino acid sequence provided in Table 2 that retains binding to natural PDE5A ligand cGMP. Preferably the sequence identity is at least 95, 97, 98, 99, or even 100%. Preferably the specified level of sequence identity is over a sequence at least 300 contiguous amino acid residues.

The term “PDE5A phosphodiesterase domain” refers to a reduced length PDE5A (i.e., shorter than a full-length PDE5A by at least 100 amino acids that includes the phosphodiesterase catalytic region in PDE5A. Highly preferably for use in this invention, the phosphodiesterase domain retains phosphodiesterase activity, preferably at least 50% the level of phosphodiesterase activity as compared to the native PDE5A, more preferably at least 60, 70, 80, 90, or 100% of the native activity.

As used herein, the terms “ligand” and “modulator” are used equivalently to refer to a compound that modulates the activity of a target biomolecule, e.g., an enzyme such as a kinase or phosphodiesterase. Generally a ligand or modulator will be a small molecule, where “small molecule refers to a compound with a molecular weight of 1500 daltons or less, or preferably 1000 daltons or less, 800 daltons or less, or 600 daltons or less. Thus, an “improved ligand” is one that possesses better pharmacological and/or pharmacokinetic properties than a reference compound, where “better” can be defined by a person for a particular biological system or therapeutic use. In terms of the development of ligands from scaffolds, a ligand is a derivative of a scaffold.

In the context of binding compounds, molecular scaffolds, and ligands, the term “derivative” or “derivative compound” refers to a compound having a chemical structure that contains a common core chemical structure as a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

Thus, the term “parent compound” refers to a reference compound for another compound, having structural features continued in the derivative compound. Often but not always, a parent compound has a simpler chemical structure than the derivative.

By “chemical structure” or “chemical substructure” is meant any definable atom or group of atoms that constitute a part of a molecule. Normally, chemical substructures of a scaffold or ligand can have a role in binding of the scaffold or ligand to a target molecule, or can influence the three-dimensional shape, electrostatic charge, and/or conformational properties of the scaffold or ligand.

The term “binds” in connection with the interaction between a target and a potential binding compound indicates that the potential binding compound associates with the target to a statistically significant degree as compared to association with proteins generally (i.e., non-specific binding). Thus, the term “binding compound” refers to a compound that has a statistically significant association with a target molecule. Preferably a binding compound interacts with a specified target with a dissociation constant (k_{d}) of 1 mM or less. A binding compound can bind with “low affinity”, “very low affinity”, “extremely low affinity”, “moderate affinity”, “moderately high affinity”, or “high affinity” as described herein.

In the context of compounds binding to a target, the term “greater affinity” indicates that the compound binds more tightly than a reference compound, or than the same compound in a reference condition, i.e., with a lower dissociation constant. In particular embodiments, the greater affinity is at least 2, 3, 4, 5, 8, 10, 50, 100, 200, 400, 500, 1000, or 10,000-fold greater affinity.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of PDE5A, other phosphodiesterases (e.g., PDE1, PDE6, and/or PDE11) or even other type of enzymes. In particular embodiments, the greater specificity is at least 2, 3, 4, 5, 8, 10, 50, 100, 200, 400, 500, or 1000-fold greater specificity.

As used in connection with binding of a compound with PDE5A, the term “interact” indicates that the distance from a bound compound to a particular amino acid residue will be 5.0 angstroms or less. In particular embodiments, the distance from the compound to the particular amino acid residue is 4.5 angstroms or less, 4.0 angstroms or less, or 3.5 angstroms or less. Such distances can be determined, for example, using co-crystallography, or estimated using computer fitting of a compound in a PDE5A active site.

For reference to particular amino acid residues in PDE5A, polypeptide residue number is defined by the numbering provided in Yanaka et al., 1998, Eur. J. Biochem. 255:391-399.

In a related aspect, the invention provides a method for developing ligands specific for PDE5A, where the method involves determining whether a derivative of a compound that binds to a plurality of phosphodiesterases (e.g., a molecular scaffold) has greater specificity for the PDE5A phosphodiesterase than the parent compound with respect to other phosphodiesterases.

As used herein in connection with binding compounds or ligands, the term “specific for PDE5A phosphodiesterase”, “specific for PDE5A” and terms of like import mean that a particular compound binds to PDE5A to a statistically greater extent than to other phosphodiesterases that may be present in a particular organism. Also, where biological activity other than binding is indicated, the term “specific for PDE5A” indicates that a particular compound has greater biological activity associated with binding PDE5A than to other phosphodiesterases. Preferably, the specificity is also with respect to other biomolecules (not limited to phosphodiesterases) that may be present from an organism.

In another aspect, the invention provides a method for obtaining improved ligands binding to PDE5A, where the method involves identifying a compound that binds to PDE5A, determining whether that compound interacts with one or more conserved PDE5A active site residues, and determining whether a derivative of that compound binds to the PDE5A with greater affinity or greater specificity or both than the parent binding compound. Binding with greater affinity or greater specificity or both than the parent compound indicates that the derivative is an improved ligand. This process can also be carried out in successive rounds of selection and derivatization and/or with multiple parent compounds to provide a compound or compounds with improved ligand characteristics. Likewise, the derivative compounds can be tested and selected to give high selectivity for PDE5A, or to give cross-reactivity to a particular set of targets, for example to a subset of phosphodiesterases that includes PDE5A. In particular embodiments, known PDE5A inhibitors can be used, and derivatives with greater affinity and/or greater specificity can be developed, preferably using PDE5A structure information; greater specificity for PDE5A relative to PDE1, PDE6, and/or PDE11 is developed.

By “molecular scaffold” or “scaffold” is meant a simple target binding molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule, preferably to a plurality of molecules in a protein family, and the target molecule can preferably be a enzyme, receptor, or other protein. Preferred characteristics of a scaffold can include binding at a target molecule binding site such that one or more substituents on the scaffold are situated in binding pockets in the target molecule binding site; having chemically tractable structures that can be chemically modified, particularly by synthetic reactions, so that a combinatorial library can be easily constructed; having chemical positions where moieties can be attached that do not interfere with binding of the scaffold to a protein binding site, such that the scaffold or library members can be modified to form ligands, to achieve additional desirable characteristics, e.g., enabling the ligand to be actively transported into cells and/or to specific organs, or enabling the ligand to be attached to a chromatography column for additional analysis. Thus, a molecular scaffold is an identified target binding molecule prior to modification to improve binding affinity and/or specificity, or other pharmacalogic properties.

The term “scaffold core” refers to the core structure of a molecular scaffold onto which various substituents can be attached. Thus, for a number of scaffold molecules of a particular chemical class, the scaffold core is common to all the scaffold molecules. In many cases, the scaffold core will consist of or include one or more ring structures.

By “binding site” is meant an area of a target molecule to which a ligand can bind non-covalently. Binding sites embody particular shapes and often contain multiple binding pockets present within the binding site. The particular shapes are often conserved within a class of molecules, such as a molecular family. Binding sites within a class also can contain conserved structures such as, for example, chemical moieties, the presence of a binding pocket, and/or an electrostatic charge at the binding site or some portion of the binding site, all of which can influence the shape of the binding site.

By “binding pocket” is meant a specific volume within a binding site. A binding pocket can often be a particular shape, indentation, or cavity in the binding site. Binding pockets can contain particular chemical groups or structures that are important in the non-covalent binding of another molecule such as, for example, groups that contribute to ionic, hydrogen bonding, or van der Waals interactions between the molecules.

By “orientation”, in reference to a binding compound bound to a target molecule is meant the spatial relationship of the binding compound (which can be defined by reference to at least some of its consitituent atoms) to the binding pocket and/or atoms of the target molecule at least partially defining the binding pocket.

In the context of target molecules in this invention, the term “crystal” refers to a regular assemblage of a target molecule of a type suitable for X-ray crystallography. That is, the assemblage produces an X-ray diffraction pattern when illuminated with a beam of X-rays. Thus, a crystal is distinguished from an aggolmeration or other complex of target molecule that does not give a diffraction pattern.

By “co-crystal” is meant a complex of the compound, molecular scaffold, or ligand bound non-covalently to the target molecule and present in a crystal form appropriate for analysis by X-ray or protein crystallography. In preferred embodiments the target molecule-ligand complex can be a protein-ligand complex.

The phrase “alter the binding affinity or binding specificity” refers to changing the binding constant of a first compound for another, or changing the level of binding of a first compound for a second compound as compared to the level of binding of the first compound for third compounds, respectively. For example, the binding specificity of a compound for a particular protein is increased if the relative level of binding to that particular protein is increased as compared to binding of the compound to unrelated proteins.

As used herein in connection with test compounds, binding compounds, and modulators (ligands), the term “synthesizing” and like terms means chemical synthesis from one or more precursor materials.

The phrase “chemical structure of the molecular scaffold is modified” means that a derivative molecule has a chemical structure that differs from that of the molecular scaffold but still contains common core chemical structural features. The phrase does not necessarily mean that the molecular scaffold is used as a precursor in the synthesis of the derivative.

By “assaying” is meant the creation of experimental conditions and the gathering of data regarding a particular result of the experimental conditions. For example, enzymes can be assayed based on their ability to act upon a detectable substrate. A compound or ligand can be assayed based on its ability to bind to a particular target molecule or molecules.

By a “set” of compounds is meant a collection of compounds. The compounds may or may not be structurally related.

In another aspect, structural information about PDE5A can also be used to assist in determining a struture for another phosphodiesterase, e.g., a PDE2, by creating a homology model from an electronic representation of a PDE5A structure.

Typically creating such a homology model involves identifying conserved amino acid residues between PDE5A and the other phosphodiesterase of interest; transferring the atomic coordinates of a plurality of conserved amino acids in the PDE5A structure to the corresponding amino acids of the other phosphodiesterase to provide a rough structure of that phosphodiesterase; and constructing structures representing the remainder of the other phosphodiesterase using electronic representations of the structures of the remaining amino acid residues in the other phosphodiesterase. In particular, coordinates from Table 1 for conserved residues can be used. Conserved residues in a binding site can be used.

To assist in developing other portions of the phosphodiesterase structure, the homology model can also utilize, or be fitted with, low resolution x-ray diffraction data from one or more crystals of the phosphodiesterase, e.g., to assist in linking conserved residues and/or to better specify coordinates for terminal portions of a polypeptide.

The PDE5A structural information used can be for a variety of different PDE5A variants, including full-length wild type, naturally-occurring variants (e.g., allelic variants and splice variants), truncated variants of wild type or naturally-occuring variants, and mutants of full-length or truncated wild-type or naturally-occurring variants (that can be mutated at one or more sites). For example, in order to provide a PDE5A structure closer to a variety of other phosphodiesterase structures, a mutated PDE5A that includes a mutation to a conserved residue in a binding site can be used.

In another aspect, the invention provides a crystalline form of PDE5A, which may be a reduced length PDE5A such as a PDE5A phosphodiesterase domain, e.g., having atomic coordinates as described in Table 1. The crystalline form can contain one or more heavy metal atoms, for example, atoms useful for X-ray crystallography. The crystalline form can also include a binding compound in a co-crystal, e.g., a binding compound that interacts with one more more conserved PDE5A active site residues, or any two, any three, any four, any five, any six of those residues, and can, for example, be a known PDE5A inhibitor. PDE5A crystals can be in various environments, e.g., in a crystallography plate, mounted for X-ray crystallography, and/or in an X-ray beam. The PDE5A may be of various forms, e.g., a wild-type, variant, truncated, and/or mutated form as described herein.

The invention further concerns co-crystals of PDE5A, which may be a reduced length PDE5A, e.g., a PDE5A phosphodiesterase domain, and a PDE5A binding compound. Advantageously, such co-crystals are of sufficient size and quality to allow structural determination of PDE5A to at least 3 Angstroms, 2.5 Angstroms, 2.0 Angstroms, or 1.8 Angstroms. The co-crystals can, for example, be in a crystallography plate, be mounted for X-ray crystallography and/or in an X-ray beam. Such co-crystals are beneficial, for example, for obtaining structural information concerning interaction between PDE5A and binding compounds.

PDE5A binding compounds can include compounds that interact with at least one of conserved PDE5A active site residues, or any 2, 3, 4, 5, or 6 of those residues. Exemplary compounds that bind to PDE5A include compounds described in references cited herein.

Likewise, in additional aspects, methods for obtaining PDE5A crystals and co-crystals are provided. In one aspect is provided a method for obtaining a crystal of PDE5A phosphodiesterase domain, by subjecting PDE5A phosphodiesterase domain protein at 5-20 mg/ml, preferably 8-12 mg/ml, to crystallization condition substantially equivalent to: 10% (w/v) PEG3000, 100 mM phosphate-citrate (pH 4.3), 200 mM NaCl, 1 mM DTT, 1 mM Sp-cAMP. In general, the PDE5A will be in a solution containing the protein and suitable buffer.

Crystallization conditions can be initially identified using a screening kit, such as a Hampton Research (Riverside, Calif.) screening kit 1. Conditions resulting in crystals can be selected and crystallization conditions optimized based on the demonstrated crystallization conditions. To assist in subsequent crystallography, the PDE5A can be seleno-methionine labeled. Also, as indicated above, the PDE5A may be any of various forms, e.g., truncated to provide a PDE5A phosphodiesterase domain, which can be selected to be of various lengths.

A related aspect provides a method for obtaining co-crystals of PDE5A, which can be a reduced length PDE5A, with a binding compound, by subjecting PDE5A protein at 5-20 mg/ml to crystallization conditions substantially equivalent to 10% (w/v) PEG3000, 100 mM phosphate-citrate (pH 4.3), 200 mM NaCl, 1 mM DTT, 1 mM Sp-cAMP, in the presence of binding compound, for a time sufficient for cystal development. The binding compound may be added at various concentrations depending on the nature of the comound, e.g., final concentration of 0.5 to 1.0 mM. In many cases, the binding compound will be in an organic solvent such as demethyl sulfoxide solution (DMSO). While not preferred, binding compound can also be soaked into a PDE5A crystal, e.g., using conventional techniques.

In another aspect, provision of compounds active on PDE5A also provides a method for modulating PDE5A activity by contacting PDE5A with a compound that binds to PDE5A and interacts with one more conserved PDE5A active site residues, where the compound has been identified using a PDE5A crystal structure. The compound is preferably provided at a level sufficient to modulate the activity of PDE5A by at least 10%, more preferably at least 20%, 30%, 40%, or 50%. In many embodiments, the compound will be at a concentration of about 1 μM, 100 μM, or 1 mM, or in a range of 1-100 nM, 100-500 nM, 500-1000 nM, 1-100 μM, 100-500 μM, or 500-1000 μM.

As used herein, the term “modulating” or “modulate” refers to an effect of altering a biological activity, especially a biological activity associated with a particular biomolecule such as PDE5A. For example, an agonist or antagonist of a particular biomolecule modulates the activity of that biomolecule, e.g., an enzyme.

The term “PDE5A activity” refers to a biological activity of PDE5A, particularly including phosphodiesterase activity.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

In a related aspect, the invention provides a method for treating a patient suffering from a disease or condition characterized by abnormal PDE5A phosphodiesterase activity, where the method involves administering to the patient a compound identified by fitting to a PDE5A crystal structure.

Specific diseases or disorders which might be treated or prevented include those described in the Detailed Description herein, and in the references cited therein.

As crystals of PDE5A have been developed and analyzed, another aspect concerns an electronic representation of PDE5A (which may be a reduced length PDE5A), for example, an electronic representation containing atomic coordinate representations corresponding to the coordinates listed for PDE5A in Table 1, or a schematic representation such as one showing secondary structure and/or chain folding, and may also show conserved active site residues. The PDE5A may be wild type, an allelic variant, a mutant form, or a modifed form, e.g., as described herein.

The electronic representation can also be modified by replacing electronic representations of particular residues with electronic representations of other residues. Thus, for example, an electronic representation containing atomic coordinate representations corresponding to the coordinates for PDE5A listed in Table 1 can be modified by the replacement of coordinates for a particular conserved residue in a binding site by a different amino acid. Likewise, a PDE5A representation can be modified by the respective substitutions, insertions, and/or deletions of amino acid residues to provide a representation of a structure for PDE6 or PDE 11. Following a modification or modifications, the representation of the overall structure can be adjusted to allow for the known interactions that would be affected by the modification or modifications. In most cases, a modification involving more than one residue will be performed in an iterative manner.

In addition, an electronic representation of a PDE5A binding compound or a test compound in the binding site can be included, e.g., a non-hydrolyzable cGMP analog.

Likewise, in a related aspect, the invention concerns an electronic representation of a portion of PDE5A, a binding site (which can be an active site) or phosphodiesterase domain, for example, residues 531-875 or other phosphodiesterase domain described herein, such as the amino acid sequence provided in Table 2. A binding site or phosphodiesterase domain can be represented in various ways, e.g., as representations of atomic coordinates of residues around the binding site and/or as a binding site surface contour, and can include representations of the binding character of particular residues at the binding site, e.g., conserved residues. As for electronic representations of PDE5A, a binding compound or test compound may be present in the binding site; the binding site may be of a wild type, variant, mutant form, or modified form of PDE5A.

In yet another aspect, the structural information of PDE5A can be used in a homology model (based on PDE5A) for another phosphodiesterase (such as PDE6 or PDE11), thus providing an electronic representation of a PDE5A based homology model for a phosphodiesterase. For example, the homology model can utilize atomic coordinates from Table 1 for conserved amino acid residues. In particular embodiments; atomic coordinates for a wild type, variant, modified form, or mutated form of PDE5A can be used, including, for example, wild type, variants, modified forms, and mutant forms as described herein. In particular, PDE5A structure provides a very close homology model for PDE6 and PDE 1. Thus, in particular embodiments the invention provides PDE5A-based homology models of PDE6 and PDE 11.

In still another aspect, the invention provides an electronic representation of a modified PDE5A crystal structure, that includes an electronic representation of the atomic coordinates of a modified PDE5A. In an exemplary embodiment, atomic coordinates of Table 1 can be modified by the replacement of atomic coordinates for a conserved residue with atomic coordinates for a different amino acid. Modifications can include substitutions, deletions (e.g., C-terminal and/or N-terminal detections), insertions (internal, C-terminal, and/or N-terminal) and/or side chain modifications.

In another aspect, the PDE5A structural information provides a method for developing useful biological agents based on PDE5A, by analyzing a PDE5A structure to identify at least one sub-structure for forming the biological agent. Such sub-structures can include epitopes for antibody formation, and the method includes developing antibodies against the epitopes, e.g., by injecting an epitope presenting composition in a mammal such as a rabbit, guinea pig, pig, goat, or horse. The sub-structure can also include a mutation site at which mutation is expected to or is known to alter the activity of the PDE5A, and the method includes creating a mutation at that site. Still further, the sub-structure can include an attachment point for attaching a separate moiety, for example, a peptide, a polypeptide, a solid phase material (e.g., beads, gels, chromatographic media, slides, chips, plates, and well surfaces), a linker, and a label (e.g., a direct label such as a fluorophore or an indirect label, such as biotin or other member of a specific binding pair). The method can include attaching the separate moiety.

In another aspect, the invention provides a method for identifying potential PDE5A, binding compounds by fitting at least one electronic representation of a compound in an electronic representation of a PDE5A binding site. The representation of the binding site may be part of an electronic representation of a larger portion(s) or all of a PDE5A molecule or may be a representation of only the binding site or active site. The electronic representation may be as described above or otherwise described herein. For example, the compound may be a molecular scaffold, a derivative of a molecular scaffold, or a compound that is structurally similar to such molecular scaffold or derivative thereof.

In particular embodiments, the method involves fitting a computer representation of a compound from a computer database with a computer representation of the active site of PDE5A, and involves removing a computer representation of a compound complexed with the PDE5A molecule and identifying compounds that best fit the active site based on favorable geometric fit and energetically favorable complementary interactions as potential binding compounds. In particular embodiments, the compound is a known PDE5A inhibitor, e.g., as described in a reference cited herein, or a derivative thereof.

In other embodiments, the method involves modifying a computer representation of a compound complexed with a PDE5A molecule, by the deletion or addition or both of one or more chemical groups; fitting a computer representation of a compound from a computer database with a computer representation of the active site of the PDE5A molecule; and identifying compounds that best fit the active site based on favorable geometric fit and energetically favorable complementary interactions as potential binding compounds.

In still other embodiments, the method involves removing a computer representation of a compound complexed with PDE5A, and searching a database for compounds having structural similarity to the complexed compound using a compound searching computer program or replacing portions of the complexed compound with similar chemical structures using a compound construction computer program.

Fitting a compound can include determining whether a compound will interact with one or more conserved PDE5A active site residues. Compounds selected for fitting or that are complexed with PDE5A can, for example, be a known PDE5A inhibitor compound.

In another aspect, the invention concerns a method for attaching a PDE5A binding compound to an attachment component, as well as a method for indentifying attachment sites on a PDE5A binding compound. The method involves identifying energetically allowed sites for attachment of an attachment component for the binding compound bound to a binding site of PDE5A; and attaching the compound or a derivative thereof to the attachment component at the energetically allowed site.

Attachment components can include, for example, linkers (including traceless linkers) for attachment to a solid phase or to another molecule or other moiety. Such attachment can be formed by synthesizing the compound or derivative on the linker attached to a solid phase medium e.g., in a combinatorial synthesis in a plurality of compound. Likewise, the attachment to a solid phase medium can provide an affinity medium (e.g., for affinity chromatography).

The attachment component can also include a label, which can be a directly detectable label such as a fluorophore, or an indirectly detectable such as a member of a specific binding pair, e.g., biotin.

The ability to identify energentically allowed sites on a PDE5A binding compound, also, in a related aspect, provides modified binding compounds that have linkers attached, preferably at an energetically allowed site for binding of the modified compound to PDE5A. The linker can be attached to an attachment component as described above.

Another aspect concerns a modified PDE5A polypeptide that includes a modification that makes the modified PDE5A more similar than native PDE5A to another phosphodiesterase, and can also include other mutations or other modifications. In various embodiments, the polypeptide includes a full-length PDE5A polypeptide, includes a modified PDE5A binding site, includes at least 20, 30, 40, 50, 60, 70, or 80 contiguous amino acid residues derived from PDE5A including a conserved site.

Still another aspect of the invention concerns a method for developing a ligand for a phosphodiesterase that includes conserved residues matching any one, 2, 3, 4, 5, or 6 of conserved PDE5A active site residues, by determining whether a compound binds to the phosphodiesterase and interacts with such active site residues in a PDE5A crystal. The method can also include determining whether the compound modulates the activity of the phosphodiesterase. Preferably the phosphodiesterase has at least 50, 55, 60, or 70% identity over an equal length phosphodiesterase domain segment.

In particular embodiments, the determining includes computer fitting the compound in a binding site of the phosphodiesterase and/or the method includes forming a co-crystal of the phosphodiesterase and the compound. Such co-crystals can be used for determing the binding orientation of the compound with the phosphodiesterase and/or provide structural information on the phosphodiesterase, e.g., on the binding site and interacting amino acid residues. Such binding orientation and/or other structural information can be accomplished using X-ray crystallography.

The invention also provides compounds that bind to and/or modulate (e.g., inhibit) PDE5A, e.g., PDE5A phosphodiesterase activity. Accordingly, in aspects and embodiments involving PDE5A binding compounds, molecular scaffolds, and ligands or modulators, the compound is a weak binding compound; a moderate binding compound; a strong binding compound; the compound interacts with one or more conserved PDE5A active site residues; the compound is a small molecule; the compound binds to a plurality of different phosphodiesterases (e.g., at least 2, 3, 4, 5, 7, 10, or more different phosphodiesterases).

Additional aspects and embodiments will be apparent from the following Detailed Description and from the claims.

### BRIEF DESCRIPTION OF THE DRAWINGS

FIG. 1 shows a ribbon diagram schematic representation of PDE5A phosphodiesterase domain having the sequence in Table 2.

### DETAILED DESCRIPTION OF THE PREFERRED EMBODIMENTS

The Tables will first be briefly described.

Table 1 provides atomic coordinates for human PDE5A phosphodiesterase domain. In this table, the various columns have the following content, beginning with the left-most column:
ATOM: Refers to the relevant moeity for the table row.
Atom number: Refers to the arbitrary atom number designation within the coordinate table.
Atom Name: Identifier for the atom present at the particular coordinates.
Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.
Residue Number: The amino acid residue number in the chain.
X, Y, Z: Respectively are the X, Y, and Z coordinate values.
Occupancy: Describes the fraction of time the atom is observed in the crystal. For example, occupancy=1 means that the atom is present all the time; occupancy=0.5 indicates that the atom is present in the location 50% of the time.
B-factor: A measure of the thermal motion of the atom.
Element: Identifier for the element.

Table 2 provides amino acid and nucleic acid sequences for a PDE5A phosphodiesterase domain. Numbering on the amino acid sequence does not correspond to standard numbering for native PDE5A.

Table 3 provides an alignment of phosphodiesterase domains for several phosphodiesterases, including human PDE5A, providing identification of residues conserved between various members of the set.

Table 4 provides the nucleic acid and amino acid sequences for human PDE5A phosphodiesterase.

I. General and PDE5 Inhibitors

The present invention concerns the use of PDE5A phosphodiesterase structures, structural information, and related compositions for identifying compounds that modulate PDE5A phosphodiesterase activity and for determining structuctures of other phosphodiesterases.

PDE5A is involved in a number of disease and conditions, and thus can be targeted in therapeutic and prophylactic methods.

A large number of compounds that are active on PDE5, from several different chemical classes, have been identified, and pharmaceutical products directed to PDE5 have been developed and approved by the Food and Drug Administration. Such compounds can be used in conjuction with crystal structure information on PDE5A to develop improved inhibitors.

The following are among the examples of descriptions of such compounds. The compounds described in the publications listed can be used in the present invention to develop improved PDE5 inhibitors, e.g., inhibitors with improved affinity, activity, and/or specificity properties. Bunnage et al., U.S. Pat. No. 6,333,330, U.S. Pat. No. 6,407,114, and U.S. Patent Publication 2001/0039271, all entitled PYRAZOLOPYRIMIDINONE CGMP PDE5 INHIBITORS FOR THE TREATEMENT OF SEXUAL DYSFUNCTION, describe some pyrazolopyrimidinone compounds and their synthesis, preparation of pharmaceutical compositions, and administration. Fryburg et al., U.S. Patent Application Publication 2002/0165237, entitled TREATMENT OF THE INSULIN RESISTANCE SYNDROME, lists a variety of PDE5 inhibitors, including compounds described in EP-A-0463756, EP-A-0526004, WO 93/06104, 93/07149, WO 93/12095. WO 94/00453, WO 98/49166, WO 99/54333, EP-A-0995751, WO 00/24745, EP-A-995750, WO 95/19978, and WO 93/07124, along with methods for formulating and administering pharmaceutical compositions. Bombrun, U.S. Pat. No. 6,043,252, entitled CARBOLINE DERIVATIVES, describes PDE5 inhibitors that are carboline derivatives. Allerton, U.S. Patent Application Publication 2002/0173502, entitled PHARMACEUTICALLY ACTIVE COMPOUNDS, describes as PDE5 inhibitors certain compounds that include four heterocyclic groups. Sperl et al., U.S. Pat. No. 6,066,634 describes substituted condensation products of N-benzyl-3-indenylacetamides herocyclic aldehydes and their use in treatment of neoplasias. Additional PDE5 inhibitors are described in Maw, U.S. Pat. No. 6,503,908; Maw et al., U.S. Pat. No. 6,440,982; Daugan et al., U.S. Pat. No. 6,143,757; Daugan et al., U.S. Pat. No. 6,143,746; Gonzalez et al., U.S. Patent Application Publication 2002/0058606. Benzimidazole derivatives with PDE5 inhibitor activity, and their preparation and use are described in Yamasaki et al., U.S. Pat. No. 6,166,219. All of the above references are incorporated herein by reference in their entireties.

Exemplary Diseases Associated with PDE5A.

PDE5A has been correlated with several conditions in which inhibition of PDE5A is useful. Best known is the involvement of PDE5A in treatment of erectile dysfunction. Erection is largely a haemodynamic event that is regulated by fascular tone and blood-flow balance in the penis. Because cGMP levels modulate vascular tone, PDE5A is a useful target for intervention. When a man is sexually stimulated, nitric oxide (NO) is released from non-cholinergic, non-adrenergic neurons in the penis as well as from endothelial cells. NO diffuses into cells, where it activates soluble guanylyl cyclase, the enzyme that converts GTP to cGMP. The cGMP then stimulates PKG, which initiates a protein phosphorylation cascade. This results in a descrease in intracellular levels of cancium oins, leading ultimately to dilation of the arteries that bring blood to the penis and compression o the spongy corpus-cavernosum tissue. This compression contracts veins, which reduces the outflow of blood and increases intracavernosal pressure resulting in an erection. A PDE5A inhibitor retards enzymatic hydrolysis of cGMP in the corpus cavemosum, leading to the same outcome. (Rotella, 2002, Phosphodiesterase 5 inhibitors: Current status and potential applications, Nature Reviews 1:674-682.) (See also, Taher et al., J. Urol. 149:285A (1993); Murray, DN&P 6(3):150-156 (1993); Emmick et al., U.S. Pat. No. 6,451,807, entitled METHODS OF TREATING SEXUAL DYSFUNCTION IN AN INDIVIDUAL SUFFEREING FROM A RETINAL DISEASE, CLASS 1 CONGESTIVE HEART FAILURE, OR MYOCARDIAL INFARCTION USING A PDE5 INHBITOR.)

In addition to treating erectile dysfunction, PDE inhibitors are described for use in treatment of premature ejaculation in individuals with normal erectile function. Boolell, U.S. Patent Application Publication 2002/0091129.

The use of PDE5A inhibitors in treatment of cystic fibrosis has also been indicated.

Treatment of Parkinson's Disease (PD) using PDE5 inhibitors has also been indicated. For example, Roylance, U.S. Pat. No. 6,492,371, indicates that PDE5 inhibitors are useful in methods for preventing and/or slowing the progression of PD or reducing or eliminating clinical symptoms of PD.

Watkins et al., U.S. Patent Application Publication 2002/0128171 describes the use of PDE5 inhibitors to treat gastrointestinal disorders, such as disorders characterized by hypomobility or hypermobility of small intesting, large intestine, colon, esphagus, or stomach.

The vasodilatory effects of PDE5A inhibitors allows their use in connection with some circulatory-disorders. In conjunction with a prostaglandin analogue (e.g., iloprost), a PDE5A inhibitor can enhance reduction of pulmonary arterial pressure, allowing such use in patients with pulmonary hypertension.

Subarachnoid haemorrhage is a significant cause of stroke in many patients. It often occurs as a consequence of reduced responsiveness to NO in cerebral srteries. To counter this effect, PDE5A inhibitors can elevate cellular levels of cGMP in cerbral arteries, thereby at least partially correcting the vascular dysfunction.

Shahinpoor et al., U.S. Patent Application Publication 2002/0168424 describes the use of PDE5 inhibitors in conjunction with a nitric oxid donor for treatment of glaucoma. The publication indicates the drugs work synergistically to reduce intraocular pressure.

PDE5A inhibitors also moderate platelet aggregation in a dose-dependent manner.

Fryburg et al., U.S. Patent Application Publication 2002/0165,237, entitled TREATEMENT OF THE INSULIN RESISTANCE SYNDROME, describes the use of selective PDE5 inhibitors in the curative, palliative, or prophylactic treatment of insulin resistance syndrome (also referred to as Syndrome X and Metabolic Syndrome). Insulin resistance syndrome means the concomitant existence of two or more of: dyslipidemia, hypertension, type 2 diabetes mellitus or a family history of type 2 diabetes mellitus, hyperuricaemia, and/or gout, a pro-coagulant state, atheroslerosis, truncal obesity.

Thompson et al., U.S. Pat. No. 6,130,053, entitled METHODS FOR SELECTING COMPOUNDS FOR INHIBITON OF NEOPLASTIC LESIONS, and Thompson et al., U.S. Patent Application Publication 2002/0009764, entitled METHODS FOR IDENTIFYING COMPOUNDS FOR INHIBITON OF NEOPLASTIC LESIONS, AND PHARMACEUTICAL COMPOSITIONS CONTAINING SUCH COMPOUNDS describes the use of PDE5 inhibitors in conjunction with inhibition of PDE2 activity, leading to cell apoptosis, and methods for identifying useful compounds. See also, Pamakcu et al., U.S. Pat. No. 6,500,610, entitled METHODS FOR IDENTIFYING COMPOUNDS FOR INHIBITING NEOPLASTIC LESIONS, AND PHARMACEUTICAL COMPOSITIONS CONTAINING SUCH COMPOUNDS. Similarly, Whitehead, U.S. Pat. No. 6,479,493 describes the use of PDE2 inhibition combined with PDE5 inhibition for treatment of Type 1 diabetes, and describes compouns fo that purpose. Use of combination PDE2 and PDE5 inhibition is also described in Earle et al., U.S. Pat. No. 6,465,494, entitled METHODS FOR TREATMENT OF CYSTIC FIBROSIS.

Bombrun, U.S. Pat. No. 6,043,252 indicates that PDE5 inhibitors are useful for treatment of stable, unstable, and variant (Prinzmetal) angina, hypertension, pulmonary hypertenion, chronic obstructive pulmonary disease, congestive heart failure, acute respiratory distress syndrome, acute and chronic renal failure, atherosclerosis, conditions of reduced blood vessel patency (e.g., post-PTCA or post-bypass graft stenosis), peripheral vascular disease, vascular disorders such as Raynaud's disease, myocardial infarction, prophylaxis of stroke, stroke, bronchitis, chronic asthma, allergic asthma, allergic rhinitis, hypertrophy, male and female erictile dysfunction, and diseases characterized by disorders of gut motility.

Davies et al., U.S. Patent Application Publication 2002/0065286 describes the use of PDE5 inhibitors in wound treatment, such chronic wounds of non-diabetic origin, as well as acute wounds, such as in the elderly.

The present methods can be used for developing ligands for treating one or more of the diseases and conditions above, or for other diseases or conditions for which PDE5A modulation is found useful.

II. Crystalline PDE5A

Crystalline PDE5A (e.g., human PDE5A) include native crystals, phosphodiesterase domain crystals, derivative crystals and co-crystals. The native crystals generally comprise substantially pure polypeptides corresponding to PDE5A in crystalline form. PDE5A phosphodiesterase domain crystals generally comprise substantially pure PDE5A phosphodiesterase domain in crystalline form. In connection with the development of inhibitors of PDE5A phosphodiesterase function, it is advantageous to use PDE5A phosphodiesterase domain for structural determination, because use of the reduced sequence simplifies structure determination. To be useful for this purpose, the phosphodiesterase domain should be active and/or retain native-type binding, thus indicating that the phosphodiesterase domain takes on substantially normal 3D structure.

It is to be understood that the crystalline phosphodiesterases and phosphodiesterase domains of the invention are not limited to naturally occurring or native phosphodiesterase. Indeed, the crystals of the invention include crystals of mutants of native phosphodiesterases. Mutants of native phosphodiesterases are obtained by replacing at least one amino acid residue in a native phosphodiesterase with a different amino acid residue, or by adding or deleting amino acid residues within the native polypeptide or at the N- or C-terminus of the native polypeptide, and have substantially the same three-dimensional structure as the native phosphodiesterase from which the mutant is derived.

By having substantially the same three-dimensional structure is meant having a set of atomic structure coordinates that have a root-mean-square deviation of less than or equal to about 2A when superimposed with the atomic structure coordinates of the native phosphodiesterase from which the mutant is derived when at least about 50% to 100% of the Ca atoms of the native phosphodiesterase domain are included in the superposition.

Amino acid substitutions, deletions and additions which do not significantly interfere with the three-dimensional structure of the phosphodiesterase will depend, in part, on the region of the phosphodiesterase where the substitution, addition or deletion occurs. In highly variable regions of the molecule, non-conservative substitutions as well as conservative substitutions may be tolerated without significantly disrupting the three-dimensional, structure of the molecule. In highly conserved regions, or regions containing significant secondary structure, conservative amino acid substitutions are preferred. Such conserved and variable regions can be identified by sequence alignment of PDE5A with other phosphodiesterases. Such alignment of PDE5A phosphodiesterase domain along with a number of other phosphodiesterase domains is provided in Table 3.

Conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

For phosphodiesterases obtained in whole or in part by chemical synthesis, the selection of amino acids available for substitution or addition is not limited to the genetically encoded amino acids. Indeed, the mutants described herein may contain non-genetically encoded amino acids. Conservative amino acid substitutions for many of the commonly known non-genetically encoded amino acids are well known in the art. Conservative substitutions for other amino acids can be determined based on their physical properties as compared to the properties of the genetically encoded amino acids.

In some instances, it may be particularly advantageous or convenient to substitute, delete and/or add amino acid residues to a native phosphodiesterase in order to provide convenient cloning sites in cDNA encoding the polypeptide, to aid in purification of the polypeptide, and for crystallization of the polypeptide. Such substitutions, deletions and/or additions which do not substantially alter the three dimensional structure of the native phosphodiesterase domain will be apparent to those of ordinary skill in the art.

It should be noted that the mutants contemplated herein need not all exhibit phosphodiesterase activity. Indeed, amino acid substitutions, additions or deletions that interfere with the phosphodiesterase activity but which do not significantly alter the three-dimensional structure of the domain are specifically contemplated by the invention. Such crystalline polypeptides, or the atomic structure coordinates obtained therefrom, can be used to identify compounds that bind to the native domain. These compounds can affect the activity of the native domain.

The derivative crystals of the invention can comprise a crystalline phosphodiesterase polypeptide in covalent association with one or more heavy metal atoms. The polypeptide may correspond to a native or a mutated phosphodiesterase. Heavy metal atoms useful for providing derivative crystals include, by way of example and not limitation, gold, mercury, selenium, etc.

The co-crystals of the invention generally comprise a crystalline phosphodiesterase domain polypeptide in association with one or more compounds. The association may be covalent or non-covalent. Such compounds include, but are not limited to, cofactors, substrates, substrate analogues, inhibitors, allosteric effectors, etc.

Exemplary mutations for PDE5A family phosphodiesterases include mutations making the phosphodiesterase active site more like the active site of PDE6 or PDE 11. Such insertion is useful, for example, to assist in using PDE5A to model PDE6 or PDE 1 1. Mutations at other sites can likewise be carried out, e.g., to make a mutated PDE5A more similar to another phosphodiesterase for structure modeling and/or compound fitting purposes, such as a phosphodiesterase in the phosphodiesterase domain alignment in Table 3.

In addition to the PDE5A crystal structure described herein, a crystal-based structure of PDE5A catalytic domain is described in Brown et al., PCT Application PCT/IB02/04426, International Publication WO 03/038080. That structure (and associated atomic coordinate sets), as well as other structures and atomic coordinate sets that may be obtained can also be used as described herein.

III. Three Dimensional Structure Determination Using X-Ray Crystallography

X-ray crystallography is a method of solving the three dimensional structures of molecules. The structure of a molecule is calculated from X-ray diffraction patterns using a crystal as a diffraction grating. Three dimensional structures of protein molecules arise from crystals grown from a concentrated aqueous solution of that protein. The process of X-ray crystallography can include the following steps:
(a) synthesizing and isolating (or otherwise obtaining) a polypeptide;
(b) growing a crystal from an aqueous solution comprising the polypeptide with or without a modulator; and
(c) collecting X-ray diffraction patterns from the crystals, determining unit cell dimensions and symmetry, determining electron density, fitting the amino acid sequence of the polypeptide to the electron density, and refining the structure.

Production of Polypeptides

The native and mutated phosphodiesterase polypeptides described herein may be chemically synthesized in whole or part using techniques that are well-known in the art (see, e.g., Creighton (1983) Biopolymers 22(1):49-58).

Alternatively, methods which are well known to those skilled in the art can be used to construct expression vectors containing the native or mutated phosphodiesterase polypeptide coding sequence and appropriate transcriptional/translational control signals. These methods include in vitro recombinant DNA techniques, synthetic techniques and in vivo recombination/genetic recombination. See, for example, the techniques described in Maniatis, T (1989). Molecular cloning: A laboratory Manual. Cold Spring Harbor Laboratory, New York. Cold Spring Harbor Laboratory Press; and Ausubel, F. M. et al. (1994) Current Protocols in Molecular Biology. John Wiley & Sons, Secaucus, N.J.

A variety of host-expression vector systems may be utilized to express the phosphodiesterase coding sequence. These include but are not limited to microorganisms such as bacteria transformed with recombinant bacteriophage DNA, plasmid DNA or cosmid DNA expression vectors containing the phosphodiesterase domain coding sequence; yeast transformed with recombinant yeast expression vectors containing the phosphodiesterase domain coding sequence; insect cell systems infected with recombinant virus expression vectors (e.g., baculovirus) containing the phosphodiesterase domain coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the phosphodiesterase domain coding sequence; or animal cell systems. The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the phosphodiesterase domain DNA, SV40-, BPV- and EBV-based vectors may be used with an appropriate selectable marker.

Exemplary methods describing methods of DNA manipulation, vectors, various types of cells used, methods of incorporating the vectors into the cells, expression techniques, protein purification and isolation methods, and protein concentration methods are disclosed in detail in PCT publication WO 96/18738. This publication is incorporated herein by reference in its entirety, including any drawings. Those skilled in the art will appreciate that such descriptions are applicable to the present invention and can be easily adapted to it.

Crystal Growth

Crystals are grown from an aqueous solution containing the purified and concentrated polypeptide by a variety of techniques. These techniques include batch, liquid, bridge, dialysis, vapor diffusion, and hanging drop methods. McPherson (1982) John Wiley, New York; McPherson (1990) Eur. J. Biochem. 189:1-23; Webber (1991) Adv. Protein Chem. 41:1-36, incorporated by reference herein in their entireties, including all figures, tables, and drawings.

The native crystals of the invention are, in general, grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

For crystals of the invention, exemplary crystallization conditions are described in the Examples. Those of ordinary skill in the art will recognize that the exemplary crystallization conditions can be varied. Such variations may be used alone or in combination. In addition, other crystallization conditions may be found, e.g., by using crystallization screening plates to identify such other conditions. Those alternate conditions can then be optimized if needed to provide larger or better quality crystals.

Derivative crystals of the invention can be obtained by soaking native crystals in mother liquor containing salts of heavy metal atoms. It has been found that soaking a native crystal in a solution containing about 0.1 mM to about 5 mM thimerosal, 4-chloromeruribenzoic acid or KAu(CN)₂ for about 2 hr to about 72 hr provides derivative crystals suitable for use as isomorphous replacements in determining the X-ray crystal structure of PDE5A.

Co-crystals of the invention can be obtained by soaking a native crystal in mother liquor containing compound that binds the phosphodiesterase, or can be obtained by co-crystallizing the phosphodiesterase polypeptide in the presence of a binding compound.

Generally, co-crystallization of phosphodiesterase and binding compound can be accomplished using conditions identified for crystallizing the corresponding phosphodiesterase without binding compound. It is advantageous if a plurality of different crystallization conditions have been identified for the phosphodiesterase, and these can be tested to determine which condition gives the best co-crystals. It may also be benficial to optimize the conditions for co-crystallization. Alternatively, new crystallization conditions can be determined for obtaining co-crystals, e.g., by screening for crystallization and then optimizing those conditions. Exemplary co-crystallization conditions are provided in the Examples.

Determining Unit Cell Dimensions and the Three Dimensional Structure of a Polypeptide or Polypeptide Complex

Once the crystal is grown, it can be placed in a glass capillary tube or other mounting device and mounted onto a holding device connected to an X-ray generator and an X-ray detection device. Collection of X-ray diffraction patterns are well documented by those in the art. See, e.g., Ducruix and Geige, (1992), IRL Press, Oxford, England, and references cited therein. A beam of X-rays enters the crystal and then diffracts from the crystal. An X-ray detection device can be utilized to record the diffraction patterns emanating from the crystal. Although the X-ray detection device on older models of these instruments is a piece of film, modem instruments digitally record X-ray diffraction scattering. X-ray sources can be of various types, but advantageously, a high intensity source is used, e.g., a synchrotron beam source.

Methods for obtaining the three dimensional structure of the crystalline form of a peptide molecule or molecule complex are well known in the art. See, e.g., Ducruix and Geige, (1992), IRL Press, Oxford, England, and references cited therein. The following are steps in the process of determining the three dimensional structure of a molecule or complex from X-ray diffraction data.

After the X-ray diffraction patterns are collected from the crystal, the unit cell dimensions and orientation in the crystal can be determined. They can be determined from the spacing between the diffraction emissions as well as the patterns made from these emissions. The unit cell dimensions are characterized in three dimensions in units of Angstroms (one Å=10⁻¹⁰ meters) and by angles at each vertices. The symmetry of the unit cell in the crystals is also characterized at this stage. The symmetry of the unit cell in the crystal simplifies the complexity of the collected data by identifying repeating patterns. Application of the symmetry and dimensions of the unit cell is described below.

Each diffraction pattern emission is characterized as a vector and the data collected at this stage of the method determines the amplitude of each vector. The phases of the vectors can be determined using multiple techniques. In one method, heavy atoms can be soaked into a crystal, a method called isomorphous replacement, and the phases of the vectors can be determined by using these heavy atoms as reference points in the X-ray analysis. (Otwinowski, (1991), Daresbury, United Kingdom, 80-86). The isomorphous replacement method usually utilizes more than one heavy atom derivative.

In another method, the amplitudes and phases of vectors from a crystalline polypeptide with an already determined structure can be applied to the amplitudes of the vectors from a crystalline polypeptide of unknown structure and consequently determine the phases of these vectors. This second method is known as molecular replacement and the protein structure which is used as a reference must have a closely related structure to the protein of interest. (Naraza (1994) Proteins 11:281-296). Thus, the vector information from a phosphodiesterase of known structure, such as those reported herein, are useful for the molecular replacement analysis of another phosphodiesterase with unknown structure.

Once the phases of the vectors describing the unit cell of a crystal are determined, the vector amplitudes and phases, unit cell dimensions, and unit cell symmetry can be used as terms in a Fourier transform function. The Fourier transform function calculates the electron density in the unit cell from these measurements. The electron density that describes one of the molecules or one of the molecule complexes in the unit cell can be referred to as an electron density map. The amino acid structures of the sequence or the molecular structures of compounds complexed with the crystalline polypeptide may then be fitted to the electron density using a variety of computer programs. This step of the process is sometimes referred to as model building and can be accomplished by using computer programs such as Turbo/FRODO or “O”. (Jones (1985) Methods in Enzymology 115:157-171).

A theoretical electron density map can then be calculated from the amino acid structures fit to the experimentally determined electron density. The theoretical and experimental electron density maps can be compared to one another and the agreement between these two maps can be described by a parameter called an R-factor. A low value for an R-factor describes a high degree of overlapping electron density between a theoretical and experimental electron density map.

The R-factor is then minimized by using computer programs that refine the theoretical electron density map. A computer program such as X-PLOR can be used for model refinement by those skilled in the art. (Brünger (1992) Nature 355:472-475.) Refinement may be achieved in an iterative process. A first step can entail altering the conformation of atoms defined in an electron density map. The conformations of the atoms can be altered by simulating a rise in temperature, which will increase the vibrational frequency of the bonds and modify positions of atoms in the structure. At a particular point in the atomic perturbation process, a force field, which typically defines interactions between atoms in terms of allowed bond angles and bond lengths, Van der Waals interactions, hydrogen bonds, ionic interactions, and hydrophobic interactions, can be applied to the system of atoms. Favorable interactions may be described in terms of free energy and the atoms can be moved over many iterations until a free energy minimum is achieved. The refinement process can be iterated until the R-factor reaches a minimum value.

The three dimensional structure of the molecule or molecule complex is described by atoms that fit the theoretical electron density characterized by a minimum R-value. A file can then be created for the three dimensional structure that defines each atom by coordinates in three dimensions. An example of such a structural coordinate file is shown in Table 1.

IV. Structures of PDE5A

The present invention provides high-resolution three-dimensional structures and atomic structure coordinates of crystalline PDE5A phosphodiesterase domain and PDE5A phosphodiesterase domain co-complexed with exemplary binding compounds as determined by X-ray crystallography. The methods used to obtain the structure coordinates are provided in the examples. The atomic structure coordinates of crystalline PDE5A are listed in Table 1. Co-crystal coordinates can be used in the same way, e.g., in the various aspects described herein, as coordinates for the protein by itself.

Those having skill in the art will recognize that atomic structure coordinates as determined by X-ray crystallography are not without error. Thus, it is to be understood that any set of structure coordinates obtained for crystals of PDE5A, whether native crystals, phosphodiesterase domain crystals, derivative crystals or co-crystals, that have a root mean square deviation (“r.m.s.d.”) of less than or equal to about 1.5 Å when superimposed, using backbone atoms (N, C_{α}, C and 0), on the structure coordinates listed in Table 1 are considered to be identical with the structure coordinates listed in the Table 1 when at least about 50% to 100% of the backbone atoms of PDE5A are included in the superposition.

As indicated above, a crystal-based PDE5A catalytic domain structure is described in Brown et al., PCT Application PCT/IB02/04426, International Publication WO 03/038080.

V. Uses of the Crystals and Atomic Structure Coordinates

The crystals of the invention, and particularly the atomic structure coordinates obtained therefrom, have a wide variety of uses. For example, the crystals described herein can be used as a starting point in any of the methods of use for phosphodiesterases known in the art or later developed. Such methods of use include, for example, identifying molecules that bind to the native or mutated catalytic domain of phosphodiesterases. The crystals and structure coordinates are particularly useful for identifying ligands that modulate phosphodiesterase activity as an approach towards developing new therapeutic agents. In particular, the crystals and structural information are useful in methods for ligand development utilizing molecular scaffolds.

The structure coordinates described herein can be used as phasing models for determining the crystal structures of additional phosphodiesterases, as well as the structures of co-crystals of such phosphodiesterases with ligands such as inhibitors, agonists, antagonists, and other molecules. The structure coordinates, as well as models of the three-dimensional structures obtained therefrom, can also be used to aid the elucidation of solution-based structures of native or mutated phosphodiesterases, such as those obtained via NMR.

VI. Electronic Representations of Phosphodiesterase Structures

Structural information of phosphodiesterases or portions of phosphodiesterases (e.g., phosphodiesterase active sites) can be represented in many different ways. Particularly useful are electronic representations, as such representations allow rapid and convenient data manipulations and structural modifications. Electronic representations can be embedded in manydifferent storage or memory media, frequently computer readable media. Examples include without limitations, computer random access memory (RAM), floppy disk, magnetic hard drive, magnetic tape (analog or digital), compact disk (CD), optical disk, CD-ROM, memory card, digital video disk (DVD), and others. The storage medium can be separate or part of a computer system. Such a computer system may be a dedicated, special purpose, or embedded system, such as a computer system that forms part of an X-ray crystallography system, or may be a general purpose computer (which may have data connection with other equipment such as a sensor device in an X-ray crystallographic system. In many cases, the information provided by such electronic representations can also be represented physically or visually in two or three dimensions, e.g., on paper, as a visual display (e.g., on a computer monitor as a two dimensional or pseudo-three dimensional image) or as a three dimensional physical model. Such physical representations can also be used, alone or in connection with electronic representations. Exemplary useful representations include, but are not limited to, the following:

Atomic Coordinate Representation

One type of representation is a list or table of atomic coordinates representing positions of particular atoms in a molecular structure, portions of a structure, or complex (e.g., a co-crystal). Such a representation may also include additional information, for example, information about occupancy of particular coordinates. One such atomic coordinate representation contains the coordinate information of Table 1 in electronic form.

Energy Surface or Surface of Interaction Representation

Another representation is an energy surface representation, e.g., of an active site or other binding site, representing an energy surface for electronic and steric interactions. Such a representation may also include other features. An example is the inclusion of representation of a particular amino acid residue(s) or group(s) on a particular amino acid residue(s), e.g., a residue or group that can participate in H-bonding or ionic interaction. Such energy surface representations can be readily generated from atomic coordinate representations using any of a variety of available computer programs.

Structural Representation

Still another representation is a structural representation, i.e., a physical representation or an electronic representation of such a physical representation. Such a structural representation includes representations of relative positions of particular features of a molecule or complex, often with linkage between structural features. For example, a structure can be represented in which all atoms are linked; atoms other than hydrogen are linked; backbone atoms, with or without representation of sidechain atoms that could participate in significant electronic interaction, are linked; among others. However, not all features need to be linked. For example, for structural representations of portions of a molecule or complex, structural features significant for that feature may be represented (e.g., atoms of amino acid residues that can have significant binding interation with a ligand at a binding site. Those amino acid residues may not be linked with each other.

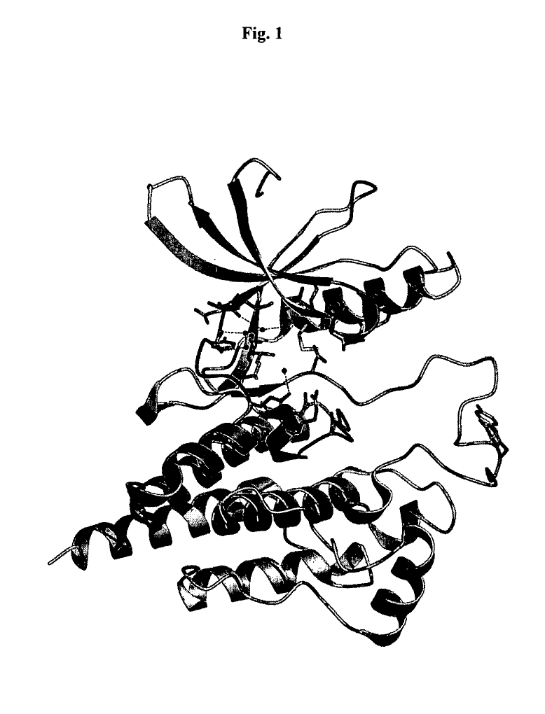

A structural representation can also be a schematic representation. For example, a schematic representation can represent secondary and/or tertiary structure in a schematic manner. Within such a schematic representation of a polypeptide, a particular amino acid residue(s) or group(s) on a residue(s) can be included, e.g., conserved residues in a binding site, and/or residue(s) or group(s) that may interact with binding compounds. Electronic structural representations can be generated, for example, from atomic coordinate information using computer programs designed for that function and/or by constructing an electronic representation with manual input based on interpretation of another form of structural information. Physical representations can be created, for example, by printing an image of a computer-generated image or by constructing a 3D model. An example of such a printed representation is the ribbon diagram presented in FIG. 1.

VII. Structure Determination for Phosphodiesterases with Unknown Structure Using Structural Coordinates

Structural coordinates, such as those set forth in Table 1, can be used to determine the three dimensional structures of phosphodiesterases with unknown structure. The methods described below can apply structural coordinates of a polypeptide with known structure to another data set, such as an amino acid sequence, X-ray crystallographic diffraction data, or nuclear magnetic resonance (NMR) data. Preferred embodiments of the invention relate to determining the three dimensional structures of other PDE5A phosphodiesterases, other phosphodiesterases, and related polypeptides.

Structures Using Amino Acid Homology

Homology modeling is a method of applying structural coordinates of a polypeptide of known structure to the amino acid sequence of a polypeptide of unknown structure. This method is accomplished using a computer representation of the three dimensional structure of a polypeptide or polypeptide complex, the computer representation of amino acid sequences of the polypeptides with known and unknown structures, and standard computer representations of the structures of amino acids. Homology modeling generally involves (a) aligning the amino acid sequences of the polypeptides with and without known structure; (b) transferring the coordinates of the conserved amino acids in the known structure to the corresponding amino acids of the polypeptide of unknown structure; refining the subsequent three dimensional structure; and (d) constructing structures of the rest of the polypeptide. One skilled in the art recognizes that conserved amino acids between two proteins can be determined from the sequence alignment step in step (a).

The above method is well known to those skilled in the art. (Greer (1985) Science 228:1055; Blundell et al A(1988) Eur. J. Biochem. 172:513. An exemplary computer program that can be utilized for homology modeling by those skilled in the art is the Homology module in the Insight II modeling package distributed by Accelerys Inc.

Alignment of the amino acid sequence is accomplished by first placing the computer representation of the amino acid sequence of a polypeptide with known structure above the amino acid sequence of the polypeptide of unknown structure. Amino acids in the sequences are then compared and groups of amino acids that are homologous (e.g., amino acid side chains that are similar in chemical nature—aliphatic, aromatic, polar, or charged) are grouped together. This method will detect conserved regions of the polypeptides and account for amino acid insertions or deletions. Such alignment and/or can also be performed fully electronically using sequence alignment and analyses software.

Once the amino acid sequences of the polypeptides with known and unknown structures are aligned, the structures of the conserved amino acids in the computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions are to be assigned manually by either using standard peptide geometries or molecular simulation techniques, such as molecular dynamics. The final step in the process is accomplished by refining the entire structure using molecular dynamics and/or energy minimization. The homology modeling method is well known to those skilled in the art and has been practiced using different protein molecules. For example, the three dimensional structure of the polypeptide corresponding to the catalytic domain of a serine/threonine protein kinase, myosin light chain protein kinase, was homology modeled from the cAMP-dependent protein kinase catalytic subunit. (Knighton et al. (1992) Science 258:130-135.)

Structures Using Molecular Replacement

Molecular replacement is a method of applying the X-ray diffraction data of a polypeptide of known structure to the X-ray diffraction data of a polypeptide of unknown sequence. This method can be utilized to define the phases describing the X-ray diffraction data of a polypeptide of unknown structure when only the amplitudes are known. X-PLOR is a commonly utilized computer software package used for molecular replacement. Brunger (1992) Nature 355:472-475. AMORE is another program used for molecular replacement. Navaza (1994) Acta Crystallogr. A50:157-163. Preferably, the resulting structure does not exhibit a root-mean-square deviation of more than 3 Å.

A goal of molecular replacement is to align the positions of atoms in the unit cell by matching electron diffraction data from two crystals. A program such as X-PLOR can involve four steps. A first step can be to determine the number of molecules in the unit cell and define the angles between them. A second step can involve rotating the diffraction data to define the orientation of the molecules in the unit cell. A third step can be to translate the electron density in three dimensions to correctly position the molecules in the unit cell. Once the amplitudes and phases of the X-ray diffraction data is determined, an R-factor can be calculated by comparing electron diffraction maps calculated experimentally from the reference data set and calculated from the new data set. An R-factor between 30-50% indicates that the orientations of the atoms in the unit cell are reasonably determined by this method. A fourth step in the process can be to decrease the R-factor to roughly 20% by refining the new electron density map using iterative refinement techniques described herein and known to those or ordinary skill in the art.

Structures Using NMR Data

Structural coordinates of a polypeptide or polypeptide complex derived from X-ray crystallographic techniques can be applied towards the elucidation of three dimensional structures of polypeptides from nuclear magnetic resonance (NMR) data. This method is used by those skilled in the art. (Wuthrich, (1986), John Wiley and Sons, New York: 176-199; Pflugrath et al. (1986) J. Mol. Biol. 189:383-386; Kline et al. (1986) J. Mol. Biol. 189:377-382.) While the secondary structure of a polypeptide is often readily determined by utilizing two-dimensional NMR data, the spatial connections between individual pieces of secondary structure are not as readily determinable. The coordinates defining a three-dimensional structure of a polypeptide derived from X-ray crystallographic techniques can guide the NMR spectroscopist to an understanding of these spatial interactions between secondary structural elements in a polypeptide of related structure.

The knowledge of spatial interactions between secondary structural elements can greatly simplify Nuclear Overhauser Effect (NOE) data from two-dimensional NMR experiments. Additionally, applying the crystallographic coordinates after the determination of secondary structure by NMR techniques only simplifies the assignment of NOEs relating to particular amino acids in the polypeptide sequence and does not greatly bias the NMR analysis of polypeptide structure. Conversely, using the crystallographic coordinates to simplify NOE data while determining secondary structure of the polypeptide would bias the NMR analysis of protein structure.

VIII. Structure-Based Design of Modulators of Phosphodiesterase Function Utilizing Structural Coordinates

Structure-based modulator design and identification methods are powerful techniques that can involve searches of computer databases containing a wide variety of potential modulators and chemical functional groups. The computerized design and identification of modulators is useful as the computer databases contain more compounds than the chemical libraries, often by an order of magnitude. For reviews of structure-based drug design and identification (see Kuntz et al. (1994), Acc. Chem. Res. 27:117; Guida (1994) Current Opinion in Struc. Biol. 4: 777; Colman (1994) Current Opinion in Struc. Biol. 4: 868).

The three dimensional structure of a polypeptide defined by structural coordinates can be utilized by these design methods, for example, the structural coordinates of Table 1. In addition, the three dimensional structures of phosphodiesterases determined by the homology, molecular replacement, and NMR techniques described herein can also be applied to modulator design and identification methods.

For identifying modulators, structural information for a native phosphodiesterase, in particular, structural information for the active site of the phosphodiesterase, can be used. However, it may be advantageous to utilize structural information from one or more co-crystals of the phosphodiesterase with one or more binding compounds. It can also be advantageous if the binding compound has a structural core in common with test compounds.

Design by Searching Molecular Data Bases

One method of rational design searches for modulators by docking the computer representations of compounds from a database of molecules. Publicly available databases include, for example:
a) ACD from Molecular Designs Limited
b) NCI from National Cancer Institute
c) CCDC from Cambridge Crystallographic Data Center
d) CAST from Chemical Abstract Service
e) Derwent from Derwent Information Limited
f) Maybridge from Maybridge Chemical Company LTD
g) Aldrich from Aldrich Chemical Company
h) Directory of Natural Products from Chapman & Hall

One such data base (ACD distributed by Molecular Designs Limited Information Systems) contains compounds that are synthetically derived or are natural products. Methods available to those skilled in the art can convert a data set represented in two dimensions to one represented in three dimensions. These methods are enabled by such computer programs as CONCORD from Tripos Associates or DE-Converter from Molecular Simulations Limited.

Multiple methods of structure-based modulator design are known to those in the art. (Kuntz et al., (1982), J. Mol. Biol. 162. 269; Kuntz et aZ., (1994), Acc. Chern. Res. 27. 117; Meng et al., (1992), J. Compt. Chem. 13: 505; Bohm, (1994), J. Comp. Aided Molec. Design 8: 623.)

A computer program widely utilized by those skilled in the art of rational modulator design is DOCK from the University of California in San Francisco. The general methods utilized by this computer program and programs like it are described in three applications below. More detailed information regarding some of these techniques can be found in the Accelerys User Guide, 1995. A typical computer program used for this purpose can perform a processes comprising the following steps or functions:
(a) remove the existing compound from the protein;
(b) dock the structure of another compound into the active-site using the computer program (such as DOCK) or by interactively moving the compound into the active-site;
(c) characterize the space between the compound and the active-site atoms;
(d) search libraries for molecular fragments which (i) can fit into the empty space between the compound and the active-site, and (ii) can be linked to the compound; and
(e) link the fragments found above to the compound and evaluate the new modified compound.

Part (c) refers to characterizing the geometry and the complementary interactions formed between the atoms of the active site and the compounds. A favorable geometric fit is attained when a significant surface area is shared between the compound and active-site atoms without forming unfavorable steric interactions. One skilled in the art would note that the method can be performed by skipping parts (d) and (e) and screening a database of many compounds.

Structure-based design and identification of modulators of phosphodiesterase function can be used in conjunction with assay screening. As large computer databases of compounds (around 10,000 compounds) can be searched in a matter of hours or even less, the computer-based method can narrow the compounds tested as potential modulators of phosphodiesterase function in biochemical or cellular assays.

The above descriptions of structure-based modulator design are not all encompassing and other methods are reported in the literature and can be used, e.g.:
(1) CAVEAT: Bartlett et al., (1989), in Chemical and Biological Problems in Molecular Recognition, Roberts, S. M.; Ley, S. V.; Campbell, M. M. eds.; Royal Society of Chemistry: Cambridge, pp.182-196.
(2) FLOG: Miller et al., (1994), J. Comp. Aided Molec. Design 8:153.
(3) PRO Modulator: Clark et al., (1995), J. Comp. Aided Molec. Design 9:13.
(4) MCSS: Miranker and Karplus, (1991), Proteins: Structure, Function, and Genetics 11:29.
(5) AUTODOCK: Goodsell and Olson, (1990), Proteins: Structure, Function, and Genetics 8:195.
(6) GRID: Goodford, (1985), J. Med. Chem. 28:849.

Design by Modifying Compounds in Complex with PDE5A

Another way of identifying compounds as potential modulators is to modify an existing modulator in the polypeptide active site. For example, the computer representation of modulators can be modified within the computer representation of a PDE5A active site. Detailed instructions for this technique can be found, for example, in the Accelerys User Manual, 1995 in LUDI. The computer representation of the modulator is typically modified by the deletion of a chemical group or groups or by the addition of a chemical group or groups.

Upon each modification to the compound, the atoms of the modified compound and active site can be shifted in conformation and the distance between the modulator and the active-site atoms may be scored along with any complementary interactions formed between the two molecules. Scoring can be complete when a favorable geometric fit and favorable complementary interactions are attained. Compounds that have favorable scores are potential modulators.

Design by Modifying the Structure of Compounds that Bind PDE5A

A third method of structure-based modulator design is to screen compounds designed by a modulator building or modulator searching computer program. Examples of these types of programs can be found in the Molecular Simulations Package, Catalyst. Descriptions for using this program are documented in the Molecular Simulations User Guide (1995). Other computer programs used in this application are ISIS/HOST, ISIS/BASE, ISIS/DRAW) from Molecular Designs Limited and UNITY from Tripos Associates.

These programs can be operated on the structure of a compound that has been removed from the active site of the three dimensional structure of a compound-phosphodiesterase complex. Operating the program on such a compound is preferable since it is in a biologically active conformation.

A modulator construction computer program is a computer program that may be used to replace computer representations of chemical groups in a compound complexed with a phosphodiesterase or other biomolecule with groups from a computer database. A modulator searching computer program is a computer program that may be used to search computer representations of compounds from a computer data base that have similar three dimensional structures and similar chemical groups as compound bound to a particular biomolecule.

A typical program can operate by using the following general steps:
(a) map the compounds by chemical features such as by hydrogen bond donors or acceptors, hydrophobic/lipophilic sites, positively ionizable sites, or negatively ionizable sites;
(b) add geometric constraints to the mapped features; and
(c) search databases with the model generated in (b).

Those skilled in the art also recognize that not all of the possible chemical features of the compound need be present in the model of (b). One can use any subset of the model to generate different models for data base searches.

Modulator Design Using Molecular Scaffolds

The present invention can also advantageously utilize methods for designing compounds, designated as molecular scaffolds, that can act broadly across families of molecules and/or for using a molecular scaffold to design ligands that target individual or multiple members of those families. Such design using molecular scaffolds is described in Hirth and Milbum, U.S. patent application Ser. No. 10/377,268, which is incorporated herein by reference in its entirety. Such design and development using molecular scaffolds is described, in part, below.

In preferred embodiments, the molecules can be proteins and a set of chemical compounds can be assembled that have properties such that they are 1) chemically designed to act on certain protein families and/or 2) behave more like molecular scaffolds, meaning that they have chemical substructures that make them specific for binding to one or more proteins in a family of interest. Alternatively, molecular scaffolds can be designed that are preferentially active on an individual target molecule.

Useful chemical properties of molecular scaffolds can include one or more of the following characteristics, but are not limited thereto: an average molecular weight below about 350 daltons, or between from about 150 to about 350 daltons, or from about 150 to about 300 daltons; having a clogP below 3; a number of rotatable bonds of less than 4; a number of hydrogen bond donors and acceptors below 5 or below 4; a polar surface area of less than 50 Å²; binding at protein binding sites in an orientation so that chemical substituents from a combinatorial library that are attached to the scaffold can be projected into pockets in the protein binding site; and possessing chemically tractable structures at its substituent attachment points that can be modified, thereby enabling rapid library construction.

By “clog P” is meant the calculated log P of a compound, “P” referring to the partition coefficient between octanol and water.

The term “Molecular Polar Surface Area (PSA)” refers to the sum of surface contributions of polar atoms (usually oxygens, nitrogens and attached hydrogens) in a molecule. The polar surface area has been shown to correlate well with drug transport properties, such as intestinal absorption, or blood-brain barrier penetration.

Additional useful chemical properties of distinct compounds for inclusion in a combinatorial library include the ability to attach chemical moieties to the compound that will not interfere with binding of the compound to at least one protein of interest, and that will impart desirable properties to the library members, for example, causing the library members to be actively transported to cells and/or organs of interest, or the ability to attach to a device such as a chromatography column (e.g., a streptavidin column through a molecule such as biotin) for uses such as tissue and proteomics profiling purposes.

A person of ordinary skill in the art will realize other properties that can be desirable for the scaffold or library members to have depending on the particular requirements of the use, and that compounds with these properties can also be sought and identified in like manner. Methods of selecting compounds for assay are known to those of ordinary skill in the art, for example, methods and compounds described in U.S. Pat. Nos. 6,288,234, 6,090,912, 5,840,485, each of which is hereby incorporated by reference in its entirety, including all charts and drawings.

In various embodiments, the present invention provides methods of designing ligands that bind to a plurality of members of a molecular family, where the ligands contain a common molecular scaffold. Thus, a compound set can be assayed for binding to a plurality of members of a molecular family, e.g., a protein family. One or more compounds that bind to a plurality of family members can be identified as molecular scaffolds. When the orientation of the scaffold at the binding site of the target molecules has been determined and chemically tractable structures have been identified, a set of ligands can be synthesized starting with one or a few molecular scaffolds to arrive at a plurality of ligands, wherein each ligand binds to a separate target molecule of the molecular family with altered or changed binding affinity or binding specificity relative to the scaffold. Thus, a plurality of drug lead molecules can be designed to preferentially target individual members of a molecular family based on the same molecular scaffold, and act on them in a specific manner.

IX. Binding Assays

The methods of the present invention can involve assays that are able to detect the binding of compounds to a target molecule. Such binding is at a statistically significant level, preferably with a confidence level of at least 90%, more preferably at least 95, 97, 98, 99% or greater confidence level that the assay signal represents binding to the target molecule, i.e., is distinguished from background. Preferably controls are used to distinguish target binding from non-specific binding. The assays of the present invention can also include assaying compounds for low affinity binding to the target molecule. A large variety of assays indicative of binding are known for different target types and can be used for this invention. Compounds that act broadly across protein families are not likely to have a high affinity against individual targets, due to the broad nature of their binding. Thus, assays described herein allow for the identification of compounds that bind with low affinity, very low affinity, and extremely low affinity. Therefore, potency (or binding affinity) is not the primary, nor even the most important, indicia of identification of a potentially useful binding compound. Rather, even those compounds that bind with low affinity, very low affinity, or extremely low affinity can be considered as molecular scaffolds that can continue to the next phase of the ligand design process.

By binding with “low affinity” is meant binding to the target molecule with a dissociation constant (k_{d}) of greater than 1 μM under standard conditions. By binding with “very low affinity” is meant binding with a k_{d} of above about 100 μM under standard conditions. By binding with “extremely low affinity” is meant binding at a k_{d} of above about 1 mM under standard conditions. By “moderate affinity” is meant binding with a k_{d} of from about 200 nM to about 1 μM under standard conditions. By “moderately high affinity” is meant binding at a k_{d} of from about 1 nM to about 200 nM. By binding at “high affinity” is meant binding at a k_{d} of below about 1 nM under standard conditions. For example, low affinity binding can occur because of a poorer fit into the binding site of the target molecule or because of a smaller number of non-covalent bonds, or weaker covalent bonds present to cause binding of the scaffold or ligand to the binding site of the target molecule relative to instances where higher affinity binding occurs. The standard conditions for binding are at pH 7.2 at 37° C. for one hour. For example, 100 μl/well can be used in HEPES 50 mM buffer at pH 7.2, NaCl 15 mM, ATP 2 μM, and bovine serum albumin 1 ug/well, 37° C. for one hour.

Binding compounds can also be characterized by their effect on the activity of the target molecule. Thus, a “low activity” compound has an inhibitory concentration (IC₅₀) or excitation concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667$$

To design or discover scaffolds that act broadly across protein families, proteins of interest can be assayed against a compound collection or set. The assays can preferably be enzymatic or binding assays. In some embodiments it may be desirable to enhance the solubility of the compounds being screened and then analyze all compounds that show activity in the assay, including those that bind with low affinity or produce a signal with greater than about three times the standard deviation of the background signal. The assays can be any suitable assay such as, for example, binding assays that measure the binding affinity between two binding partners. Various types of screening assays that can be useful in the practice of the present invention are known in the art, such as those described in U.S. Pat. Nos. 5,763,198, 5,747,276, 5,877,007, 6,243,980, 6,294,330, and 6,294,330, each of which is hereby incorporated by reference in its entirety, including all charts and drawings.

In various embodiments of the assays at least one compound, at least about 5%, at least about 10%, at least about 15%, at least about 20%, or at least about 25% of the compounds can bind with low affinity. In general, up to about 20% of the compounds can show activity in the screening assay and these compounds can then be analyzed directly with high-throughput co-crystallography, computational analysis to group the compounds into classes with common structural properties (e.g., structural core and/or shape and polarity characteristics), and the identification of common chemical structures between compounds that show activity.

The person of ordinary skill in the art will realize that decisions can be based on criteria that are appropriate for the needs of the particular situation, and that the decisions can be made by computer software programs. Classes can be created containing almost any number of scaffolds, and the criteria selected can be based on increasingly exacting criteria until an arbitrary number of scaffolds is arrived at for each class that is deemed to be advantageous.

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121:313-21; Liparoto et al., (1999) Biosensor analysis of the interleukin-2 receptor complex, Journal of Molecular Recognition. 12:316-21; Lipschultz et al., (2000) Experimental design for analysis of complex kinetics using surface plasmon resonance, Methods. 20(3):310-8; Malmqvist., (1999) BIACORE: an affinity biosensor system for characterization of biomolecular interactions, Biochemical Society Transactions 27:335-40; Alfthan, (1998) Surface plasmon resonance biosensors as a tool in antibody engineering, Biosensors & Bioelectronics. 13:653-63; Fivash et al., (1998) BIAcore for macromolecular interaction, Current Opinion in Biotechnology. 9:97-101; Price et al.; (1998) Summary report on the ISOBM TD-4 Workshop: analysis of 56 monoclonal antibodies against the MUC 1 mucin. Tumour Biology 19 Suppl 1: 1-20; Malmqvist et al, (1997) Biomolecular interaction analysis: affinity biosensor technologies for functional analysis of proteins, Current Opinion in Chemical Biology. 1:378-83; O'Shannessy et al., (1996) Interpretation of deviations from pseudo-first-order kinetic behavior in the characterization of ligand binding by biosensor technology, Analytical Biochemistry. 236:275-83; Malmborg et al., (1995) BIAcore as a tool in antibody engineering, Journal of Immunological Methods. 183:7-13; Van Regenmortel, (1994) Use of biosensors to characterize recombinant proteins, Developments in Biological Standardization. 83:143-51; and O'Shannessy, (1994) Determination of kinetic rate and equilibrium binding constants for macromolecular interactions: a critique of the surface plasmon resonance literature, Current Opinions in Biotechnology. 5:65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a sphingolipid target are sought, known ligands of the target can be present in control/calibration assay wells.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., (1972) The Chemist's Companion: A Handbook Of Practical Data, Techniques And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistry, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owickiet al., (1997), Application of Fluorescence Polarization Assays in High-Throughput Screening, Genetic Engineering News, 17:27.

FP is particularly desirable since its readout is independent of the emission intensity (Checovich, W. J., et al., (1995) Nature 375:254-256; Dandliker, W. B., et al., (1981) Methods in Enzymology 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5:77-88.

Fluorophores derived from sphingolipids that may be used in FP assays are commercially available. For example, Molecular Probes (Eugene, Oreg.) currently sells sphingomyelin and one ceramide flurophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan A G, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6:178-182; Mitra et al., (1996) Gene 173:13-17; and Selvin et al., (1995) Meth. Enzymol. 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38:2365-2373; Kahl et al., (1996) Anal. Biochem. 243:282-283; Undenfriend et al., (1987) Anal. Biochem. 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257:112-119).

Assay Compounds and Molecular Scaffolds

Preferred characteristics of a scaffold include being of low molecular weight (e.g., less than 350 Da, or from about 100 to about 350 daltons, or from about 150 to about 300 daltons). Preferably clog P of a scaffold is from −1 to 8, more preferably less than 6, 5, or 4, most preferably less than 3. In particular embodiments the clogP is in a range−1 to an upper limit of 2, 3, 4, 5, 6, or 8; or is in a range of 0 to an upper limit of 2, 3, 4, 5, 6, or 8. Preferably the number of rotatable bonds is less than 5, more preferably less than 4. Preferably the number of hydrogen bond donors and acceptors is below 6, more preferably below 5. An additional criterion that can be useful is a polar surface area of less than 5. Guidance that can be useful in identifying criteria for a particular application can be found in Lipinski et al., (1997) Advanced Drug Delivery Reviews 23 3-25, which is hereby incorporated by reference in its entirety.

A scaffold may preferably bind to a given protein binding site in a configuration that causes substituent moieties of the scaffold to be situated in pockets of the protein binding site. Also, possessing chemically tractable groups that can be chemically modified, particularly through synthetic reactions, to easily create a combinatorial library can be a preferred characteristic of the scaffold. Also preferred can be having positions on the scaffold to which other moieties can be attached, which do not interfere with binding of the scaffold to the protein(s) of interest but do cause the scaffold to achieve a desirable property, for example, active transport of the scaffold to cells and/or organs, enabling the scaffold to be attached to a chromatographic column to facilitate analysis, or another desirable property. A molecular scaffold can bind to a target molecule with any affinity, such as binding at high affinity, moderate affinity, low affinity, very low affinity, or extremely low affinity.

Thus, the above criteria can be utilized to select many compounds for testing that have the desired attributes. Many compounds having the criteria described are available in the commercial market, and may be selected for assaying depending on the specific needs to which the methods are to be applied.

A “compound library” or “library” is a collection of different compounds having different chemical structures. A compound library is screenable, that is, the compound library members therein may be subject to screening assays. In preferred embodiments, the library members can have a molecular weight of from about 100 to about 350 daltons, or from about 150 to about 350 daltons. Examples of libraries are provided aove.

Libraries of the present invention can contain at least one compound than binds to the target molecule at low affinity. Libraries of candidate compounds can be assayed by many different assays, such as those described above, e.g., a fluorescence polarization assay. Libraries may consist of chemically synthesized peptides, peptidomimetics, or arrays of combinatorial chemicals that are large or small, focused or nonfocused. By “focused” it is meant that the collection of compounds is prepared using the structure of previously characterized compounds and/or pharmacophores.

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries of the invention may be purchased on the commercial market or prepared or obtained by any means including, but not limited to, combinatorial chemistry techniques, fermentation methods, plant and cellular extraction procedures and the like (see, e.g., Cwirla et al., (1990) Biochemistry, 87, 6378-6382; Houghten et al., (1991) Nature, 354, 84-86; Lam et al., (1991) Nature, 354, 82-84; Brenner et al., (1992) Proc. Natl. Acad. Sci. USA, 89, 5381-5383; R. A. Houghten, (1993) Trends Genet., 9, 235-239; E. R. Felder, (1994) Chimia, 48, 512-541; Gallop et al., (1994) J. Med. Chem., 37, 1233-1251; Gordon et al., (1994) J. Med. Chem., 37,1385-1401; Carell et al., (1995) Chem. Biol., 3, 171-183; Madden et al., Perspectives in Drug Discovery and Design 2, 269-282; Lebl et al., (1995) Biopolymers, 37 177-198); small molecules assembled around a shared molecular structure; collections of chemicals that have been assembled by various commercial and noncommercial groups, natural products; extracts of marine organisms, fungi, bacteria, and plants.

Preferred libraries can be prepared in a homogenous reaction mixture, and separation of unreacted reagents from members of the library is not required prior to screening. Although many combinatorial chemistry approaches are based on solid state chemistry, liquid phase combinatorial chemistry is capable of generating libraries (Sun CM., (1999) Recent advances in liquid-phase combinatorial chemistry, Combinatorial Chemistry & High Throughput Screening. 2:299-318).

Libraries of a variety of types of molecules are prepared in order to obtain members therefrom having one or more preselected attributes that can be prepared by a variety of techniques, including but not limited to parallel array synthesis (Houghton, (2000) Annu Rev Pharmacol Toxicol 40:273-82, Parallel array and mixture-based synthetic combinatorial chemistry; solution-phase combinatorial chemistry (Merritt, (1998) Comb Chem High Throughput Screen 1(2):57-72, Solution phase combinatorial chemistry, Coe et al., (1998-99) Mol Divers;4(1):31-8, Solution-phase combinatorial chemistry, Sun, (1999) Comb Chem High Throughput Screen 2(6):299-318, Recent advances in liquid-phase combinatorial chemistry); synthesis on soluble polymer (Gravert et al., (1997) Curr Opin Chem Biol 1(1):107-13, Synthesis on soluble polymers: new reactions and the construction of small molecules); and the like. See, e.g., Dolle et al., (1999) J Comb Chem 1(4):235-82, Comprehensive survey of cominatorial library synthesis: 1998. Freidinger R M., (1999) Nonpeptidic ligands for peptide and protein receptors, Current Opinion in Chemical Biology; and Kundu et al., Prog Drug Res;53:89-156, Combinatorial chemistry: polymer supported synthesis of peptide and non-peptide libraries). Compounds may be clinically tagged for ease of identification (Chabala, (1995) Curr Opin Biotechnol 6(6):633-9, Solid-phase combinatorial chemistry and novel tagging methods for identifying leads).

The combinatorial synthesis of carbohydrates and libraries containing oligosaccharides have been described (Schweizer et al., (1999) Curr Opin Chem Biol 3(3):291-8, Combinatorial synthesis of carbohydrates). The synthesis of natural-product based compound libraries has been described (Wessjohann, (2000) Curr Opin Chem Biol 4(3):303-9, Synthesis of natural-product based compound libraries).

Libraries of nucleic acids are prepared by various techniques, including by way of non-limiting example the ones described herein, for the isolation of aptamers. Libraries that include oligonucleotides and polyaminooligonucleotides (Markiewicz et al., (2000) Synthetic oligonucleotide combinatorial libraries and their applications, Farmaco. 55:174-7) displayed on streptavidin magnetic beads are known. Nucleic acid libraries are known that can be coupled to parallel sampling and be deconvoluted without complex procedures such as automated mass spectrometry (Enjalbal C. Martinez J. Aubagnac J L, (2000) Mass spectrometry in combinatorial chemistry, Mass Spectrometry Reviews. 19:139-61) and parallel tagging. (Perrin D M., Nucleic acids for recognition and catalysis: landmarks, limitations, and looking to the future, Combinatorial Chemistry & High Throughput Screening 3:243-69).

Peptidomimetics are identified using combinatorial chemistry and solid phase synthesis (Kim H O. Kahn M., (2000) A merger of rational drug design and combinatorial chemistry: development and application of peptide secondary structure mimetics, Combinatorial Chemistry & High Throughput Screening 3:167-83; al-Obeidi, (1998) Mol Biotechnol 9(3):205-23, Peptide and peptidomimetric libraries. Molecular diversity and drug design). The synthesis may be entirely random or based in part on a known polypeptide.

Polypeptide libraries can be prepared according to various techniques. In brief, phage display techniques can be used to produce polypeptide ligands (Gram H., (1999) Phage display in proteolysis and signal transduction, Combinatorial Chemistry & High Throughput Screening. 2:19-28) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel R W. Allen B. Pavlik P. Marks J D. Bradbury A., (2000) Mass spectral analysis of a protein complex using single-chain antibodies selected on a peptide target: applications to functional genomics, Journal of Molecular Biology 302:285-93; Poul M A. Becerril B. Nielsen U B. Morisson P. Marks J D., (2000) Selection of tumor-specific internalizing human antibodies from phage libraries. Source Journal of Molecular Biology. 301:1149-61; Amersdorfer P. Marks J D., (2001) Phage libraries for generation of anti-botulinum scFv antibodies, Methods in Molecular Biology. 145:219-40; Hughes-Jones N C. Bye J M. Gorick B D. Marks J D. Ouwehand W H., (1999) Synthesis of Rh Fv phage-antibodies using VH and VL germline genes, British Journal of Haematology. 105:811-6; McCall A M. Amoroso A R. Sautes C. Marks J D. Weiner L M., (1998) Characterization of anti-mouse Fc gamma RII single-chain Fv fragments derived from human phage display libraries, Immunotechnology. 4:71-87; Sheets M D. Amersdorfer P. Finnern R. Sargent P. Lindquist E. Schier R. Hemingsen G. Wong C. Gerhart J C. Marks J D. Lindquist E., (1998) Efficient construction of a large nonimmune phage antibody library: the production of high-affinity human single-chain antibodies to protein antigens (published erratum appears in Proc Natl Acad Sci USA 1999 96:795), Proc Natl Acad Sci USA 95:6157-62).

Focused or smart chemical and pharmacophore libraries can be designed with the help of sophisticated strategies involving computational chemistry (e.g., Kundu B. Khare S K. Rastogi S K., (1999) Combinatorial chemistry: polymer supported synthesis of peptide and non-peptide libraries, Progress in Drug Research 53:89-156) and the use of structure-based ligands using database searching and docking, de novo drug design and estimation of ligand binding affinities (Joseph-McCarthy D., (1999) Computational approaches to structure-based ligand design, Pharmacology & Therapeutics 84:179-91; Kirkpatrick D L. Watson S. Ulhaq S., (1999) Structure-based drug design: combinatorial chemistry and molecular modeling, Combinatorial Chemistry & High Throughput Screening. 2:211-21; Eliseev A V. Lehn J M., (1999) Dynamic combinatorial chemistry: evolutionary formation and screening of molecular libraries, Current Topics in Microbiology & Immunology 243:159-72; Bolger et al., (1991) Methods Enz. 203:21-45; Martin, (1991) Methods Enz. 203:587-613; Neidle et al., (1991) Methods Enz. 203:433-458; U.S. Pat. No. 6,178,384).

X. Crystallography

After binding compounds have been determined, the orientation of compound bound to target is determined. Preferably this determination involves crystallography on co-crystals of molecular scaffold compounds with target. Most protein crystallographic platforms can preferably be designed to analyze up to about 500 co-complexes of compounds, ligands, or molecular scaffolds bound to protein targets due to the physical parameters of the instruments and convenience of operation. If the number of scaffolds that have binding activity exceeds a number convenient for the application of crystallography methods, the scaffolds can be placed into groups based on having at least one common chemical structure or other desirable characteristics, and representative compounds can be selected from one or more of the classes. Classes can be made with increasingly exacting criteria until a desired number of classes (e.g., 500) is obtained. The classes can be based on chemical structure similarities between molecular scaffolds in the class, e.g., all possess a pyrrole ring, benzene ring, or other chemical feature. Likewise, classes can be based on shape characteristics, e.g., space-filling characteristics.

The co-crystallography analysis can be performed by co-complexing each scaffold with its target at concentrations of the scaffold that showed activity in the screening assay. This co-complexing can be accomplished with the use of low percentage organic solvents with the target molecule and then concentrating the target with each of the scaffolds. In preferred embodiments these solvents are less than 5% organic solvent such as dimethyl sulfoxide (DMSO), ethanol, methanol, or ethylene glycol in water or another aqueous solvent. Each scaffold complexed to the target molecule can then be screened with a suitable number of crystallization screening conditions at both 4 and 20 degrees. In preferred embodiments, about 96 crystallization screening conditions can be performed in order to obtain sufficient information about the co-complexation and crystallization conditions, and the orientation of the scaffold at the binding site of the target molecule. Crystal structures can then be analyzed to determine how the bound scaffold is oriented physically within the binding site or within one or more binding pockets of the molecular family member.

It is desirable to determine the atomic coordinates of the compounds bound to the target proteins in order to determine which is a most suitable scaffold for the protein family. X-ray crystallographic analysis is therefore most preferable for determining the atomic coordinates. Those compounds selected can be further tested with the application of medicinal chemistry. Compounds can be selected for medicinal chemistry testing based on their binding position in the target molecule. For example, when the compound binds at a binding site, the compound's binding position in the binding site of the target molecule can be considered with respect to the chemistry that can be performed on chemically tractable structures or sub-structures of the compound, and how such modifications on the compound might interact with structures or sub-structures on the binding site of the target. Thus, one can explore the binding site of the target and the chemistry of the scaffold in order to make decisions on how to modify the scaffold to arrive at a ligand with higher potency and/or selectivity. This process allows for more direct design of ligands, by utilizing structural and chemical information obtained directly from the co-complex, thereby enabling one to more efficiently and quickly design lead compounds that are likely to lead to beneficial drug products. In various embodiments it may be desirable to perform co-crystallography on all scaffolds that bind, or only those that bind with a particular affinity, for example, only those that bind with high affinity, moderate affinity, low affinity, very low affinity, or extremely low affinity. It may also be advantageous to perform co-crystallography on a selection of scaffolds that bind with any combination of affinities.

Standard X-ray protein diffraction studies such as by using a Rigaku RU-200® (Rigaku, Tokyo, Japan) with an X-ray imaging plate detector or a synchrotron beam-line can be performed on co-crystals and the diffraction data measured on a standard X-ray detector, such as a CCD detector or an X-ray imaging plate detector.

Performing X-ray crystallography on about 200 co-crystals should generally lead to about 50 co-crystals structures, which should provide about 10 scaffolds for validation in chemistry, which should finally result in about 5 selective leads for target molecules.

Virtual Assays

Commercially available software that generates three-dimensional graphical representations of the complexed target and compound from a set of coordinates provided can be used to illustrate and study how a compound is oriented when bound to a target. (e.g., QUANTA®, Accelerys, San Diego, Calif.). Thus, the existence of binding pockets at the binding site of the targets can be particularly useful in the present invention. These binding pockets are revealed by the crystallographic structure determination and show the precise chemical interactions involved in binding the compound to the binding site of the target. The person of ordinary skill will realize that the illustrations can also be used to decide where chemical groups might be added, substituted, modified, or deleted from the scaffold to enhance binding or another desirable effect, by considering where unoccupied space is located in the complex and which chemical substructures might have suitable size and/or charge characteristics to fill it. The person of ordinary skill will also realize that regions within the binding site can be flexible and its properties can change as a result of scaffold binding, and that chemical groups can be specifically targeted to those regions to achieve a desired effect. Specific locations on the molecular scaffold can be considered with reference to where a suitable chemical substructure can be attached and in which conformation, and which site has the most advantageous chemistry available.

An understanding of the forces that bind the compounds to the target proteins reveals which compounds can most advantageously be used as scaffolds, and which properties can most effectively be manipulated in the design of ligands. The person of ordinary skill will realize that steric, ionic, hydrogen bond, and other forces can be considered for their contribution to the maintenance or enhancement of the target-compound complex. Additional data can be obtained with automated computational methods, such as docking and/or Free Energy Perturbations (FEP), to account for other energetic effects such as desolvation penalties. The compounds selected can be used to generate information about the chemical interactions with the target or for elucidating chemical modifications that can enhance selectivity of binding of the compound.

Computer models, such as homology models (i.e., based on a known, experimentally derived structure) can be constructed using data from the co-crystal structures. When the target molecule is a protein or enzyme, preferred co-crystal structures for making homology models contain high sequence identity in the binding site of the protein sequence being modeled, and the proteins will preferentially also be within the same class and/or fold family. Knowledge of conserved residues in active sites of a protein class can be used to select homology models that accurately represent the binding site. Homology models can also be used to map structural information from a surrogate protein where an apo or co-crystal structure exists to the target protein.

Virtual screening methods, such as docking, can also be used to predict the binding configuration and affinity of scaffolds, compounds, and/or combinatorial library members to homology models. Using this data, and carrying out “virtual experiments” using computer software can save substantial resources and allow the person of ordinary skill to make decisions about which compounds can be suitable scaffolds or ligands, without having to actually synthesize the ligand and perform co-crystallization. Decisions thus can be made about which compounds merit actual synthesis and co-crystallization. An understanding of such chemical interactions aids in the discovery and design of drugs that interact more advantageously with target proteins and/or are more selective for one protein family member over others. Thus, applying these principles, compounds with superior properties can be discovered.

Additives that promote co-crystallization can of course be included in the target molecule formulation in order to enhance the formation of co-crystals. In the case of proteins or enzymes, the scaffold to be tested can be added to the protein formulation, which is preferably present at a concentration of approximately 1 mg/ml. The formulation can also contain between 0%-10% (v/v) organic solvent, e.g. DMSO, methanol, ethanol, propane diol, or 1,3 dimethyl propane diol (MPD) or some combination of those organic solvents. Compounds are preferably solubilized in the organic solvent at a concentration of about 10 mM and added to the protein sample at a concentration of about 100 mM. The protein-compound complex is then concentrated to a final concentration of protein of from about 5 to about 20 mg/ml. The complexation and concentration steps can conveniently be performed using a 96-well formatted concentration apparatus (e.g., Amicon Inc., Piscataway, N.J.). Buffers and other reagents present in the formulation being crystallized can contain other components that promote crystallization or are compatible with crystallization conditions, such as DTT, propane diol, glycerol.

The crystallization experiment can be set-up by placing small aliquots of the concentrated protein-compound complex (1 μl) in a 96 well format and sampling under 96 crystallization conditions. (Other screening formats can also be used, e.g., plates with greater than 96 wells.) Crystals can typically be obtained using standard crystallization protocols that can involve the 96 well crystallization plate being placed at different temperatures. Co-crystallization varying factors other than temperature can also be considered for each protein-compound complex if desirable. For example, atmospheric pressure, the presence or absence of light or oxygen, a change in gravity, and many other variables can all be tested. The person of ordinary skill in the art will realize other variables that can advantageously be varied and considered.

Ligand Design and Preparation

The design and preparation of ligands can be performed with or without structural and/or co-crystallization data by considering the chemical structures in common between the active scaffolds of a set. In this process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, carboxamide, nitro, and ester moieties; an amine of formula —NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX₄, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, carboxamide, nitro, and ester moieties; an aldehyde of formula —CHO; a sulfone of formula —SO₂X₁₃, where X₁₃ is selected from the group consisting of saturated or unsaturated alkyl and homocyclic or heterocyclic ring moieties; and a nitro of formula —NO₂.

Identification of Attachment Sites on Molecular Scaffolds and Ligands

In addition to the identification and development of ligands for phosphodiesterases and other enzymes, determination of the orientation of a molecular scaffold or other binding compound in a binding site allows identification of energetically allowed sites for attachment of the binding molecule to another component. For such sites, any free energy change associated with the presence of the attached component should not destablize the binding of the compound to the phosphodiesterase to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a grahic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a dlrect or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately using any of a variety of available software or by manual calculation. An example is the following:

Calculations were performed to estimate binding energies of different organic molecules to two Kinases: PIM-1 and CDK2. The organic molecules considered included Staurosporine, identified compounds that bind to PDE5A, and several linkers.

Calculated binding energies between protein-ligand complexes were obtained using the FlexX score (an implementation of the Bohm scoring function) within the Tripos software suite. The form for that equation is shown in the equation below:
ΔGbind=ΔGtr+ΔGhb+ΔGion+ΔGlipo+ΔGarom+ΔGrot
where: ΔGtr is a constant term that accounts for the overall loss of rotational and translational entropy of the lignand, ΔGhb accounts for hydrogen bonds formed between the ligand and protein, ΔGion accounts for the ionic interactions between the ligand and protein, ΔGlipo accounts for the lipophilic interaction that corresponds to the protein-ligand contact surface, ΔGarom accounts for interactions between aromatic rings in the protein and ligand, and ΔGrot accounts for the entropic penalty of restricting rotatable bonds in the ligand upon binding.

This method estimates the free energy that a lead compound should have to a target protein for which there is a crystal structure, and it accounts for the entropic penalty of flexible linkers. It can therefore be used to estimate the free energy penalty incurred by attaching linkers to molecules being screened and the binding energy that a lead compound should have in order to overcome the free energy penalty of the linker. The method does not account for solvation and the entropic penalty is likely overestimated for cases where the linker is bound to a solid phase through another binding complex, such as a biotin:streptavidin complex.

Co-crystals were aligned by superimposing residues of PIM-1 with corresponding residues in CDK2. The PIM-1 structure used for these calculations was a co-crystal of PIM-1 with a binding compound. The CDK2:Staurosporine co-crystal used was from the Brookhaven database file 1aq1. Hydrogen atoms were added to the proteins and atomic charges were assigned using the AMBER95 parameters within Sybyl. Modifications to the compounds described were made within the Sybyl modeling suite from Tripos.

These calcualtions indicate that the calculated binding energy for compounds that bind strongly to a given target (such as Staurosporine:CDK2) can be lower than −25 kcal/mol, while the calculated binding affinity for a good scaffold or an unoptimized binding compound can be in the range of −15 to −20. The free energy penalty for attachment to a linker such as the ethylene glycol or hexatriene is estimated as typically being in the range of +5 to +15 kcal/mol.

Linkers

Linkers suitable for use in the invention can be of many different types. Linkers can be selected for particular applications based on factors such as linker chemistry compatible for attachment to a binding compound and to another component utilized in the particular application. Additional factors can include, without limitation, linker length, linker stability, and ability to remove the linker at an appropriate time. Exemplary linkers include, but are not limited to, hexyl, hexatrienyl, ethylene glycol, and peptide linkers. Traceless linkers can also be used, e.g., as described in Plunkett, M. J., and Ellman, J. A., (1995), J. Org. Chem., 60:6006.

Typical functional groups, that are utilized to link binding compound(s), include, but not limited to, carboxylic acid, amine, hydroxyl, and thiol. (Examples can be found in Solid-supported combinatorial and parallel synthesis of small molecular weight compound libraries; (1998) Tetrahedron organic chemistry series Vol.17; Pergamon; p85).

Labels

As indicated above, labels can also be attached to a binding compound or to a linker attached to a binding compound. Such attachment may be direct (attached directly to the binding compound) or indirect (attached to a component that is directly or indirectly attached to the binding compound). Such labels allow detection of the compound either directly or indirectly. Attachement of labels can be performed using conventional chemistries. Labels can include, for example, fluorescent labels, radiolabels, light scattering particles, light absorbent particles, magnetic particles, enzymes, and specific binding agents (e.g., biotin or an antibody target moiety).

Solid Phase Media

Additional examples of components that can be attached directly or indirectly to a binding compound include various solid phase media. Similar to attachment of linkers and labels, attachment to solid phase media can be performed using conventional chemistries. Such solid phase media can include, for example, small components such as beads, nanoparticles, and fibers (e.g., in suspension or in a gel or chromatographic matrix). Likewise, solid phase media can include larger objects such as plates, chips, slides, and tubes. In many cases, the binding compound will be attached in only a portion of such an objects, e.g., in a spot or other local element on a generally flat surface or in a well or portion of a well.

Identification of Biological Agents

The posession of structural information about a protein also provides for the identification of useful biological agents, such as epitpose for development of antibodies, identification of mutation sites expected to affect activity, and identification of attachment sites allowing attachment of the protein to materials such as labels, linkers, peptides, and solid phase media.

Antibodies (Abs) finds multiple applications in a variety of areas including biotechnology, medicine and diagnosis, and indeed they are one of the most powerful tools for life science research. Abs directed against protein antigens can recognize either linear or native three-dimensional (3D) epitopes. The obtention of Abs that recognize 3D epitopes require the use of whole native protein (or of a portion that assumes a native conformation) as immunogens. Unfortunately, this not always a choice due to various technical reasons: for example the native protein is just not available, the protein is toxic, or its is desirable to utilize a high density antigen presentation. In such cases, immunization with peptides is the alternative. Of course, Abs generated in this manner will recognize linear epitopes, and they might or might not recognize the source native protein, but yet they will be useful for standard laboratory applications such as western blots. The selection of peptides to use as immunogens can be accomplished by following particular selection rules and/or use of epitope prediction software.

Though methods to predict antigenic peptides are not infallible, there are several rules that can be followed to determine what peptide fragments from a protein are likely to be antigenic. These rules are also dictated to increase the likelihood that an Ab to a particular peptide will recognize the native protein.
1. Antigenic peptides should be located in solvent accessible regions and contain both hydrophobic and hydrophilic residues.
   For proteins of known 3D structure, solvent accessibility can be determined using a variety of programs such as DSSP, NACESS, or WHATIF, among others.
   If the 3D structure is not known, use any of the following web servers to predict accessibilities: PHD, JPRED, PredAcc (c) ACCpro
2. Preferably select peptides lying in long loops connecting Secondary Structure (SS) motifs, avoiding peptides located in helical regions. This will increase the odds that the Ab recognizes the native protein. Such peptides can, for example, be identified from a crystal structure or crystal structure-based homology model.
   For protein with known 3D coordinates, SS can be obtained from the sequence link of the relevant entry at the Brookhaven data bank. The PDBsum server also offer SS analysis of pdb records.
   When no structure is available secondary structure predictions can be obtained from any of the following servers: PHD, JPRED, PSI—PRED, NNSP, etc
3. When possible, choose peptides that are in the N- and C-terminal region of the protein. Because the N- and C-terminal regions of proteins are usually solvent accessible and unstructured, Abs against those regions are also likely to recognize the native protein.
4. For cell surface glycoproteins, eliminate from initial peptides those containing consesus sites for N-glycosilation.
   N-glycosilation sites can be detected using Scanprosite, or NetNGlyc

In addition, several methods based on various physio-chemical properties of experimental determined epitopes (flexibility, hydrophibility, accessibility) have been published for the prediction of antigenic determinants and can be used. The antigenic index and Preditop are example.

Perhaps the simplest method for the prediction of antigenic determinants is that of Kolaskar and Tongaonkar, which is based on the occurrence of amino acid residues in experimentally determined epitopes. (Kolaskar and Tongaonkar (1990) A semi-empirical method for prediction of antigenic determinants on protein antigens. FEBBS Lett. 276(1-2):172-174.) The prediction algorithm works as follows:
1. Calculate the average propensity for each overlapping 7-mer and assign the result to the central residue (i+3) of the 7-mer.
2. Calculate the average for the whole protein.
3. (a) If the average for the whole protein is above 1.0 then all residues having average propensity above 1.0 are potentially antigenic.
3. (b) If the average for the whole protein is below 1.0 then all residues having above the average for the whole protein are potentially antigenic.
4. Find 8-mers where all residues are selected by step 3 above (6-mers in the original paper)

The Kolaskar and Tongaonkar method is also available from the GCG package, and it runs using the command egcg.

Crystal structures also allow identification of residues at which mutation is likely to alter the activity of the protein. Such residues include, for example, residues that interact with susbtrate, conserved active site residues, and residues that are in a region of ordered secondary structure of involved in tertiary interactions. The mutations that are likely to affect activity will vary for different molecular contexts. Mutations in an active site that will affect activity are typically substitutions or deletions that eliminate a charge-charge or hydrogen bonding interaction, or introduce a steric interference. Mutations in secondary structure regions or molecular interaction regions that are likely to affect activity include, for example, substitutions that alter the hydrophobicity/hydrophilicity of a region, or that introduce a sufficient strain in a region near or including the active site so that critical residue(s) in the active site are displaced. Such substitutions and/or deletions and/or insertions are recognized, and the predicted structural and/or energetic effects of mutations can be calculated using conventional software.

IX. Phosphodiesterase Activity Assays

A number of different assays for phosphodiesterase activity can be utilized for assaying for active modulators and/or determining specificity of a modulator for a particular phosphodiesterase or group or phosphodiesterases. In addition to the assay mentioned in the Examples below, one of ordinary skill in the art will know of other assays that can be utilized and can modify an assay for a particular application. For example, numerous papers concerning PDE5 as well as papers concerning other PDEs described assays that can be used. For example, useful assays are described in Fryburg et al., U.S. Patent Application Publication 2002/0165237, Thompson et al., U.S. Patent Application Publication 2002/0009764, Pamukcu et al., U.S. patent application Ser. No. 09/046,739, and Pamukcu et al., U.S. Pat. No. 6,500,610.

An assay for phosphodiesterase activity that can be used for PDE5A, can be performed according to the following procedure using purified PDE5A using the procedure described in Example 6.

Additional alternative assays can employ binding determinations. For example, this sort of assay can be formatted either in a fluorescence resonance energy transfer (FRET) format, or using an AlphaScreen (amplified luminescent proximity homogeneous assay) format by varying the donor and acceptor reagents that are attached to streptavidin or the phosphor-specific antibody.

X. Organic Synthetic Techniques

The versatility of computer-based modulator design and identification lies in the diversity of structures screened by the computer programs. The computer programs can search databases that contain very large numbers of molecules and can modify modulators already complexed with the enzyme with a wide variety of chemical functional groups. A consequence of this chemical diversity is that a potential modulator of phosphodiesterase function may take a chemical form that is not predictable. A wide array of organic synthetic techniques exist in the art to meet the challenge of constructing these potential modulators. Many of these organic synthetic methods are described in detail in standard reference sources utilized by those skilled in the art. One example of suh a reference is March, 1994, Advanced Organic Chemistry; Reactions, Mechanisms and Structure, New York, McGraw Hill. Thus, the techniques useful to synthesize a potential modulator of phosphodiesterase function identified by computer-based methods are readily available to those skilled in the art of organic chemical synthesis.

XI. Administration

The methods and compounds will typically be used in therapy for human patients. However, they may also be used to treat similar or identical diseases in other vertebrates such as other primates, sports animals, and pets such as horses, dogs and cats.

Suitable dosage forms, in part, depend upon the use or the route of administration, for example, oral, transdermal, transmucosal, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington's Pharmaceutical Sciences, 18^{th} ed., Mack Publishing Co., Easton, Pa., 1990 (hereby incorporated by reference herein).

Compounds can be formulated as pharmaceutically acceptable salts. Pharmaceutically acceptable salts are non-toxic salts in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound is dissolved in a suitable solvent, such as an aqueous or aqueous-alcohol in solution containing the appropriate acid and then isolated by evaporating the solution. In another example, a salt is prepared by reacting the free base and acid in an organic solvent.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Carriers or excipients can be used to produce pharmaceutical compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, or transdermal. Oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/orsubcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The amounts of various compound to be administered can be determined by standard procedures taking into account factors such as the compound IC₅₀, the biological half-life of the compound, the age, size, and weight of the patient, and the disorder associated with the patient. The importance of these and other factors are well known to those of ordinary skill in the art. Generally, a dose will be between about 0.01 and 50 mg/kg, preferably 0.1 and 20 mg/kg of the patient being treated. Multiple doses may be used.

Manipulation of PDE5A

As the full-length coding sequence and amino acid sequence of PDE5A is known, cloning, construction of recombinant hPIM-3, production and purification of recombinant protein, introduction of PDE5A into other organisms, and other molecular biological manipulations of PDE5A are readily performed.

Techniques for the manipulation of nucleic acids, such as, e.g., subcloning, labeling probes (e.g., random-primer labeling using Klenow polymerase, nick translation, amplification), sequencing, hybridization and the like are well disclosed in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Nucleic acid sequences can be amplified as necessary for further use using amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1;29(11):E54-E54; Hafner et al., Biotechniques 2001 April;30(4):852-6, 858, 860 passim; Zhong et al., Biotechniques 2001 April;30(4):852-6, 858, 860 passim.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be performed by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The nucleic acids of the invention can also be provided in expression vectors and cloning vehicles, e.g., sequences encoding the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are disclosed, e.g., U.S. Pat. No. 5,426,039. To facilitate cloning of amplified sequences, restriction enzyme sites can be “built into” a PCR primer pair. Vectors may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (AAV)-based vectors can be used to transduce cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada (1996) Gene Ther. 3:957-964.

The present invention also relates to fusion proteins, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well disclosed in the scientific and patent literature, see e.g., Kroll (1993) DNA Cell. Biol. 12:441-53.

The nucleic acids and polypeptides of the invention can be bound to a solid support, e.g., for use in screening and diagnostic methods. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of molecules to a solid support can be direct (i.e., the molecule contacts the solid support) or indirect (a “linker” is bound to the support and the molecule of interest binds to this linker). Molecules can be immobilized either covalently (e.g., utilizing single reactive thiol groups of cysteine residues (see, e.g., Colliuod (1993) Bioconjugate Chem. 4:528-536) or non-covalently but specifically (e.g., via immobilized antibodies (see, e.g., Schuhmann (1991) Adv. Mater. 3:388-391; Lu (1995) Anal. Chem. 67:83-87; the biotin/strepavidin system (see, e.g., Iwane (1997) Biophys. Biochem. Res. Comm. 230:76-80); metal chelating, e.g., Langmuir-Blodgett films (see, e.g., Ng (1995) Langmuir 11:4048-55); metal-chelating self-assembled monolayers (see, e.g., Sigal (1996) Anal. Chem. 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can also be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377:525-531 (1989).

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Host Cells and Transformed Cells

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

Vectors may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

For transient expression in mammalian cells, cDNA encoding a polypeptide of interest may be incorporated into a mammalian expression vector, e.g. pcDNA1, which is available commercially from Invitrogen Corporation (San Diego, Calif., U.S.A.; catalogue number V490-20). This is a multifunctional 4.2 kb plasmid vector designed for cDNA expression in eukaryotic systems, and cDNA analysis in prokaryotes, incorporated on the vector are the CMV promoter and enhancer, splice segment and polyadenylation signal, an SV40 and Polyoma virus origin of replication, and M13 origin to rescue single strand DNA for sequencing and mutagenesis, Sp6 and T7 RNA promoters for the production of sense and anti-sense RNA transcripts and a Col E1-like high copy plasmid origin. A polylinker is located appropriately downstream of the CMV promoter (and 3′ of the T7 promoter).

The cDNA insert may be first released from the above phagemid incorporated at appropriate restriction sites in the pcDNAI polylinker. Sequencing across the junctions may be performed to confirm proper insert orientation in pcDNAI. The resulting plasmid may then be introduced for transient expression into a selected mammalian cell host, for example, the monkey-derived, fibroblast like cells of the COS-1 lineage (available from the American Type Culture Collection, Rockville, Md. as ATCC CRL 1650).

For transient expression of the protein-encoding DNA, for example, COS-1cells may be transfected with approximately 8 μg DNA per 106 COS cells, by DEAE-mediated DNA transfection and treated with chloroquine according to the procedures described by Sambrook et al, Molecular Cloning: A Laboratory Manual, 1989, Cold Spring Harbor Laboratory Press, Cold Spring Harbor N.Y, pp. 16.30-16.37. An exemplary method is as follows. Briefly, COS-1 cells are plated at a density of 5×10⁶ cells/dish and then grown for 24 hours in FBS-supplemented DMEM/F12 medium. Medium is then removed and cells are washed in PBS and then in medium. A transfection solution containing DEAE dextran (0.4 mg/ml), 100 μM chloroquine, 10% NuSerum, DNA (0.4 mg/ml) in DMEM/F12 medium is then applied on the cells 10 ml volume. After incubation for 3 hours at 37° C., cells are washed in PBS and medium as just described and then shocked for 1 minute with 10% DMSO in DMEM/F12 medium. Cells are allowed to grow for 2-3 days in 10% FBS-supplemented medium, and at the end of incubation dishes are placed on ice, washed with ice cold PBS and then removed by scraping. Cells are then harvested by centrifugation at 1000 rpm for 10 minutes and the cellular pellet is frozen in liquid nitrogen, for subsequent use in protein expression. Northern blot analysis of a thawed aliquot of frozen cells may be used to confirm expression of receptor-encoding cDNA in cells under storage.

In a like manner, stably transfected cell lines can also prepared, for example, using two different cell types as host: CHO K1 and CHO Pro5. To construct these cell lines, cDNA coding for the relevant protein may be incorporated into the mammalian expression vector pRC/CMV (Invitrogen), which enables stable expression. Insertion at this site places the cDNA under the expression control of the cytomegalovirus promoter and upstream of the polyadenylation site and terminator of the bovine growth hormone gene, and into a vector background comprising the neomycin resistance gene (driven by the SV40 early promoter) as selectable marker.

An exemplary protocol to introduce plasmids constructed as described above is as follows. The host CHO cells are first seeded at a density of 5×10⁵ in 10% FBS-supplemented MEM medium. After growth for 24 hours, fresh medium is added to the plates and three hours later, the cells are transfected using the calcium phosphate-DNA co-precipitation procedure (Sambrook et al, supra). Briefly, 3 μg of DNA is mixed and incubated with buffered calcium solution for 10 minutes at room temperature. An equal volume of buffered phosphate solution is added and the suspension is incubated for 15 minutes at room temperature. Next, the incubated suspension is applied to the cells for 4 hours, removed and cells were shocked with medium containing 15% glycerol. Three minutes later, cells are washed with medium and incubated for 24 hours at normal growth conditions. Cells resistant to neomycin are selected in 10% FBS-supplemented alpha-MEM medium containing G418 (1 mg/ml). Individual colonies of G418-resistant cells are isolated about 2-3 weeks later, clonally selected and then propagated for assay purposes.

### EXAMPLES

A number of examples involved in the present invention are described below. In most cases, alternative techniques could also be used. For example, techniques, methods, and other information described in Whitaker et al., U.S. Patent Application 2001/0053780 can be used in the present invention. Such techniques and information include, without limitation, cloning, culturing, purification, assaying, screening, use of modulators, sequence information, and information concerning biological role of PDE5A. Each of these references is incorporated by reference herein in its entirety, including drawings.

### Example 1

### Cloning of PDE5A Phosphodiesterase Domain

PDE5A cDNA sequence was amplified from a Human Kidney QUICK-Clone cDNA library (Clontech, #7112-1) by PCR using the following primers:

PDE5A-S:
5′-GTCGTAT CATATG TCAGCAGCAGAGGAAGAAAC-3′ 33 mer
PDE5A-A:
5′-TCTGCA GTCGAC AGGCCACTCAGTTCCGCTTG-3′ 32 mer

The resulting PCR fragment was digested with NdeI and SalI and subcloned into the pET15S vector (shown below). In this expression plasmid, residues 531-875 of PDE5A are in frame with an N-terminal His-tag followed by a thrombin cleavage site.

The sequence of pET15S, with multi-cloning site is shown below:

[image]

pET15S vector is derived from pET15b vector (Novagen) for bacterial expression to produce the proteins with N-terminal His6. This vector was modified by replacement of NdeI-BamHI fragment to others to create a SalI site and stop codon (TAG). Vector size is 5814 bp. Insertion can be performed using NdeI-SalI site.

The nucleotide and amino acid sequences for the PDE5A phosphodiesterase domain utilized encompass amino acids 531-875 of the amino acid sequence provided in Table 4.

### Example 2

### Expression and Purification of PDE5A Phosphodiesterase Domain

PDE 5A is purified from E. coli cells [BL21(DE3)Codon Plus(RIL) (Novagen)] grown in Terrific broth that has been supplemented with 0.2 mM Zinc Acetate and 1 mM MgCl2 and induced for 16-20h with 1 mM IPTG at 22 C. The centrifuged bacterial pellet (typically 200-250g from 16 L) is suspended in lysis buffer (0.1M potassium phosphate buffer, pH 8.0, 10% glycerol, 1 mM PMSF). 100 ug/ml of lysozyme is added to the lysate and the cells are lysed in a Cell Disruptor (MircoFluidics). The cell extract is clarified at 5000 rpm in a Sorvall SA6000 rotor for 1 h, and the supernatant is recentrifuged for another hour at 17000 rpm in a Sorvall SA 600 rotor. 5 mM imidazole (pH 8.0) is added to the clarified supernatant and 2 ml of cobalt beads (50% slurry) is added to each 35 ml of extract. The beads are mixed at 4 C for 3-4 h on a Nutator and the beads are recovered by centrifugation at 4000 rpm for 3 min. The pelleted beads are washed several times with lysis buffer and the beads are packed on a BioRad disposable column. The bound protein is eluted with 3-4 column volumes of 0.1M imidazole followed by 0.25M imidazole, both prepared in lysis buffer. The protein eluted from the cobalt beads is concentrated on Centriprep-10 membranes (Amicon) and separated on a Pharmacia Superdex 200 column (26/60) in low salt buffer (25 mM Tris-HCl, pH 8.0, 150 mM NaCl, 14 mM beta-mercaptoethanol). The uncleaved PDE5A is purified by hydroxyapatite chromatography eluted with a phosphate gradient. A final buffer exchange is done on a Pharmacia Superdex 200 column (26/60) in 25 mM Tris-HCl, pH 8.0, 150 mM NaCl, 14 mM beta-mercaptoethanol.

### Example 3

### Crystallization of PDE5A Phosphodiesterase Domain

Crystals of purified PDE5 were grown in 10% (w/v) PEG3000, 100 mM phosphate-citrate (pH 4.3), 200 mM NaCl, 1 mM DTT, 1 mM Sp-cAMP and 8 mg/ml protein at 4° C., using an Intelliplate (Robbins Scientific, Hampton) by mixing one microliter of protein with one microliter of precipitant, also at 4° C.

### Example 4

### Diffraction Analysis of PDE5A

Synchrotron X-ray data for PDE5A was collected at beamline 8.3.1 of the Advanced Light Source (ALS, Lawrence Berkeley National Laboratory, Berkeley) on a Quantum 210 charge-coupled device detector (λ=1.10 Å). The data were processed using Mosflm ( ) and scaled and reduced with Scala ( ) in CCP4 ( ). The data processing process was driven by the ELVES automation scripts.

A ribbon diagram of the PDE5A catalytic domain is shown in FIG. 1. Atomic coordinates for the apo protein are provided in Table 1.

### Example 5

### PDE5A Binding Assays

Binding assays can be performed in a variety of ways, including a variety of ways known in the art. For example, as indicated above, binding assays can be performed using fluorescence resonance energy transfer (FRET) format, or using an AlphaScreen

Alternatively, any method which can measure binding of a ligand to the cGMP-binding site can be used. For example, a fluorescent ligand can be used. When bound to PDE5A, the emitted fluorescence is polarized. Once displaced by inhibitor binding, the polarization decreases.

Determination of IC50 for compounds by competitive binding assays. (Note that K₁ is the dissociation constant for inhibitor binding; K_{D} is the dissociation constant for substrate binding.) For this system, the IC50, inhibitor binding constant and substrate binding constant can be interrelated according to the following formula:

When using radiolabeled substrate

$${K}_{I} = \frac{IC 50}{1 + [ {L}^{*} ] / {K}_{D}}$$

the IC50˜K₁ when there is a small amount of labeled substrate.

### Example 6

### PDE5A Activity Assay

As an exemplary phosphodiesterase assay, the effect of potential modulators phosphodiesterase activity of PDE5A and other PDEs was measured in the following assay format:

Reagents

Assay Buffer

50 mM Tris, 7.5
8.3 mM MgCl₂
1.7 mM EGTA
0.01% BSA
Store @ 4 degrees
   RNA Binding YSi SPA Beads

Beads are 100 mg/ml in water. Dilute to 5 mg/ml in 18 mM Zn using 1M ZnAcetate/ZnSO₄ solution(3:1) and water. Store @ 4 degrees.

| Low control compounds | Concentration of 20X DMSO Stock |
|---|---|
| PDE1B: 8-methoxymethyl IBMX | 20 mM |
| PDE2A: EHNA | 10 mM |
| PDE3B: Milrinone | 2 mM |
| PDE4D: Rolipram | 10 mM |
| PDE5A: Zaprinast | 10 mM |
| PDE7B: IBMX | 40 mM |
| PDE10A: Dipyridamole | 4 mM |

Enzyme Concentrations (2× Final Concentration. Diluted in Assay Buffer)
PDE1B 50 ng/ml
PDE2A 50 ng/ml
PDE3B 10 ng/ml
PDE4D 5 ng/ml
PDE5A 20 ng/ml
PDE7B 25 ng/ml
PDE10A 5 ng/ml)
   Radioligands
[³H] cAMP (Amersham TRK559). Dilute 2000× in assay buffer.
[³H] cGMP (Amersham TRK392). For PDE5A assay only. Dilute 2000× in assay buffer.
   Protocol
   Make assay plates from 2 mM, 96 well master plates by transferring 1 ul of
   compound to 384 well plate using BiomekFx. Final concentration of compounds will be ˜100 μM. Duplicate assay plates are prepared from each master plate so that compounds are assayed in duplicate.
   To column 23 of the assay plate add 1 ul of 20× DMSO stock of appropriate control compound. These will be the low controls.
   Columns 1 and 2 of Chembridge library assay plates and columns 21 and 22 of the Maybridge library assay plates have 1 ul DMSO. These are the high controls.
   Using BiomekFx, pipet 10 μl of radioligand into each assay well, then, using the same tips, pipet 10 μl of enzyme into each well.
   Seal assay plate with transparent cover. Centrifuge briefly @ 1000 RPM, them mix on plate shaker for 10 s.
   Incubate @ 30° for 30 min.
   Using BiomekFx, add 10 μl of bead mixture to each assay well. Mix beads thoroughly in reservoir immediately prior to each assay plate addition.
   Re-seal plate with fresh transparent cover. Mix on plate shaker for 10 s, then centrifuge for 1 min. @ 1000 RPM.
   Place plates in counting racks. Let stand for ≧30 min, then count on Wallac TriLux using program 8.
   Analyze data as % inhibition of enzyme activity. Average of high controls=0% inhibition. Average of low controls=100% inhibition.

### Example 9

### Site-Directed Mutagenesis of PDE5A

Mutagenesis of PDE5A can be carried out according to the following procedure as described in Molecular Biology: Current Innovations and Future Trends. Eds. A. M. Griffin and H. G. Griffin. (1995) ISBN 1-898486-01-8, Horizon Scientific Press, PO Box 1, Wymondham, Norfolk, U.K., among others.

In vitro site-directed mutagenesis is an invaluable technique for studying protein structure-function relationships, gene expression and vector modification. Several methods have appeared in the literature, but many of these methods require single-stranded DNA as the template. The reason for this, historically, has been the need for separating the complementary strands to prevent reannealing. Use of PCR in site-directed mutagenesis accomplishes strand separation by using a denaturing step to separate the complementing strands and allowing efficient polymerization of the PCR primers. PCR site-directed methods thus allow site-specific mutations to be incorporated in virtually any double-stranded plasmid; eliminating the need for M13-based vectors or single-stranded rescue.

It is often desirable to reduce the number of cycles during PCR when performing PCR-based site-directed mutagenesis to prevent clonal expansion of any (undesired) second-site mutations. Limited cycling which would result in reduced product yield, is offset by increasing the starting template concentration. A selection is used to reduce the number of parental molecules coming through the reaction. Also, in order to use a single PCR primer set, it is desirable to optimize the long PCR method. Further, because of the extendase activity of some thermostable polymerases it is often necessary to incorporate an end-polishing step into the procedure prior to end-to-end ligation of the PCR-generated product containing the incorporated mutations in one or both PCR primers.

The following protocol provides a facile method for site-directed mutagenesis and accomplishes the above desired features by the incorporation of the following steps: (i) increasing template concentration approximately 1000-fold over conventional PCR conditions; (ii) reducing the number of cycles from 25-30 to 5-10; (iii) adding the restriction endonuclease DpnI (recognition target sequence: 5-Gm6ATC-3, where the A residue is methylated) to select against parental DNA (note: DNA isolated from almost all common strains of E. coli is Dam-methylated at the sequence 5-GATC-3); (iv) using Taq Extender in the PCR mix for increased reliability for PCR to 10 kb; (v) using Pfu DNA polymerase to polish the ends of the PCR product, and (vi) efficient intramolecular ligation in the presence of T4 DNA ligase.

Plasmid template DNA (approximately 0.5 pmole) is added to a PCR cocktail containing, in 25 ul of 1×mutagenesis buffer: (20 mM Tris HCl, pH 7.5; 8 mM MgCl2; 40 ug/ml BSA); 12-20 pmole of each primer (one of which must contain a 5-prime phosphate), 250 uM each dNTP, 2.5 U Taq DNA polymerase, 2.5 U of Taq Extender (Stratagene).

The PCR cycling parameters are 1 cycle of: 4 min at 94 C, 2 min at 50 C and 2 min at 72 C; followed by 5-10 cycles of 1 min at 94 C, 2 min at 54 C and 1 min at 72 C (step 1).

The parental template DNA and the linear, mutagenesis-primer incorporating newly synthesized DNA are treated with DpnI (10 U) and Pfu DNA polymerase (2.5U). This results in the DpnI digestion of the in vivo methylated parental template and hybrid DNA and the removal, by Pfu DNA polymerase, of the Taq DNA polymerase-extended base(s) on the linear PCR product.

The reaction is incubated at 37 C for 30 min and then transferred to 72 C for an additional 30 min (step 2).

Mutagenesis buffer (1×, 115 ul, containing 0.5 mM ATP) is added to the DpnI-digested, Pfu DNA polymerase-polished PCR products.

The solution is mixed and 10 ul is removed to a new microfuge tube and T4 DNA ligase (2-4 U) added.

The ligation is incubated for greater than 60 min at 37 C (step 3).

The treated solution is transformed into competent E. coli (step 4).

In addition to the PCR-based site-directed mutagenesis described above, other methods are available. Examples include those described in Kunkel (1985) Proc. Natl. Acad. Sci. 82:488-492; Eckstein et al. (1985) Nucl. Acids Res. 13:8764-8785; and using the GeneEditor™ Site-Directed Mutageneis Sytem from Promega.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to crystallization or co-crystallization conditions for PDE5A proteins and/or various phosphodiesterase domain sequences can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different specified values as the endpoints of a range. Such ranges are also within the scope of the present described invention.

Thus, additional embodiments are within the scope of the invention and within the following claims.

TABLE 1

| HEADER | | ---- | XX-XXX-XX xxxx |
|---|---|---|---|
| COMPND | | --- | |
| REMARK | 3 | | |
| REMARK | 3 | REFINEMENT. | |
| REMARK | 3 | PROGRAM: REFMAC 5.1.25 | |
| REMARK | 3 | AUTHORS: MURSHUDOV, VAGIN, DODSON | |
| REMARK | 3 | | |
| REMARK | 3 | REFINEMENT TARGET: MAXIMUM LIKELIHOOD | |
| REMARK | 3 | | |
| REMARK | 3 | DATA USED IN REFINEMENT. | |
| REMARK | 3 | RESOLUTION RANGE HIGH (ANGSTROMS): | 2.10 |
| REMARK | 3 | RESOLUTION RANGE LOW (ANGSTROMS): | 84.51 |
| REMARK | 3 | DATA CUTOFF (SIGMA(F)): | NONE |
| REMARK | 3 | COMPLETENESS FOR RANGE (%): | 99.35 |
| REMARK | 3 | NUMBER OF REFLECTIONS: | 23081 |
| REMARK | 3 | | |
| REMARK | 3 | FIT TO DATA USED IN REFINEMENT. | |
| REMARK | 3 | CROSS-VALIDATION METHOD: | THROUGHOUT |
| REMARK | 3 | FREE R VALUE TEST SET SELECTION: | RANDOM |
| REMARK | 3 | R VALUE (WORKING + TEST SET): | 0.20593 |
| REMARK | 3 | R VALUE (WORKING SET): | 0.20404 |
| REMARK | 3 | FREE R VALUE: | 0.24234 |
| REMARK | 3 | FREE R VALUE TEST SET SIZE (%): | 5.0 |
| REMARK | 3 | FREE R VALUE TEST SET COUNT: | 1227 |
| REMARK | 3 | | |
| REMARK | 3 | FIT IN THE HIGHEST RESOLUTION BIN. | |
| REMARK | 3 | TOTAL NUMBER OF BINS USED: | 20 |
| REMARK | 3 | BIN RESOLUTION RANGE HIGH: | 2.100 |
| REMARK | 3 | BIN RESOLUTION RANGE LOW: | 2.155 |
| REMARK | 3 | REFLECTION IN BIN (WORKING SET): | 1696 |
| REMARK | 3 | BIN R VALUE (WORKING SET): | 0.296 |
| REMARK | 3 | BIN FREE R VALUE SET COUNT: | 84 |
| REMARK | 3 | BIN FREE R VALUE: | 0.336 |
| REMARK | 3 | | |
| REMARK | 3 | NUMBER OF NON-HYDROGEN ATOMS USED IN REFINEMENT. | |
| REMARK | 3 | ALL ATOMS: 2555 | |
| REMARK | 3 | | |
| REMARK | 3 | B VALUES. | |
| REMARK | 3 | FROM WILSON PLOT (A**2): | NULL |
| REMARK | 3 | MEAN B VALUE (OVERALL, A**2): | 32.944 |

| REMARK | 3 | OVERALL ANISOTROPIC B VALUE. | |
|---|---|---|---|
| REMARK | 3 | B11 (A**2): | −1.34 |
| REMARK | 3 | B22 (A**2): | −1.34 |
| REMARK | 3 | B33 (A**2): | 2.01 |
| REMARK | 3 | B12 (A**2): | −0.67 |
| REMARK | 3 | B13 (A**2): | 0.00 |
| REMARK | 3 | B23 (A**2): | 0.00 |
| REMARK | 3 | | |

| REMARK | 3 | ESTIMATED OVERALL COORDINATE ERROR. | | | |
|---|---|---|---|---|---|
| REMARK | 3 | ESU BASED ON R VALUE (A): | | | 0.195 |
| REMARK | 3 | ESU BASED ON FREE R VALUE (A): | | | 0.173 |
| REMARK | 3 | ESU BASED ON MAXIMUM LIKELIHOOD (A): | | | 0.131 |
| REMARK | 3 | ESU FOR B VALUES BASED ON MAXIMUM LIKELIHOOD (A**2): | | | 5.040 |
| REMARK | 3 | | | | |
| REMARK | 3 | CORRELATION COEFFICIENTS. | | | |
| REMARK | 3 | CORRELATION COEFFICIENT FO-FC: | 0.957 | | |
| REMARK | 3 | CORRELATION COEFFICIENT FO-FC FREE: | 0.946 | | |
| REMARK | 3 | | | | |
| REMARK | 3 | RMS DEVIATIONS FROM IDEAL VALUES | COUNT | RMS | WEIGHT |
| REMARK | 3 | BOND LENGTHS REFINED ATOMS (A): | 2506; | 0.014; | 0.021 |
| REMARK | 3 | BOND LENGTHS OTHERS (A): | 2282; | 0.002; | 0.020 |
| REMARK | 3 | BOND ANGLES REFINED ATOMS (DEGREES): | 3376; | 1.501; | 1.953 |
| REMARK | 3 | BOND ANGLES OTHERS (DEGREES): | 5327; | 0.947; | 3.000 |
| REMARK | 3 | TORSION ANGLES, PERIOD 1 (DEGREES): | 297; | 5.771; | 5.000 |
| REMARK | 3 | CHIRAL-CENTER RESTRAINTS (A**3): | 382; | 0.086; | 0.200 |
| REMARK | 3 | GENERAL PLANES REFINED ATOMS (A): | 2718; | 0.010; | 0.020 |
| REMARK | 3 | GENERAL PLANES OTHERS (A): | 489; | 0.036; | 0.020 |
| REMARK | 3 | NON-BONDED CONTACTS REFINED ATOMS (A): | 643; | 0.239; | 0.200 |
| REMARK | 3 | NON-BONDED CONTACTS OTHERS (A): | 2492; | 0.232; | 0.200 |
| REMARK | 3 | NON-BONDED TORSION OTHERS (A): | 1437; | 0.087; | 0.200 |
| REMARK | 3 | H-BOND (X . . . Y) REFINED ATOMS (A): | 72; | 0.156; | 0.200 |
| REMARK | 3 | POTENTIAL METAL-ION REFINED ATOMS (A): | 1; | 0.041; | 0.200 |
| REMARK | 3 | SYMMETRY VDW REFINED ATOMS (A): | 40; | 0.636; | 0.200 |
| REMARK | 3 | SYMMETRY VDW OTHERS (A): | 74; | 0.393; | 0.200 |
| REMARK | 3 | SYMMETRY H-BOND REFINED ATOMS (A): | 18; | 0.558; | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 1502; | 0.616; | 1.500 |
| REMARK | 3 | MAIN-CHAIN ANGLE REFINED ATOMS (A**2): | 2417; | 1.182; | 2.000 |
| REMARK | 3 | SIDE-CHAIN BOND REFINED ATOMS (A**2): | 1004; | 1.967; | 3.000 |
| REMARK | 3 | SIDE-CHAIN ANGLE REFINED ATOMS (A**2): | 959; | 3.150; | 4.500 |

| REMARK | 3 | | | | | | |
|---|---|---|---|---|---|---|---|
| REMARK | 3 | NCS RESTRAINTS STATISTICS | | | | | |
| REMARK | 3 | NUMBER OF DIFFERENT NCS GROUPS: 1 | | | | | |
| REMARK | 3 | | | | | | |
| REMARK | 3 | NCS GROUP NUMBER: 1 | | | | | |
| REMARK | 3 | CHAIN NAMES: A B | | | | | |
| REMARK | 3 | NUMBER OF COMPONENTS NCS GROUP: 3 | | | | | |
| REMARK | 3 | COMPONENT | C | SSSEQI | TO C | SSSEQI | CODE |
| REMARK | 3 | 1 | A | 534 | A | 657 | 6 |
| REMARK | 3 | 1 | B | 534 | B | 657 | 6 |
| REMARK | 3 | 2 | A | 672 | A | 686 | 6 |
| REMARK | 3 | 2 | B | 672 | B | 686 | 6 |
| REMARK | 3 | 3 | A | 687 | A | 789 | 6 |
| REMARK | 3 | 3 | B | 687 | B | 789 | 6 |

| | | | | | | | | |
|---|---|---|---|---|---|---|---|---|
| REMARK | 3 | | GROUP | CHAIN | | COUNT | RMS | WEIGHT |
| REMARK | 3 | LOOSE POSITIONAL | 1 | A | (A): | 11; | 0.14; | 5.00 |
| REMARK | 3 | LOOSE THERMAL | 1 | A | (A**2): | 11; | 4.73; | 10.00 |

| REMARK | 3 | | | | | | | |
|---|---|---|---|---|---|---|---|---|
| REMARK | 3 | | | | | | | |
| REMARK | 3 | TLS DETAILS | | | | | | |
| REMARK | 3 | NUMBER OF TLS GROUPS: 2 | | | | | | |
| REMARK | 3 | | | | | | | |
| REMARK | 3 | TLS GROUP: 1 | | | | | | |
| REMARK | 3 | NUMBER OF COMPONENTS GROUP: 4 | | | | | | |
| REMARK | 3 | COMPONENTS | C | SSSEQI | TO C | SSSEQI | | |
| REMARK | 3 | RESIDUE RANGE: | A | 534 | A | 657 | | |
| REMARK | 3 | RESIDUE RANGE: | A | 672 | A | 686 | | |
| REMARK | 3 | RESIDUE RANGE: | A | 687 | A | 789 | | |
| REMARK | 3 | RESIDUE RANGE: | A | 804 | A | 862 | | |
| REMARK | 3 | ORIGIN FOR THE GROUP (A): | | | 29.9285 | 0.5264 | 7.4989 | |
| REMARK | 3 | T TENSOR | | | | | | |
| REMARK | 3 | T11: | 0.1470 | T22: | 0.1360 | | | |
| REMARK | 3 | T33: | 0.1011 | T12: | 0.0029 | | | |
| REMARK | 3 | T13: | 0.0027 | T23: | −0.1172 | | | |
| REMARK | 3 | L TENSOR | | | | | | |
| REMARK | 3 | L11: | 4.8960 | L22: | 2.7854 | | | |
| REMARK | 3 | L33: | 1.2544 | L12: | 0.7354 | | | |
| REMARK | 3 | L13: | −0.5427 | L23: | −0.0242 | | | |
| REMARK | 3 | S TENSOR | | | | | | |
| REMARK | 3 | S11: | 0.3148 | S12: | −0.0276 | S13: | 0.1681 | |
| REMARK | 3 | S21: | 0.0297 | S22: | −0.3301 | S23: | 0.3850 | |
| REMARK | 3 | S31: | −0.0433 | S32: | −0.0292 | S33: | 0.0152 | |
| REMARK | 3 | | | | | | | |
| REMARK | 3 | TLS GROUP: 2 | | | | | | |
| REMARK | 3 | NUMBER OF COMPONENTS GROUP: 1 | | | | | | |
| REMARK | 3 | COMPONENTS | C | SSSEQI | TO C | SSSEQI | | |
| REMARK | 3 | RESIDUE RANGE: | B | 686 | B | 686 | | |
| REMARK | 3 | ORIGIN FOR THE GROUP (A): | | | 28.9451 | −15.8239 | 8.3689 | |
| REMARK | 3 | T TENSOR | | | | | | |
| REMARK | 3 | T11: | 0.3135 | T22: | 0.3143 | | | |
| REMARK | 3 | T33: | 0.3134 | T12: | −0.0006 | | | |
| REMARK | 3 | T13: | 0.0010 | T23: | 0.0001 | | | |
| REMARK | 3 | L TENSOR | | | | | | |
| REMARK | 3 | L11: | 20.8295 | L22: | 24.8368 | | | |
| REMARK | 3 | L33: | 60.0722 | L12: | −4.3409 | | | |
| REMARK | 3 | L13: | −27.2632 | L23: | −5.7801 | | | |
| REMARK | 3 | S TENSOR | | | | | | |
| REMARK | 3 | S11: | 0.0102 | S12: | −0.2842 | S13: | 0.2176 | |
| REMARK | 3 | S21: | 0.1711 | S22: | −0.0107 | S23: | −0.6694 | |
| REMARK | 3 | S31: | −0.3272 | S32: | 1.0664 | S33: | 0.0006 | |
| REMARK | 3 | | | | | | | |
| REMARK | 3 | | | | | | | |
| REMARK | 3 | BULK SOLVENT MODELLING. | | | | | | |
| REMARK | 3 | METHOD USED: BABINET MODEL WITH MASK | | | | | | |
| REMARK | 3 | PARAMETERS FOR MASK CALCULATION | | | | | | |
| REMARK | 3 | VDW PROBE RADIUS: | | 1.40 | | | | |
| REMARK | 3 | ION PROBE RADIUS: | | 0.80 | | | | |
| REMARK | 3 | SHRINKAGE RADIUS: | | 0.80 | | | | |
| REMARK | 3 | | | | | | | |
| REMARK | 3 | OTHER REFINEMENT REMARKS: | | | | | | |
| REMARK | 3 | HYDROGENS HAVE BEEN ADDED IN THE RIDING POSITIONS | | | | | | |
| REMARK | 3 | | | | | | | |
| LINK | | HIS A 657 | | LEU A 672 | | | | gap |
| LINK | | GLN A 789 | | LEU A 804 | | | | gap |
| CRYST1 | | 96.411 | 96.411 | 79.026 | 90.00 | 90.00 | 120.00 P 62 | |
| SCALE1 | | 0.010372 | 0.005988 | 0.000000 | | 0.00000 | | |
| SCALE2 | | 0.000000 | 0.011977 | 0.000000 | | 0.00000 | | |
| SCALE3 | | 0.000000 | 0.000000 | 0.012654 | | 0.00000 | | |

| | | | | | | | | | | | |
|---|---|---|---|---|---|---|---|---|---|---|---|
| ATOM | 1 | N | GLU | A | 534 | 13.637 | −6.977 | 34.115 | 1.00 | 46.59 | N |
| ATOM | 3 | CA | GLU | A | 534 | 14.989 | −7.244 | 33.549 | 1.00 | 46.71 | C |
| ATOM | 5 | CB | GLU | A | 534 | 15.061 | −8.661 | 32.955 | 1.00 | 46.74 | C |
| ATOM | 8 | CG | GLU | A | 534 | 16.480 | −9.164 | 32.666 | 1.00 | 46.21 | C |
| ATOM | 11 | CD | GLU | A | 534 | 16.501 | −10.377 | 31.756 | 1.00 | 45.94 | C |
| ATOM | 12 | OE1 | GLU | A | 534 | 15.606 | −11.233 | 31.869 | 1.00 | 48.25 | O |
| ATOM | 13 | OE2 | GLU | A | 534 | 17.409 | −10.481 | 30.922 | 1.00 | 47.41 | O |
| ATOM | 14 | C | GLU | A | 534 | 15.355 | −6.213 | 32.478 | 1.00 | 47.04 | C |
| ATOM | 15 | O | GLU | A | 534 | 14.494 | −5.684 | 31.767 | 1.00 | 47.18 | O |
| ATOM | 18 | N | GLU | A | 535 | 16.652 | −5.954 | 32.367 | 1.00 | 47.23 | N |
| ATOM | 20 | CA | GLU | A | 535 | 17.198 | −5.021 | 31.392 | 1.00 | 47.53 | C |
| ATOM | 22 | CB | GLU | A | 535 | 18.708 | −4.862 | 31.658 | 1.00 | 47.84 | C |
| ATOM | 25 | CG | GLU | A | 535 | 19.347 | −3.589 | 31.116 | 1.00 | 48.85 | C |
| ATOM | 28 | CD | GLU | A | 535 | 20.293 | −3.849 | 29.958 | 1.00 | 51.55 | C |
| ATOM | 29 | OE1 | GLU | A | 535 | 19.948 | −4.658 | 29.058 | 1.00 | 53.04 | O |
| ATOM | 30 | OE2 | GLU | A | 535 | 21.386 | −3.234 | 29.945 | 1.00 | 53.75 | O |
| ATOM | 31 | C | GLU | A | 535 | 16.954 | −5.432 | 29.918 | 1.00 | 47.41 | C |
| ATOM | 32 | O | GLU | A | 535 | 16.977 | −4.580 | 29.022 | 1.00 | 47.72 | O |
| ATOM | 33 | N | GLU | A | 536 | 16.721 | −6.720 | 29.661 | 1.00 | 47.05 | N |
| ATOM | 35 | CA | GLU | A | 536 | 16.712 | −7.237 | 28.282 | 1.00 | 46.74 | C |
| ATOM | 37 | CB | GLU | A | 536 | 17.271 | −8.664 | 28.272 | 1.00 | 47.26 | C |
| ATOM | 40 | CG | GLU | A | 536 | 17.608 | −9.202 | 26.890 | 1.00 | 49.14 | C |
| ATOM | 43 | CD | GLU | A | 536 | 18.981 | −9.865 | 26.810 | 1.00 | 52.58 | C |
| ATOM | 44 | OE1 | GLU | A | 536 | 19.163 | −10.733 | 25.916 | 1.00 | 55.06 | O |
| ATOM | 45 | OE2 | GLU | A | 536 | 19.884 | −9.510 | 27.615 | 1.00 | 53.60 | O |
| ATOM | 46 | C | GLU | A | 536 | 15.320 | −7.214 | 27.641 | 1.00 | 45.58 | C |
| ATOM | 47 | O | GLU | A | 536 | 15.148 | −6.735 | 26.517 | 1.00 | 45.20 | O |
| ATOM | 48 | N | THR | A | 537 | 14.339 | −7.747 | 28.363 | 1.00 | 44.47 | N |
| ATOM | 50 | CA | THR | A | 537 | 12.949 | −7.784 | 27.903 | 1.00 | 43.56 | C |
| ATOM | 52 | CB | THR | A | 537 | 12.045 | −8.452 | 28.958 | 1.00 | 43.67 | C |
| ATOM | 54 | OG1 | THR | A | 537 | 12.382 | −7.963 | 30.264 | 1.00 | 44.49 | O |
| ATOM | 56 | CG2 | THR | A | 537 | 12.289 | −9.955 | 29.035 | 1.00 | 44.07 | C |
| ATOM | 60 | C | THR | A | 537 | 12.388 | −6.394 | 27.619 | 1.00 | 42.43 | C |
| ATOM | 61 | O | THR | A | 537 | 11.610 | −6.221 | 26.689 | 1.00 | 42.65 | O |
| ATOM | 62 | N | ARG | A | 538 | 12.769 | −5.413 | 28.433 | 1.00 | 41.09 | N |
| ATOM | 64 | CA | ARG | A | 538 | 12.226 | −4.062 | 28.303 | 1.00 | 40.04 | C |
| ATOM | 66 | CB | ARG | A | 538 | 12.529 | −3.221 | 29.552 | 1.00 | 40.12 | C |
| ATOM | 69 | CG | ARG | A | 538 | 11.534 | −3.401 | 30.690 | 1.00 | 39.78 | C |
| ATOM | 72 | CD | ARG | A | 538 | 11.540 | −2.271 | 31.725 | 1.00 | 40.15 | C |
| ATOM | 75 | NE | ARG | A | 538 | 11.185 | −2.748 | 33.070 | 1.00 | 40.21 | N |
| ATOM | 77 | CZ | ARG | A | 538 | 12.031 | −3.339 | 33.918 | 1.00 | 40.09 | C |
| ATOM | 78 | NH1 | ARG | A | 538 | 13.302 | −3.546 | 33.583 | 1.00 | 40.79 | N |
| ATOM | 81 | NH2 | ARG | A | 538 | 11.605 | −3.728 | 35.111 | 1.00 | 40.74 | N |
| ATOM | 84 | C | ARG | A | 538 | 12.721 | −3.345 | 27.038 | 1.00 | 39.10 | C |
| ATOM | 85 | O | ARG | A | 538 | 11.927 | −2.719 | 26.339 | 1.00 | 38.29 | O |
| ATOM | 86 | N | GLU | A | 539 | 14.013 | −3.439 | 26.729 | 1.00 | 38.12 | N |
| ATOM | 88 | CA | GLU | A | 539 | 14.513 | −2.825 | 25.510 | 1.00 | 37.97 | C |
| ATOM | 90 | CB | GLU | A | 539 | 15.985 | −3.171 | 25.262 | 1.00 | 38.18 | C |
| ATOM | 93 | CG | GLU | A | 539 | 16.978 | −2.098 | 25.671 | 1.00 | 39.15 | C |
| ATOM | 96 | CD | GLU | A | 539 | 18.404 | −2.487 | 25.300 | 1.00 | 40.30 | C |
| ATOM | 97 | OE1 | GLU | A | 539 | 18.552 | −3.462 | 24.550 | 1.00 | 40.77 | O |
| ATOM | 98 | OE2 | GLU | A | 539 | 19.367 | −1.846 | 25.778 | 1.00 | 42.40 | O |
| ATOM | 99 | C | GLU | A | 539 | 13.688 | −3.284 | 24.309 | 1.00 | 37.73 | C |
| ATOM | 100 | O | GLU | A | 539 | 13.366 | −2.489 | 23.425 | 1.00 | 37.24 | O |
| ATOM | 101 | N | LEU | A | 540 | 13.348 | −4.573 | 24.284 | 1.00 | 37.69 | N |
| ATOM | 103 | CA | LEU | A | 540 | 12.592 | −5.156 | 23.177 | 1.00 | 37.31 | C |
| ATOM | 105 | CB | LEU | A | 540 | 12.498 | −6.677 | 23.339 | 1.00 | 37.36 | C |
| ATOM | 108 | CG | LEU | A | 540 | 12.356 | −7.576 | 22.110 | 1.00 | 37.64 | C |
| ATOM | 110 | CD1 | LEU | A | 540 | 11.465 | −8.736 | 22.453 | 1.00 | 37.32 | C |
| ATOM | 114 | CD2 | LEU | A | 540 | 11.849 | −6.885 | 20.847 | 1.00 | 38.06 | C |
| ATOM | 118 | C | LEU | A | 540 | 11.196 | −4.548 | 23.060 | 1.00 | 37.13 | C |
| ATOM | 119 | O | LEU | A | 540 | 10.781 | −4.169 | 21.975 | 1.00 | 36.13 | O |
| ATOM | 120 | N | GLN | A | 541 | 10.475 | −4.468 | 24.178 | 1.00 | 38.03 | N |
| ATOM | 122 | CA | GLN | A | 541 | 9.124 | −3.896 | 24.200 | 1.00 | 38.21 | C |
| ATOM | 124 | CB | GLN | A | 541 | 8.564 | −3.886 | 25.624 | 1.00 | 38.94 | C |
| ATOM | 127 | CG | GLN | A | 541 | 8.343 | −5.265 | 26.267 | 1.00 | 40.24 | C |
| ATOM | 130 | CD | GLN | A | 541 | 7.319 | −6.120 | 25.543 | 1.00 | 41.38 | C |
| ATOM | 131 | OE1 | GLN | A | 541 | 6.254 | −6.416 | 26.097 | 1.00 | 43.49 | O |
| ATOM | 132 | NE2 | GLN | A | 541 | 7.650 | −6.550 | 24.318 | 1.00 | 42.15 | N |
| ATOM | 135 | C | GLN | A | 541 | 9.106 | −2.471 | 23.643 | 1.00 | 38.18 | C |
| ATOM | 136 | O | GLN | A | 541 | 8.320 | −2.163 | 22.751 | 1.00 | 38.20 | O |
| ATOM | 137 | N | SER | A | 542 | 9.977 | −1.616 | 24.176 | 1.00 | 38.16 | N |
| ATOM | 139 | CA | SER | A | 542 | 10.171 | −0.259 | 23.658 | 1.00 | 38.24 | C |
| ATOM | 141 | CB | SER | A | 542 | 11.413 | 0.405 | 24.294 | 1.00 | 38.29 | C |
| ATOM | 144 | OG | SER | A | 542 | 11.168 | 0.706 | 25.667 | 1.00 | 40.50 | O |
| ATOM | 146 | C | SER | A | 542 | 10.346 | −0.268 | 22.146 | 1.00 | 37.34 | C |
| ATOM | 147 | O | SER | A | 542 | 9.620 | 0.397 | 21.419 | 1.00 | 37.32 | O |
| ATOM | 148 | N | LEU | A | 543 | 11.303 | −1.050 | 21.676 | 1.00 | 36.77 | N |
| ATOM | 150 | CA | LEU | A | 543 | 11.681 | −1.020 | 20.268 | 1.00 | 36.27 | C |
| ATOM | 152 | CB | LEU | A | 543 | 12.926 | −1.889 | 20.028 | 1.00 | 35.78 | C |
| ATOM | 155 | CG | LEU | A | 543 | 13.961 | −1.395 | 19.028 | 1.00 | 35.13 | C |
| ATOM | 157 | CD1 | LEU | A | 543 | 14.656 | −2.564 | 18.401 | 1.00 | 35.18 | C |
| ATOM | 161 | CD2 | LEU | A | 543 | 13.429 | −0.468 | 17.943 | 1.00 | 35.56 | C |
| ATOM | 165 | C | LEU | A | 543 | 10.552 | −1.488 | 19.353 | 1.00 | 36.22 | C |
| ATOM | 166 | O | LEU | A | 543 | 10.233 | −0.838 | 18.364 | 1.00 | 36.70 | O |
| ATOM | 167 | N | ALA | A | 544 | 9.980 | −2.636 | 19.681 | 1.00 | 36.30 | N |
| ATOM | 169 | CA | ALA | A | 544 | 8.928 | −3.259 | 18.885 | 1.00 | 36.40 | C |
| ATOM | 171 | CB | ALA | A | 544 | 8.591 | −4.635 | 19.455 | 1.00 | 36.48 | C |
| ATOM | 175 | C | ALA | A | 544 | 7.663 | −2.416 | 18.793 | 1.00 | 36.41 | C |
| ATOM | 176 | O | ALA | A | 544 | 6.968 | −2.451 | 17.779 | 1.00 | 36.62 | O |
| ATOM | 177 | N | ALA | A | 545 | 7.363 | −1.662 | 19.842 | 1.00 | 36.50 | N |
| ATOM | 179 | CA | ALA | A | 545 | 6.118 | −0.890 | 19.887 | 1.00 | 36.47 | C |
| ATOM | 181 | CB | ALA | A | 545 | 5.498 | −0.939 | 21.290 | 1.00 | 36.49 | C |
| ATOM | 185 | C | ALA | A | 545 | 6.352 | 0.554 | 19.459 | 1.00 | 36.41 | C |
| ATOM | 186 | O | ALA | A | 545 | 5.414 | 1.316 | 19.365 | 1.00 | 35.99 | O |
| ATOM | 187 | N | ALA | A | 546 | 7.599 | 0.929 | 19.164 | 1.00 | 36.49 | N |
| ATOM | 189 | CA | ALA | A | 546 | 7.867 | 2.315 | 18.792 | 1.00 | 36.48 | C |
| ATOM | 191 | CB | ALA | A | 546 | 9.334 | 2.687 | 19.042 | 1.00 | 36.01 | C |
| ATOM | 195 | C | ALA | A | 546 | 7.480 | 2.532 | 17.338 | 1.00 | 36.52 | C |
| ATOM | 196 | O | ALA | A | 546 | 7.583 | 1.618 | 16.524 | 1.00 | 36.69 | O |
| ATOM | 197 | N | VAL | A | 547 | 6.991 | 3.731 | 17.037 | 1.00 | 36.54 | N |
| ATOM | 199 | CA | VAL | A | 547 | 6.740 | 4.160 | 15.667 | 1.00 | 36.75 | C |
| ATOM | 201 | CB | VAL | A | 547 | 5.976 | 5.520 | 15.623 | 1.00 | 37.32 | C |
| ATOM | 203 | CG1 | VAL | A | 547 | 5.990 | 6.140 | 14.214 | 1.00 | 38.11 | C |
| ATOM | 207 | CG2 | VAL | A | 547 | 4.547 | 5.353 | 16.124 | 1.00 | 37.43 | C |
| ATOM | 211 | C | VAL | A | 547 | 8.089 | 4.313 | 14.996 | 1.00 | 36.37 | C |
| ATOM | 212 | O | VAL | A | 547 | 8.997 | 4.931 | 15.547 | 1.00 | 37.11 | O |
| ATOM | 213 | N | VAL | A | 548 | 8.247 | 3.700 | 13.830 | 1.00 | 35.84 | N |
| ATOM | 215 | CA | VAL | A | 548 | 9.474 | 3.842 | 13.059 | 1.00 | 34.92 | C |
| ATOM | 217 | CB | VAL | A | 548 | 9.743 | 2.597 | 12.203 | 1.00 | 35.05 | C |
| ATOM | 219 | CG1 | VAL | A | 548 | 11.064 | 2.729 | 11.489 | 1.00 | 34.26 | C |
| ATOM | 223 | CG2 | VAL | A | 548 | 9.721 | 1.338 | 13.065 | 1.00 | 35.00 | C |
| ATOM | 227 | C | VAL | A | 548 | 9.303 | 5.069 | 12.167 | 1.00 | 34.49 | C |
| ATOM | 228 | O | VAL | A | 548 | 8.531 | 5.019 | 11.219 | 1.00 | 34.01 | O |
| ATOM | 229 | N | PRO | A | 549 | 9.976 | 6.180 | 12.459 | 1.00 | 33.90 | N |
| ATOM | 230 | CA | PRO | A | 549 | 9.824 | 7.360 | 11.603 | 1.00 | 33.96 | C |
| ATOM | 232 | CB | PRO | A | 549 | 10.636 | 8.456 | 12.325 | 1.00 | 33.76 | C |
| ATOM | 235 | CG | PRO | A | 549 | 10.862 | 7.955 | 13.695 | 1.00 | 34.32 | C |
| ATOM | 238 | CD | PRO | A | 549 | 10.901 | 6.438 | 13.574 | 1.00 | 34.16 | C |
| ATOM | 241 | C | PRO | A | 549 | 10.351 | 7.104 | 10.195 | 1.00 | 33.88 | C |
| ATOM | 242 | O | PRO | A | 549 | 11.107 | 6.139 | 9.961 | 1.00 | 34.16 | O |
| ATOM | 243 | N | SER | A | 550 | 9.952 | 7.968 | 9.267 | 1.00 | 33.64 | N |
| ATOM | 245 | CA | SER | A | 550 | 10.307 | 7.816 | 7.871 | 1.00 | 34.15 | C |
| ATOM | 247 | CB | SER | A | 550 | 9.515 | 8.800 | 7.022 | 1.00 | 34.02 | C |
| ATOM | 250 | OG | SER | A | 550 | 10.072 | 10.094 | 7.126 | 1.00 | 33.86 | O |
| ATOM | 252 | C | SER | A | 550 | 11.807 | 8.026 | 7.653 | 1.00 | 34.44 | C |
| ATOM | 253 | O | SER | A | 550 | 12.498 | 8.601 | 8.480 | 1.00 | 35.07 | O |
| ATOM | 254 | N | ALA | A | 551 | 12.309 | 7.537 | 6.536 | 1.00 | 34.56 | N |
| ATOM | 256 | CA | ALA | A | 551 | 13.690 | 7.773 | 6.158 | 1.00 | 34.63 | C |
| ATOM | 258 | CB | ALA | A | 551 | 13.983 | 7.122 | 4.805 | 1.00 | 34.69 | C |
| ATOM | 262 | C | ALA | A | 551 | 14.025 | 9.262 | 6.117 | 1.00 | 34.98 | C |
| ATOM | 263 | O | ALA | A | 551 | 15.087 | 9.675 | 6.604 | 1.00 | 34.64 | O |
| ATOM | 264 | N | GLN | A | 552 | 13.134 | 10.049 | 5.518 | 1.00 | 35.27 | N |
| ATOM | 266 | CA | GLN | A | 552 | 13.305 | 11.495 | 5.424 | 1.00 | 36.31 | C |
| ATOM | 268 | CB | GLN | A | 552 | 12.108 | 12.129 | 4.698 | 1.00 | 36.61 | C |
| ATOM | 271 | CG | GLN | A | 552 | 12.240 | 13.658 | 4.434 | 1.00 | 38.76 | C |
| ATOM | 274 | CD | GLN | A | 552 | 10.971 | 14.236 | 3.807 | 1.00 | 41.89 | C |
| ATOM | 275 | OE1 | GLN | A | 552 | 10.537 | 13.767 | 2.751 | 1.00 | 43.18 | O |
| ATOM | 276 | NE2 | GLN | A | 552 | 10.361 | 15.230 | 4.465 | 1.00 | 44.04 | N |
| ATOM | 279 | C | GLN | A | 552 | 13.476 | 12.162 | 6.800 | 1.00 | 36.32 | C |
| ATOM | 280 | O | GLN | A | 552 | 14.398 | 12.938 | 6.990 | 1.00 | 36.63 | O |
| ATOM | 281 | N | THR | A | 553 | 12.566 | 11.868 | 7.725 | 1.00 | 36.31 | N |
| ATOM | 283 | CA | THR | A | 553 | 12.619 | 12.377 | 9.098 | 1.00 | 36.39 | C |
| ATOM | 285 | CB | THR | A | 553 | 11.422 | 11.841 | 9.922 | 1.00 | 36.22 | C |
| ATOM | 287 | OG1 | THR | A | 553 | 10.178 | 12.197 | 9.301 | 1.00 | 36.41 | O |
| ATOM | 289 | CG2 | THR | A | 553 | 11.359 | 12.516 | 11.300 | 1.00 | 36.57 | C |
| ATOM | 293 | C | THR | A | 553 | 13.916 | 11.958 | 9.804 | 1.00 | 36.49 | C |
| ATOM | 294 | O | THR | A | 553 | 14.472 | 12.719 | 10.589 | 1.00 | 36.70 | O |
| ATOM | 295 | N | LEU | A | 554 | 14.390 | 10.747 | 9.541 | 1.00 | 36.10 | N |
| ATOM | 297 | CA | LEU | A | 554 | 15.578 | 10.252 | 10.242 | 1.00 | 36.39 | C |
| ATOM | 299 | CB | LEU | A | 554 | 15.532 | 8.735 | 10.373 | 1.00 | 36.37 | C |
| ATOM | 302 | CG | LEU | A | 554 | 14.432 | 8.145 | 11.243 | 1.00 | 36.80 | C |
| ATOM | 304 | CD1 | LEU | A | 554 | 14.471 | 6.628 | 11.089 | 1.00 | 37.50 | C |
| ATOM | 308 | CD2 | LEU | A | 554 | 14.582 | 8.566 | 12.701 | 1.00 | 36.05 | C |
| ATOM | 312 | C | LEU | A | 554 | 16.905 | 10.692 | 9.604 | 1.00 | 36.32 | C |
| ATOM | 313 | O | LEU | A | 554 | 17.956 | 10.487 | 10.191 | 1.00 | 36.86 | O |
| ATOM | 314 | N | LYS | A | 555 | 16.845 | 11.311 | 8.427 | 1.00 | 36.57 | N |
| ATOM | 316 | CA | LYS | A | 555 | 18.021 | 11.840 | 7.727 | 1.00 | 36.89 | C |
| ATOM | 318 | CB | LYS | A | 555 | 18.772 | 12.862 | 8.597 | 1.00 | 37.14 | C |
| ATOM | 321 | CG | LYS | A | 555 | 17.922 | 13.972 | 9.133 | 1.00 | 38.89 | C |
| ATOM | 324 | CD | LYS | A | 555 | 18.742 | 14.950 | 9.972 | 1.00 | 42.02 | C |
| ATOM | 327 | CE | LYS | A | 555 | 17.839 | 16.017 | 10.608 | 1.00 | 43.95 | C |
| ATOM | 330 | NZ | LYS | A | 555 | 18.583 | 17.280 | 11.057 | 1.00 | 46.49 | N |
| ATOM | 334 | C | LYS | A | 555 | 19.007 | 10.769 | 7.275 | 1.00 | 36.71 | C |
| ATOM | 335 | O | LYS | A | 555 | 20.136 | 11.089 | 6.959 | 1.00 | 37.24 | O |
| ATOM | 336 | N | ILE | A | 556 | 18.592 | 9.507 | 7.242 | 1.00 | 36.27 | N |
| ATOM | 338 | CA | ILE | A | 556 | 19.500 | 8.415 | 6.944 | 1.00 | 35.92 | C |
| ATOM | 340 | CB | ILE | A | 556 | 18.930 | 7.099 | 7.436 | 1.00 | 36.15 | C |
| ATOM | 342 | CG1 | ILE | A | 556 | 17.578 | 6.808 | 6.762 | 1.00 | 35.82 | C |
| ATOM | 345 | CD1 | ILE | A | 556 | 17.211 | 5.371 | 6.785 | 1.00 | 36.65 | C |
| ATOM | 349 | CG2 | ILE | A | 556 | 18.808 | 7.132 | 8.926 | 1.00 | 37.25 | C |
| ATOM | 353 | C | ILE | A | 556 | 19.875 | 8.267 | 5.476 | 1.00 | 35.83 | C |
| ATOM | 354 | O | ILE | A | 556 | 20.796 | 7.523 | 5.164 | 1.00 | 35.57 | O |
| ATOM | 355 | N | THR | A | 557 | 19.145 | 8.925 | 4.585 | 1.00 | 36.11 | N |
| ATOM | 357 | CA | THR | A | 557 | 19.498 | 8.982 | 3.163 | 1.00 | 37.13 | C |
| ATOM | 359 | CB | THR | A | 557 | 18.291 | 9.458 | 2.315 | 1.00 | 37.47 | C |
| ATOM | 361 | OG1 | THR | A | 557 | 17.101 | 8.764 | 2.713 | 1.00 | 38.12 | O |
| ATOM | 363 | CG2 | THR | A | 557 | 18.447 | 9.061 | 0.838 | 1.00 | 38.48 | C |
| ATOM | 367 | C | THR | A | 557 | 20.707 | 9.891 | 2.904 | 1.00 | 37.31 | C |
| ATOM | 368 | O | THR | A | 557 | 21.322 | 9.813 | 1.846 | 1.00 | 37.89 | O |
| ATOM | 369 | N | ASP | A | 558 | 21.067 | 10.717 | 3.885 | 1.00 | 37.34 | N |
| ATOM | 371 | CA | ASP | A | 558 | 22.131 | 11.705 | 3.731 | 1.00 | 37.90 | C |
| ATOM | 373 | CB | ASP | A | 558 | 21.983 | 12.865 | 4.748 | 1.00 | 38.69 | C |
| ATOM | 376 | CG | ASP | A | 558 | 20.629 | 13.574 | 4.660 | 1.00 | 41.60 | C |
| ATOM | 377 | OD1 | ASP | A | 558 | 19.745 | 13.072 | 3.924 | 1.00 | 46.07 | O |
| ATOM | 378 | OD2 | ASP | A | 558 | 20.363 | 14.638 | 5.284 | 1.00 | 44.02 | O |
| ATOM | 379 | C | ASP | A | 558 | 23.501 | 11.103 | 3.942 | 1.00 | 37.29 | C |
| ATOM | 380 | O | ASP | A | 558 | 23.745 | 10.454 | 4.960 | 1.00 | 36.55 | O |
| ATOM | 381 | N | PHE | A | 559 | 24.409 | 11.371 | 3.009 | 1.00 | 36.83 | N |
| ATOM | 383 | CA | PHE | A | 559 | 25.811 | 11.066 | 3.216 | 1.00 | 37.19 | C |
| ATOM | 385 | CB | PHE | A | 559 | 26.616 | 11.405 | 1.961 | 1.00 | 37.33 | C |
| ATOM | 388 | CG | PHE | A | 559 | 26.453 | 10.399 | 0.852 | 1.00 | 36.15 | C |
| ATOM | 389 | CD1 | PHE | A | 559 | 25.982 | 10.791 | −0.392 | 1.00 | 34.77 | C |
| ATOM | 391 | CE1 | PHE | A | 559 | 25.840 | 9.878 | −1.408 | 1.00 | 34.14 | C |
| ATOM | 393 | CZ | PHE | A | 559 | 26.139 | 8.550 | −1.191 | 1.00 | 32.43 | C |
| ATOM | 395 | CE2 | PHE | A | 559 | 26.609 | 8.131 | 0.050 | 1.00 | 33.75 | C |
| ATOM | 397 | CD2 | PHE | A | 559 | 26.759 | 9.062 | 1.066 | 1.00 | 35.34 | C |
| ATOM | 399 | C | PHE | A | 559 | 26.397 | 11.784 | 4.458 | 1.00 | 37.80 | C |
| ATOM | 400 | O | PHE | A | 559 | 27.284 | 11.258 | 5.139 | 1.00 | 38.12 | O |
| ATOM | 401 | N | SER | A | 560 | 25.858 | 12.947 | 4.757 | 1.00 | 37.70 | N |
| ATOM | 403 | CA | SER | A | 560 | 26.293 | 13.784 | 5.879 | 1.00 | 39.03 | C |
| ATOM | 405 | CB | SER | A | 560 | 25.835 | 15.215 | 5.579 | 1.00 | 39.04 | C |
| ATOM | 408 | OG | SER | A | 560 | 26.651 | 15.717 | 4.538 | 1.00 | 41.01 | O |
| ATOM | 410 | C | SER | A | 560 | 25.807 | 13.405 | 7.300 | 1.00 | 39.09 | C |
| ATOM | 411 | O | SER | A | 560 | 26.199 | 14.033 | 8.285 | 1.00 | 39.31 | O |
| ATOM | 412 | N | PHE | A | 561 | 24.947 | 12.402 | 7.382 | 1.00 | 39.13 | N |
| ATOM | 414 | CA | PHE | A | 561 | 24.382 | 11.907 | 8.629 | 1.00 | 38.97 | C |
| ATOM | 416 | CB | PHE | A | 561 | 23.823 | 10.514 | 8.364 | 1.00 | 38.71 | C |
| ATOM | 419 | CG | PHE | A | 561 | 23.201 | 9.868 | 9.550 | 1.00 | 37.66 | C |
| ATOM | 420 | CD1 | PHE | A | 561 | 21.877 | 10.112 | 9.864 | 1.00 | 36.57 | C |
| ATOM | 422 | CE1 | PHE | A | 561 | 21.280 | 9.486 | 10.925 | 1.00 | 36.89 | C |
| ATOM | 424 | CZ | PHE | A | 561 | 22.004 | 8.579 | 11.680 | 1.00 | 37.15 | C |
| ATOM | 426 | CE2 | PHE | A | 561 | 23.319 | 8.309 | 11.380 | 1.00 | 35.98 | C |
| ATOM | 428 | CD2 | PHE | A | 561 | 23.922 | 8.947 | 10.310 | 1.00 | 37.17 | C |
| ATOM | 430 | C | PHE | A | 561 | 25.392 | 11.802 | 9.755 | 1.00 | 39.21 | C |
| ATOM | 431 | O | PHE | A | 561 | 26.518 | 11.347 | 9.516 | 1.00 | 38.44 | O |
| ATOM | 432 | N | SER | A | 562 | 24.970 | 12.166 | 10.975 | 1.00 | 39.53 | N |
| ATOM | 434 | CA | SER | A | 562 | 25.845 | 12.056 | 12.148 | 1.00 | 40.79 | C |
| ATOM | 436 | CB | SER | A | 562 | 25.934 | 13.376 | 12.916 | 1.00 | 41.03 | C |
| ATOM | 439 | OG | SER | A | 562 | 26.984 | 13.244 | 13.855 | 1.00 | 40.22 | O |
| ATOM | 441 | C | SER | A | 562 | 25.615 | 10.895 | 13.153 | 1.00 | 41.03 | C |
| ATOM | 442 | O | SER | A | 562 | 26.440 | 10.016 | 13.224 | 1.00 | 45.01 | O |
| ATOM | 443 | N | ASP | A | 563 | 24.573 | 10.864 | 13.933 | 1.00 | 40.48 | N |
| ATOM | 445 | CA | ASP | A | 563 | 24.485 | 9.895 | 15.082 | 1.00 | 40.78 | C |
| ATOM | 447 | CB | ASP | A | 563 | 24.996 | 8.476 | 14.761 | 1.00 | 40.79 | C |
| ATOM | 450 | CG | ASP | A | 563 | 26.311 | 8.095 | 15.496 | 1.00 | 41.91 | C |
| ATOM | 451 | OD1 | ASP | A | 563 | 27.385 | 8.617 | 15.150 | 1.00 | 42.85 | O |
| ATOM | 452 | OD2 | ASP | A | 563 | 26.383 | 7.226 | 16.396 | 1.00 | 42.36 | O |
| ATOM | 453 | C | ASP | A | 563 | 25.019 | 10.357 | 16.478 | 1.00 | 40.06 | C |
| ATOM | 454 | O | ASP | A | 563 | 24.619 | 9.825 | 17.504 | 1.00 | 38.82 | O |
| ATOM | 455 | N | PHE | A | 564 | 25.904 | 11.340 | 16.513 | 1.00 | 39.33 | N |
| ATOM | 457 | CA | PHE | A | 564 | 26.386 | 11.857 | 17.795 | 1.00 | 39.36 | C |
| ATOM | 459 | CB | PHE | A | 564 | 27.203 | 13.141 | 17.603 | 1.00 | 39.66 | C |
| ATOM | 462 | CG | PHE | A | 564 | 28.657 | 12.870 | 17.381 | 1.00 | 43.24 | C |
| ATOM | 463 | CD1 | PHE | A | 564 | 29.200 | 12.874 | 16.098 | 1.00 | 47.67 | C |
| ATOM | 465 | CE1 | PHE | A | 564 | 30.563 | 12.592 | 15.875 | 1.00 | 47.93 | C |
| ATOM | 467 | CZ | PHE | A | 564 | 31.378 | 12.287 | 16.947 | 1.00 | 48.79 | C |
| ATOM | 469 | CE2 | PHE | A | 564 | 30.834 | 12.254 | 18.254 | 1.00 | 49.45 | C |
| ATOM | 471 | CD2 | PHE | A | 564 | 29.478 | 12.544 | 18.455 | 1.00 | 47.83 | C |
| ATOM | 473 | C | PHE | A | 564 | 25.261 | 12.052 | 18.811 | 1.00 | 37.82 | C |
| ATOM | 474 | O | PHE | A | 564 | 25.335 | 11.532 | 19.915 | 1.00 | 38.99 | O |
| ATOM | 475 | N | GLU | A | 565 | 24.192 | 12.714 | 18.402 | 1.00 | 35.36 | N |
| ATOM | 477 | CA | GLU | A | 565 | 23.147 | 13.112 | 19.326 | 1.00 | 33.95 | C |
| ATOM | 479 | CB | GLU | A | 565 | 22.502 | 14.398 | 18.801 | 1.00 | 34.14 | C |
| ATOM | 482 | CG | GLU | A | 565 | 23.501 | 15.529 | 18.592 | 1.00 | 34.68 | C |
| ATOM | 485 | CD | GLU | A | 565 | 24.101 | 16.014 | 19.908 | 1.00 | 36.90 | C |
| ATOM | 486 | OE1 | GLU | A | 565 | 23.466 | 16.857 | 20.627 | 1.00 | 35.83 | O |
| ATOM | 487 | OE2 | GLU | A | 565 | 25.209 | 15.534 | 20.224 | 1.00 | 37.58 | O |
| ATOM | 488 | C | GLU | A | 565 | 22.088 | 12.037 | 19.601 | 1.00 | 31.86 | C |
| ATOM | 489 | O | GLU | A | 565 | 21.295 | 12.176 | 20.524 | 1.00 | 29.83 | O |
| ATOM | 490 | N | LEU | A | 566 | 22.100 | 10.964 | 18.819 | 1.00 | 30.24 | N |
| ATOM | 492 | CA | LEU | A | 566 | 21.068 | 9.928 | 18.904 | 1.00 | 29.66 | C |
| ATOM | 494 | CB | LEU | A | 566 | 20.992 | 9.104 | 17.604 | 1.00 | 29.51 | C |
| ATOM | 497 | CG | LEU | A | 566 | 20.779 | 9.878 | 16.294 | 1.00 | 30.52 | C |
| ATOM | 499 | CD1 | LEU | A | 566 | 20.689 | 8.857 | 15.168 | 1.00 | 32.77 | C |
| ATOM | 503 | CD2 | LEU | A | 566 | 19.525 | 10.745 | 16.304 | 1.00 | 32.40 | C |
| ATOM | 507 | C | LEU | A | 566 | 21.274 | 8.966 | 20.059 | 1.00 | 28.90 | C |
| ATOM | 508 | O | LEU | A | 566 | 22.398 | 8.657 | 20.420 | 1.00 | 29.20 | O |
| ATOM | 509 | N | SER | A | 567 | 20.171 | 8.507 | 20.629 | 1.00 | 27.75 | N |
| ATOM | 511 | CA | SER | A | 567 | 20.153 | 7.424 | 21.601 | 1.00 | 27.51 | C |
| ATOM | 513 | CB | SER | A | 567 | 18.820 | 7.439 | 22.353 | 1.00 | 27.43 | C |
| ATOM | 516 | OG | SER | A | 567 | 17.737 | 7.060 | 21.502 | 1.00 | 25.32 | O |
| ATOM | 518 | C | SER | A | 567 | 20.298 | 6.072 | 20.908 | 1.00 | 28.05 | C |
| ATOM | 519 | O | SER | A | 567 | 20.132 | 5.978 | 19.682 | 1.00 | 28.56 | O |
| ATOM | 520 | N | ASP | A | 568 | 20.573 | 5.021 | 21.674 | 1.00 | 28.29 | N |
| ATOM | 522 | CA | ASP | A | 568 | 20.710 | 3.684 | 21.078 | 1.00 | 29.26 | C |
| ATOM | 524 | CB | ASP | A | 568 | 21.188 | 2.640 | 22.108 | 1.00 | 29.83 | C |
| ATOM | 527 | CG | ASP | A | 568 | 22.661 | 2.767 | 22.434 | 1.00 | 29.32 | C |
| ATOM | 528 | OD1 | ASP | A | 568 | 23.368 | 3.481 | 21.729 | 1.00 | 29.71 | O |
| ATOM | 529 | OD2 | ASP | A | 568 | 23.187 | 2.210 | 23.405 | 1.00 | 32.67 | O |
| ATOM | 530 | C | ASP | A | 568 | 19.376 | 3.227 | 20.468 | 1.00 | 29.47 | C |
| ATOM | 531 | O | ASP | A | 568 | 19.348 | 2.608 | 19.410 | 1.00 | 27.71 | O |
| ATOM | 532 | N | LEU | A | 569 | 18.283 | 3.536 | 21.155 | 1.00 | 29.32 | N |
| ATOM | 534 | CA | LEU | A | 569 | 16.958 | 3.252 | 20.624 | 1.00 | 29.88 | C |
| ATOM | 536 | CB | LEU | A | 569 | 15.869 | 3.644 | 21.619 | 1.00 | 29.64 | C |
| ATOM | 539 | CG | LEU | A | 569 | 14.454 | 3.796 | 21.041 | 1.00 | 31.25 | C |
| ATOM | 541 | CD1 | LEU | A | 569 | 13.901 | 2.447 | 20.638 | 1.00 | 32.64 | C |
| ATOM | 545 | CD2 | LEU | A | 569 | 13.527 | 4.504 | 22.040 | 1.00 | 33.35 | C |
| ATOM | 549 | C | LEU | A | 569 | 16.738 | 3.950 | 19.273 | 1.00 | 29.85 | C |
| ATOM | 550 | O | LEU | A | 569 | 16.241 | 3.314 | 18.340 | 1.00 | 30.01 | O |
| ATOM | 551 | N | GLU | A | 570 | 17.098 | 5.235 | 19.167 | 1.00 | 29.92 | N |
| ATOM | 553 | CA | GLU | A | 570 | 16.980 | 5.962 | 17.894 | 1.00 | 30.00 | C |
| ATOM | 555 | CB | GLU | A | 570 | 17.325 | 7.451 | 18.031 | 1.00 | 30.17 | C |
| ATOM | 558 | CG | GLU | A | 570 | 16.156 | 8.339 | 18.478 | 1.00 | 30.37 | C |
| ATOM | 561 | CD | GLU | A | 570 | 16.589 | 9.717 | 18.960 | 1.00 | 32.39 | C |
| ATOM | 562 | OE1 | GLU | A | 570 | 17.695 | 9.844 | 19.501 | 1.00 | 30.13 | O |
| ATOM | 563 | OE2 | GLU | A | 570 | 15.814 | 10.692 | 18.831 | 1.00 | 37.62 | O |
| ATOM | 564 | C | GLU | A | 570 | 17.798 | 5.296 | 16.770 | 1.00 | 30.28 | C |
| ATOM | 565 | O | GLU | A | 570 | 17.308 | 5.200 | 15.629 | 1.00 | 30.35 | O |
| ATOM | 566 | N | THR | A | 571 | 19.001 | 4.794 | 17.080 | 1.00 | 29.57 | N |
| ATOM | 568 | CA | THR | A | 571 | 19.794 | 4.091 | 16.066 | 1.00 | 29.67 | C |
| ATOM | 570 | CB | THR | A | 571 | 21.272 | 3.816 | 16.506 | 1.00 | 29.48 | C |
| ATOM | 572 | OG1 | THR | A | 571 | 21.314 | 2.935 | 17.629 | 1.00 | 28.30 | O |
| ATOM | 574 | CG2 | THR | A | 571 | 21.957 | 5.100 | 16.974 | 1.00 | 28.02 | C |
| ATOM | 578 | C | THR | A | 571 | 19.130 | 2.784 | 15.640 | 1.00 | 29.33 | C |
| ATOM | 579 | O | THR | A | 571 | 19.221 | 2.379 | 14.466 | 1.00 | 30.05 | O |
| ATOM | 580 | N | ALA | A | 572 | 18.492 | 2.124 | 16.590 | 1.00 | 29.18 | N |
| ATOM | 582 | CA | ALA | A | 572 | 17.756 | 0.891 | 16.318 | 1.00 | 29.80 | C |
| ATOM | 584 | CB | ALA | A | 572 | 17.308 | 0.240 | 17.602 | 1.00 | 29.64 | C |
| ATOM | 588 | C | ALA | A | 572 | 16.567 | 1.167 | 15.401 | 1.00 | 30.05 | C |
| ATOM | 589 | O | ALA | A | 572 | 16.316 | 0.391 | 14.495 | 1.00 | 30.93 | O |
| ATOM | 590 | N | LEU | A | 573 | 15.869 | 2.277 | 15.612 | 1.00 | 30.31 | N |
| ATOM | 592 | CA | LEU | A | 573 | 14.760 | 2.688 | 14.741 | 1.00 | 31.26 | C |
| ATOM | 594 | CB | LEU | A | 573 | 13.975 | 3.869 | 15.344 | 1.00 | 31.12 | C |
| ATOM | 597 | CG | LEU | A | 573 | 13.214 | 3.543 | 16.635 | 1.00 | 32.60 | C |
| ATOM | 599 | CD1 | LEU | A | 573 | 12.544 | 4.815 | 17.262 | 1.00 | 33.72 | C |
| ATOM | 603 | CD2 | LEU | A | 573 | 12.173 | 2.452 | 16.404 | 1.00 | 31.36 | C |
| ATOM | 607 | C | LEU | A | 573 | 15.291 | 3.033 | 13.339 | 1.00 | 31.87 | C |
| ATOM | 608 | O | LEU | A | 573 | 14.715 | 2.619 | 12.325 | 1.00 | 31.11 | O |
| ATOM | 609 | N | CYS | A | 574 | 16.420 | 3.735 | 13.286 | 1.00 | 31.88 | N |
| ATOM | 611 | CA | CYS | A | 574 | 17.080 | 3.994 | 12.016 | 1.00 | 32.11 | C |
| ATOM | 613 | CB | CYS | A | 574 | 18.368 | 4.798 | 12.228 | 1.00 | 32.66 | C |
| ATOM | 616 | SG | CYS | A | 574 | 18.138 | 6.536 | 12.617 | 1.00 | 33.97 | S |
| ATOM | 617 | C | CYS | A | 574 | 17.390 | 2.690 | 11.277 | 1.00 | 32.33 | C |
| ATOM | 618 | O | CYS | A | 574 | 17.255 | 2.609 | 10.047 | 1.00 | 31.91 | O |
| ATOM | 619 | N | THR | A | 575 | 17.804 | 1.669 | 12.023 | 1.00 | 32.03 | N |
| ATOM | 621 | CA | THR | A | 575 | 18.196 | 0.407 | 11.427 | 1.00 | 31.37 | C |
| ATOM | 623 | CB | THR | A | 575 | 18.929 | −0.460 | 12.452 | 1.00 | 31.74 | C |
| ATOM | 625 | OG1 | THR | A | 575 | 20.116 | 0.231 | 12.896 | 1.00 | 30.30 | O |
| ATOM | 627 | CG2 | THR | A | 575 | 19.419 | −1.765 | 11.804 | 1.00 | 32.94 | C |
| ATOM | 631 | C | THR | A | 575 | 16.979 | −0.332 | 10.864 | 1.00 | 31.23 | C |
| ATOM | 632 | O | THR | A | 575 | 17.053 | −0.878 | 9.783 | 1.00 | 30.31 | O |
| ATOM | 633 | N | ILE | A | 576 | 15.866 | −0.317 | 11.589 | 1.00 | 30.87 | N |
| ATOM | 635 | CA | ILE | A | 576 | 14.598 | −0.848 | 11.083 | 1.00 | 31.22 | C |
| ATOM | 637 | CB | ILE | A | 576 | 13.497 | −0.787 | 12.139 | 1.00 | 30.84 | C |
| ATOM | 639 | CG1 | ILE | A | 576 | 13.882 | −1.665 | 13.329 | 1.00 | 30.99 | C |
| ATOM | 642 | CD1 | ILE | A | 576 | 12.992 | −1.437 | 14.558 | 1.00 | 33.62 | C |
| ATOM | 646 | CG2 | ILE | A | 576 | 12.119 | −1.233 | 11.549 | 1.00 | 31.41 | C |
| ATOM | 650 | C | ILE | A | 576 | 14.168 | −0.119 | 9.813 | 1.00 | 31.43 | C |
| ATOM | 651 | O | ILE | A | 576 | 13.757 | −0.759 | 8.853 | 1.00 | 31.90 | O |
| ATOM | 652 | N | ARG | A | 577 | 14.301 | 1.194 | 9.780 | 1.00 | 30.79 | N |
| ATOM | 654 | CA | ARG | A | 577 | 13.964 | 1.937 | 8.585 | 1.00 | 31.23 | C |
| ATOM | 656 | CB | ARG | A | 577 | 14.058 | 3.437 | 8.821 | 1.00 | 31.07 | C |
| ATOM | 659 | CG | ARG | A | 577 | 13.601 | 4.319 | 7.656 | 1.00 | 30.28 | C |
| ATOM | 662 | CD | ARG | A | 577 | 12.315 | 3.885 | 6.972 | 1.00 | 29.17 | C |
| ATOM | 665 | NE | ARG | A | 577 | 11.145 | 4.167 | 7.796 | 1.00 | 29.79 | N |
| ATOM | 667 | CZ | ARG | A | 577 | 9.928 | 3.698 | 7.577 | 1.00 | 28.87 | C |
| ATOM | 668 | NH1 | ARG | A | 577 | 9.679 | 2.875 | 6.571 | 1.00 | 29.13 | N |
| ATOM | 671 | NH2 | ARG | A | 577 | 8.947 | 4.051 | 8.388 | 1.00 | 28.52 | N |
| ATOM | 674 | C | ARG | A | 577 | 14.827 | 1.544 | 7.378 | 1.00 | 32.02 | C |
| ATOM | 675 | O | ARG | A | 577 | 14.318 | 1.486 | 6.255 | 1.00 | 31.52 | O |
| ATOM | 676 | N | MET | A | 578 | 16.117 | 1.299 | 7.602 | 1.00 | 32.31 | N |
| ATOM | 678 | CA | MET | A | 578 | 17.036 | 0.882 | 6.525 | 1.00 | 32.58 | C |
| ATOM | 680 | CB | MET | A | 578 | 18.449 | 0.716 | 7.061 | 1.00 | 32.59 | C |
| ATOM | 683 | CG | MET | A | 578 | 19.157 | 2.010 | 7.334 | 1.00 | 35.68 | C |
| ATOM | 686 | SD | MET | A | 578 | 20.767 | 1.762 | 8.093 | 1.00 | 38.66 | S |
| ATOM | 687 | CE | MET | A | 578 | 20.727 | 3.087 | 9.249 | 1.00 | 37.18 | C |
| ATOM | 691 | C | MET | A | 578 | 16.587 | −0.447 | 5.895 | 1.00 | 32.70 | C |
| ATOM | 692 | O | MET | A | 578 | 16.530 | −0.574 | 4.660 | 1.00 | 33.48 | O |
| ATOM | 693 | N | PHE | A | 579 | 16.270 | −1.425 | 6.739 | 1.00 | 32.11 | N |
| ATOM | 695 | CA | PHE | A | 579 | 15.767 | −2.719 | 6.277 | 1.00 | 32.29 | C |
| ATOM | 697 | CB | PHE | A | 579 | 15.582 | −3.708 | 7.452 | 1.00 | 31.89 | C |
| ATOM | 700 | CG | PHE | A | 579 | 16.839 | −4.434 | 7.859 | 1.00 | 31.60 | C |
| ATOM | 701 | CD1 | PHE | A | 579 | 17.675 | −3.926 | 8.835 | 1.00 | 33.58 | C |
| ATOM | 703 | CE1 | PHE | A | 579 | 18.841 | −4.613 | 9.215 | 1.00 | 32.33 | C |
| ATOM | 705 | CZ | PHE | A | 579 | 19.162 | −5.807 | 8.612 | 1.00 | 31.30 | C |
| ATOM | 707 | CE2 | PHE | A | 579 | 18.321 | −6.319 | 7.656 | 1.00 | 31.22 | C |
| ATOM | 709 | CD2 | PHE | A | 579 | 17.178 | −5.632 | 7.281 | 1.00 | 31.99 | C |
| ATOM | 711 | C | PHE | A | 579 | 14.441 | −2.553 | 5.510 | 1.00 | 32.24 | C |
| ATOM | 712 | O | PHE | A | 579 | 14.185 | −3.257 | 4.523 | 1.00 | 32.02 | O |
| ATOM | 713 | N | THR | A | 580 | 13.604 | −1.635 | 5.970 | 1.00 | 32.08 | N |
| ATOM | 715 | CA | THR | A | 580 | 12.278 | −1.430 | 5.416 | 1.00 | 32.46 | C |
| ATOM | 717 | CB | THR | A | 580 | 11.408 | −0.621 | 6.410 | 1.00 | 32.61 | C |
| ATOM | 719 | OG1 | THR | A | 580 | 11.337 | −1.312 | 7.668 | 1.00 | 31.75 | O |
| ATOM | 721 | CG2 | THR | A | 580 | 9.934 | −0.550 | 5.950 | 1.00 | 33.38 | C |
| ATOM | 725 | C | THR | A | 580 | 12.318 | −0.753 | 4.043 | 1.00 | 32.81 | C |
| ATOM | 726 | O | THR | A | 580 | 11.677 | −1.215 | 3.108 | 1.00 | 33.63 | O |
| ATOM | 727 | N | ASP | A | 581 | 13.080 | 0.325 | 3.922 | 1.00 | 33.22 | N |
| ATOM | 729 | CA | ASP | A | 581 | 13.091 | 1.144 | 2.710 | 1.00 | 33.25 | C |
| ATOM | 731 | CB | ASP | A | 581 | 13.577 | 2.557 | 3.023 | 1.00 | 32.63 | C |
| ATOM | 734 | CG | ASP | A | 581 | 12.445 | 3.489 | 3.515 | 1.00 | 34.46 | C |
| ATOM | 735 | OD1 | ASP | A | 581 | 11.400 | 2.992 | 4.031 | 1.00 | 32.82 | O |
| ATOM | 736 | OD2 | ASP | A | 581 | 12.532 | 4.741 | 3.404 | 1.00 | 32.84 | O |
| ATOM | 737 | C | ASP | A | 581 | 13.963 | 0.518 | 1.615 | 1.00 | 33.04 | C |
| ATOM | 738 | O | ASP | A | 581 | 13.937 | 0.956 | 0.482 | 1.00 | 32.09 | O |
| ATOM | 739 | N | LEU | A | 582 | 14.767 | −0.477 | 1.981 | 1.00 | 34.07 | N |
| ATOM | 741 | CA | LEU | A | 582 | 15.502 | −1.291 | 1.016 | 1.00 | 34.19 | C |
| ATOM | 743 | CB | LEU | A | 582 | 16.827 | −1.781 | 1.606 | 1.00 | 34.53 | C |
| ATOM | 746 | CG | LEU | A | 582 | 17.906 | −0.693 | 1.643 | 1.00 | 34.93 | C |
| ATOM | 748 | CD1 | LEU | A | 582 | 19.138 | −1.108 | 2.422 | 1.00 | 33.63 | C |
| ATOM | 752 | CD2 | LEU | A | 582 | 18.282 | −0.297 | 0.212 | 1.00 | 36.11 | C |
| ATOM | 756 | C | LEU | A | 582 | 14.639 | −2.472 | 0.590 | 1.00 | 34.48 | C |
| ATOM | 757 | O | LEU | A | 582 | 15.118 | −3.352 | −0.109 | 1.00 | 34.23 | O |
| ATOM | 758 | N | ASN | A | 583 | 13.381 | −2.469 | 1.031 | 1.00 | 34.36 | N |
| ATOM | 760 | CA | ASN | A | 583 | 12.406 | −3.546 | 0.818 | 1.00 | 34.99 | C |
| ATOM | 762 | CB | ASN | A | 583 | 12.008 | −3.640 | −0.668 | 1.00 | 35.09 | C |
| ATOM | 765 | CG | ASN | A | 583 | 11.132 | −2.495 | −1.088 | 1.00 | 37.78 | C |
| ATOM | 766 | OD1 | ASN | A | 583 | 10.004 | −2.356 | −0.609 | 1.00 | 39.93 | O |
| ATOM | 767 | ND2 | ASN | A | 583 | 11.647 | −1.645 | −1.962 | 1.00 | 41.11 | N |
| ATOM | 770 | C | ASN | A | 583 | 12.804 | −4.914 | 1.360 | 1.00 | 34.32 | C |
| ATOM | 771 | O | ASN | A | 583 | 12.303 | −5.946 | 0.882 | 1.00 | 34.23 | O |
| ATOM | 772 | N | LEU | A | 584 | 13.710 | −4.947 | 2.331 | 1.00 | 33.18 | N |
| ATOM | 774 | CA | LEU | A | 584 | 14.185 | −6.230 | 2.854 | 1.00 | 33.64 | C |
| ATOM | 776 | CB | LEU | A | 584 | 15.523 | −6.099 | 3.584 | 1.00 | 33.24 | C |
| ATOM | 779 | CG | LEU | A | 584 | 16.669 | −5.565 | 2.722 | 1.00 | 32.96 | C |
| ATOM | 781 | CD1 | LEU | A | 584 | 17.913 | −5.315 | 3.557 | 1.00 | 31.66 | C |
| ATOM | 785 | CD2 | LEU | A | 584 | 16.954 | −6.513 | 1.562 | 1.00 | 32.83 | C |
| ATOM | 789 | C | LEU | A | 584 | 13.141 | −6.937 | 3.728 | 1.00 | 34.16 | C |
| ATOM | 790 | O | LEU | A | 584 | 12.989 | −8.153 | 3.647 | 1.00 | 34.32 | O |
| ATOM | 791 | N | VAL | A | 585 | 12.410 | −6.178 | 4.527 | 1.00 | 34.42 | N |
| ATOM | 793 | CA | VAL | A | 585 | 11.444 | −6.748 | 5.461 | 1.00 | 35.24 | C |
| ATOM | 795 | CB | VAL | A | 585 | 10.860 | −5.665 | 6.387 | 1.00 | 35.15 | C |
| ATOM | 797 | CG1 | VAL | A | 585 | 9.643 | −6.188 | 7.169 | 1.00 | 35.52 | C |
| ATOM | 801 | CG2 | VAL | A | 585 | 11.961 | −5.140 | 7.346 | 1.00 | 35.21 | C |
| ATOM | 805 | C | VAL | A | 585 | 10.290 | −7.447 | 4.730 | 1.00 | 36.18 | C |
| ATOM | 806 | O | VAL | A | 585 | 9.806 | −8.526 | 5.149 | 1.00 | 35.50 | O |
| ATOM | 807 | N | GLN | A | 586 | 9.846 | −6.823 | 3.647 | 1.00 | 36.68 | N |
| ATOM | 809 | CA | GLN | A | 586 | 8.700 | −7.340 | 2.930 | 1.00 | 37.07 | C |
| ATOM | 811 | CB | GLN | A | 586 | 7.877 | −6.192 | 2.338 | 1.00 | 37.74 | C |
| ATOM | 814 | CG | GLN | A | 586 | 8.403 | −5.499 | 1.083 | 1.00 | 39.21 | C |
| ATOM | 817 | CD | GLN | A | 586 | 7.248 | −4.869 | 0.298 | 1.00 | 42.46 | C |
| ATOM | 818 | OE1 | GLN | A | 586 | 6.256 | −5.540 | 0.002 | 1.00 | 41.35 | O |
| ATOM | 819 | NE2 | GLN | A | 586 | 7.362 | −3.575 | −0.006 | 1.00 | 46.17 | N |
| ATOM | 822 | C | GLN | A | 586 | 9.099 | −8.413 | 1.901 | 1.00 | 36.36 | C |
| ATOM | 823 | O | GLN | A | 586 | 8.424 | −9.427 | 1.791 | 1.00 | 37.00 | O |
| ATOM | 824 | N | ASN | A | 587 | 10.194 | −8.206 | 1.176 | 1.00 | 35.61 | N |
| ATOM | 826 | CA | ASN | A | 587 | 10.638 | −9.191 | 0.176 | 1.00 | 35.71 | C |
| ATOM | 828 | CB | ASN | A | 587 | 11.765 | −8.615 | −0.702 | 1.00 | 35.65 | C |
| ATOM | 831 | CG | ASN | A | 587 | 11.305 | −7.460 | −1.590 | 1.00 | 36.24 | C |
| ATOM | 832 | OD1 | ASN | A | 587 | 10.134 | −7.102 | −1.611 | 1.00 | 39.75 | O |
| ATOM | 833 | ND2 | ASN | A | 587 | 12.247 | −6.871 | −2.328 | 1.00 | 36.37 | N |
| ATOM | 836 | C | ASN | A | 587 | 11.114 | −10.533 | 0.786 | 1.00 | 35.57 | C |
| ATOM | 837 | O | ASN | A | 587 | 11.109 | −11.579 | 0.104 | 1.00 | 34.41 | O |
| ATOM | 838 | N | PHE | A | 588 | 11.578 | −10.483 | 2.038 | 1.00 | 35.12 | N |
| ATOM | 840 | CA | PHE | A | 588 | 12.088 | −11.661 | 2.750 | 1.00 | 35.20 | C |
| ATOM | 842 | CB | PHE | A | 588 | 13.535 | −11.407 | 3.179 | 1.00 | 34.99 | C |
| ATOM | 845 | CG | PHE | A | 588 | 14.465 | −11.258 | 2.018 | 1.00 | 34.40 | C |
| ATOM | 846 | CD1 | PHE | A | 588 | 15.075 | −10.060 | 1.740 | 1.00 | 32.27 | C |
| ATOM | 848 | CE1 | PHE | A | 588 | 15.895 | −9.933 | 0.634 | 1.00 | 34.30 | C |
| ATOM | 850 | CZ | PHE | A | 588 | 16.102 | −11.016 | −0.210 | 1.00 | 34.81 | C |
| ATOM | 852 | CE2 | PHE | A | 588 | 15.473 | −12.207 | 0.045 | 1.00 | 34.03 | C |
| ATOM | 854 | CD2 | PHE | A | 588 | 14.652 | −12.321 | 1.145 | 1.00 | 34.98 | C |
| ATOM | 856 | C | PHE | A | 588 | 11.225 | −12.084 | 3.948 | 1.00 | 35.69 | C |
| ATOM | 857 | O | PHE | A | 588 | 11.635 | −12.940 | 4.735 | 1.00 | 35.71 | O |
| ATOM | 858 | N | GLN | A | 589 | 10.045 | −11.476 | 4.079 | 1.00 | 36.23 | N |
| ATOM | 860 | CA | GLN | A | 589 | 9.063 | −11.824 | 5.119 | 1.00 | 37.00 | C |
| ATOM | 862 | CB | GLN | A | 589 | 8.457 | −13.211 | 4.840 | 1.00 | 37.03 | C |
| ATOM | 865 | CG | GLN | A | 589 | 7.561 | −13.284 | 3.632 | 1.00 | 38.23 | C |
| ATOM | 868 | CD | GLN | A | 589 | 6.518 | −14.388 | 3.787 | 1.00 | 41.94 | C |
| ATOM | 869 | OE1 | GLN | A | 589 | 5.522 | −14.216 | 4.506 | 1.00 | 45.87 | O |
| ATOM | 870 | NE2 | GLN | A | 589 | 6.749 | −15.526 | 3.139 | 1.00 | 41.94 | N |
| ATOM | 873 | C | GLN | A | 589 | 9.625 | −11.791 | 6.541 | 1.00 | 36.91 | C |
| ATOM | 874 | O | GLN | A | 589 | 9.285 | −12.617 | 7.379 | 1.00 | 36.83 | O |
| ATOM | 875 | N | MET | A | 590 | 10.466 | −10.816 | 6.826 | 1.00 | 37.58 | N |
| ATOM | 877 | CA | MET | A | 590 | 10.983 | −10.659 | 8.180 | 1.00 | 37.70 | C |
| ATOM | 879 | CB | MET | A | 590 | 12.018 | −9.572 | 8.217 | 1.00 | 37.85 | C |
| ATOM | 882 | CG | MET | A | 590 | 13.186 | −9.849 | 7.341 | 1.00 | 38.04 | C |
| ATOM | 885 | SD | MET | A | 590 | 14.419 | −8.644 | 7.693 | 1.00 | 35.14 | S |
| ATOM | 886 | CE | MET | A | 590 | 15.717 | −9.257 | 6.669 | 1.00 | 35.41 | C |
| ATOM | 890 | C | MET | A | 590 | 9.872 | −10.288 | 9.128 | 1.00 | 37.86 | C |
| ATOM | 891 | O | MET | A | 590 | 9.052 | −9.443 | 8.813 | 1.00 | 38.38 | O |
| ATOM | 892 | N | LYS | A | 591 | 9.837 | −10.945 | 10.279 | 1.00 | 38.15 | N |
| ATOM | 894 | CA | LYS | A | 591 | 8.843 | −10.663 | 11.296 | 1.00 | 38.30 | C |
| ATOM | 896 | CB | LYS | A | 591 | 8.629 | −11.889 | 12.170 | 1.00 | 38.99 | C |
| ATOM | 899 | CG | LYS | A | 591 | 7.890 | −13.003 | 11.411 | 1.00 | 41.53 | C |
| ATOM | 902 | CD | LYS | A | 591 | 7.746 | −14.298 | 12.210 | 1.00 | 44.02 | C |
| ATOM | 905 | CE | LYS | A | 591 | 7.783 | −15.538 | 11.299 | 1.00 | 45.54 | C |
| ATOM | 908 | NZ | LYS | A | 591 | 7.982 | −16.789 | 12.087 | 1.00 | 46.91 | N |
| ATOM | 912 | C | LYS | A | 591 | 9.345 | −9.496 | 12.115 | 1.00 | 37.85 | C |
| ATOM | 913 | O | LYS | A | 591 | 10.519 | −9.454 | 12.465 | 1.00 | 37.03 | O |
| ATOM | 914 | N | HIS | A | 592 | 8.463 | −8.544 | 12.402 | 1.00 | 37.27 | N |
| ATOM | 916 | CA | HIS | A | 592 | 8.871 | −7.309 | 13.057 | 1.00 | 37.50 | C |
| ATOM | 918 | CB | HIS | A | 592 | 7.672 | −6.412 | 13.363 | 1.00 | 37.66 | C |
| ATOM | 921 | CG | HIS | A | 592 | 8.051 | −5.064 | 13.905 | 1.00 | 38.68 | C |
| ATOM | 922 | ND1 | HIS | A | 592 | 8.522 | −4.046 | 13.101 | 1.00 | 40.07 | N |
| ATOM | 924 | CE1 | HIS | A | 592 | 8.765 | −2.976 | 13.842 | 1.00 | 40.15 | C |
| ATOM | 926 | NE2 | HIS | A | 592 | 8.478 | −3.265 | 15.100 | 1.00 | 39.72 | N |
| ATOM | 928 | CD2 | HIS | A | 592 | 8.041 | −4.571 | 15.168 | 1.00 | 40.44 | C |
| ATOM | 930 | C | HIS | A | 592 | 9.649 | −7.558 | 14.344 | 1.00 | 36.99 | C |
| ATOM | 931 | O | HIS | A | 592 | 10.750 | −7.036 | 14.515 | 1.00 | 37.35 | O |
| ATOM | 932 | N | GLU | A | 593 | 9.082 | −8.355 | 15.239 | 1.00 | 36.02 | N |
| ATOM | 934 | CA | GLU | A | 593 | 9.681 | −8.581 | 16.550 | 1.00 | 35.63 | C |
| ATOM | 936 | CB | GLU | A | 593 | 8.704 | −9.334 | 17.453 | 1.00 | 35.97 | C |
| ATOM | 939 | CG | GLU | A | 593 | 9.338 | −9.921 | 18.702 | 1.00 | 37.96 | C |
| ATOM | 942 | CD | GLU | A | 593 | 8.332 | −10.124 | 19.810 | 1.00 | 41.88 | C |
| ATOM | 943 | OE1 | GLU | A | 593 | 7.598 | −11.140 | 19.759 | 1.00 | 41.81 | O |
| ATOM | 944 | OE2 | GLU | A | 593 | 8.282 | −9.260 | 20.718 | 1.00 | 44.16 | O |
| ATOM | 945 | C | GLU | A | 593 | 11.018 | −9.329 | 16.471 | 1.00 | 34.35 | C |
| ATOM | 946 | O | GLU | A | 593 | 11.894 | −9.161 | 17.332 | 1.00 | 34.16 | O |
| ATOM | 947 | N | VAL | A | 594 | 11.153 | −10.162 | 15.448 | 1.00 | 33.24 | N |
| ATOM | 949 | CA | VAL | A | 594 | 12.380 | −10.908 | 15.190 | 1.00 | 32.19 | C |
| ATOM | 951 | CB | VAL | A | 594 | 12.153 | −12.063 | 14.144 | 1.00 | 31.94 | C |
| ATOM | 953 | CG1 | VAL | A | 594 | 13.463 | −12.690 | 13.701 | 1.00 | 32.10 | C |
| ATOM | 957 | CG2 | VAL | A | 594 | 11.245 | −13.152 | 14.729 | 1.00 | 31.70 | C |
| ATOM | 961 | C | VAL | A | 594 | 13.473 | −9.950 | 14.727 | 1.00 | 31.67 | C |
| ATOM | 962 | O | VAL | A | 594 | 14.589 | −10.006 | 15.222 | 1.00 | 30.91 | O |
| ATOM | 963 | N | LEU | A | 595 | 13.139 | −9.062 | 13.796 | 1.00 | 31.23 | N |
| ATOM | 965 | CA | LEU | A | 595 | 14.072 | −8.051 | 13.343 | 1.00 | 31.69 | C |
| ATOM | 967 | CB | LEU | A | 595 | 13.452 | −7.192 | 12.247 | 1.00 | 32.15 | C |
| ATOM | 970 | CG | LEU | A | 595 | 14.354 | −6.039 | 11.779 | 1.00 | 32.94 | C |
| ATOM | 972 | CD1 | LEU | A | 595 | 15.661 | −6.583 | 11.252 | 1.00 | 34.64 | C |
| ATOM | 976 | CD2 | LEU | A | 595 | 13.628 | −5.216 | 10.716 | 1.00 | 33.64 | C |
| ATOM | 980 | C | LEU | A | 595 | 14.510 | −7.159 | 14.509 | 1.00 | 31.38 | C |
| ATOM | 981 | O | LEU | A | 595 | 15.700 | −6.873 | 14.654 | 1.00 | 30.61 | O |
| ATOM | 982 | N | CYS | A | 596 | 13.551 | −6.749 | 15.338 | 1.00 | 30.97 | N |
| ATOM | 984 | CA | CYS | A | 596 | 13.847 | −5.941 | 16.527 | 1.00 | 31.39 | C |
| ATOM | 986 | CB | CYS | A | 596 | 12.575 | −5.557 | 17.280 | 1.00 | 31.32 | C |
| ATOM | 989 | SG | CYS | A | 596 | 11.599 | −4.292 | 16.461 | 1.00 | 34.15 | S |
| ATOM | 990 | C | CYS | A | 596 | 14.798 | −6.652 | 17.481 | 1.00 | 30.86 | C |
| ATOM | 991 | O | CYS | A | 596 | 15.764 | −6.037 | 17.972 | 1.00 | 31.85 | O |
| ATOM | 992 | N | ARG | A | 597 | 14.542 | −7.939 | 17.716 | 1.00 | 30.17 | N |
| ATOM | 994 | CA | ARG | A | 597 | 15.348 | −8.752 | 18.630 | 1.00 | 29.68 | C |
| ATOM | 996 | CB | ARG | A | 597 | 14.703 | −10.111 | 18.898 | 1.00 | 29.75 | C |
| ATOM | 999 | CG | ARG | A | 597 | 15.395 | −10.929 | 19.982 | 1.00 | 30.24 | C |
| ATOM | 1002 | CD | ARG | A | 597 | 14.737 | −12.269 | 20.280 | 1.00 | 31.74 | C |
| ATOM | 1005 | NE | ARG | A | 597 | 13.494 | −12.168 | 21.053 | 1.00 | 32.07 | N |
| ATOM | 1007 | CZ | ARG | A | 597 | 12.256 | −12.327 | 20.567 | 1.00 | 34.88 | C |
| ATOM | 1008 | NH1 | ARG | A | 597 | 12.021 | −12.579 | 19.272 | 1.00 | 35.26 | N |
| ATOM | 1011 | NH2 | ARG | A | 597 | 11.223 | −12.235 | 21.392 | 1.00 | 36.29 | N |
| ATOM | 1014 | C | ARG | A | 597 | 16.742 | −8.967 | 18.079 | 1.00 | 29.40 | C |
| ATOM | 1015 | O | ARG | A | 597 | 17.702 | −8.986 | 18.837 | 1.00 | 28.15 | O |
| ATOM | 1016 | N | TRP | A | 598 | 16.835 | −9.163 | 16.764 | 1.00 | 29.37 | N |
| ATOM | 1018 | CA | TRP | A | 598 | 18.120 | −9.347 | 16.095 | 1.00 | 28.82 | C |
| ATOM | 1020 | CB | TRP | A | 598 | 17.928 | −9.739 | 14.621 | 1.00 | 29.27 | C |
| ATOM | 1023 | CG | TRP | A | 598 | 19.248 | −9.848 | 13.909 | 1.00 | 28.69 | C |
| ATOM | 1024 | CD1 | TRP | A | 598 | 20.113 | −10.900 | 13.937 | 1.00 | 29.65 | C |
| ATOM | 1026 | NE1 | TRP | A | 598 | 21.242 | −10.601 | 13.207 | 1.00 | 31.90 | N |
| ATOM | 1028 | CE2 | TRP | A | 598 | 21.118 | −9.332 | 12.699 | 1.00 | 29.14 | C |
| ATOM | 1029 | CD2 | TRP | A | 598 | 19.879 | −8.827 | 13.131 | 1.00 | 29.44 | C |
| ATOM | 1030 | CE3 | TRP | A | 598 | 19.512 | −7.531 | 12.749 | 1.00 | 29.44 | C |
| ATOM | 1032 | CZ3 | TRP | A | 598 | 20.357 | −6.821 | 11.944 | 1.00 | 29.79 | C |
| ATOM | 1034 | CH2 | TRP | A | 598 | 21.589 | −7.355 | 11.540 | 1.00 | 29.29 | C |
| ATOM | 1036 | CZ2 | TRP | A | 598 | 21.971 | −8.609 | 11.894 | 1.00 | 28.46 | C |
| ATOM | 1038 | C | TRP | A | 598 | 18.979 | −8.087 | 16.215 | 1.00 | 28.83 | C |
| ATOM | 1039 | O | TRP | A | 598 | 20.137 | −8.164 | 16.601 | 1.00 | 29.05 | O |
| ATOM | 1040 | N | ILE | A | 599 | 18.397 | −6.929 | 15.928 | 1.00 | 28.80 | N |
| ATOM | 1042 | CA | ILE | A | 599 | 19.080 | −5.646 | 16.078 | 1.00 | 28.57 | C |
| ATOM | 1044 | CB | ILE | A | 599 | 18.170 | −4.467 | 15.670 | 1.00 | 28.39 | C |
| ATOM | 1046 | CG1 | ILE | A | 599 | 17.855 | −4.500 | 14.172 | 1.00 | 29.63 | C |
| ATOM | 1049 | CD1 | ILE | A | 599 | 16.764 | −3.554 | 13.747 | 1.00 | 30.52 | C |
| ATOM | 1053 | CG2 | ILE | A | 599 | 18.860 | −3.137 | 16.006 | 1.00 | 30.68 | C |
| ATOM | 1057 | C | ILE | A | 599 | 19.602 | −5.451 | 17.495 | 1.00 | 28.27 | C |
| ATOM | 1058 | O | ILE | A | 599 | 20.755 | −5.054 | 17.680 | 1.00 | 28.75 | O |
| ATOM | 1059 | N | LEU | A | 600 | 18.760 | −5.742 | 18.482 | 1.00 | 27.81 | N |
| ATOM | 1061 | CA | LEU | A | 600 | 19.121 | −5.596 | 19.879 | 1.00 | 27.28 | C |
| ATOM | 1063 | CB | LEU | A | 600 | 17.880 | −5.666 | 20.769 | 1.00 | 27.30 | C |
| ATOM | 1066 | CG | LEU | A | 600 | 16.934 | −4.451 | 20.673 | 1.00 | 27.61 | C |
| ATOM | 1068 | CD1 | LEU | A | 600 | 15.643 | −4.734 | 21.372 | 1.00 | 27.09 | C |
| ATOM | 1072 | CD2 | LEU | A | 600 | 17.568 | −3.184 | 21.253 | 1.00 | 28.63 | C |
| ATOM | 1076 | C | LEU | A | 600 | 20.187 | −6.617 | 20.327 | 1.00 | 27.27 | C |
| ATOM | 1077 | O | LEU | A | 600 | 21.028 | −6.290 | 21.161 | 1.00 | 26.24 | O |
| ATOM | 1078 | N | SER | A | 601 | 20.173 | −7.826 | 19.767 | 1.00 | 26.57 | N |
| ATOM | 1080 | CA | SER | A | 601 | 21.213 | −8.837 | 20.051 | 1.00 | 26.67 | C |
| ATOM | 1082 | CB | SER | A | 601 | 20.821 | −10.215 | 19.479 | 1.00 | 26.15 | C |
| ATOM | 1085 | OG | SER | A | 601 | 19.720 | −10.813 | 20.166 | 1.00 | 25.67 | O |
| ATOM | 1087 | C | SER | A | 601 | 22.573 | −8.411 | 19.453 | 1.00 | 26.88 | C |
| ATOM | 1088 | O | SER | A | 601 | 23.628 | −8.653 | 20.017 | 1.00 | 26.05 | O |
| ATOM | 1089 | N | VAL | A | 602 | 22.549 | −7.797 | 18.283 | 1.00 | 27.58 | N |
| ATOM | 1091 | CA | VAL | A | 602 | 23.780 | −7.352 | 17.656 | 1.00 | 27.77 | C |
| ATOM | 1093 | CB | VAL | A | 602 | 23.508 | −6.832 | 16.231 | 1.00 | 27.87 | C |
| ATOM | 1095 | CG1 | VAL | A | 602 | 24.653 | −5.965 | 15.737 | 1.00 | 29.12 | C |
| ATOM | 1099 | CG2 | VAL | A | 602 | 23.240 | −7.994 | 15.253 | 1.00 | 28.50 | C |
| ATOM | 1103 | C | VAL | A | 602 | 24.377 | −6.242 | 18.541 | 1.00 | 28.34 | C |
| ATOM | 1104 | O | VAL | A | 602 | 25.548 | −6.286 | 18.926 | 1.00 | 27.60 | O |
| ATOM | 1105 | N | LYS | A | 603 | 23.556 | −5.254 | 18.872 | 1.00 | 28.70 | N |
| ATOM | 1107 | CA | LYS | A | 603 | 24.003 | −4.148 | 19.717 | 1.00 | 29.38 | C |
| ATOM | 1109 | CB | LYS | A | 603 | 22.872 | −3.155 | 19.943 | 1.00 | 29.42 | C |
| ATOM | 1112 | CG | LYS | A | 603 | 23.262 | −1.941 | 20.782 | 1.00 | 31.62 | C |
| ATOM | 1115 | CD | LYS | A | 603 | 22.194 | −0.897 | 20.738 | 1.00 | 32.61 | C |
| ATOM | 1118 | CE | LYS | A | 603 | 21.027 | −1.283 | 21.599 | 1.00 | 35.34 | C |
| ATOM | 1121 | NZ | LYS | A | 603 | 19.869 | −0.628 | 21.043 | 1.00 | 39.66 | N |
| ATOM | 1125 | C | LYS | A | 603 | 24.540 | −4.661 | 21.056 | 1.00 | 29.88 | C |
| ATOM | 1126 | O | LYS | A | 603 | 25.572 | −4.211 | 21.514 | 1.00 | 30.29 | O |
| ATOM | 1127 | N | LYS | A | 604 | 23.850 | −5.613 | 21.671 | 1.00 | 30.04 | N |
| ATOM | 1129 | CA | LYS | A | 604 | 24.324 | −6.207 | 22.923 | 1.00 | 30.95 | C |
| ATOM | 1131 | CB | LYS | A | 604 | 23.352 | −7.286 | 23.424 | 1.00 | 30.85 | C |
| ATOM | 1134 | CG | LYS | A | 604 | 22.126 | −6.725 | 24.009 | 1.00 | 35.33 | C |
| ATOM | 1137 | CD | LYS | A | 604 | 21.072 | −7.789 | 24.380 | 1.00 | 38.98 | C |
| ATOM | 1140 | CE | LYS | A | 604 | 19.936 | −7.184 | 25.234 | 1.00 | 40.81 | C |
| ATOM | 1143 | NZ | LYS | A | 604 | 20.026 | −5.712 | 25.462 | 1.00 | 41.19 | N |
| ATOM | 1147 | C | LYS | A | 604 | 25.708 | −6.837 | 22.784 | 1.00 | 30.29 | C |
| ATOM | 1148 | O | LYS | A | 604 | 26.537 | −6.703 | 23.665 | 1.00 | 29.81 | O |
| ATOM | 1149 | N | ASN | A | 605 | 25.928 | −7.572 | 21.699 | 1.00 | 30.06 | N |
| ATOM | 1151 | CA | ASN | A | 605 | 27.205 | −8.263 | 21.505 | 1.00 | 30.33 | C |
| ATOM | 1153 | CB | ASN | A | 605 | 27.054 | −9.347 | 20.438 | 1.00 | 30.19 | C |
| ATOM | 1156 | CG | ASN | A | 605 | 26.464 | −10.614 | 21.027 | 1.00 | 31.60 | C |
| ATOM | 1157 | CD1 | ASN | A | 605 | 27.171 | −11.381 | 21.662 | 1.00 | 34.48 | O |
| ATOM | 1158 | ND2 | ASN | A | 605 | 25.154 | −10.782 | 20.910 | 1.00 | 29.75 | N |
| ATOM | 1161 | C | ASN | A | 605 | 28.393 | −7.351 | 21.222 | 1.00 | 30.31 | C |
| ATOM | 1162 | O | ASN | A | 605 | 29.541 | −7.779 | 21.325 | 1.00 | 29.93 | O |
| ATOM | 1163 | N | TYR | A | 606 | 28.096 | −6.101 | 20.864 | 1.00 | 30.77 | N |
| ATOM | 1165 | CA | TYR | A | 606 | 29.094 | −5.027 | 20.714 | 1.00 | 31.16 | C |
| ATOM | 1167 | CB | TYR | A | 606 | 28.524 | −3.899 | 19.833 | 1.00 | 30.63 | C |
| ATOM | 1170 | CG | TYR | A | 606 | 28.757 | −4.129 | 18.378 | 1.00 | 31.32 | C |
| ATOM | 1171 | CD1 | TYR | A | 606 | 30.042 | −4.101 | 17.865 | 1.00 | 31.76 | C |
| ATOM | 1173 | CE1 | TYR | A | 606 | 30.287 | −4.334 | 16.537 | 1.00 | 32.42 | C |
| ATOM | 1175 | CZ | TYR | A | 606 | 29.238 | −4.600 | 15.683 | 1.00 | 31.75 | C |
| ATOM | 1176 | OH | TYR | A | 606 | 29.521 | −4.835 | 14.353 | 1.00 | 31.75 | O |
| ATOM | 1178 | CE2 | TYR | A | 606 | 27.950 | −4.623 | 16.154 | 1.00 | 30.16 | C |
| ATOM | 1180 | CD2 | TYR | A | 606 | 27.713 | −4.394 | 17.511 | 1.00 | 30.01 | C |
| ATOM | 1182 | C | TYR | A | 606 | 29.553 | −4.389 | 22.023 | 1.00 | 31.98 | C |
| ATOM | 1183 | O | TYR | A | 606 | 30.468 | −3.594 | 21.996 | 1.00 | 31.56 | O |
| ATOM | 1184 | N | ARG | A | 607 | 28.898 | −4.693 | 23.142 | 1.00 | 33.28 | N |
| ATOM | 1186 | CA | ARG | A | 607 | 29.146 | −4.016 | 24.429 | 1.00 | 35.02 | C |
| ATOM | 1188 | CB | ARG | A | 607 | 28.336 | −4.684 | 25.551 | 1.00 | 35.04 | C |
| ATOM | 1191 | CG | ARG | A | 607 | 26.959 | −4.155 | 25.727 | 1.00 | 37.70 | C |
| ATOM | 1194 | CD | ARG | A | 607 | 26.294 | −4.678 | 26.991 | 1.00 | 39.36 | C |
| ATOM | 1197 | NE | ARG | A | 607 | 24.844 | −4.703 | 26.835 | 1.00 | 42.67 | N |
| ATOM | 1199 | CZ | ARG | A | 607 | 24.010 | −5.430 | 27.581 | 1.00 | 43.78 | C |
| ATOM | 1200 | NH1 | ARG | A | 607 | 24.466 | −6.224 | 28.552 | 1.00 | 43.30 | N |
| ATOM | 1203 | NH2 | ARG | A | 607 | 22.700 | −5.370 | 27.345 | 1.00 | 45.74 | N |
| ATOM | 1206 | C | ARG | A | 607 | 30.577 | −3.987 | 24.939 | 1.00 | 35.46 | C |
| ATOM | 1207 | O | ARG | A | 607 | 31.006 | −3.004 | 25.514 | 1.00 | 35.88 | O |
| ATOM | 1208 | N | LYS | A | 608 | 31.290 | −5.096 | 24.824 | 1.00 | 36.93 | N |
| ATOM | 1210 | CA | LYS | A | 608 | 32.666 | −5.142 | 25.335 | 1.00 | 38.15 | C |
| ATOM | 1212 | CB | LYS | A | 608 | 33.068 | −6.578 | 25.711 | 1.00 | 38.79 | C |
| ATOM | 1215 | CG | LYS | A | 608 | 32.304 | −7.171 | 26.889 | 1.00 | 39.97 | C |
| ATOM | 1218 | CD | LYS | A | 608 | 33.209 | −7.466 | 28.089 | 1.00 | 41.39 | C |
| ATOM | 1221 | CE | LYS | A | 608 | 32.865 | −8.784 | 28.755 | 1.00 | 41.54 | C |
| ATOM | 1224 | NZ | LYS | A | 608 | 33.799 | −9.043 | 29.896 | 1.00 | 42.55 | N |
| ATOM | 1228 | C | LYS | A | 608 | 33.696 | −4.570 | 24.353 | 1.00 | 37.89 | C |
| ATOM | 1229 | O | LYS | A | 608 | 34.874 | −4.506 | 24.682 | 1.00 | 38.34 | O |
| ATOM | 1230 | N | ASN | A | 609 | 33.274 | −4.159 | 23.161 | 1.00 | 37.47 | N |
| ATOM | 1232 | CA | ASN | A | 609 | 34.227 | −3.613 | 22.187 | 1.00 | 37.88 | C |
| ATOM | 1234 | CB | ASN | A | 609 | 33.629 | −3.620 | 20.788 | 1.00 | 37.01 | C |
| ATOM | 1237 | CG | ASN | A | 609 | 33.576 | −5.002 | 20.180 | 1.00 | 37.42 | C |
| ATOM | 1238 | OD1 | ASN | A | 609 | 33.620 | −6.011 | 20.880 | 1.00 | 38.35 | O |
| ATOM | 1239 | ND2 | ASN | A | 609 | 33.471 | −5.053 | 18.871 | 1.00 | 32.99 | N |
| ATOM | 1242 | C | ASN | A | 609 | 34.701 | −2.169 | 22.517 | 1.00 | 38.36 | C |
| ATOM | 1243 | O | ASN | A | 609 | 33.946 | −1.387 | 23.070 | 1.00 | 36.98 | O |
| ATOM | 1244 | N | VAL | A | 610 | 35.930 | −1.824 | 22.091 | 1.00 | 39.12 | N |
| ATOM | 1246 | CA | VAL | A | 610 | 36.442 | −0.452 | 22.196 | 1.00 | 39.82 | C |
| ATOM | 1248 | CB | VAL | A | 610 | 37.884 | −0.297 | 21.640 | 1.00 | 41.13 | C |
| ATOM | 1250 | CG1 | VAL | A | 610 | 38.517 | 1.049 | 22.100 | 1.00 | 40.50 | C |
| ATOM | 1254 | CG2 | VAL | A | 610 | 38.757 | −1.484 | 22.068 | 1.00 | 43.89 | C |
| ATOM | 1258 | C | VAL | A | 610 | 35.551 | 0.386 | 21.319 | 1.00 | 39.18 | C |
| ATOM | 1259 | O | VAL | A | 610 | 34.918 | −0.153 | 20.399 | 1.00 | 38.76 | O |
| ATOM | 1260 | N | ALA | A | 611 | 35.527 | 1.687 | 21.572 | 1.00 | 37.98 | N |
| ATOM | 1262 | CA | ALA | A | 611 | 34.487 | 2.544 | 21.029 | 1.00 | 37.77 | C |
| ATOM | 1264 | CB | ALA | A | 611 | 34.527 | 3.959 | 21.638 | 1.00 | 38.26 | C |
| ATOM | 1268 | C | ALA | A | 611 | 34.569 | 2.609 | 19.562 | 1.00 | 37.90 | C |
| ATOM | 1269 | O | ALA | A | 611 | 33.529 | 2.668 | 18.886 | 1.00 | 39.09 | O |
| ATOM | 1270 | N | TYR | A | 612 | 35.789 | 2.555 | 19.026 | 1.00 | 37.10 | N |
| ATOM | 1272 | CA | TYR | A | 612 | 35.907 | 2.589 | 17.593 | 1.00 | 36.21 | C |
| ATOM | 1274 | CB | TYR | A | 612 | 37.279 | 3.132 | 17.144 | 1.00 | 35.83 | C |
| ATOM | 1277 | CG | TYR | A | 612 | 38.548 | 2.537 | 17.706 | 1.00 | 31.94 | C |
| ATOM | 1278 | CD1 | TYR | A | 612 | 39.280 | 3.193 | 18.678 | 1.00 | 30.63 | C |
| ATOM | 1280 | CE1 | TYR | A | 612 | 40.486 | 2.685 | 19.128 | 1.00 | 29.64 | C |
| ATOM | 1282 | CZ | TYR | A | 612 | 40.997 | 1.524 | 18.566 | 1.00 | 30.05 | C |
| ATOM | 1283 | OH | TYR | A | 612 | 42.199 | 0.984 | 18.976 | 1.00 | 27.48 | O |
| ATOM | 1285 | CE2 | TYR | A | 612 | 40.280 | 0.859 | 17.593 | 1.00 | 28.47 | C |
| ATOM | 1287 | CD2 | TYR | A | 612 | 39.090 | 1.383 | 17.152 | 1.00 | 31.42 | C |
| ATOM | 1289 | C | TYR | A | 612 | 35.504 | 1.301 | 16.870 | 1.00 | 36.03 | C |
| ATOM | 1290 | O | TYR | A | 612 | 35.376 | 1.316 | 15.645 | 1.00 | 36.29 | O |
| ATOM | 1291 | N | HIS | A | 613 | 35.285 | 0.190 | 17.585 | 1.00 | 35.22 | N |
| ATOM | 1293 | CA | HIS | A | 613 | 34.680 | −1.018 | 16.950 | 1.00 | 34.43 | C |
| ATOM | 1295 | CB | HIS | A | 613 | 35.592 | −2.255 | 17.069 | 1.00 | 34.90 | C |
| ATOM | 1298 | CG | HIS | A | 613 | 36.922 | −2.105 | 16.400 | 1.00 | 36.25 | C |
| ATOM | 1299 | ND1 | HIS | A | 613 | 37.063 | −1.612 | 15.113 | 1.00 | 35.47 | N |
| ATOM | 1301 | CE1 | HIS | A | 613 | 38.348 | −1.602 | 14.792 | 1.00 | 38.87 | C |
| ATOM | 1303 | NE2 | HIS | A | 613 | 39.038 | −2.116 | 15.798 | 1.00 | 37.92 | N |
| ATOM | 1305 | CD2 | HIS | A | 613 | 38.168 | −2.442 | 16.818 | 1.00 | 37.07 | C |
| ATOM | 1307 | C | HIS | A | 613 | 33.342 | −1.367 | 17.591 | 1.00 | 33.82 | C |
| ATOM | 1308 | O | HIS | A | 613 | 32.970 | −2.551 | 17.697 | 1.00 | 32.40 | O |
| ATOM | 1309 | N | ASN | A | 614 | 32.651 | −0.328 | 18.049 | 1.00 | 33.04 | N |
| ATOM | 1311 | CA | ASN | A | 614 | 31.435 | −0.486 | 18.822 | 1.00 | 32.15 | C |
| ATOM | 1313 | CB | ASN | A | 614 | 31.384 | 0.536 | 19.980 | 1.00 | 31.90 | C |
| ATOM | 1316 | CG | ASN | A | 614 | 31.107 | 1.966 | 19.513 | 1.00 | 32.57 | C |
| ATOM | 1317 | OD1 | ASN | A | 614 | 30.893 | 2.227 | 18.324 | 1.00 | 34.59 | O |
| ATOM | 1318 | ND2 | ASN | A | 614 | 31.137 | 2.895 | 20.446 | 1.00 | 29.51 | N |
| ATOM | 1321 | C | ASN | A | 614 | 30.202 | −0.410 | 17.902 | 1.00 | 31.23 | C |
| ATOM | 1322 | O | ASN | A | 614 | 30.323 | −0.220 | 16.664 | 1.00 | 29.60 | O |
| ATOM | 1323 | N | TRP | A | 615 | 29.035 | −0.579 | 18.517 | 1.00 | 30.30 | N |
| ATOM | 1325 | CA | TRP | A | 615 | 27.760 | −0.531 | 17.812 | 1.00 | 30.70 | C |
| ATOM | 1327 | CB | TRP | A | 615 | 26.585 | −0.716 | 18.788 | 1.00 | 30.92 | C |
| ATOM | 1330 | CG | TRP | A | 615 | 25.286 | −0.217 | 18.237 | 1.00 | 31.99 | C |
| ATOM | 1331 | CD1 | TRP | A | 615 | 24.620 | 0.880 | 18.640 | 1.00 | 32.83 | C |
| ATOM | 1333 | NE1 | TRP | A | 615 | 23.474 | 1.035 | 17.900 | 1.00 | 33.36 | N |
| ATOM | 1335 | CE2 | TRP | A | 615 | 23.378 | 0.012 | 17.001 | 1.00 | 31.11 | C |
| ATOM | 1336 | CD2 | TRP | A | 615 | 24.506 | −0.802 | 17.188 | 1.00 | 30.42 | C |
| ATOM | 1337 | CE3 | TRP | A | 615 | 24.647 | −1.937 | 16.381 | 1.00 | 32.01 | C |
| ATOM | 1339 | CZ3 | TRP | A | 615 | 23.658 | −2.215 | 15.428 | 1.00 | 30.94 | C |
| ATOM | 1341 | CH2 | TRP | A | 615 | 22.556 | −1.376 | 15.269 | 1.00 | 31.69 | C |
| ATOM | 1343 | CZ2 | TRP | A | 615 | 22.384 | −0.270 | 16.057 | 1.00 | 31.08 | C |
| ATOM | 1345 | C | TRP | A | 615 | 27.583 | 0.755 | 17.002 | 1.00 | 30.51 | C |
| ATOM | 1346 | O | TRP | A | 615 | 27.164 | 0.694 | 15.873 | 1.00 | 31.12 | O |
| ATOM | 1347 | N | ARG | A | 616 | 27.963 | 1.902 | 17.542 | 1.00 | 30.28 | N |
| ATOM | 1349 | CA | ARG | A | 616 | 27.832 | 3.144 | 16.785 | 1.00 | 31.43 | C |
| ATOM | 1351 | CB | ARG | A | 616 | 28.192 | 4.379 | 17.621 | 1.00 | 31.30 | C |
| ATOM | 1354 | CG | ARG | A | 616 | 27.202 | 4.659 | 18.759 | 1.00 | 33.10 | C |
| ATOM | 1357 | CD | ARG | A | 616 | 25.719 | 4.547 | 18.363 | 1.00 | 35.82 | C |
| ATOM | 1360 | NE | ARG | A | 616 | 24.840 | 4.907 | 19.468 | 1.00 | 37.45 | N |
| ATOM | 1362 | CZ | ARG | A | 616 | 24.435 | 6.147 | 19.764 | 1.00 | 37.21 | C |
| ATOM | 1363 | NH1 | ARG | A | 616 | 24.797 | 7.196 | 19.031 | 1.00 | 35.24 | N |
| ATOM | 1366 | NH2 | ARG | A | 616 | 23.641 | 6.326 | 20.806 | 1.00 | 36.40 | N |
| ATOM | 1369 | C | ARG | A | 616 | 28.647 | 3.113 | 15.502 | 1.00 | 31.51 | C |
| ATOM | 1370 | O | ARG | A | 616 | 28.148 | 3.501 | 14.447 | 1.00 | 32.82 | O |
| ATOM | 1371 | N | HIS | A | 617 | 29.875 | 2.614 | 15.546 | 1.00 | 31.18 | N |
| ATOM | 1373 | CA | HIS | A | 617 | 30.606 | 2.498 | 14.312 | 1.00 | 30.51 | C |
| ATOM | 1375 | CB | HIS | A | 617 | 32.016 | 2.029 | 14.543 | 1.00 | 31.02 | C |
| ATOM | 1378 | CG | HIS | A | 617 | 32.728 | 1.695 | 13.269 | 1.00 | 30.16 | C |
| ATOM | 1379 | ND1 | HIS | A | 617 | 33.123 | 2.656 | 12.380 | 1.00 | 27.25 | N |
| ATOM | 1381 | CE1 | HIS | A | 617 | 33.698 | 2.080 | 11.344 | 1.00 | 31.29 | C |
| ATOM | 1383 | NE2 | HIS | A | 617 | 33.608 | 0.781 | 11.493 | 1.00 | 28.84 | N |
| ATOM | 1385 | CD2 | HIS | A | 617 | 33.017 | 0.511 | 12.696 | 1.00 | 29.49 | C |
| ATOM | 1387 | C | HIS | A | 617 | 29.929 | 1.577 | 13.292 | 1.00 | 30.71 | C |
| ATOM | 1388 | O | HIS | A | 617 | 29.861 | 1.885 | 12.099 | 1.00 | 30.52 | O |
| ATOM | 1389 | N | ALA | A | 618 | 29.453 | 0.440 | 13.750 | 1.00 | 30.71 | N |
| ATOM | 1391 | CA | ALA | A | 618 | 28.829 | −0.536 | 12.861 | 1.00 | 30.45 | C |
| ATOM | 1393 | CB | ALA | A | 618 | 28.498 | −1.823 | 13.630 | 1.00 | 30.32 | C |
| ATOM | 1397 | C | ALA | A | 618 | 27.565 | 0.061 | 12.258 | 1.00 | 30.85 | C |
| ATOM | 1398 | O | ALA | A | 618 | 27.290 | −0.087 | 11.071 | 1.00 | 31.51 | O |
| ATOM | 1399 | N | PHE | A | 619 | 26.803 | 0.755 | 13.093 | 1.00 | 31.16 | N |
| ATOM | 1401 | CA | PHE | A | 619 | 25.562 | 1.393 | 12.682 | 1.00 | 30.81 | C |
| ATOM | 1403 | CB | PHE | A | 619 | 24.853 | 1.997 | 13.896 | 1.00 | 30.88 | C |
| ATOM | 1406 | CG | PHE | A | 619 | 23.783 | 2.974 | 13.526 | 1.00 | 30.88 | C |
| ATOM | 1407 | CD1 | PHE | A | 619 | 22.616 | 2.546 | 12.947 | 1.00 | 29.68 | C |
| ATOM | 1409 | CE1 | PHE | A | 619 | 21.635 | 3.459 | 12.578 | 1.00 | 30.57 | C |
| ATOM | 1411 | CZ | PHE | A | 619 | 21.850 | 4.791 | 12.751 | 1.00 | 31.51 | C |
| ATOM | 1413 | CE2 | PHE | A | 619 | 23.019 | 5.230 | 13.319 | 1.00 | 32.18 | C |
| ATOM | 1415 | CD2 | PHE | A | 619 | 23.986 | 4.332 | 13.689 | 1.00 | 31.89 | C |
| ATOM | 1417 | C | PHE | A | 619 | 25.849 | 2.469 | 11.616 | 1.00 | 31.44 | C |
| ATOM | 1418 | O | PHE | A | 619 | 25.139 | 2.536 | 10.606 | 1.00 | 31.44 | O |
| ATOM | 1419 | N | ASN | A | 620 | 26.896 | 3.274 | 11.832 | 1.00 | 30.85 | N |
| ATOM | 1421 | CA | ASN | A | 620 | 27.363 | 4.262 | 10.836 | 1.00 | 31.22 | C |
| ATOM | 1423 | CB | ASN | A | 620 | 28.507 | 5.126 | 11.395 | 1.00 | 31.15 | C |
| ATOM | 1426 | CG | ASN | A | 620 | 28.010 | 6.191 | 12.378 | 1.00 | 34.02 | C |
| ATOM | 1427 | OD1 | ASN | A | 620 | 27.080 | 6.934 | 12.076 | 1.00 | 39.28 | O |
| ATOM | 1428 | ND2 | ASN | A | 620 | 28.657 | 6.290 | 13.543 | 1.00 | 35.23 | N |
| ATOM | 1431 | C | ASN | A | 620 | 27.803 | 3.663 | 9.498 | 1.00 | 31.18 | C |
| ATOM | 1432 | O | ASN | A | 620 | 27.540 | 4.239 | 8.454 | 1.00 | 31.47 | O |
| ATOM | 1433 | N | THR | A | 621 | 28.484 | 2.524 | 9.549 | 1.00 | 30.75 | N |
| ATOM | 1435 | CA | THR | A | 621 | 28.868 | 1.786 | 8.378 | 1.00 | 31.18 | C |
| ATOM | 1437 | CB | THR | A | 621 | 29.655 | 0.560 | 8.788 | 1.00 | 31.37 | C |
| ATOM | 1439 | OG1 | THR | A | 621 | 30.820 | 0.922 | 9.574 | 1.00 | 33.41 | O |
| ATOM | 1441 | CG2 | THR | A | 621 | 30.229 | −0.137 | 7.558 | 1.00 | 31.84 | C |
| ATOM | 1445 | C | THR | A | 621 | 27.614 | 1.362 | 7.575 | 1.00 | 31.48 | C |
| ATOM | 1446 | O | THR | A | 621 | 27.571 | 1.507 | 6.374 | 1.00 | 30.62 | O |
| ATOM | 1447 | N | ALA | A | 622 | 26.584 | 0.893 | 8.266 | 1.00 | 32.36 | N |
| ATOM | 1449 | CA | ALA | A | 622 | 25.315 | 0.494 | 7.643 | 1.00 | 32.22 | C |
| ATOM | 1451 | CB | ALA | A | 622 | 24.433 | −0.195 | 8.662 | 1.00 | 32.79 | C |
| ATOM | 1455 | C | ALA | A | 622 | 24.573 | 1.674 | 7.064 | 1.00 | 32.14 | C |
| ATOM | 1456 | O | ALA | A | 622 | 24.025 | 1.562 | 5.973 | 1.00 | 31.93 | O |
| ATOM | 1457 | N | GLN | A | 623 | 24.567 | 2.811 | 7.769 | 1.00 | 31.86 | N |
| ATOM | 1459 | CA | GLN | A | 623 | 23.886 | 3.999 | 7.274 | 1.00 | 31.61 | C |
| ATOM | 1461 | CB | GLN | A | 623 | 23.809 | 5.080 | 8.332 | 1.00 | 31.93 | C |
| ATOM | 1464 | CG | GLN | A | 623 | 23.074 | 6.375 | 7.924 | 1.00 | 31.37 | C |
| ATOM | 1467 | CD | GLN | A | 623 | 23.927 | 7.312 | 7.084 | 1.00 | 30.66 | C |
| ATOM | 1468 | OE1 | GLN | A | 623 | 25.155 | 7.428 | 7.290 | 1.00 | 31.16 | O |
| ATOM | 1469 | NE2 | GLN | A | 623 | 23.293 | 7.974 | 6.136 | 1.00 | 29.92 | N |
| ATOM | 1472 | C | GLN | A | 623 | 24.554 | 4.525 | 6.012 | 1.00 | 31.95 | C |
| ATOM | 1473 | O | GLN | A | 623 | 23.859 | 4.910 | 5.061 | 1.00 | 32.19 | O |
| ATOM | 1474 | N | CYS | A | 624 | 25.884 | 4.503 | 5.970 | 1.00 | 31.30 | N |
| ATOM | 1476 | CA | CYS | A | 624 | 26.592 | 4.885 | 4.759 | 1.00 | 31.66 | C |
| ATOM | 1478 | CB | CYS | A | 624 | 28.100 | 4.862 | 4.950 | 1.00 | 31.87 | C |
| ATOM | 1481 | SG | CYS | A | 624 | 29.005 | 5.620 | 3.568 | 1.00 | 32.91 | S |
| ATOM | 1482 | C | CYS | A | 624 | 26.217 | 3.949 | 3.590 | 1.00 | 31.58 | C |
| ATOM | 1483 | O | CYS | A | 624 | 26.085 | 4.406 | 2.473 | 1.00 | 30.48 | O |
| ATOM | 1484 | N | MET | A | 625 | 26.073 | 2.653 | 3.862 | 1.00 | 31.89 | N |
| ATOM | 1486 | CA | MET | A | 625 | 25.606 | 1.680 | 2.850 | 1.00 | 32.02 | C |
| ATOM | 1488 | CB | MET | A | 625 | 25.611 | 0.262 | 3.449 | 1.00 | 32.34 | C |
| ATOM | 1491 | CG | MET | A | 625 | 25.292 | −0.885 | 2.503 | 1.00 | 32.88 | C |
| ATOM | 1494 | SD | MET | A | 625 | 26.447 | −1.020 | 1.189 | 1.00 | 35.04 | S |
| ATOM | 1495 | CE | MET | A | 625 | 27.759 | −1.743 | 1.958 | 1.00 | 34.33 | C |
| ATOM | 1499 | C | MET | A | 625 | 24.216 | 2.056 | 2.326 | 1.00 | 32.02 | C |
| ATOM | 1500 | O | MET | A | 625 | 23.997 | 2.110 | 1.121 | 1.00 | 32.01 | O |
| ATOM | 1501 | N | PHE | A | 626 | 23.283 | 2.357 | 3.228 | 1.00 | 32.02 | N |
| ATOM | 1503 | CA | PHE | A | 626 | 21.938 | 2.757 | 2.838 | 1.00 | 31.43 | C |
| ATOM | 1505 | CB | PHE | A | 626 | 21.050 | 2.992 | 4.077 | 1.00 | 31.75 | C |
| ATOM | 1508 | CG | PHE | A | 626 | 19.652 | 3.449 | 3.755 | 1.00 | 31.75 | C |
| ATOM | 1509 | CD1 | PHE | A | 626 | 18.638 | 2.523 | 3.525 | 1.00 | 30.61 | C |
| ATOM | 1511 | CE1 | PHE | A | 626 | 17.364 | 2.939 | 3.231 | 1.00 | 32.72 | C |
| ATOM | 1513 | CZ | PHE | A | 626 | 17.066 | 4.312 | 3.168 | 1.00 | 32.65 | C |
| ATOM | 1515 | CE2 | PHE | A | 626 | 18.073 | 5.235 | 3.404 | 1.00 | 33.06 | C |
| ATOM | 1517 | CD2 | PHE | A | 626 | 19.355 | 4.805 | 3.689 | 1.00 | 31.65 | C |
| ATOM | 1519 | C | PHE | A | 626 | 22.008 | 4.016 | 1.987 | 1.00 | 31.60 | C |
| ATOM | 1520 | O | PHE | A | 626 | 21.331 | 4.119 | 0.960 | 1.00 | 31.35 | O |
| ATOM | 1521 | N | ALA | A | 627 | 22.825 | 4.971 | 2.409 | 1.00 | 31.24 | N |
| ATOM | 1523 | CA | ALA | A | 627 | 22.931 | 6.228 | 1.697 | 1.00 | 31.47 | C |
| ATOM | 1525 | CB | ALA | A | 627 | 23.790 | 7.240 | 2.471 | 1.00 | 31.67 | C |
| ATOM | 1529 | C | ALA | A | 627 | 23.520 | 5.958 | 0.321 | 1.00 | 31.49 | C |
| ATOM | 1530 | O | ALA | A | 627 | 23.007 | 6.465 | −0.679 | 1.00 | 31.23 | O |
| ATOM | 1531 | N | ALA | A | 628 | 24.569 | 5.140 | 0.266 | 1.00 | 30.86 | N |
| ATOM | 1533 | CA | ALA | A | 628 | 25.179 | 4.800 | −1.024 | 1.00 | 31.71 | C |
| ATOM | 1535 | CB | ALA | A | 628 | 26.466 | 4.007 | −0.843 | 1.00 | 31.46 | C |
| ATOM | 1539 | C | ALA | A | 628 | 24.193 | 4.067 | −1.955 | 1.00 | 31.94 | C |
| ATOM | 1540 | O | ALA | A | 628 | 24.194 | 4.311 | −3.149 | 1.00 | 32.27 | O |
| ATOM | 1541 | N | LEU | A | 629 | 23.305 | 3.243 | −1.401 | 1.00 | 32.55 | N |
| ATOM | 1543 | CA | LEU | A | 629 | 22.332 | 2.515 | −2.204 | 1.00 | 32.89 | C |
| ATOM | 1545 | CB | LEU | A | 629 | 21.719 | 1.357 | −1.403 | 1.00 | 33.12 | C |
| ATOM | 1548 | CG | LEU | A | 629 | 22.663 | 0.204 | −1.000 | 1.00 | 34.14 | C |
| ATOM | 1550 | CD1 | LEU | A | 629 | 22.092 | −0.664 | 0.149 | 1.00 | 34.32 | C |
| ATOM | 1554 | CD2 | LEU | A | 629 | 22.978 | −0.669 | −2.144 | 1.00 | 34.96 | C |
| ATOM | 1558 | C | LEU | A | 629 | 21.243 | 3.455 | −2.707 | 1.00 | 32.75 | C |
| ATOM | 1559 | O | LEU | A | 629 | 20.725 | 3.285 | −3.820 | 1.00 | 32.52 | O |
| ATOM | 1560 | N | LYS | A | 630 | 20.895 | 4.456 | −1.897 | 1.00 | 32.92 | N |
| ATOM | 1562 | CA | LYS | A | 630 | 19.762 | 5.340 | −2.192 | 1.00 | 32.40 | C |
| ATOM | 1564 | CB | LYS | A | 630 | 18.985 | 5.661 | −0.916 | 1.00 | 32.67 | C |
| ATOM | 1567 | CG | LYS | A | 630 | 18.220 | 4.478 | −0.352 | 1.00 | 34.05 | C |
| ATOM | 1570 | CD | LYS | A | 630 | 17.077 | 4.068 | −1.296 | 1.00 | 35.47 | C |
| ATOM | 1573 | CE | LYS | A | 630 | 16.130 | 3.083 | −0.647 | 1.00 | 36.74 | C |
| ATOM | 1576 | NZ | LYS | A | 630 | 14.939 | 2.850 | −1.507 | 1.00 | 37.93 | N |
| ATOM | 1580 | C | LYS | A | 630 | 20.199 | 6.626 | −2.881 | 1.00 | 32.33 | C |
| ATOM | 1581 | O | LYS | A | 630 | 19.934 | 6.834 | −4.055 | 1.00 | 32.10 | O |
| ATOM | 1582 | N | ALA | A | 631 | 20.865 | 7.502 | −2.150 | 1.00 | 32.35 | N |
| ATOM | 1584 | CA | ALA | A | 631 | 21.341 | 8.762 | −2.723 | 1.00 | 31.80 | C |
| ATOM | 1586 | CB | ALA | A | 631 | 21.944 | 9.623 | −1.632 | 1.00 | 32.01 | C |
| ATOM | 1590 | C | ALA | A | 631 | 22.385 | 8.487 | −3.810 | 1.00 | 31.40 | C |
| ATOM | 1591 | O | ALA | A | 631 | 22.409 | 9.145 | −4.848 | 1.00 | 29.37 | O |
| ATOM | 1592 | N | GLY | A | 632 | 23.249 | 7.510 | −3.547 | 1.00 | 31.00 | N |
| ATOM | 1594 | CA | GLY | A | 632 | 24.308 | 7.152 | −4.474 | 1.00 | 31.53 | C |
| ATOM | 1597 | C | GLY | A | 632 | 23.842 | 6.238 | −5.599 | 1.00 | 31.81 | C |
| ATOM | 1598 | O | GLY | A | 632 | 24.651 | 5.877 | −6.453 | 1.00 | 32.08 | O |
| ATOM | 1599 | N | LYS | A | 633 | 22.566 | 5.842 | −5.575 | 1.00 | 32.51 | N |
| ATOM | 1601 | CA | LYS | A | 633 | 21.926 | 5.037 | −6.631 | 1.00 | 33.28 | C |
| ATOM | 1603 | CB | LYS | A | 633 | 21.631 | 5.921 | −7.843 | 1.00 | 34.01 | C |
| ATOM | 1606 | CG | LYS | A | 633 | 20.450 | 6.887 | −7.619 | 1.00 | 34.40 | C |
| ATOM | 1609 | CD | LYS | A | 633 | 20.242 | 7.895 | −8.775 | 1.00 | 35.77 | C |
| ATOM | 1612 | CE | LYS | A | 633 | 20.244 | 7.226 | −10.165 | 1.00 | 36.91 | C |
| ATOM | 1615 | NZ | LYS | A | 633 | 19.667 | 8.095 | −11.259 | 1.00 | 34.73 | N |
| ATOM | 1619 | C | LYS | A | 633 | 22.674 | 3.751 | −7.031 | 1.00 | 33.99 | C |
| ATOM | 1620 | O | LYS | A | 633 | 22.746 | 3.386 | −8.200 | 1.00 | 34.69 | O |
| ATOM | 1621 | N | ILE | A | 634 | 23.227 | 3.060 | −6.039 | 1.00 | 34.33 | N |
| ATOM | 1623 | CA | ILE | A | 634 | 23.870 | 1.772 | −6.260 | 1.00 | 34.38 | C |
| ATOM | 1625 | CB | ILE | A | 634 | 25.100 | 1.652 | −5.329 | 1.00 | 34.92 | C |
| ATOM | 1627 | CG1 | ILE | A | 634 | 26.218 | 2.516 | −5.884 | 1.00 | 35.10 | C |
| ATOM | 1630 | CD1 | ILE | A | 634 | 27.081 | 2.989 | −4.854 | 1.00 | 37.95 | C |
| ATOM | 1634 | CG2 | ILE | A | 634 | 25.615 | 0.176 | −5.155 | 1.00 | 35.62 | C |
| ATOM | 1638 | C | ILE | A | 634 | 22.855 | 0.623 | −6.091 | 1.00 | 34.47 | C |
| ATOM | 1639 | O | ILE | A | 634 | 23.134 | −0.519 | −6.458 | 1.00 | 33.68 | O |
| ATOM | 1640 | N | GLN | A | 635 | 21.671 | 0.943 | −5.567 | 1.00 | 34.21 | N |
| ATOM | 1642 | CA | GLN | A | 635 | 20.625 | −0.049 | −5.353 | 1.00 | 34.80 | C |
| ATOM | 1644 | CB | GLN | A | 635 | 19.403 | 0.601 | −4.706 | 1.00 | 35.03 | C |
| ATOM | 1647 | CG | GLN | A | 635 | 18.209 | −0.315 | −4.540 | 1.00 | 36.49 | C |
| ATOM | 1650 | CD | GLN | A | 635 | 17.084 | 0.307 | −3.721 | 1.00 | 39.00 | C |
| ATOM | 1651 | OE1 | GLN | A | 635 | 16.900 | 1.526 | −3.705 | 1.00 | 39.26 | O |
| ATOM | 1652 | NE2 | GLN | A | 635 | 16.318 | −0.540 | −3.055 | 1.00 | 42.25 | N |
| ATOM | 1655 | C | GLN | A | 635 | 20.231 | −0.754 | −6.670 | 1.00 | 35.04 | C |
| ATOM | 1656 | O | GLN | A | 635 | 20.076 | −1.975 | −6.685 | 1.00 | 33.84 | O |
| ATOM | 1657 | N | ASN | A | 636 | 20.085 | 0.041 | −7.741 | 1.00 | 35.20 | N |
| ATOM | 1659 | CA | ASN | A | 636 | 19.770 | −0.431 | −9.090 | 1.00 | 36.02 | C |
| ATOM | 1661 | CB | ASN | A | 636 | 19.655 | 0.738 | −10.103 | 1.00 | 36.68 | C |
| ATOM | 1664 | CG | ASN | A | 636 | 18.818 | 1.904 | −9.595 | 1.00 | 39.99 | C |
| ATOM | 1665 | OD1 | ASN | A | 636 | 18.140 | 1.803 | −8.565 | 1.00 | 46.78 | O |
| ATOM | 1666 | ND2 | ASN | A | 636 | 18.861 | 3.030 | −10.321 | 1.00 | 42.53 | N |
| ATOM | 1669 | C | ASN | A | 636 | 20.802 | −1.389 | −9.666 | 1.00 | 35.44 | C |
| ATOM | 1670 | O | ASN | A | 636 | 20.492 | −2.168 | −10.552 | 1.00 | 35.08 | O |
| ATOM | 1671 | N | LYS | A | 637 | 22.038 | −1.296 | −9.192 | 1.00 | 35.20 | N |
| ATOM | 1673 | CA | LYS | A | 637 | 23.119 | −2.130 | −9.682 | 1.00 | 34.87 | C |
| ATOM | 1675 | CB | LYS | A | 637 | 24.436 | −1.348 | −9.650 | 1.00 | 35.40 | C |
| ATOM | 1678 | CG | LYS | A | 637 | 24.484 | −0.150 | −10.616 | 1.00 | 36.49 | C |
| ATOM | 1681 | CD | LYS | A | 637 | 25.718 | 0.730 | −10.327 | 1.00 | 38.37 | C |
| ATOM | 1684 | CE | LYS | A | 637 | 25.539 | 2.148 | −10.877 | 1.00 | 38.75 | C |
| ATOM | 1687 | NZ | LYS | A | 637 | 25.609 | 2.142 | −12.360 | 1.00 | 38.23 | N |
| ATOM | 1691 | C | LYS | A | 637 | 23.291 | −3.435 | −8.913 | 1.00 | 34.18 | C |
| ATOM | 1692 | O | LYS | A | 637 | 24.107 | −4.258 | −9.304 | 1.00 | 33.84 | O |
| ATOM | 1693 | N | LEU | A | 638 | 22.542 | −3.628 | −7.834 | 1.00 | 33.19 | N |
| ATOM | 1695 | CA | LEU | A | 638 | 22.712 | −4.805 | −6.979 | 1.00 | 32.72 | C |
| ATOM | 1697 | CB | LEU | A | 638 | 23.186 | −4.369 | −5.581 | 1.00 | 32.57 | C |
| ATOM | 1700 | CG | LEU | A | 638 | 24.543 | −3.658 | −5.477 | 1.00 | 32.78 | C |
| ATOM | 1702 | CD1 | LEU | A | 638 | 24.915 | −3.512 | −4.038 | 1.00 | 33.08 | C |
| ATOM | 1706 | CD2 | LEU | A | 638 | 25.653 | −4.387 | −6.192 | 1.00 | 34.28 | C |
| ATOM | 1710 | C | LEU | A | 638 | 21.423 | −5.635 | −6.858 | 1.00 | 32.44 | C |
| ATOM | 1711 | O | LEU | A | 638 | 20.320 | −5.133 | −7.095 | 1.00 | 32.46 | O |
| ATOM | 1712 | N | THR | A | 639 | 21.558 | −6.904 | −6.477 | 1.00 | 31.41 | N |
| ATOM | 1714 | CA | THR | A | 639 | 20.383 | −7.763 | −6.278 | 1.00 | 31.03 | C |
| ATOM | 1716 | CB | THR | A | 639 | 20.717 | −9.246 | −6.548 | 1.00 | 30.99 | C |
| ATOM | 1718 | OG1 | THR | A | 639 | 21.643 | −9.717 | −5.552 | 1.00 | 29.09 | O |
| ATOM | 1720 | CG2 | THR | A | 639 | 21.409 | −9.440 | −7.920 | 1.00 | 30.02 | C |
| ATOM | 1724 | C | THR | A | 639 | 19.885 | −7.644 | −4.848 | 1.00 | 30.57 | C |
| ATOM | 1725 | O | THR | A | 639 | 20.586 | −7.138 | −4.004 | 1.00 | 30.89 | O |
| ATOM | 1726 | N | ASP | A | 640 | 18.691 | −8.158 | −4.585 | 1.00 | 30.85 | N |
| ATOM | 1728 | CA | ASP | A | 640 | 18.121 | −8.202 | −3.245 | 1.00 | 31.53 | C |
| ATOM | 1730 | CB | ASP | A | 640 | 16.742 | −8.853 | −3.244 | 1.00 | 32.21 | C |
| ATOM | 1733 | CG | ASP | A | 640 | 15.663 | −7.950 | −3.781 | 1.00 | 36.04 | C |
| ATOM | 1734 | OD1 | ASP | A | 640 | 15.767 | −6.715 | −3.599 | 1.00 | 38.91 | O |
| ATOM | 1735 | OD2 | ASP | A | 640 | 14.668 | −8.407 | −4.387 | 1.00 | 41.38 | O |
| ATOM | 1736 | C | ASP | A | 640 | 18.997 | −8.962 | −2.263 | 1.00 | 31.29 | C |
| ATOM | 1737 | O | ASP | A | 640 | 19.163 | −8.503 | −1.146 | 1.00 | 31.11 | O |
| ATOM | 1738 | N | LEU | A | 641 | 19.533 | −10.115 | −2.665 | 1.00 | 31.09 | N |
| ATOM | 1740 | CA | LEU | A | 641 | 20.416 | −10.897 | −1.787 | 1.00 | 31.26 | C |
| ATOM | 1742 | CB | LEU | A | 641 | 20.779 | −12.249 | −2.421 | 1.00 | 31.33 | C |
| ATOM | 1745 | CG | LEU | A | 641 | 19.675 | −13.291 | −2.564 | 1.00 | 30.70 | C |
| ATOM | 1747 | CD1 | LEU | A | 641 | 20.271 | −14.613 | −3.042 | 1.00 | 30.42 | C |
| ATOM | 1751 | CD2 | LEU | A | 641 | 18.875 | −13.482 | −1.261 | 1.00 | 29.87 | C |
| ATOM | 1755 | C | LEU | A | 641 | 21.685 | −10.134 | −1.449 | 1.00 | 30.81 | C |
| ATOM | 1756 | O | LEU | A | 641 | 22.119 | −10.146 | −0.312 | 1.00 | 31.86 | O |
| ATOM | 1757 | N | GLU | A | 642 | 22.259 | −9.449 | −2.431 | 1.00 | 30.40 | N |
| ATOM | 1759 | CA | GLU | A | 642 | 23.483 | −8.674 | −2.225 | 1.00 | 30.27 | C |
| ATOM | 1761 | CB | GLU | A | 642 | 24.032 | −8.166 | −3.558 | 1.00 | 30.21 | C |
| ATOM | 1764 | CG | GLU | A | 642 | 24.558 | −9.287 | −4.455 | 1.00 | 30.03 | C |
| ATOM | 1767 | CD | GLU | A | 642 | 24.788 | −8.881 | −5.910 | 1.00 | 32.15 | C |
| ATOM | 1768 | OE1 | GLU | A | 642 | 24.321 | −7.805 | −6.335 | 1.00 | 33.95 | O |
| ATOM | 1769 | OE2 | GLU | A | 642 | 25.416 | −9.657 | −6.666 | 1.00 | 32.81 | O |
| ATOM | 1770 | C | GLU | A | 642 | 23.245 | −7.528 | −1.250 | 1.00 | 30.47 | C |
| ATOM | 1771 | O | GLU | A | 642 | 24.048 | −7.293 | −0.342 | 1.00 | 30.71 | O |
| ATOM | 1772 | N | ILE | A | 643 | 22.099 | −6.873 | −1.401 | 1.00 | 30.77 | N |
| ATOM | 1774 | CA | ILE | A | 643 | 21.713 | −5.760 | −0.569 | 1.00 | 30.94 | C |
| ATOM | 1776 | CB | ILE | A | 643 | 20.501 | −5.040 | −1.122 | 1.00 | 30.64 | C |
| ATOM | 1778 | CG1 | ILE | A | 643 | 20.882 | −4.330 | −2.424 | 1.00 | 31.48 | C |
| ATOM | 1781 | CD1 | ILE | A | 643 | 19.691 | −3.841 | −3.235 | 1.00 | 30.95 | C |
| ATOM | 1785 | CG2 | ILE | A | 643 | 19.980 | −4.033 | −0.117 | 1.00 | 31.85 | C |
| ATOM | 1789 | C | ILE | A | 643 | 21.482 | −6.207 | 0.866 | 1.00 | 31.22 | C |
| ATOM | 1790 | O | ILE | A | 643 | 21.996 | −5.568 | 1.776 | 1.00 | 30.90 | O |
| ATOM | 1791 | N | LEU | A | 644 | 20.753 | −7.302 | 1.048 | 1.00 | 30.89 | N |
| ATOM | 1793 | CA | LEU | A | 644 | 20.533 | −7.934 | 2.357 | 1.00 | 31.35 | C |
| ATOM | 1795 | CB | LEU | A | 644 | 19.684 | −9.204 | 2.158 | 1.00 | 31.85 | C |
| ATOM | 1798 | CG | LEU | A | 644 | 19.344 | −10.070 | 3.376 | 1.00 | 32.51 | C |
| ATOM | 1800 | CD1 | LEU | A | 644 | 18.561 | −9.281 | 4.419 | 1.00 | 31.68 | C |
| ATOM | 1804 | CD2 | LEU | A | 644 | 18.557 | −11.302 | 2.924 | 1.00 | 34.35 | C |
| ATOM | 1808 | C | LEU | A | 644 | 21.837 | −8.298 | 3.053 | 1.00 | 31.35 | C |
| ATOM | 1809 | O | LEU | A | 644 | 22.039 | −7.976 | 4.214 | 1.00 | 32.00 | O |
| ATOM | 1810 | N | ALA | A | 645 | 22.746 | −8.922 | 2.320 | 1.00 | 30.91 | N |
| ATOM | 1812 | CA | ALA | A | 645 | 23.987 | −9.374 | 2.897 | 1.00 | 31.28 | C |
| ATOM | 1814 | CB | ALA | A | 645 | 24.735 | −10.286 | 1.944 | 1.00 | 30.79 | C |
| ATOM | 1818 | C | ALA | A | 645 | 24.864 | −8.181 | 3.254 | 1.00 | 31.60 | C |
| ATOM | 1819 | O | ALA | A | 645 | 25.548 | −8.234 | 4.244 | 1.00 | 32.24 | O |
| ATOM | 1820 | N | LEU | A | 646 | 24.854 | −7.138 | 2.431 | 1.00 | 31.70 | N |
| ATOM | 1822 | CA | LEU | A | 646 | 25.726 | −5.985 | 2.633 | 1.00 | 32.69 | C |
| ATOM | 1824 | CB | LEU | A | 646 | 25.701 | −5.004 | 1.465 | 1.00 | 33.10 | C |
| ATOM | 1827 | CG | LEU | A | 646 | 26.465 | −5.375 | 0.202 | 1.00 | 36.19 | C |
| ATOM | 1829 | CD1 | LEU | A | 646 | 25.967 | −4.482 | −0.948 | 1.00 | 37.84 | C |
| ATOM | 1833 | CD2 | LEU | A | 646 | 27.950 | −5.267 | 0.411 | 1.00 | 37.50 | C |
| ATOM | 1837 | C | LEU | A | 646 | 25.291 | −5.242 | 3.861 | 1.00 | 32.76 | C |
| ATOM | 1838 | O | LEU | A | 646 | 26.122 | −4.810 | 4.627 | 1.00 | 32.57 | O |
| ATOM | 1839 | N | LEU | A | 647 | 23.985 | −5.094 | 4.035 | 1.00 | 33.13 | N |
| ATOM | 1841 | CA | LEU | A | 647 | 23.457 | −4.468 | 5.237 | 1.00 | 33.42 | C |
| ATOM | 1843 | CB | LEU | A | 647 | 21.947 | −4.256 | 5.101 | 1.00 | 33.13 | C |
| ATOM | 1846 | CG | LEU | A | 647 | 21.357 | −3.326 | 6.155 | 1.00 | 34.76 | C |
| ATOM | 1848 | CD1 | LEU | A | 647 | 22.086 | −1.969 | 6.182 | 1.00 | 34.70 | C |
| ATOM | 1852 | CD2 | LEU | A | 647 | 19.850 | −3.117 | 5.959 | 1.00 | 35.72 | C |
| ATOM | 1856 | C | LEU | A | 647 | 23.815 | −5.284 | 6.509 | 1.00 | 33.49 | C |
| ATOM | 1857 | O | LEU | A | 647 | 24.288 | −4.735 | 7.519 | 1.00 | 33.49 | O |
| ATOM | 1858 | N | ILE | A | 648 | 23.657 | −6.596 | 6.450 | 1.00 | 33.09 | N |
| ATOM | 1860 | CA | ILE | A | 648 | 23.917 | −7.413 | 7.618 | 1.00 | 32.95 | C |
| ATOM | 1862 | CB | ILE | A | 648 | 23.412 | −8.832 | 7.423 | 1.00 | 32.70 | C |
| ATOM | 1864 | CG1 | ILE | A | 648 | 21.876 | −8.857 | 7.471 | 1.00 | 32.59 | C |
| ATOM | 1867 | CD1 | ILE | A | 648 | 21.271 | −10.103 | 6.822 | 1.00 | 33.18 | C |
| ATOM | 1871 | CG2 | ILE | A | 648 | 24.015 | −9.759 | 8.477 | 1.00 | 33.03 | C |
| ATOM | 1875 | C | ILE | A | 648 | 25.405 | −7.411 | 7.965 | 1.00 | 33.03 | C |
| ATOM | 1876 | O | ILE | A | 648 | 25.755 | −7.312 | 9.138 | 1.00 | 32.99 | O |
| ATOM | 1877 | N | ALA | A | 649 | 26.252 | −7.529 | 6.943 | 1.00 | 32.46 | N |
| ATOM | 1879 | CA | ALA | A | 649 | 27.694 | −7.441 | 7.091 | 1.00 | 32.19 | C |
| ATOM | 1881 | CB | ALA | A | 649 | 28.376 | −7.690 | 5.747 | 1.00 | 31.91 | C |
| ATOM | 1885 | C | ALA | A | 649 | 28.166 | −6.112 | 7.683 | 1.00 | 31.99 | C |
| ATOM | 1886 | O | ALA | A | 649 | 28.959 | −6.097 | 8.603 | 1.00 | 31.02 | O |
| ATOM | 1887 | N | ALA | A | 650 | 27.667 | −4.998 | 7.162 | 1.00 | 32.64 | N |
| ATOM | 1889 | CA | ALA | A | 650 | 28.030 | −3.672 | 7.686 | 1.00 | 32.16 | C |
| ATOM | 1891 | CB | ALA | A | 650 | 27.265 | −2.592 | 6.957 | 1.00 | 32.87 | C |
| ATOM | 1895 | C | ALA | A | 650 | 27.754 | −3.599 | 9.167 | 1.00 | 31.97 | C |
| ATOM | 1896 | O | ALA | A | 650 | 28.619 | −3.171 | 9.962 | 1.00 | 31.46 | O |
| ATOM | 1897 | N | LEU | A | 651 | 26.582 | −4.081 | 9.556 | 1.00 | 31.43 | N |
| ATOM | 1899 | CA | LEU | A | 651 | 26.159 | −4.076 | 10.963 | 1.00 | 31.21 | C |
| ATOM | 1901 | CB | LEU | A | 651 | 24.673 | −4.404 | 11.083 | 1.00 | 31.32 | C |
| ATOM | 1904 | CG | LEU | A | 651 | 23.742 | −3.326 | 10.542 | 1.00 | 32.12 | C |
| ATOM | 1906 | CD1 | LEU | A | 651 | 22.344 | −3.843 | 10.355 | 1.00 | 32.83 | C |
| ATOM | 1910 | CD2 | LEU | A | 651 | 23.743 | −2.125 | 11.438 | 1.00 | 32.23 | C |
| ATOM | 1914 | C | LEU | A | 651 | 26.957 | −5.047 | 11.850 | 1.00 | 31.12 | C |
| ATOM | 1915 | O | LEU | A | 651 | 27.210 | −4.751 | 13.011 | 1.00 | 29.74 | O |
| ATOM | 1916 | N | SER | A | 652 | 27.396 | −6.167 | 11.277 | 1.00 | 31.07 | N |
| ATOM | 1918 | CA | SER | A | 652 | 28.029 | −7.252 | 12.038 | 1.00 | 31.40 | C |
| ATOM | 1920 | CB | SER | A | 652 | 27.488 | −8.590 | 11.545 | 1.00 | 31.66 | C |
| ATOM | 1923 | OG | SER | A | 652 | 26.075 | −8.596 | 11.563 | 1.00 | 33.22 | O |
| ATOM | 1925 | C | SER | A | 652 | 29.551 | −7.369 | 11.982 | 1.00 | 30.56 | C |
| ATOM | 1926 | O | SER | A | 652 | 30.117 | −8.177 | 12.697 | 1.00 | 28.98 | O |
| ATOM | 1927 | N | HIS | A | 653 | 30.212 | −6.586 | 11.144 | 1.00 | 30.93 | N |
| ATOM | 1929 | CA | HIS | A | 653 | 31.583 | −6.901 | 10.760 | 1.00 | 31.59 | C |
| ATOM | 1931 | CB | HIS | A | 653 | 32.037 | −6.037 | 9.578 | 1.00 | 31.74 | C |
| ATOM | 1934 | CG | HIS | A | 653 | 32.262 | −4.609 | 9.935 | 1.00 | 32.86 | C |
| ATOM | 1935 | ND1 | HIS | A | 653 | 31.236 | −3.705 | 10.043 | 1.00 | 34.83 | N |
| ATOM | 1937 | CE1 | HIS | A | 653 | 31.724 | −2.526 | 10.380 | 1.00 | 33.06 | C |
| ATOM | 1939 | NE2 | HIS | A | 653 | 33.035 | −2.632 | 10.474 | 1.00 | 34.35 | N |
| ATOM | 1941 | CD2 | HIS | A | 653 | 33.397 | −3.922 | 10.205 | 1.00 | 34.04 | C |
| ATOM | 1943 | C | HIS | A | 653 | 32.610 | −6.765 | 11.900 | 1.00 | 31.77 | C |
| ATOM | 1944 | O | HIS | A | 653 | 33.694 | −7.343 | 11.786 | 1.00 | 31.53 | O |
| ATOM | 1945 | N | ASP | A | 654 | 32.287 | −6.016 | 12.967 | 1.00 | 31.02 | N |
| ATOM | 1947 | CA | ASP | A | 654 | 33.203 | −5.869 | 14.110 | 1.00 | 32.06 | C |
| ATOM | 1949 | CB | ASP | A | 654 | 33.267 | −4.406 | 14.541 | 1.00 | 31.95 | C |
| ATOM | 1952 | CG | ASP | A | 654 | 34.342 | −3.613 | 13.798 | 1.00 | 34.24 | C |
| ATOM | 1953 | OD1 | ASP | A | 654 | 35.199 | −4.243 | 13.103 | 1.00 | 32.30 | O |
| ATOM | 1954 | OD2 | ASP | A | 654 | 34.419 | −2.361 | 13.876 | 1.00 | 34.59 | O |
| ATOM | 1955 | C | ASP | A | 654 | 32.867 | −6.772 | 15.296 | 1.00 | 33.06 | C |
| ATOM | 1956 | O | ASP | A | 654 | 33.422 | −6.624 | 16.379 | 1.00 | 31.77 | O |
| ATOM | 1957 | N | LEU | A | 655 | 31.933 | −7.703 | 15.108 | 1.00 | 34.41 | N |
| ATOM | 1959 | CA | LEU | A | 655 | 31.696 | −8.763 | 16.103 | 1.00 | 36.09 | C |
| ATOM | 1961 | CB | LEU | A | 655 | 30.302 | −9.352 | 15.905 | 1.00 | 35.24 | C |
| ATOM | 1964 | CG | LEU | A | 655 | 29.159 | −8.361 | 16.026 | 1.00 | 35.88 | C |
| ATOM | 1966 | CD1 | LEU | A | 655 | 27.882 | −8.973 | 15.468 | 1.00 | 37.30 | C |
| ATOM | 1970 | CD2 | LEU | A | 655 | 29.000 | −7.937 | 17.479 | 1.00 | 36.08 | C |
| ATOM | 1974 | C | LEU | A | 655 | 32.749 | −9.857 | 15.921 | 1.00 | 37.80 | C |
| ATOM | 1975 | O | LEU | A | 655 | 32.686 | −10.587 | 14.946 | 1.00 | 38.36 | O |
| ATOM | 1976 | N | ASP | A | 656 | 33.700 | −10.006 | 16.838 | 1.00 | 40.55 | N |
| ATOM | 1978 | CA | ASP | A | 656 | 34.920 | −10.783 | 16.530 | 1.00 | 42.89 | C |
| ATOM | 1980 | CB | ASP | A | 656 | 36.142 | −9.844 | 16.644 | 1.00 | 44.08 | C |
| ATOM | 1983 | CG | ASP | A | 656 | 37.298 | −10.225 | 15.710 | 1.00 | 48.10 | C |
| ATOM | 1984 | OD1 | ASP | A | 656 | 37.051 | −10.684 | 14.565 | 1.00 | 52.44 | O |
| ATOM | 1985 | OD2 | ASP | A | 656 | 38.508 | −10.062 | 16.043 | 1.00 | 54.53 | O |
| ATOM | 1986 | C | ASP | A | 656 | 35.176 | −12.045 | 17.381 | 1.00 | 43.63 | C |
| ATOM | 1987 | O | ASP | A | 656 | 36.266 | −12.637 | 17.299 | 1.00 | 43.86 | O |
| ATOM | 1988 | N | HIS | A | 657 | 34.202 | −12.481 | 18.176 | 1.00 | 44.15 | N |
| ATOM | 1990 | CA | HIS | A | 657 | 34.493 | −13.496 | 19.209 | 1.00 | 44.68 | C |
| ATOM | 1992 | CB | HIS | A | 657 | 33.333 | −13.615 | 20.208 | 1.00 | 44.74 | C |
| ATOM | 1995 | CG | HIS | A | 657 | 33.742 | −14.123 | 21.562 | 1.00 | 46.30 | C |
| ATOM | 1996 | ND1 | HIS | A | 657 | 32.944 | −14.961 | 22.319 | 1.00 | 47.41 | N |
| ATOM | 1998 | CE1 | HIS | A | 657 | 33.557 | −15.248 | 23.455 | 1.00 | 47.05 | C |
| ATOM | 2000 | NE2 | HIS | A | 657 | 34.726 | −14.630 | 23.465 | 1.00 | 47.08 | N |
| ATOM | 2002 | CD2 | HIS | A | 657 | 34.866 | −13.920 | 22.294 | 1.00 | 47.25 | C |
| ATOM | 2004 | C | HIS | A | 657 | 34.902 | −14.885 | 18.649 | 1.00 | 44.72 | C |
| ATOM | 2005 | O | HIS | A | 657 | 34.141 | −15.568 | 17.959 | 1.00 | 44.45 | O |
| ATOM | 2006 | N | LEU | A | 672 | 45.942 | −7.901 | 25.006 | 1.00 | 40.22 | N |
| ATOM | 2008 | CA | LEU | A | 672 | 44.765 | −7.106 | 25.373 | 1.00 | 39.72 | C |
| ATOM | 2010 | CB | LEU | A | 672 | 44.699 | −6.859 | 26.913 | 1.00 | 39.47 | C |
| ATOM | 2013 | CG | LEU | A | 672 | 43.415 | −7.305 | 27.648 | 1.00 | 41.50 | C |
| ATOM | 2015 | CD1 | LEU | A | 672 | 43.452 | −6.944 | 29.155 | 1.00 | 42.61 | C |
| ATOM | 2019 | CD2 | LEU | A | 672 | 42.112 | −6.783 | 27.000 | 1.00 | 42.23 | C |
| ATOM | 2023 | C | LEU | A | 672 | 44.744 | −5.790 | 24.557 | 1.00 | 38.16 | C |
| ATOM | 2024 | O | LEU | A | 672 | 43.725 | −5.460 | 23.975 | 1.00 | 39.19 | O |
| ATOM | 2025 | N | ALA | A | 673 | 45.862 | −5.067 | 24.492 | 1.00 | 37.22 | N |
| ATOM | 2027 | CA | ALA | A | 673 | 45.987 | −3.837 | 23.650 | 1.00 | 35.47 | C |
| ATOM | 2029 | CB | ALA | A | 673 | 47.445 | −3.430 | 23.512 | 1.00 | 35.51 | C |
| ATOM | 2033 | C | ALA | A | 673 | 45.363 | −3.964 | 22.279 | 1.00 | 34.63 | C |
| ATOM | 2034 | O | ALA | A | 673 | 45.567 | −4.963 | 21.609 | 1.00 | 34.37 | O |
| ATOM | 2035 | N | GLN | A | 674 | 44.559 | −2.973 | 21.877 | 1.00 | 33.43 | N |
| ATOM | 2037 | CA | GLN | A | 674 | 43.895 | −2.994 | 20.595 | 1.00 | 33.60 | C |
| ATOM | 2039 | CB | GLN | A | 674 | 42.441 | −2.497 | 20.707 | 1.00 | 34.24 | C |
| ATOM | 2042 | CG | GLN | A | 674 | 41.513 | −3.452 | 21.486 | 1.00 | 37.75 | C |
| ATOM | 2045 | CD | GLN | A | 674 | 40.544 | −4.242 | 20.641 | 1.00 | 38.80 | C |
| ATOM | 2046 | OE1 | GLN | A | 674 | 40.254 | −3.910 | 19.474 | 1.00 | 44.44 | O |
| ATOM | 2047 | NE2 | GLN | A | 674 | 39.998 | −5.288 | 21.241 | 1.00 | 43.56 | N |
| ATOM | 2050 | C | GLN | A | 674 | 44.636 | −2.149 | 19.546 | 1.00 | 32.40 | C |
| ATOM | 2051 | O | GLN | A | 674 | 45.186 | −1.073 | 19.851 | 1.00 | 31.00 | O |
| ATOM | 2052 | N | LEU | A | 675 | 44.624 | −2.667 | 18.328 | 1.00 | 30.90 | N |
| ATOM | 2054 | CA | LEU | A | 675 | 45.211 | −2.030 | 17.161 | 1.00 | 31.32 | C |
| ATOM | 2056 | CB | LEU | A | 675 | 45.047 | −2.949 | 15.927 | 1.00 | 30.92 | C |
| ATOM | 2059 | CG | LEU | A | 675 | 45.868 | −4.231 | 15.922 | 1.00 | 31.96 | C |
| ATOM | 2061 | CD1 | LEU | A | 675 | 45.312 | −5.271 | 14.935 | 1.00 | 32.94 | C |
| ATOM | 2065 | CD2 | LEU | A | 675 | 47.374 | −3.924 | 15.643 | 1.00 | 31.58 | C |
| ATOM | 2069 | C | LEU | A | 675 | 44.462 | −0.744 | 16.887 | 1.00 | 31.01 | C |
| ATOM | 2070 | O | LEU | A | 675 | 43.268 | −0.643 | 17.182 | 1.00 | 31.24 | O |
| ATOM | 2071 | N | TYR | A | 676 | 45.154 | 0.234 | 16.326 | 1.00 | 30.42 | N |
| ATOM | 2073 | CA | TYR | A | 676 | 44.500 | 1.431 | 15.814 | 1.00 | 30.40 | C |
| ATOM | 2075 | CB | TYR | A | 676 | 45.470 | 2.333 | 15.026 | 1.00 | 30.49 | C |
| ATOM | 2078 | CG | TYR | A | 676 | 45.057 | 3.757 | 15.120 | 1.00 | 29.31 | C |
| ATOM | 2079 | CD1 | TYR | A | 676 | 45.421 | 4.496 | 16.204 | 1.00 | 29.50 | C |
| ATOM | 2081 | CE1 | TYR | A | 676 | 45.044 | 5.813 | 16.339 | 1.00 | 31.10 | C |
| ATOM | 2083 | CZ | TYR | A | 676 | 44.234 | 6.398 | 15.399 | 1.00 | 28.90 | C |
| ATOM | 2084 | OH | TYR | A | 676 | 43.874 | 7.712 | 15.604 | 1.00 | 33.89 | O |
| ATOM | 2086 | CE2 | TYR | A | 676 | 43.817 | 5.686 | 14.301 | 1.00 | 30.81 | C |
| ATOM | 2088 | CD2 | TYR | A | 676 | 44.240 | 4.351 | 14.151 | 1.00 | 30.43 | C |
| ATOM | 2090 | C | TYR | A | 676 | 43.347 | 1.066 | 14.920 | 1.00 | 31.11 | C |
| ATOM | 2091 | O | TYR | A | 676 | 43.381 | 0.042 | 14.239 | 1.00 | 30.91 | O |
| ATOM | 2092 | N | CYS | A | 677 | 42.325 | 1.903 | 14.918 | 1.00 | 32.33 | N |
| ATOM | 2094 | CA | CYS | A | 677 | 41.246 | 1.853 | 13.902 | 1.00 | 34.86 | C |
| ATOM | 2096 | CB | CYS | A | 677 | 40.555 | 3.228 | 13.918 | 1.00 | 35.17 | C |
| ATOM | 2099 | SG | CYS | A | 677 | 38.936 | 3.136 | 13.268 | 1.00 | 45.40 | S |
| ATOM | 2100 | C | CYS | A | 677 | 41.718 | 1.627 | 12.430 | 1.00 | 33.82 | C |
| ATOM | 2101 | O | CYS | A | 677 | 42.504 | 2.406 | 11.920 | 1.00 | 33.14 | O |
| ATOM | 2102 | N | HIS | A | 678 | 41.188 | 0.596 | 11.765 | 1.00 | 34.07 | N |
| ATOM | 2104 | CA | HIS | A | 678 | 41.471 | 0.227 | 10.363 | 1.00 | 33.24 | C |
| ATOM | 2106 | CB | HIS | A | 678 | 41.112 | 1.358 | 9.401 | 1.00 | 34.61 | C |
| ATOM | 2109 | CG | HIS | A | 678 | 39.763 | 1.949 | 9.632 | 1.00 | 35.54 | C |
| ATOM | 2110 | ND1 | HIS | A | 678 | 38.602 | 1.205 | 9.588 | 1.00 | 37.52 | N |
| ATOM | 2112 | CE1 | HIS | A | 678 | 37.569 | 1.999 | 9.805 | 1.00 | 34.44 | C |
| ATOM | 2114 | NE2 | HIS | A | 678 | 38.017 | 3.225 | 9.985 | 1.00 | 35.87 | N |
| ATOM | 2116 | CD2 | HIS | A | 678 | 39.386 | 3.220 | 9.885 | 1.00 | 35.92 | C |
| ATOM | 2118 | C | HIS | A | 678 | 42.899 | −0.232 | 10.041 | 1.00 | 33.03 | C |
| ATOM | 2119 | O | HIS | A | 678 | 43.323 | −0.198 | 8.892 | 1.00 | 33.36 | O |
| ATOM | 2120 | N | SER | A | 679 | 43.630 | −0.677 | 11.049 | 1.00 | 31.57 | N |
| ATOM | 2122 | CA | SER | A | 679 | 44.947 | −1.228 | 10.870 | 1.00 | 30.05 | C |
| ATOM | 2124 | CB | SER | A | 679 | 45.432 | −1.789 | 12.208 | 1.00 | 30.03 | C |
| ATOM | 2127 | OG | SER | A | 679 | 46.508 | −2.685 | 12.022 | 1.00 | 27.55 | O |
| ATOM | 2129 | C | SER | A | 679 | 44.933 | −2.374 | 9.854 | 1.00 | 30.06 | C |
| ATOM | 2130 | O | SER | A | 679 | 44.007 | −3.161 | 9.826 | 1.00 | 28.59 | O |
| ATOM | 2131 | N | ILE | A | 680 | 46.001 | −2.458 | 9.064 | 1.00 | 29.54 | N |
| ATOM | 2133 | CA | ILE | A | 680 | 46.276 | −3.552 | 8.133 | 1.00 | 30.65 | C |
| ATOM | 2135 | CB | ILE | A | 680 | 47.564 | −3.218 | 7.299 | 1.00 | 31.62 | C |
| ATOM | 2137 | CG1 | ILE | A | 680 | 47.396 | −1.920 | 6.501 | 1.00 | 35.56 | C |
| ATOM | 2140 | CD1 | ILE | A | 680 | 48.607 | −1.711 | 5.515 | 1.00 | 38.92 | C |
| ATOM | 2144 | CG2 | ILE | A | 680 | 47.878 | −4.299 | 6.251 | 1.00 | 34.26 | C |
| ATOM | 2148 | C | ILE | A | 680 | 46.435 | −4.895 | 8.846 | 1.00 | 30.33 | C |
| ATOM | 2149 | O | ILE | A | 680 | 46.355 | −5.951 | 8.221 | 1.00 | 30.79 | O |
| ATOM | 2150 | N | MET | A | 681 | 46.642 | −4.857 | 10.158 | 1.00 | 29.68 | N |
| ATOM | 2152 | CA | MET | A | 681 | 46.814 | −6.057 | 10.931 | 1.00 | 30.17 | C |
| ATOM | 2154 | CB | MET | A | 681 | 47.828 | −5.794 | 12.070 | 1.00 | 29.84 | C |
| ATOM | 2157 | CG | MET | A | 681 | 49.212 | −5.461 | 11.539 | 1.00 | 31.17 | C |
| ATOM | 2160 | SD | MET | A | 681 | 49.737 | −6.803 | 10.421 | 1.00 | 33.29 | S |
| ATOM | 2161 | CE | MET | A | 681 | 51.480 | −6.883 | 10.639 | 1.00 | 34.00 | C |
| ATOM | 2165 | C | MET | A | 681 | 45.498 | −6.637 | 11.473 | 1.00 | 30.11 | C |
| ATOM | 2166 | O | MET | A | 681 | 45.526 | −7.716 | 11.991 | 1.00 | 29.99 | O |
| ATOM | 2167 | N | GLU | A | 682 | 44.371 | −5.941 | 11.318 | 1.00 | 30.83 | N |
| ATOM | 2169 | CA | GLU | A | 682 | 43.083 | −6.433 | 11.782 | 1.00 | 32.14 | C |
| ATOM | 2171 | CB | GLU | A | 682 | 42.002 | −5.357 | 11.801 | 1.00 | 32.53 | C |
| ATOM | 2174 | CG | GLU | A | 682 | 42.241 | −4.109 | 12.598 | 1.00 | 35.57 | C |
| ATOM | 2177 | CD | GLU | A | 682 | 41.150 | −3.074 | 12.378 | 1.00 | 38.08 | C |
| ATOM | 2178 | OE1 | GLU | A | 682 | 40.351 | −3.244 | 11.428 | 1.00 | 38.05 | O |
| ATOM | 2179 | OE2 | GLU | A | 682 | 41.092 | −2.089 | 13.152 | 1.00 | 38.67 | O |
| ATOM | 2180 | C | GLU | A | 682 | 42.562 | −7.526 | 10.868 | 1.00 | 32.35 | C |
| ATOM | 2181 | O | GLU | A | 682 | 42.722 | −7.455 | 9.649 | 1.00 | 32.87 | O |
| ATOM | 2182 | N | HIS | A | 683 | 41.912 | −8.515 | 11.468 | 1.00 | 32.50 | N |
| ATOM | 2184 | CA | HIS | A | 683 | 41.286 | −9.593 | 10.721 | 1.00 | 33.32 | C |
| ATOM | 2186 | CB | HIS | A | 683 | 42.201 | −10.824 | 10.637 | 1.00 | 32.66 | C |
| ATOM | 2189 | CG | HIS | A | 683 | 43.316 | −10.596 | 9.677 | 1.00 | 34.89 | C |
| ATOM | 2190 | ND1 | HIS | A | 683 | 43.082 | −10.378 | 8.328 | 1.00 | 34.82 | N |
| ATOM | 2192 | CE1 | HIS | A | 683 | 44.226 | −10.085 | 7.733 | 1.00 | 34.74 | C |
| ATOM | 2194 | NE2 | HIS | A | 683 | 45.183 | −10.065 | 8.649 | 1.00 | 36.27 | N |
| ATOM | 2196 | CD2 | HIS | A | 683 | 44.631 | −10.343 | 9.880 | 1.00 | 34.25 | C |
| ATOM | 2198 | C | HIS | A | 683 | 39.924 | −9.873 | 11.311 | 1.00 | 33.32 | C |
| ATOM | 2199 | O | HIS | A | 683 | 39.811 | −10.225 | 12.450 | 1.00 | 33.83 | O |
| ATOM | 2200 | N | HIS | A | 684 | 38.899 | −9.696 | 10.503 | 1.00 | 33.32 | N |
| ATOM | 2202 | CA | HIS | A | 684 | 37.542 | −9.800 | 10.968 | 1.00 | 33.77 | C |
| ATOM | 2204 | CB | HIS | A | 684 | 36.660 | −8.837 | 10.181 | 1.00 | 33.24 | C |
| ATOM | 2207 | CG | HIS | A | 684 | 37.150 | −7.446 | 10.227 | 1.00 | 35.04 | C |
| ATOM | 2208 | ND1 | HIS | A | 684 | 36.610 | −6.499 | 11.072 | 1.00 | 37.86 | N |
| ATOM | 2210 | CE1 | HIS | A | 684 | 37.266 | −5.365 | 10.921 | 1.00 | 34.96 | C |
| ATOM | 2212 | NE2 | HIS | A | 684 | 38.233 | −5.553 | 10.043 | 1.00 | 33.74 | N |
| ATOM | 2214 | CD2 | HIS | A | 684 | 38.188 | −6.850 | 9.604 | 1.00 | 32.56 | C |
| ATOM | 2216 | C | HIS | A | 684 | 37.073 | −11.221 | 10.762 | 1.00 | 33.63 | C |
| ATOM | 2217 | O | HIS | A | 684 | 37.579 | −11.936 | 9.898 | 1.00 | 34.39 | O |
| ATOM | 2218 | N | HIS | A | 685 | 36.103 | −11.618 | 11.563 | 1.00 | 33.78 | N |
| ATOM | 2220 | CA | HIS | A | 685 | 35.509 | −12.946 | 11.462 | 1.00 | 34.15 | C |
| ATOM | 2222 | CB | HIS | A | 685 | 35.955 | −13.834 | 12.635 | 1.00 | 34.73 | C |
| ATOM | 2225 | CG | HIS | A | 685 | 37.444 | −13.941 | 12.772 | 1.00 | 37.59 | C |
| ATOM | 2226 | ND1 | HIS | A | 685 | 38.212 | −14.721 | 11.933 | 1.00 | 42.39 | N |
| ATOM | 2228 | CE1 | HIS | A | 685 | 39.486 | −14.589 | 12.255 | 1.00 | 41.15 | C |
| ATOM | 2230 | NE2 | HIS | A | 685 | 39.574 | −13.749 | 13.268 | 1.00 | 40.09 | N |
| ATOM | 2232 | CD2 | HIS | A | 685 | 38.314 | −13.318 | 13.603 | 1.00 | 39.95 | C |
| ATOM | 2234 | C | HIS | A | 685 | 33.974 | −12.866 | 11.360 | 1.00 | 32.94 | C |
| ATOM | 2235 | O | HIS | A | 685 | 33.335 | −11.946 | 11.820 | 1.00 | 32.00 | O |
| ATOM | 2236 | N | PHE | A | 686 | 33.412 | −13.870 | 10.741 | 1.00 | 33.17 | N |
| ATOM | 2238 | CA | PHE | A | 686 | 32.008 | −13.945 | 10.489 | 1.00 | 33.88 | C |
| ATOM | 2240 | CB | PHE | A | 686 | 31.803 | −14.491 | 9.088 | 1.00 | 35.01 | C |
| ATOM | 2243 | CG | PHE | A | 686 | 32.525 | −15.772 | 8.780 | 1.00 | 38.39 | C |
| ATOM | 2244 | CD1 | PHE | A | 686 | 32.167 | −16.981 | 9.392 | 1.00 | 41.03 | C |
| ATOM | 2246 | CE1 | PHE | A | 686 | 32.803 | −18.183 | 9.051 | 1.00 | 40.91 | C |
| ATOM | 2248 | CZ | PHE | A | 686 | 33.804 | −18.187 | 8.078 | 1.00 | 42.36 | C |
| ATOM | 2250 | CE2 | PHE | A | 686 | 34.157 | −17.002 | 7.438 | 1.00 | 41.72 | C |
| ATOM | 2252 | CD2 | PHE | A | 686 | 33.515 | −15.795 | 7.787 | 1.00 | 42.86 | C |
| ATOM | 2254 | C | PHE | A | 686 | 31.292 | −14.815 | 11.511 | 1.00 | 33.38 | C |
| ATOM | 2255 | O | PHE | A | 686 | 30.065 | −14.879 | 11.546 | 1.00 | 33.04 | O |
| ATOM | 2259 | CG | PHE | B | 686 | 29.676 | −15.568 | 8.280 | 1.00 | 50.77 | C |
| ATOM | 2260 | CD1 | PHE | B | 686 | 28.658 | −14.648 | 8.564 | 1.00 | 51.19 | C |
| ATOM | 2262 | CE1 | PHE | B | 686 | 27.557 | −14.841 | 7.742 | 1.00 | 51.49 | C |
| ATOM | 2264 | CZ | PHE | B | 686 | 27.349 | −16.162 | 7.300 | 1.00 | 51.14 | C |
| ATOM | 2266 | CE2 | PHE | B | 686 | 27.994 | −17.198 | 8.051 | 1.00 | 51.13 | C |
| ATOM | 2268 | CD2 | PHE | B | 686 | 29.294 | −16.908 | 8.397 | 1.00 | 50.89 | C |
| ATOM | 2272 | N | ASP | A | 687 | 32.092 | −15.582 | 12.599 | 1.00 | 31.50 | N |
| ATOM | 2274 | CA | ASP | A | 687 | 31.473 | −16.638 | 13.419 | 1.00 | 32.97 | C |
| ATOM | 2276 | CB | ASP | A | 687 | 32.527 | −17.298 | 14.298 | 1.00 | 33.51 | C |
| ATOM | 2279 | CG | ASP | A | 687 | 33.782 | −17.750 | 13.505 | 1.00 | 37.66 | C |
| ATOM | 2280 | OD1 | ASP | A | 687 | 34.365 | −16.976 | 12.688 | 1.00 | 41.70 | O |
| ATOM | 2281 | OD2 | ASP | A | 687 | 34.275 | −18.883 | 13.661 | 1.00 | 43.47 | O |
| ATOM | 2282 | C | ASP | A | 687 | 30.352 | −16.037 | 14.265 | 1.00 | 32.03 | C |
| ATOM | 2283 | O | ASP | A | 687 | 29.286 | −16.621 | 14.357 | 1.00 | 32.15 | O |
| ATOM | 2286 | N | GLN | A | 688 | 30.565 | −14.851 | 14.836 | 1.00 | 31.16 | N |
| ATOM | 2288 | CA | GLN | A | 688 | 29.589 | −14.264 | 15.750 | 1.00 | 31.25 | C |
| ATOM | 2290 | CB | GLN | A | 688 | 30.225 | −13.154 | 16.582 | 1.00 | 31.77 | C |
| ATOM | 2293 | CG | GLN | A | 688 | 29.374 | −12.759 | 17.799 | 1.00 | 34.88 | C |
| ATOM | 2296 | CD | GLN | A | 688 | 30.119 | −11.885 | 18.796 | 1.00 | 38.18 | C |
| ATOM | 2297 | OE1 | GLN | A | 688 | 31.181 | −11.329 | 18.485 | 1.00 | 40.54 | O |
| ATOM | 2298 | NE2 | GLN | A | 688 | 29.559 | −11.755 | 19.992 | 1.00 | 39.53 | N |
| ATOM | 2301 | C | GLN | A | 688 | 28.353 | −13.730 | 15.017 | 1.00 | 31.05 | C |
| ATOM | 2302 | O | GLN | A | 688 | 27.220 | −13.846 | 15.518 | 1.00 | 30.38 | O |
| ATOM | 2303 | N | CYS | A | 689 | 28.555 | −13.163 | 13.827 | 1.00 | 30.12 | N |
| ATOM | 2305 | CA | CYS | A | 689 | 27.435 | −12.763 | 12.967 | 1.00 | 30.44 | C |
| ATOM | 2307 | CB | CYS | A | 689 | 27.949 | −12.200 | 11.644 | 1.00 | 30.42 | C |
| ATOM | 2310 | SG | CYS | A | 689 | 26.668 | −11.902 | 10.426 | 1.00 | 30.66 | S |
| ATOM | 2311 | C | CYS | A | 689 | 26.467 | −13.923 | 12.677 | 1.00 | 30.97 | C |
| ATOM | 2312 | O | CYS | A | 689 | 25.250 | −13.779 | 12.825 | 1.00 | 31.28 | O |
| ATOM | 2313 | N | LEU | A | 690 | 27.015 | −15.064 | 12.276 | 1.00 | 31.79 | N |
| ATOM | 2315 | CA | LEU | A | 690 | 26.236 | −16.264 | 11.933 | 1.00 | 32.94 | C |
| ATOM | 2317 | CB | LEU | A | 690 | 27.184 | −17.331 | 11.368 | 1.00 | 33.46 | C |
| ATOM | 2320 | CG | LEU | A | 690 | 26.757 | −18.703 | 10.824 | 1.00 | 37.99 | C |
| ATOM | 2322 | CD1 | LEU | A | 690 | 26.703 | −19.815 | 11.914 | 1.00 | 40.57 | C |
| ATOM | 2326 | CD2 | LEU | A | 690 | 25.420 | −18.617 | 10.058 | 1.00 | 40.70 | C |
| ATOM | 2330 | C | LEU | A | 690 | 25.538 | −16.818 | 13.171 | 1.00 | 32.70 | C |
| ATOM | 2331 | O | LEU | A | 690 | 24.375 | −17.172 | 13.118 | 1.00 | 32.47 | O |
| ATOM | 2332 | N | MET | A | 691 | 26.265 | −16.907 | 14.284 | 1.00 | 33.18 | N |
| ATOM | 2334 | CA | MET | A | 691 | 25.691 | −17.403 | 15.534 | 1.00 | 34.16 | C |
| ATOM | 2336 | CB | MET | A | 691 | 26.723 | −17.390 | 16.651 | 1.00 | 35.01 | C |
| ATOM | 2339 | CG | MET | A | 691 | 26.091 | −17.561 | 18.050 | 1.00 | 40.17 | C |
| ATOM | 2342 | SD | MET | A | 691 | 27.311 | −17.341 | 19.371 | 1.00 | 50.88 | S |
| ATOM | 2343 | CE | MET | A | 691 | 27.597 | −15.483 | 19.419 | 1.00 | 47.95 | C |
| ATOM | 2347 | C | MET | A | 691 | 24.449 | −16.590 | 15.950 | 1.00 | 33.27 | C |
| ATOM | 2348 | O | MET | A | 691 | 23.448 | −17.160 | 16.358 | 1.00 | 32.30 | O |
| ATOM | 2349 | N | ILE | A | 692 | 24.506 | −15.266 | 15.816 | 1.00 | 32.39 | N |
| ATOM | 2351 | CA | ILE | A | 692 | 23.360 | −14.430 | 16.191 | 1.00 | 32.77 | C |
| ATOM | 2353 | CB | ILE | A | 692 | 23.768 | −12.937 | 16.271 | 1.00 | 32.53 | C |
| ATOM | 2355 | CG1 | ILE | A | 692 | 24.784 | −12.735 | 17.400 | 1.00 | 32.90 | C |
| ATOM | 2358 | CD1 | ILE | A | 692 | 25.376 | −11.322 | 17.429 | 1.00 | 34.91 | C |
| ATOM | 2362 | CG2 | ILE | A | 692 | 22.547 | −12.060 | 16.506 | 1.00 | 33.18 | C |
| ATOM | 2366 | C | ILE | A | 692 | 22.173 | −14.636 | 15.235 | 1.00 | 32.75 | C |
| ATOM | 2367 | O | ILE | A | 692 | 21.019 | −14.721 | 15.668 | 1.00 | 32.34 | O |
| ATOM | 2368 | N | LEU | A | 693 | 22.476 | −14.732 | 13.942 | 1.00 | 33.49 | N |
| ATOM | 2370 | CA | LEU | A | 693 | 21.493 | −14.980 | 12.882 | 1.00 | 33.88 | C |
| ATOM | 2372 | CB | LEU | A | 693 | 22.191 | −15.052 | 11.524 | 1.00 | 33.93 | C |
| ATOM | 2375 | CG | LEU | A | 693 | 22.548 | −13.756 | 10.805 | 1.00 | 35.20 | C |
| ATOM | 2377 | CD1 | LEU | A | 693 | 23.319 | −14.084 | 9.540 | 1.00 | 35.75 | C |
| ATOM | 2381 | CD2 | LEU | A | 693 | 21.313 | −12.992 | 10.457 | 1.00 | 37.26 | C |
| ATOM | 2385 | C | LEU | A | 693 | 20.739 | −16.291 | 13.056 | 1.00 | 34.18 | C |
| ATOM | 2386 | O | LEU | A | 693 | 19.615 | −16.427 | 12.571 | 1.00 | 34.35 | O |
| ATOM | 2387 | N | ASN | A | 694 | 21.379 | −17.266 | 13.690 | 1.00 | 34.61 | N |
| ATOM | 2389 | CA | ASN | A | 694 | 20.787 | −18.578 | 13.893 | 1.00 | 35.26 | C |
| ATOM | 2391 | CB | ASN | A | 694 | 21.823 | −19.662 | 13.585 | 1.00 | 36.11 | C |
| ATOM | 2394 | CG | ASN | A | 694 | 22.160 | −19.729 | 12.109 | 1.00 | 39.88 | C |
| ATOM | 2395 | OD1 | ASN | A | 694 | 21.322 | −19.374 | 11.254 | 1.00 | 44.53 | O |
| ATOM | 2396 | ND2 | ASN | A | 694 | 23.390 | −20.182 | 11.789 | 1.00 | 41.14 | N |
| ATOM | 2399 | C | ASN | A | 694 | 20.231 | −18.782 | 15.305 | 1.00 | 34.38 | C |
| ATOM | 2400 | O | ASN | A | 694 | 19.838 | −19.874 | 15.660 | 1.00 | 33.37 | O |
| ATOM | 2401 | N | SER | A | 695 | 20.202 | −17.715 | 16.089 | 1.00 | 33.65 | N |
| ATOM | 2403 | CA | SER | A | 695 | 19.858 | −17.780 | 17.499 | 1.00 | 33.45 | C |
| ATOM | 2405 | CB | SER | A | 695 | 20.478 | −16.562 | 18.191 | 1.00 | 33.53 | C |
| ATOM | 2408 | OG | SER | A | 695 | 20.716 | −16.834 | 19.539 | 1.00 | 36.56 | O |
| ATOM | 2410 | C | SER | A | 695 | 18.347 | −17.737 | 17.615 | 1.00 | 32.46 | C |
| ATOM | 2411 | O | SER | A | 695 | 17.735 | −16.981 | 16.874 | 1.00 | 31.34 | O |
| ATOM | 2412 | N | PRO | A | 696 | 17.726 | −18.528 | 18.506 | 1.00 | 32.72 | N |
| ATOM | 2413 | CA | PRO | A | 696 | 16.253 | −18.548 | 18.596 | 1.00 | 32.48 | C |
| ATOM | 2415 | CB | PRO | A | 696 | 15.977 | −19.410 | 19.837 | 1.00 | 32.89 | C |
| ATOM | 2418 | CG | PRO | A | 696 | 17.170 | −20.303 | 19.949 | 1.00 | 33.37 | C |
| ATOM | 2421 | CD | PRO | A | 696 | 18.342 | −19.465 | 19.468 | 1.00 | 32.62 | C |
| ATOM | 2424 | C | PRO | A | 696 | 15.662 | −17.153 | 18.773 | 1.00 | 32.13 | C |
| ATOM | 2425 | O | PRO | A | 696 | 16.197 | −16.391 | 19.560 | 1.00 | 32.27 | O |
| ATOM | 2426 | N | GLY | A | 697 | 14.611 | −16.825 | 18.023 | 1.00 | 31.88 | N |
| ATOM | 2428 | CA | GLY | A | 697 | 13.997 | −15.503 | 18.056 | 1.00 | 31.92 | C |
| ATOM | 2431 | C | GLY | A | 697 | 14.697 | −14.389 | 17.276 | 1.00 | 31.76 | C |
| ATOM | 2432 | O | GLY | A | 697 | 14.146 | −13.287 | 17.133 | 1.00 | 31.96 | O |
| ATOM | 2433 | N | ASN | A | 698 | 15.890 | −14.665 | 16.754 | 1.00 | 31.26 | N |
| ATOM | 2435 | CA | ASN | A | 698 | 16.699 | −13.663 | 16.060 | 1.00 | 31.31 | C |
| ATOM | 2437 | CB | ASN | A | 698 | 18.093 | −13.574 | 16.697 | 1.00 | 31.05 | C |
| ATOM | 2440 | CG | ASN | A | 698 | 18.073 | −13.015 | 18.099 | 1.00 | 30.32 | C |
| ATOM | 2441 | OD1 | ASN | A | 698 | 18.380 | −11.869 | 18.298 | 1.00 | 30.29 | O |
| ATOM | 2442 | ND2 | ASN | A | 698 | 17.731 | −13.836 | 19.078 | 1.00 | 32.05 | N |
| ATOM | 2445 | C | ASN | A | 698 | 16.877 | −13.999 | 14.580 | 1.00 | 31.57 | C |
| ATOM | 2446 | O | ASN | A | 698 | 17.687 | −13.374 | 13.900 | 1.00 | 31.67 | O |
| ATOM | 2447 | N | GLN | A | 699 | 16.134 | −14.990 | 14.084 | 1.00 | 31.49 | N |
| ATOM | 2449 | CA | GLN | A | 699 | 16.391 | −15.548 | 12.763 | 1.00 | 31.46 | C |
| ATOM | 2451 | CB | GLN | A | 699 | 15.949 | −17.022 | 12.691 | 1.00 | 32.07 | C |
| ATOM | 2454 | CG | GLN | A | 699 | 16.781 | −17.939 | 13.585 | 1.00 | 33.35 | C |
| ATOM | 2457 | CD | GLN | A | 699 | 16.171 | −19.315 | 13.780 | 1.00 | 36.39 | C |
| ATOM | 2458 | OE1 | GLN | A | 699 | 16.710 | −20.309 | 13.298 | 1.00 | 37.88 | O |
| ATOM | 2459 | NE2 | GLN | A | 699 | 15.057 | −19.378 | 14.500 | 1.00 | 38.71 | N |
| ATOM | 2462 | C | GLN | A | 699 | 15.747 | −14.709 | 11.674 | 1.00 | 31.10 | C |
| ATOM | 2463 | O | GLN | A | 699 | 14.718 | −15.079 | 11.104 | 1.00 | 30.82 | O |
| ATOM | 2464 | N | ILE | A | 700 | 16.387 | −13.592 | 11.346 | 1.00 | 30.47 | N |
| ATOM | 2466 | CA | ILE | A | 700 | 15.834 | −12.693 | 10.338 | 1.00 | 30.47 | C |
| ATOM | 2468 | CB | ILE | A | 700 | 16.514 | −11.300 | 10.394 | 1.00 | 30.43 | C |
| ATOM | 2470 | CG1 | ILE | A | 700 | 18.009 | −11.385 | 10.130 | 1.00 | 32.01 | C |
| ATOM | 2473 | CD1 | ILE | A | 700 | 18.675 | −10.020 | 9.846 | 1.00 | 32.43 | C |
| ATOM | 2477 | CG2 | ILE | A | 700 | 16.250 | −10.653 | 11.752 | 1.00 | 29.96 | C |
| ATOM | 2481 | C | ILE | A | 700 | 15.870 | −13.260 | 8.908 | 1.00 | 30.26 | C |
| ATOM | 2482 | O | ILE | A | 700 | 15.255 | −12.701 | 8.037 | 1.00 | 29.34 | O |
| ATOM | 2483 | N | LEU | A | 701 | 16.606 | −14.346 | 8.679 | 1.00 | 30.75 | N |
| ATOM | 2485 | CA | LEU | A | 701 | 16.675 | −14.984 | 7.362 | 1.00 | 31.28 | C |
| ATOM | 2487 | CB | LEU | A | 701 | 18.131 | −15.364 | 7.008 | 1.00 | 30.89 | C |
| ATOM | 2490 | CG | LEU | A | 701 | 19.172 | −14.244 | 7.069 | 1.00 | 32.55 | C |
| ATOM | 2492 | CD1 | LEU | A | 701 | 20.528 | −14.746 | 6.632 | 1.00 | 33.04 | C |
| ATOM | 2496 | CD2 | LEU | A | 701 | 18.737 | −13.067 | 6.230 | 1.00 | 32.79 | C |
| ATOM | 2500 | C | LEU | A | 701 | 15.795 | −16.219 | 7.268 | 1.00 | 31.50 | C |
| ATOM | 2501 | O | LEU | A | 701 | 15.999 | −17.024 | 6.371 | 1.00 | 31.76 | O |
| ATOM | 2502 | N | SER | A | 702 | 14.805 | −16.361 | 8.157 | 1.00 | 31.90 | N |
| ATOM | 2504 | CA | SER | A | 702 | 13.963 | −17.574 | 8.201 | 1.00 | 31.30 | C |
| ATOM | 2506 | CB | SER | A | 702 | 13.101 | −17.613 | 9.480 | 1.00 | 31.65 | C |
| ATOM | 2509 | OG | SER | A | 702 | 12.202 | −16.514 | 9.552 | 1.00 | 32.37 | O |
| ATOM | 2511 | C | SER | A | 702 | 13.071 | −17.704 | 6.966 | 1.00 | 30.98 | C |
| ATOM | 2512 | O | SER | A | 702 | 12.822 | −18.811 | 6.494 | 1.00 | 30.26 | O |
| ATOM | 2513 | N | GLY | A | 703 | 12.652 | −16.567 | 6.423 | 1.00 | 30.27 | N |
| ATOM | 2515 | CA | GLY | A | 703 | 11.878 | −16.506 | 5.196 | 1.00 | 30.37 | C |
| ATOM | 2518 | C | GLY | A | 703 | 12.606 | −16.916 | 3.907 | 1.00 | 30.71 | C |
| ATOM | 2519 | O | GLY | A | 703 | 11.953 | −17.174 | 2.893 | 1.00 | 30.10 | O |
| ATOM | 2520 | N | LEU | A | 704 | 13.946 | −16.971 | 3.936 | 1.00 | 30.77 | N |
| ATOM | 2522 | CA | LEU | A | 704 | 14.726 | −17.371 | 2.765 | 1.00 | 30.56 | C |
| ATOM | 2524 | CB | LEU | A | 704 | 16.210 | −16.996 | 2.909 | 1.00 | 30.10 | C |
| ATOM | 2527 | CG | LEU | A | 704 | 16.723 | −15.587 | 3.249 | 1.00 | 34.32 | C |
| ATOM | 2529 | CD1 | LEU | A | 704 | 18.193 | −15.367 | 2.788 | 1.00 | 33.13 | C |
| ATOM | 2533 | CD2 | LEU | A | 704 | 15.867 | −14.494 | 2.720 | 1.00 | 37.04 | C |
| ATOM | 2537 | C | LEU | A | 704 | 14.635 | −18.884 | 2.523 | 1.00 | 29.50 | C |
| ATOM | 2538 | O | LEU | A | 704 | 14.684 | −19.668 | 3.452 | 1.00 | 29.10 | O |
| ATOM | 2539 | N | SER | A | 705 | 14.531 | −19.281 | 1.261 | 1.00 | 28.82 | N |
| ATOM | 2541 | CA | SER | A | 705 | 14.737 | −20.665 | 0.851 | 1.00 | 28.42 | C |
| ATOM | 2543 | CB | SER | A | 705 | 14.463 | −20.819 | −0.649 | 1.00 | 28.44 | C |
| ATOM | 2546 | OG | SER | A | 705 | 15.424 | −20.071 | −1.386 | 1.00 | 28.13 | O |
| ATOM | 2548 | C | SER | A | 705 | 16.175 | −21.096 | 1.156 | 1.00 | 28.17 | C |
| ATOM | 2549 | O | SER | A | 705 | 17.065 | −20.251 | 1.338 | 1.00 | 27.96 | O |
| ATOM | 2550 | N | ILE | A | 706 | 16.418 | −22.400 | 1.213 | 1.00 | 28.12 | N |
| ATOM | 2552 | CA | ILE | A | 706 | 17.751 | −22.857 | 1.578 | 1.00 | 28.61 | C |
| ATOM | 2554 | CB | ILE | A | 706 | 17.826 | −24.376 | 1.797 | 1.00 | 29.31 | C |
| ATOM | 2556 | CG1 | ILE | A | 706 | 17.437 | −25.161 | 0.549 | 1.00 | 29.59 | C |
| ATOM | 2559 | CD1 | ILE | A | 706 | 17.422 | −26.641 | 0.794 | 1.00 | 33.51 | C |
| ATOM | 2563 | CG2 | ILE | A | 706 | 16.906 | −24.781 | 2.941 | 1.00 | 30.87 | C |
| ATOM | 2567 | C | ILE | A | 706 | 18.819 | −22.337 | 0.614 | 1.00 | 28.18 | C |
| ATOM | 2568 | O | ILE | A | 706 | 19.894 | −21.953 | 1.060 | 1.00 | 27.65 | O |
| ATOM | 2569 | N | GLU | A | 707 | 18.491 | −22.243 | −0.671 | 1.00 | 28.07 | N |
| ATOM | 2571 | CA | GLU | A | 707 | 19.419 | −21.740 | −1.692 | 1.00 | 28.72 | C |
| ATOM | 2573 | CB | GLU | A | 707 | 18.894 | −22.020 | −3.106 | 1.00 | 29.03 | C |
| ATOM | 2576 | CG | GLU | A | 707 | 18.752 | −23.486 | −3.462 | 1.00 | 31.17 | C |
| ATOM | 2579 | CD | GLU | A | 707 | 17.385 | −24.097 | −3.137 | 1.00 | 32.70 | C |
| ATOM | 2580 | OE1 | GLU | A | 707 | 16.497 | −23.358 | −2.611 | 1.00 | 31.18 | O |
| ATOM | 2581 | OE2 | GLU | A | 707 | 17.213 | −25.329 | −3.416 | 1.00 | 31.40 | O |
| ATOM | 2582 | C | GLU | A | 707 | 19.704 | −20.229 | −1.517 | 1.00 | 28.17 | C |
| ATOM | 2583 | O | GLU | A | 707 | 20.868 | −19.790 | −1.558 | 1.00 | 28.17 | O |
| ATOM | 2584 | N | GLU | A | 708 | 18.657 | −19.443 | −1.313 | 1.00 | 27.81 | N |
| ATOM | 2586 | CA | GLU | A | 708 | 18.801 | −18.037 | −0.910 | 1.00 | 28.54 | C |
| ATOM | 2588 | CB | GLU | A | 708 | 17.451 | −17.413 | −0.577 | 1.00 | 28.31 | C |
| ATOM | 2591 | CG | GLU | A | 708 | 16.613 | −16.992 | −1.766 | 1.00 | 30.10 | C |
| ATOM | 2594 | CD | GLU | A | 708 | 15.335 | −16.313 | −1.319 | 1.00 | 29.90 | C |
| ATOM | 2595 | OE1 | GLU | A | 708 | 14.625 | −16.911 | −0.502 | 1.00 | 28.02 | O |
| ATOM | 2596 | OE2 | GLU | A | 708 | 15.061 | −15.175 | −1.751 | 1.00 | 32.16 | O |
| ATOM | 2597 | C | GLU | A | 708 | 19.665 | −17.873 | 0.348 | 1.00 | 28.37 | C |
| ATOM | 2598 | O | GLU | A | 708 | 20.496 | −16.971 | 0.436 | 1.00 | 27.41 | O |
| ATOM | 2599 | N | TYR | A | 709 | 19.428 | −18.739 | 1.327 | 1.00 | 28.74 | N |
| ATOM | 2601 | CA | TYR | A | 709 | 20.109 | −18.658 | 2.628 | 1.00 | 29.07 | C |
| ATOM | 2603 | CB | TYR | A | 709 | 19.453 | −19.633 | 3.636 | 1.00 | 29.15 | C |
| ATOM | 2606 | CG | TYR | A | 709 | 20.124 | −19.744 | 4.977 | 1.00 | 30.08 | C |
| ATOM | 2607 | CD1 | TYR | A | 709 | 19.868 | −18.822 | 5.986 | 1.00 | 30.25 | C |
| ATOM | 2609 | CE1 | TYR | A | 709 | 20.487 | −18.935 | 7.224 | 1.00 | 32.05 | C |
| ATOM | 2611 | CZ | TYR | A | 709 | 21.352 | −19.981 | 7.455 | 1.00 | 32.81 | C |
| ATOM | 2612 | OH | TYR | A | 709 | 21.976 | −20.115 | 8.665 | 1.00 | 36.64 | O |
| ATOM | 2614 | CE2 | TYR | A | 709 | 21.620 | −20.907 | 6.470 | 1.00 | 31.98 | C |
| ATOM | 2616 | CD2 | TYR | A | 709 | 21.002 | −20.790 | 5.248 | 1.00 | 30.61 | C |
| ATOM | 2618 | C | TYR | A | 709 | 21.595 | −18.948 | 2.419 | 1.00 | 28.39 | C |
| ATOM | 2619 | O | TYR | A | 709 | 22.426 | −18.153 | 2.792 | 1.00 | 27.85 | O |
| ATOM | 2620 | N | LYS | A | 710 | 21.924 | −20.058 | 1.773 | 1.00 | 29.18 | N |
| ATOM | 2622 | CA | LYS | A | 710 | 23.326 | −20.390 | 1.544 | 1.00 | 30.08 | C |
| ATOM | 2624 | CB | LYS | A | 710 | 23.482 | −21.735 | 0.879 | 1.00 | 30.51 | C |
| ATOM | 2627 | CG | LYS | A | 710 | 23.191 | −22.928 | 1.801 | 1.00 | 32.10 | C |
| ATOM | 2630 | CD | LYS | A | 710 | 23.256 | −24.197 | 0.984 | 1.00 | 34.82 | C |
| ATOM | 2633 | CE | LYS | A | 710 | 23.178 | −25.471 | 1.823 | 1.00 | 36.19 | C |
| ATOM | 2636 | NZ | LYS | A | 710 | 23.048 | −26.659 | 0.962 | 1.00 | 36.81 | N |
| ATOM | 2640 | C | LYS | A | 710 | 24.077 | −19.287 | 0.758 | 1.00 | 30.38 | C |
| ATOM | 2641 | O | LYS | A | 710 | 25.222 | −18.983 | 1.078 | 1.00 | 30.06 | O |
| ATOM | 2642 | N | THR | A | 711 | 23.419 | −18.666 | −0.222 | 1.00 | 29.86 | N |
| ATOM | 2644 | CA | THR | A | 711 | 24.062 | −17.629 | −1.032 | 1.00 | 30.01 | C |
| ATOM | 2646 | CB | THR | A | 711 | 23.217 | −17.304 | −2.280 | 1.00 | 30.66 | C |
| ATOM | 2648 | OG1 | THR | A | 711 | 23.216 | −18.436 | −3.175 | 1.00 | 30.28 | O |
| ATOM | 2650 | CG2 | THR | A | 711 | 23.857 | −16.163 | −3.073 | 1.00 | 30.73 | C |
| ATOM | 2654 | C | THR | A | 711 | 24.280 | −16.369 | −0.211 | 1.00 | 29.27 | C |
| ATOM | 2655 | O | THR | A | 711 | 25.323 | −15.703 | −0.301 | 1.00 | 28.15 | O |
| ATOM | 2656 | N | THR | A | 712 | 23.279 | −16.032 | 0.594 | 1.00 | 29.05 | N |
| ATOM | 2658 | CA | THR | A | 712 | 23.342 | −14.843 | 1.409 | 1.00 | 28.20 | C |
| ATOM | 2660 | CB | THR | A | 712 | 21.986 | −14.624 | 2.128 | 1.00 | 28.55 | C |
| ATOM | 2662 | OG1 | THR | A | 712 | 20.947 | −14.394 | 1.162 | 1.00 | 26.56 | O |
| ATOM | 2664 | CG2 | THR | A | 712 | 22.014 | −13.347 | 2.972 | 1.00 | 28.61 | C |
| ATOM | 2668 | C | THR | A | 712 | 24.486 | −14.963 | 2.418 | 1.00 | 29.25 | C |
| ATOM | 2669 | O | THR | A | 712 | 25.247 | −14.001 | 2.655 | 1.00 | 28.91 | O |
| ATOM | 2670 | N | LEU | A | 713 | 24.615 | −16.130 | 3.030 | 1.00 | 28.84 | N |
| ATOM | 2672 | CA | LEU | A | 713 | 25.679 | −16.335 | 4.014 | 1.00 | 29.18 | C |
| ATOM | 2674 | CB | LEU | A | 713 | 25.562 | −17.704 | 4.709 | 1.00 | 29.57 | C |
| ATOM | 2677 | CG | LEU | A | 713 | 24.344 | −18.001 | 5.625 | 1.00 | 30.84 | C |
| ATOM | 2679 | CD1 | LEU | A | 713 | 24.694 | −19.174 | 6.592 | 1.00 | 33.33 | C |
| ATOM | 2683 | CD2 | LEU | A | 713 | 23.816 | −16.841 | 6.360 | 1.00 | 32.03 | C |
| ATOM | 2687 | C | LEU | A | 713 | 27.054 | −16.201 | 3.365 | 1.00 | 29.27 | C |
| ATOM | 2688 | O | LEU | A | 713 | 27.971 | −15.605 | 3.954 | 1.00 | 27.98 | O |
| ATOM | 2689 | N | LYS | A | 714 | 27.203 | −16.745 | 2.162 | 1.00 | 28.99 | N |
| ATOM | 2691 | CA | LYS | A | 714 | 28.458 | −16.634 | 1.431 | 1.00 | 29.77 | C |
| ATOM | 2693 | CB | LYS | A | 714 | 28.397 | −17.435 | 0.129 | 1.00 | 30.50 | C |
| ATOM | 2696 | CG | LYS | A | 714 | 29.555 | −17.190 | −0.797 | 1.00 | 33.47 | C |
| ATOM | 2699 | CD | LYS | A | 714 | 29.542 | −18.049 | −2.061 | 1.00 | 37.61 | C |
| ATOM | 2702 | CE | LYS | A | 714 | 28.298 | −17.795 | −2.928 | 1.00 | 41.48 | C |
| ATOM | 2705 | NZ | LYS | A | 714 | 27.560 | −19.063 | −3.353 | 1.00 | 42.85 | N |
| ATOM | 2709 | C | LYS | A | 714 | 28.792 | −15.172 | 1.154 | 1.00 | 29.88 | C |
| ATOM | 2710 | O | LYS | A | 714 | 29.921 | −14.753 | 1.379 | 1.00 | 30.34 | O |
| ATOM | 2711 | N | ILE | A | 715 | 27.818 | −14.368 | 0.724 | 1.00 | 29.41 | N |
| ATOM | 2713 | CA | ILE | A | 715 | 28.087 | −12.952 | 0.466 | 1.00 | 29.58 | C |
| ATOM | 2715 | CB | ILE | A | 715 | 26.899 | −12.252 | −0.272 | 1.00 | 29.54 | C |
| ATOM | 2717 | CG1 | ILE | A | 715 | 26.630 | −12.898 | −1.640 | 1.00 | 30.27 | C |
| ATOM | 2720 | CD1 | ILE | A | 715 | 25.236 | −12.628 | −2.172 | 1.00 | 32.00 | C |
| ATOM | 2724 | CG2 | ILE | A | 715 | 27.202 | −10.752 | −0.452 | 1.00 | 30.82 | C |
| ATOM | 2728 | C | ILE | A | 715 | 28.395 | −12.199 | 1.773 | 1.00 | 30.15 | C |
| ATOM | 2729 | O | ILE | A | 715 | 29.217 | −11.283 | 1.780 | 1.00 | 29.32 | O |
| ATOM | 2730 | N | ILE | A | 716 | 27.695 | −12.540 | 2.865 | 1.00 | 30.66 | N |
| ATOM | 2732 | CA | ILE | A | 716 | 27.948 | −11.911 | 4.159 | 1.00 | 30.81 | C |
| ATOM | 2734 | CB | ILE | A | 716 | 26.953 | −12.400 | 5.284 | 1.00 | 30.93 | C |
| ATOM | 2736 | CG1 | ILE | A | 716 | 25.522 | −11.939 | 5.007 | 1.00 | 29.96 | C |
| ATOM | 2739 | CD1 | ILE | A | 716 | 24.456 | −12.555 | 5.941 | 1.00 | 28.36 | C |
| ATOM | 2743 | CG2 | ILE | A | 716 | 27.377 | −11.825 | 6.666 | 1.00 | 32.78 | C |
| ATOM | 2747 | C | ILE | A | 716 | 29.389 | −12.147 | 4.618 | 1.00 | 30.65 | C |
| ATOM | 2748 | O | ILE | A | 716 | 30.026 | −11.204 | 5.071 | 1.00 | 31.73 | O |
| ATOM | 2749 | N | LYS | A | 717 | 29.861 | −13.389 | 4.530 | 1.00 | 31.34 | N |
| ATOM | 2751 | CA | LYS | A | 717 | 31.224 | −13.777 | 4.906 | 1.00 | 32.36 | C |
| ATOM | 2753 | CB | LYS | A | 717 | 31.507 | −15.261 | 4.657 | 1.00 | 32.51 | C |
| ATOM | 2756 | CG | LYS | A | 717 | 30.760 | −16.269 | 5.531 | 1.00 | 35.68 | C |
| ATOM | 2759 | CD | LYS | A | 717 | 31.032 | −17.766 | 5.106 | 1.00 | 38.43 | C |
| ATOM | 2762 | CE | LYS | A | 717 | 30.092 | −18.775 | 5.818 | 1.00 | 39.94 | C |
| ATOM | 2765 | NZ | LYS | A | 717 | 29.895 | −20.095 | 5.141 | 1.00 | 39.44 | N |
| ATOM | 2769 | C | LYS | A | 717 | 32.239 | −12.986 | 4.095 | 1.00 | 32.41 | C |
| ATOM | 2770 | O | LYS | A | 717 | 33.140 | −12.387 | 4.670 | 1.00 | 31.82 | O |
| ATOM | 2771 | N | GLN | A | 718 | 32.091 | −13.001 | 2.769 | 1.00 | 31.49 | N |
| ATOM | 2773 | CA | GLN | A | 718 | 32.952 | −12.216 | 1.880 | 1.00 | 32.27 | C |
| ATOM | 2775 | CB | GLN | A | 718 | 32.541 | −12.381 | 0.410 | 1.00 | 32.06 | C |
| ATOM | 2778 | CG | GLN | A | 718 | 32.935 | −13.729 | −0.165 | 1.00 | 33.82 | C |
| ATOM | 2781 | CD | GLN | A | 718 | 32.343 | −14.005 | −1.544 | 1.00 | 36.85 | C |
| ATOM | 2782 | OE1 | GLN | A | 718 | 31.380 | −13.347 | −1.972 | 1.00 | 39.56 | O |
| ATOM | 2783 | NE2 | GLN | A | 718 | 32.923 | −14.983 | −2.246 | 1.00 | 37.92 | N |
| ATOM | 2786 | C | GLN | A | 718 | 32.958 | −10.738 | 2.257 | 1.00 | 31.81 | C |
| ATOM | 2787 | O | GLN | A | 718 | 34.004 | −10.123 | 2.318 | 1.00 | 31.71 | O |
| ATOM | 2788 | N | ALA | A | 719 | 31.792 | −10.182 | 2.549 | 1.00 | 31.68 | N |
| ATOM | 2790 | CA | ALA | A | 719 | 31.683 | −8.762 | 2.853 | 1.00 | 31.63 | C |
| ATOM | 2792 | CB | ALA | A | 719 | 30.234 | −8.325 | 2.816 | 1.00 | 32.00 | C |
| ATOM | 2796 | C | ALA | A | 719 | 32.312 | −8.380 | 4.189 | 1.00 | 32.00 | C |
| ATOM | 2797 | O | ALA | A | 719 | 32.860 | −7.270 | 4.358 | 1.00 | 31.34 | O |
| ATOM | 2798 | N | ILE | A | 720 | 32.228 | −9.284 | 5.158 | 1.00 | 32.32 | N |
| ATOM | 2800 | CA | ILE | A | 720 | 32.846 | −9.021 | 6.455 | 1.00 | 31.42 | C |
| ATOM | 2802 | CB | ILE | A | 720 | 32.323 | −9.983 | 7.534 | 1.00 | 30.69 | C |
| ATOM | 2804 | CG1 | ILE | A | 720 | 30.901 | −9.615 | 7.919 | 1.00 | 27.83 | C |
| ATOM | 2807 | CD1 | ILE | A | 720 | 30.313 | −10.502 | 8.957 | 1.00 | 27.29 | C |
| ATOM | 2811 | CG2 | ILE | A | 720 | 33.235 | −9.947 | 8.747 | 1.00 | 31.46 | C |
| ATOM | 2815 | C | ILE | A | 720 | 34.354 | −9.146 | 6.316 | 1.00 | 31.68 | C |
| ATOM | 2816 | O | ILE | A | 720 | 35.095 | −8.321 | 6.851 | 1.00 | 31.13 | O |
| ATOM | 2817 | N | LEU | A | 721 | 34.803 | −10.186 | 5.612 | 1.00 | 32.18 | N |
| ATOM | 2819 | CA | LEU | A | 721 | 36.223 | −10.374 | 5.345 | 1.00 | 32.91 | C |
| ATOM | 2821 | CB | LEU | A | 721 | 36.529 | −11.711 | 4.675 | 1.00 | 32.99 | C |
| ATOM | 2824 | CG | LEU | A | 721 | 36.297 | −12.971 | 5.525 | 1.00 | 37.70 | C |
| ATOM | 2826 | CD1 | LEU | A | 721 | 36.514 | −14.245 | 4.683 | 1.00 | 38.76 | C |
| ATOM | 2830 | CD2 | LEU | A | 721 | 37.162 | −13.046 | 6.745 | 1.00 | 40.11 | C |
| ATOM | 2834 | C | LEU | A | 721 | 36.794 | −9.216 | 4.524 | 1.00 | 32.85 | C |
| ATOM | 2835 | O | LEU | A | 721 | 37.952 | −8.836 | 4.754 | 1.00 | 33.57 | O |
| ATOM | 2836 | N | ALA | A | 722 | 35.986 | −8.634 | 3.630 | 1.00 | 31.29 | N |
| ATOM | 2838 | CA | ALA | A | 722 | 36.363 | −7.441 | 2.873 | 1.00 | 31.32 | C |
| ATOM | 2840 | CB | ALA | A | 722 | 35.201 | −6.986 | 1.947 | 1.00 | 31.27 | C |
| ATOM | 2844 | C | ALA | A | 722 | 36.773 | −6.233 | 3.752 | 1.00 | 30.62 | C |
| ATOM | 2845 | O | ALA | A | 722 | 37.454 | −5.358 | 3.264 | 1.00 | 29.59 | O |
| ATOM | 2846 | N | THR | A | 723 | 36.306 | −6.143 | 5.000 | 1.00 | 30.85 | N |
| ATOM | 2848 | CA | THR | A | 723 | 36.697 | −5.038 | 5.883 | 1.00 | 31.23 | C |
| ATOM | 2850 | CB | THR | A | 723 | 35.712 | −4.775 | 7.046 | 1.00 | 30.40 | C |
| ATOM | 2852 | OG1 | THR | A | 723 | 35.606 | −5.914 | 7.911 | 1.00 | 32.47 | O |
| ATOM | 2854 | CG2 | THR | A | 723 | 34.344 | −4.558 | 6.528 | 1.00 | 32.10 | C |
| ATOM | 2858 | C | THR | A | 723 | 38.086 | −5.173 | 6.447 | 1.00 | 30.74 | C |
| ATOM | 2859 | O | THR | A | 723 | 38.563 | −4.269 | 7.109 | 1.00 | 30.67 | O |
| ATOM | 2860 | N | ASP | A | 724 | 38.748 | −6.280 | 6.178 | 1.00 | 31.33 | N |
| ATOM | 2862 | CA | ASP | A | 724 | 40.189 | −6.380 | 6.428 | 1.00 | 31.24 | C |
| ATOM | 2864 | CB | ASP | A | 724 | 40.733 | −7.764 | 6.088 | 1.00 | 30.66 | C |
| ATOM | 2867 | CG | ASP | A | 724 | 40.290 | −8.837 | 7.030 | 1.00 | 29.75 | C |
| ATOM | 2868 | OD1 | ASP | A | 724 | 39.440 | −8.600 | 7.884 | 1.00 | 29.76 | O |
| ATOM | 2869 | OD2 | ASP | A | 724 | 40.762 | −9.987 | 6.987 | 1.00 | 30.86 | O |
| ATOM | 2870 | C | ASP | A | 724 | 40.851 | −5.405 | 5.454 | 1.00 | 32.00 | C |
| ATOM | 2871 | O | ASP | A | 724 | 40.796 | −5.626 | 4.228 | 1.00 | 31.56 | O |
| ATOM | 2872 | N | LEU | A | 725 | 41.451 | −4.324 | 5.949 | 1.00 | 30.56 | N |
| ATOM | 2874 | CA | LEU | A | 725 | 42.126 | −3.402 | 5.030 | 1.00 | 30.51 | C |
| ATOM | 2876 | CB | LEU | A | 725 | 42.814 | −2.254 | 5.771 | 1.00 | 31.33 | C |
| ATOM | 2879 | CG | LEU | A | 725 | 42.583 | −0.759 | 5.485 | 1.00 | 34.63 | C |
| ATOM | 2881 | CD1 | LEU | A | 725 | 43.914 | −0.011 | 5.423 | 1.00 | 36.43 | C |
| ATOM | 2885 | CD2 | LEU | A | 725 | 41.630 | −0.346 | 4.388 | 1.00 | 32.58 | C |
| ATOM | 2889 | C | LEU | A | 725 | 43.204 | −4.053 | 4.203 | 1.00 | 29.44 | C |
| ATOM | 2890 | O | LEU | A | 725 | 43.438 | −3.601 | 3.108 | 1.00 | 29.73 | O |
| ATOM | 2891 | N | ALA | A | 726 | 43.911 | −5.052 | 4.731 | 1.00 | 28.27 | N |
| ATOM | 2893 | CA | ALA | A | 726 | 44.933 | −5.746 | 3.953 | 1.00 | 29.03 | C |
| ATOM | 2895 | CB | ALA | A | 726 | 45.675 | −6.803 | 4.775 | 1.00 | 28.55 | C |
| ATOM | 2899 | C | ALA | A | 726 | 44.357 | −6.370 | 2.667 | 1.00 | 29.70 | C |
| ATOM | 2900 | O | ALA | A | 726 | 45.053 | −6.475 | 1.677 | 1.00 | 28.98 | O |
| ATOM | 2901 | N | LEU | A | 727 | 43.099 | −6.779 | 2.706 | 1.00 | 30.51 | N |
| ATOM | 2903 | CA | LEU | A | 727 | 42.458 | −7.400 | 1.552 | 1.00 | 32.05 | C |
| ATOM | 2905 | CB | LEU | A | 727 | 41.218 | −8.177 | 1.982 | 1.00 | 32.66 | C |
| ATOM | 2908 | CG | LEU | A | 727 | 41.369 | −9.660 | 2.296 | 1.00 | 37.39 | C |
| ATOM | 2910 | CD1 | LEU | A | 727 | 39.942 | −10.265 | 2.429 | 1.00 | 39.88 | C |
| ATOM | 2914 | CD2 | LEU | A | 727 | 42.170 | −10.453 | 1.259 | 1.00 | 38.45 | C |
| ATOM | 2918 | C | LEU | A | 727 | 42.044 | −6.313 | 0.546 | 1.00 | 30.69 | C |
| ATOM | 2919 | O | LEU | A | 727 | 42.123 | −6.514 | −0.637 | 1.00 | 29.90 | O |
| ATOM | 2920 | N | TYR | A | 728 | 41.626 | −5.166 | 1.060 | 1.00 | 30.15 | N |
| ATOM | 2922 | CA | TYR | A | 728 | 41.334 | −4.004 | 0.242 | 1.00 | 29.75 | C |
| ATOM | 2924 | CB | TYR | A | 728 | 40.822 | −2.832 | 1.087 | 1.00 | 30.44 | C |
| ATOM | 2927 | CG | TYR | A | 728 | 40.951 | −1.498 | 0.372 | 1.00 | 31.40 | C |
| ATOM | 2928 | CD1 | TYR | A | 728 | 40.157 | −1.192 | −0.715 | 1.00 | 32.68 | C |
| ATOM | 2930 | CE1 | TYR | A | 728 | 40.296 | 0.029 | −1.386 | 1.00 | 33.60 | C |
| ATOM | 2932 | CZ | TYR | A | 728 | 41.232 | 0.939 | −0.956 | 1.00 | 32.26 | C |
| ATOM | 2933 | OH | TYR | A | 728 | 41.386 | 2.148 | −1.597 | 1.00 | 33.27 | O |
| ATOM | 2935 | CE2 | TYR | A | 728 | 42.044 | 0.640 | 0.111 | 1.00 | 32.66 | C |
| ATOM | 2937 | CD2 | TYR | A | 728 | 41.888 | −0.555 | 0.782 | 1.00 | 31.84 | C |
| ATOM | 2939 | C | TYR | A | 728 | 42.582 | −3.607 | −0.521 | 1.00 | 29.20 | C |
| ATOM | 2940 | O | TYR | A | 728 | 42.541 | −3.458 | −1.739 | 1.00 | 28.20 | O |
| ATOM | 2941 | N | ILE | A | 729 | 43.713 | −3.516 | 0.173 | 1.00 | 27.85 | N |
| ATOM | 2943 | CA | ILE | A | 729 | 44.937 | −3.036 | −0.461 | 1.00 | 27.07 | C |
| ATOM | 2945 | CB | ILE | A | 729 | 46.028 | −2.741 | 0.603 | 1.00 | 26.18 | C |
| ATOM | 2947 | CG1 | ILE | A | 729 | 45.600 | −1.552 | 1.458 | 1.00 | 27.32 | C |
| ATOM | 2950 | CD1 | ILE | A | 729 | 46.362 | −1.426 | 2.761 | 1.00 | 29.88 | C |
| ATOM | 2954 | CG2 | ILE | A | 729 | 47.358 | −2.423 | −0.087 | 1.00 | 25.82 | C |
| ATOM | 2958 | C | ILE | A | 729 | 45.428 | −4.032 | −1.491 | 1.00 | 26.41 | C |
| ATOM | 2959 | O | ILE | A | 729 | 45.980 | −3.646 | −2.520 | 1.00 | 25.30 | O |
| ATOM | 2960 | N | LYS | A | 730 | 45.234 | −5.309 | −1.183 | 1.00 | 26.14 | N |
| ATOM | 2962 | CA | LYS | A | 730 | 45.673 | −6.381 | −2.046 | 1.00 | 27.40 | C |
| ATOM | 2964 | CB | LYS | A | 730 | 45.487 | −7.734 | −1.346 | 1.00 | 26.89 | C |
| ATOM | 2967 | CG | LYS | A | 730 | 45.929 | −8.938 | −2.203 | 1.00 | 29.59 | C |
| ATOM | 2970 | CD | LYS | A | 730 | 45.470 | −10.258 | −1.580 | 1.00 | 33.15 | C |
| ATOM | 2973 | CE | LYS | A | 730 | 45.912 | −11.470 | −2.371 | 1.00 | 34.65 | C |
| ATOM | 2976 | NZ | LYS | A | 730 | 45.545 | −12.673 | −1.583 | 1.00 | 38.38 | N |
| ATOM | 2980 | C | LYS | A | 730 | 44.904 | −6.360 | −3.389 | 1.00 | 27.18 | C |
| ATOM | 2981 | O | LYS | A | 730 | 45.489 | −6.603 | −4.464 | 1.00 | 26.31 | O |
| ATOM | 2982 | N | ARG | A | 731 | 43.606 | −6.106 | −3.316 | 1.00 | 27.73 | N |
| ATOM | 2984 | CA | ARG | A | 731 | 42.702 | −6.333 | −4.469 | 1.00 | 29.48 | C |
| ATOM | 2986 | CB | ARG | A | 731 | 41.423 | −7.017 | −3.999 | 1.00 | 29.63 | C |
| ATOM | 2989 | CG | ARG | A | 731 | 41.621 | −8.516 | −3.662 | 1.00 | 34.45 | C |
| ATOM | 2992 | CD | ARG | A | 731 | 40.333 | −9.179 | −3.157 | 1.00 | 39.61 | C |
| ATOM | 2995 | NE | ARG | A | 731 | 39.558 | −9.710 | −4.292 | 1.00 | 45.03 | N |
| ATOM | 2997 | CZ | ARG | A | 731 | 38.213 | −9.788 | −4.350 | 1.00 | 45.41 | C |
| ATOM | 2998 | NH1 | ARG | A | 731 | 37.438 | −9.436 | −3.329 | 1.00 | 45.49 | N |
| ATOM | 3001 | NH2 | ARG | A | 731 | 37.644 | −10.258 | −5.447 | 1.00 | 47.21 | N |
| ATOM | 3004 | C | ARG | A | 731 | 42.354 | −5.067 | −5.266 | 1.00 | 29.31 | C |
| ATOM | 3005 | O | ARG | A | 731 | 41.908 | −5.152 | −6.416 | 1.00 | 28.60 | O |
| ATOM | 3006 | N | ARG | A | 732 | 42.573 | −3.902 | −4.661 | 1.00 | 29.07 | N |
| ATOM | 3008 | CA | ARG | A | 732 | 42.095 | −2.660 | −5.242 | 1.00 | 29.47 | C |
| ATOM | 3010 | CB | ARG | A | 732 | 42.269 | −1.481 | −4.300 | 1.00 | 29.23 | C |
| ATOM | 3013 | CG | ARG | A | 732 | 43.695 | −1.030 | −4.066 | 1.00 | 29.95 | C |
| ATOM | 3016 | CD | ARG | A | 732 | 43.791 | 0.013 | −2.935 | 1.00 | 28.49 | C |
| ATOM | 3019 | NE | ARG | A | 732 | 45.155 | 0.519 | −2.808 | 1.00 | 31.38 | N |
| ATOM | 3021 | CZ | ARG | A | 732 | 45.491 | 1.713 | −2.325 | 1.00 | 28.56 | C |
| ATOM | 3022 | NH1 | ARG | A | 732 | 44.585 | 2.561 | −1.890 | 1.00 | 29.84 | N |
| ATOM | 3025 | NH2 | ARG | A | 732 | 46.751 | 2.056 | −2.301 | 1.00 | 30.13 | N |
| ATOM | 3028 | C | ARG | A | 732 | 42.718 | −2.324 | −6.580 | 1.00 | 30.07 | C |
| ATOM | 3029 | O | ARG | A | 732 | 42.039 | −1.750 | −7.401 | 1.00 | 30.71 | O |
| ATOM | 3030 | N | GLY | A | 733 | 43.987 | −2.663 | −6.797 | 1.00 | 29.98 | N |
| ATOM | 3032 | CA | GLY | A | 733 | 44.666 | −2.401 | −8.063 | 1.00 | 30.89 | C |
| ATOM | 3035 | C | GLY | A | 733 | 43.915 | −2.924 | −9.281 | 1.00 | 32.03 | C |
| ATOM | 3036 | O | GLY | A | 733 | 43.810 | −2.237 | −10.323 | 1.00 | 31.34 | O |
| ATOM | 3037 | N | GLU | A | 734 | 43.384 | −4.141 | −9.150 | 1.00 | 33.29 | N |
| ATOM | 3039 | CA | GLU | A | 734 | 42.573 | −4.741 | −10.209 | 1.00 | 34.28 | C |
| ATOM | 3041 | CB | GLU | A | 734 | 42.197 | −6.194 | −9.861 | 1.00 | 34.83 | C |
| ATOM | 3044 | CG | GLU | A | 734 | 41.219 | −6.849 | −10.858 | 1.00 | 36.43 | C |
| ATOM | 3047 | CD | GLU | A | 734 | 40.859 | −8.284 | −10.513 | 1.00 | 37.93 | C |
| ATOM | 3048 | OE1 | GLU | A | 734 | 40.857 | −8.644 | −9.318 | 1.00 | 38.78 | O |
| ATOM | 3049 | OE2 | GLU | A | 734 | 40.574 | −9.058 | −11.458 | 1.00 | 41.92 | O |
| ATOM | 3050 | C | GLU | A | 734 | 41.303 | −3.904 | −10.450 | 1.00 | 33.90 | C |
| ATOM | 3051 | O | GLU | A | 734 | 40.929 | −3.652 | −11.592 | 1.00 | 34.11 | O |
| ATOM | 3052 | N | PHE | A | 735 | 40.635 | −3.489 | −9.374 | 1.00 | 34.01 | N |
| ATOM | 3054 | CA | PHE | A | 735 | 39.474 | −2.601 | −9.476 | 1.00 | 33.89 | C |
| ATOM | 3056 | CB | PHE | A | 735 | 38.942 | −2.296 | −8.078 | 1.00 | 34.08 | C |
| ATOM | 3059 | CG | PHE | A | 735 | 37.713 | −1.439 | −8.042 | 1.00 | 36.84 | C |
| ATOM | 3060 | CD1 | PHE | A | 735 | 36.594 | −1.745 | −8.815 | 1.00 | 39.84 | C |
| ATOM | 3062 | CE1 | PHE | A | 735 | 35.452 | −0.962 | −8.756 | 1.00 | 40.64 | C |
| ATOM | 3064 | CZ | PHE | A | 735 | 35.407 | 0.133 | −7.910 | 1.00 | 41.90 | C |
| ATOM | 3066 | CE2 | PHE | A | 735 | 36.512 | 0.437 | −7.105 | 1.00 | 40.61 | C |
| ATOM | 3068 | CD2 | PHE | A | 735 | 37.650 | −0.347 | −7.180 | 1.00 | 39.10 | C |
| ATOM | 3070 | C | PHE | A | 735 | 39.814 | −1.302 | −10.191 | 1.00 | 33.50 | C |
| ATOM | 3071 | O | PHE | A | 735 | 39.151 | −0.934 | −11.180 | 1.00 | 33.37 | O |
| ATOM | 3072 | N | PHE | A | 736 | 40.844 | −0.616 | −9.704 | 1.00 | 33.05 | N |
| ATOM | 3074 | CA | PHE | A | 736 | 41.217 | 0.699 | −10.206 | 1.00 | 34.06 | C |
| ATOM | 3076 | CB | PHE | A | 736 | 42.366 | 1.295 | −9.392 | 1.00 | 34.30 | C |
| ATOM | 3079 | CG | PHE | A | 736 | 42.024 | 1.593 | −7.941 | 1.00 | 35.16 | C |
| ATOM | 3080 | CD1 | PHE | A | 736 | 43.043 | 1.852 | −7.027 | 1.00 | 35.56 | C |
| ATOM | 3082 | CE1 | PHE | A | 736 | 42.751 | 2.126 | −5.726 | 1.00 | 37.00 | C |
| ATOM | 3084 | CZ | PHE | A | 736 | 41.436 | 2.123 | −5.300 | 1.00 | 38.26 | C |
| ATOM | 3086 | CE2 | PHE | A | 736 | 40.421 | 1.854 | −6.195 | 1.00 | 36.37 | C |
| ATOM | 3088 | CD2 | PHE | A | 736 | 40.714 | 1.598 | −7.490 | 1.00 | 34.99 | C |
| ATOM | 3090 | C | PHE | A | 736 | 41.612 | 0.665 | −11.689 | 1.00 | 35.47 | C |
| ATOM | 3091 | O | PHE | A | 736 | 41.337 | 1.620 | −12.416 | 1.00 | 35.44 | O |
| ATOM | 3092 | N | GLU | A | 737 | 42.224 | −0.447 | −12.116 | 1.00 | 35.60 | N |
| ATOM | 3094 | CA | GLU | A | 737 | 42.700 | −0.632 | −13.476 | 1.00 | 36.57 | C |
| ATOM | 3096 | CB | GLU | A | 737 | 43.742 | −1.765 | −13.529 | 1.00 | 36.39 | C |
| ATOM | 3099 | CG | GLU | A | 737 | 44.284 | −2.096 | −14.912 | 1.00 | 38.88 | C |
| ATOM | 3102 | CD | GLU | A | 737 | 45.512 | −1.281 | −15.295 | 1.00 | 41.05 | C |
| ATOM | 3103 | OE1 | GLU | A | 737 | 45.690 | −0.154 | −14.778 | 1.00 | 42.96 | O |
| ATOM | 3104 | OE2 | GLU | A | 737 | 46.300 | −1.772 | −16.131 | 1.00 | 42.28 | O |
| ATOM | 3105 | C | GLU | A | 737 | 41.535 | −0.931 | −14.421 | 1.00 | 36.83 | C |
| ATOM | 3106 | O | GLU | A | 737 | 41.483 | −0.395 | −15.521 | 1.00 | 35.58 | O |
| ATOM | 3107 | N | LEU | A | 738 | 40.615 | −1.790 | −13.994 | 1..00 | 37.36 | N |
| ATOM | 3109 | CA | LEU | A | 738 | 39.409 | −2.021 | −14.773 | 1.00 | 37.62 | C |
| ATOM | 3111 | CB | LEU | A | 738 | 38.474 | −3.008 | −14.064 | 1.00 | 37.25 | C |
| ATOM | 3114 | CG | LEU | A | 738 | 38.872 | −4.481 | −13.963 | 1.00 | 36.35 | C |
| ATOM | 3116 | CD1 | LEU | A | 738 | 37.998 | −5.192 | −12.939 | 1.00 | 36.30 | C |
| ATOM | 3120 | CD2 | LEU | A | 738 | 38.785 | −5.189 | −15.300 | 1.00 | 37.05 | C |
| ATOM | 3124 | C | LEU | A | 738 | 38.685 | −0.688 | −15.054 | 1.00 | 38.70 | C |
| ATOM | 3125 | O | LEU | A | 738 | 38.291 | −0.438 | −16.174 | 1.00 | 39.15 | O |
| ATOM | 3126 | N | ILE | A | 739 | 38.523 | 0.172 | −14.050 | 1.00 | 40.41 | N |
| ATOM | 3128 | CA | ILE | A | 739 | 37.810 | 1.447 | −14.227 | 1.00 | 41.58 | C |
| ATOM | 3130 | CB | ILE | A | 739 | 37.419 | 2.036 | −12.858 | 1.00 | 41.98 | C |
| ATOM | 3132 | CG1 | ILE | A | 739 | 36.243 | 1.242 | −12.282 | 1.00 | 41.05 | C |
| ATOM | 3135 | CD1 | ILE | A | 739 | 36.094 | 1.368 | −10.859 | 1.00 | 41.49 | C |
| ATOM | 3139 | CG2 | ILE | A | 739 | 37.024 | 3.520 | −12.973 | 1.00 | 42.55 | C |
| ATOM | 3143 | C | ILE | A | 739 | 38.594 | 2.472 | −15.070 | 1.00 | 43.09 | C |
| ATOM | 3144 | O | ILE | A | 739 | 38.010 | 3.155 | −15.921 | 1.00 | 43.46 | O |
| ATOM | 3145 | N | ARG | A | 740 | 39.902 | 2.565 | −14.828 | 1.00 | 43.89 | N |
| ATOM | 3147 | CA | ARG | A | 740 | 40.805 | 3.523 | −15.480 | 1.00 | 44.29 | C |
| ATOM | 3149 | CB | ARG | A | 740 | 42.229 | 3.264 | −14.978 | 1.00 | 44.62 | C |
| ATOM | 3152 | CG | ARG | A | 740 | 43.352 | 4.163 | −15.486 | 1.00 | 45.76 | C |
| ATOM | 3155 | CD | ARG | A | 740 | 44.704 | 3.845 | −14.808 | 1.00 | 46.92 | C |
| ATOM | 3158 | NE | ARG | A | 740 | 44.528 | 3.637 | −13.357 | 1.00 | 49.06 | N |
| ATOM | 3160 | CZ | ARG | A | 740 | 45.175 | 2.735 | −12.593 | 1.00 | 47.38 | C |
| ATOM | 3161 | NH1 | ARG | A | 740 | 46.087 | 1.913 | −13.098 | 1.00 | 47.75 | N |
| ATOM | 3164 | NH2 | ARG | A | 740 | 44.900 | 2.671 | −11.306 | 1.00 | 46.41 | N |
| ATOM | 3167 | C | ARG | A | 740 | 40.731 | 3.329 | −16.979 | 1.00 | 44.59 | C |
| ATOM | 3168 | O | ARG | A | 740 | 40.689 | 4.294 | −17.759 | 1.00 | 44.64 | O |
| ATOM | 3169 | N | LYS | A | 741 | 40.714 | 2.060 | −17.369 | 1.00 | 44.87 | N |
| ATOM | 3171 | CA | LYS | A | 741 | 40.325 | 1.653 | −18.706 | 1.00 | 45.02 | C |
| ATOM | 3173 | CB | LYS | A | 741 | 40.644 | 0.170 | −18.883 | 1.00 | 45.13 | C |
| ATOM | 3176 | CG | LYS | A | 741 | 42.115 | −0.177 | −18.715 | 1.00 | 44.76 | C |
| ATOM | 3179 | CD | LYS | A | 741 | 42.381 | −1.583 | −19.222 | 1.00 | 44.18 | C |
| ATOM | 3182 | CE | LYS | A | 741 | 43.806 | −2.043 | −18.913 | 1.00 | 44.31 | C |
| ATOM | 3185 | NZ | LYS | A | 741 | 44.462 | −2.620 | −20.128 | 1.00 | 43.46 | N |
| ATOM | 3189 | C | LYS | A | 741 | 38.816 | 1.933 | −18.812 | 1.00 | 45.27 | C |
| ATOM | 3190 | O | LYS | A | 741 | 38.364 | 3.001 | −18.383 | 1.00 | 45.85 | O |
| ATOM | 3191 | N | ASN | A | 742 | 38.028 | 1.010 | −19.358 | 1.00 | 44.90 | N |
| ATOM | 3193 | CA | ASN | A | 742 | 36.571 | 1.065 | −19.175 | 1.00 | 44.48 | C |
| ATOM | 3195 | CB | ASN | A | 742 | 35.915 | 2.030 | −20.184 | 1.00 | 44.33 | C |
| ATOM | 3198 | CG | ASN | A | 742 | 35.926 | 3.472 | −19.702 | 1.00 | 44.37 | C |
| ATOM | 3199 | OD1 | ASN | A | 742 | 35.169 | 3.857 | −18.800 | 1.00 | 43.50 | O |
| ATOM | 3200 | ND2 | ASN | A | 742 | 36.807 | 4.274 | −20.284 | 1.00 | 44.38 | N |
| ATOM | 3203 | C | ASN | A | 742 | 36.016 | −0.336 | −19.333 | 1.00 | 44.23 | C |
| ATOM | 3204 | O | ASN | A | 742 | 35.114 | −0.581 | −20.155 | 1.00 | 44.09 | O |
| ATOM | 3205 | N | GLN | A | 743 | 36.598 | −1.257 | −18.572 | 1.00 | 43.32 | N |
| ATOM | 3207 | CA | GLN | A | 743 | 36.328 | −2.679 | −18.738 | 1.00 | 43.18 | C |
| ATOM | 3209 | CB | GLN | A | 743 | 37.643 | −3.457 | −18.878 | 1.00 | 42.99 | C |
| ATOM | 3212 | CG | GLN | A | 743 | 38.443 | −3.088 | −20.134 | 1.00 | 44.00 | C |
| ATOM | 3215 | CD | GLN | A | 743 | 39.778 | −3.818 | −20.243 | 1.00 | 44.62 | C |
| ATOM | 3216 | OE1 | GLN | A | 743 | 40.174 | −4.554 | −19.332 | 1.00 | 45.85 | O |
| ATOM | 3217 | NE2 | GLN | A | 743 | 40.478 | −3.604 | −21.351 | 1.00 | 44.62 | N |
| ATOM | 3220 | C | GLN | A | 743 | 35.517 | −3.216 | −17.584 | 1.00 | 42.81 | C |
| ATOM | 3221 | O | GLN | A | 743 | 35.214 | −4.411 | −17.539 | 1.00 | 42.59 | O |
| ATOM | 3222 | N | PHE | A | 744 | 35.150 | −2.328 | −16.657 | 1.00 | 43.16 | N |
| ATOM | 3224 | CA | PHE | A | 744 | 34.518 | −2.740 | −15.422 | 1.00 | 42.76 | C |
| ATOM | 3226 | CB | PHE | A | 744 | 34.455 | −1.587 | −14.429 | 1.00 | 43.01 | C |
| ATOM | 3229 | CG | PHE | A | 744 | 33.629 | −1.899 | −13.220 | 1.00 | 43.65 | C |
| ATOM | 3230 | CD1 | PHE | A | 744 | 33.922 | −3.012 | −12.438 | 1.00 | 43.33 | C |
| ATOM | 3232 | CE1 | PHE | A | 744 | 33.161 | −3.319 | −11.324 | 1.00 | 43.62 | C |
| ATOM | 3234 | CZ | PHE | A | 744 | 32.083 | −2.523 | −10.987 | 1.00 | 44.49 | C |
| ATOM | 3236 | CE2 | PHE | A | 744 | 31.767 | −1.417 | −11.772 | 1.00 | 45.02 | C |
| ATOM | 3238 | CD2 | PHE | A | 744 | 32.535 | −1.115 | −12.886 | 1.00 | 44.45 | C |
| ATOM | 3240 | C | PHE | A | 744 | 33.123 | −3.218 | −15.731 | 1.00 | 42.74 | C |
| ATOM | 3241 | O | PHE | A | 744 | 32.390 | −2.523 | −16.412 | 1.00 | 42.85 | O |
| ATOM | 3242 | N | ASN | A | 745 | 32.757 | −4.384 | −15.202 | 1.00 | 42.84 | N |
| ATOM | 3244 | CA | ASN | A | 745 | 31.540 | −5.082 | −15.592 | 1.00 | 43.14 | C |
| ATOM | 3246 | CB | ASN | A | 745 | 31.825 | −5.887 | −16.871 | 1.00 | 43.72 | C |
| ATOM | 3249 | CG | ASN | A | 745 | 30.967 | −5.444 | −18.051 | 1.00 | 44.57 | C |
| ATOM | 3250 | OD1 | ASN | A | 745 | 29.836 | −4.996 | −17.874 | 1.00 | 47.27 | O |
| ATOM | 3251 | ND2 | ASN | A | 745 | 31.506 | −5.571 | −19.265 | 1.00 | 46.68 | N |
| ATOM | 3254 | C | ASN | A | 745 | 30.936 | −5.994 | −14.498 | 1.00 | 43.33 | C |
| ATOM | 3255 | O | ASN | A | 745 | 31.463 | −7.082 | −14.212 | 1.00 | 43.25 | O |
| ATOM | 3256 | N | LEU | A | 746 | 29.817 | −5.538 | −13.914 | 1.00 | 43.48 | N |
| ATOM | 3258 | CA | LEU | A | 746 | 29.080 | −6.217 | −12.834 | 1.00 | 43.30 | C |
| ATOM | 3260 | CB | LEU | A | 746 | 27.926 | −5.322 | −12.379 | 1.00 | 43.40 | C |
| ATOM | 3263 | CG | LEU | A | 746 | 28.272 | −4.048 | −11.577 | 1.00 | 43.36 | C |
| ATOM | 3265 | CD1 | LEU | A | 746 | 28.023 | −2.727 | −12.339 | 1.00 | 43.92 | C |
| ATOM | 3269 | CD2 | LEU | A | 746 | 27.460 | −4.041 | −10.334 | 1.00 | 43.29 | C |
| ATOM | 3273 | C | LEU | A | 746 | 28.512 | −7.603 | −13.176 | 1.00 | 43.97 | C |
| ATOM | 3274 | O | LEU | A | 746 | 28.046 | −8.334 | −12.298 | 1.00 | 44.14 | O |
| ATOM | 3275 | N | GLU | A | 747 | 28.510 | −7.927 | −14.462 | 1.00 | 44.34 | N |
| ATOM | 3277 | CA | GLU | A | 747 | 28.263 | −9.275 | −14.966 | 1.00 | 44.73 | C |
| ATOM | 3279 | CB | GLU | A | 747 | 28.358 | −9.226 | −16.491 | 1.00 | 44.60 | C |
| ATOM | 3282 | CG | GLU | A | 747 | 29.512 | −8.370 | −16.999 | 1.00 | 44.39 | C |
| ATOM | 3285 | CD | GLU | A | 747 | 30.170 | −8.919 | −18.260 | 1.00 | 45.57 | C |
| ATOM | 3286 | OE1 | GLU | A | 747 | 29.449 | −9.389 | −19.204 | 1.00 | 42.97 | O |
| ATOM | 3287 | OE2 | GLU | A | 747 | 31.427 | −8.864 | −18.300 | 1.00 | 45.91 | O |
| ATOM | 3288 | C | GLU | A | 747 | 29.154 | −10.451 | −14.464 | 1.00 | 45.39 | C |
| ATOM | 3289 | O | GLU | A | 747 | 28.641 | −11.570 | −14.274 | 1.00 | 45.81 | O |
| ATOM | 3290 | N | ASP | A | 748 | 30.467 | −10.252 | −14.308 | 1.00 | 45.36 | N |
| ATOM | 3292 | CA | ASP | A | 748 | 31.318 | −11.384 | −13.935 | 1.00 | 45.44 | C |
| ATOM | 3294 | CB | ASP | A | 748 | 32.860 | −11.184 | −14.061 | 1.00 | 45.79 | C |
| ATOM | 3297 | CG | ASP | A | 748 | 33.291 | −10.175 | −15.111 | 1.00 | 47.52 | C |
| ATOM | 3298 | OD1 | ASP | A | 748 | 34.526 | −10.021 | −15.246 | 1.00 | 50.96 | O |
| ATOM | 3299 | OD2 | ASP | A | 748 | 32.531 | −9.496 | −15.839 | 1.00 | 49.90 | O |
| ATOM | 3300 | C | ASP | A | 748 | 31.042 | −11.589 | −12.460 | 1.00 | 44.95 | C |
| ATOM | 3301 | O | ASP | A | 748 | 31.201 | −10.649 | −11.686 | 1.00 | 44.92 | O |
| ATOM | 3302 | N | PRO | A | 749 | 30.710 | −12.814 | −12.057 | 1.00 | 44.38 | N |
| ATOM | 3303 | CA | PRO | A | 749 | 30.700 | −13.157 | −10.633 | 1.00 | 44.02 | C |
| ATOM | 3305 | CB | PRO | A | 749 | 30.837 | −14.686 | −10.632 | 1.00 | 44.29 | C |
| ATOM | 3308 | CG | PRO | A | 749 | 30.281 | −15.142 | −11.954 | 1.00 | 43.95 | C |
| ATOM | 3311 | CD | PRO | A | 749 | 30.386 | −13.975 | −12.909 | 1.00 | 44.40 | C |
| ATOM | 3314 | C | PRO | A | 749 | 31.897 | −12.524 | −9.906 | 1.00 | 43.81 | C |
| ATOM | 3315 | O | PRO | A | 749 | 31.727 | −12.026 | −8.792 | 1.00 | 43.90 | O |
| ATOM | 3316 | N | HIS | A | 750 | 33.068 | −12.525 | −10.545 | 1.00 | 43.08 | N |
| ATOM | 3318 | CA | HIS | A | 750 | 34.294 | −12.034 | −9.926 | 1.00 | 42.92 | C |
| ATOM | 3320 | CB | HIS | A | 750 | 35.543 | −12.469 | −10.734 | 1.00 | 43.34 | C |
| ATOM | 3323 | CG | HIS | A | 750 | 36.816 | −12.062 | −10.070 | 1.00 | 45.39 | C |
| ATOM | 3324 | ND1 | HIS | A | 750 | 37.534 | −10.951 | −10.458 | 1.00 | 46.61 | N |
| ATOM | 3326 | CE1 | HIS | A | 750 | 38.566 | −10.800 | −9.649 | 1.00 | 47.97 | C |
| ATOM | 3328 | NE2 | HIS | A | 750 | 38.526 | −11.750 | −8.732 | 1.00 | 48.77 | N |
| ATOM | 3330 | CD2 | HIS | A | 750 | 37.432 | −12.544 | −8.964 | 1.00 | 48.45 | C |
| ATOM | 3332 | C | HIS | A | 750 | 34.311 | −10.509 | −9.732 | 1.00 | 41.70 | C |
| ATOM | 3333 | O | HIS | A | 750 | 34.591 | −10.027 | −8.646 | 1.00 | 41.56 | O |
| ATOM | 3334 | N | GLN | A | 751 | 34.038 | −9.758 | −10.791 | 1.00 | 40.61 | N |
| ATOM | 3336 | CA | GLN | A | 751 | 34.014 | −8.301 | −10.705 | 1.00 | 39.95 | C |
| ATOM | 3338 | CB | GLN | A | 751 | 33.881 | −7.693 | −12.098 | 1.00 | 39.30 | C |
| ATOM | 3341 | CG | GLN | A | 751 | 35.122 | −7.943 | −12.951 | 1.00 | 38.86 | C |
| ATOM | 3344 | CD | GLN | A | 751 | 35.153 | −7.152 | −14.235 | 1.00 | 36.74 | C |
| ATOM | 3345 | OE1 | GLN | A | 751 | 34.985 | −5.946 | −14.232 | 1.00 | 38.17 | O |
| ATOM | 3346 | NE2 | GLN | A | 751 | 35.388 | −7.826 | −15.325 | 1.00 | 35.87 | N |
| ATOM | 3349 | C | GLN | A | 751 | 32.914 | −7.793 | −9.762 | 1.00 | 40.04 | C |
| ATOM | 3350 | O | GLN | A | 751 | 33.059 | −6.721 | −9.140 | 1.00 | 40.25 | O |
| ATOM | 3351 | N | LYS | A | 752 | 31.842 | −8.581 | −9.638 | 1.00 | 39.43 | N |
| ATOM | 3353 | CA | LYS | A | 752 | 30.769 | −8.341 | −8.658 | 1.00 | 38.83 | C |
| ATOM | 3355 | CB | LYS | A | 752 | 29.564 | −9.302 | −8.911 | 1.00 | 39.25 | C |
| ATOM | 3358 | CG | LYS | A | 752 | 28.455 | −9.313 | −7.841 | 1.00 | 38.89 | C |
| ATOM | 3361 | CD | LYS | A | 752 | 27.855 | −7.931 | −7.595 | 1.00 | 39.51 | C |
| ATOM | 3364 | CE | LYS | A | 752 | 26.957 | −7.457 | −8.782 | 1.00 | 38.66 | C |
| ATOM | 3367 | NZ | LYS | A | 752 | 25.767 | −8.330 | −8.981 | 1.00 | 38.40 | N |
| ATOM | 3371 | C | LYS | A | 752 | 31.267 | −8.492 | −7.246 | 1.00 | 38.37 | C |
| ATOM | 3372 | O | LYS | A | 752 | 31.015 | −7.605 | −6.402 | 1.00 | 38.69 | O |
| ATOM | 3373 | N | GLU | A | 753 | 31.947 | −9.599 | −6.947 | 1.00 | 38.00 | N |
| ATOM | 3375 | CA | GLU | A | 753 | 32.392 | −9.855 | −5.574 | 1.00 | 39.11 | C |
| ATOM | 3377 | CB | GLU | A | 753 | 33.066 | −11.235 | −5.422 | 1.00 | 40.06 | C |
| ATOM | 3380 | CG | GLU | A | 753 | 32.071 | −12.421 | −5.462 | 1.00 | 43.59 | C |
| ATOM | 3383 | CD | GLU | A | 753 | 32.680 | −13.799 | −5.799 | 1.00 | 48.13 | C |
| ATOM | 3384 | OE1 | GLU | A | 753 | 33.907 | −13.899 | −6.081 | 1.00 | 51.83 | O |
| ATOM | 3385 | OE2 | GLU | A | 753 | 31.910 | −14.806 | −5.784 | 1.00 | 48.81 | O |
| ATOM | 3386 | C | GLU | A | 753 | 33.328 | −8.705 | −5.159 | 1.00 | 38.39 | C |
| ATOM | 3387 | O | GLU | A | 753 | 33.232 | −8.168 | −4.074 | 1.00 | 37.95 | O |
| ATOM | 3388 | N | LEU | A | 754 | 34.154 | −8.304 | −6.110 | 1.00 | 37.59 | N |
| ATOM | 3390 | CA | LEU | A | 754 | 35.181 | −7.295 | −5.950 | 1.00 | 37.06 | C |
| ATOM | 3392 | CB | LEU | A | 754 | 35.993 | −7.307 | −7.233 | 1.00 | 36.87 | C |
| ATOM | 3395 | CG | LEU | A | 754 | 37.238 | −6.492 | −7.427 | 1.00 | 39.55 | C |
| ATOM | 3397 | CD1 | LEU | A | 754 | 36.787 | −5.122 | −7.839 | 1.00 | 42.06 | C |
| ATOM | 3401 | CD2 | LEU | A | 754 | 38.169 | −6.458 | −6.171 | 1.00 | 41.44 | C |
| ATOM | 3405 | C | LEU | A | 754 | 34.575 | −5.908 | −5.693 | 1.00 | 36.10 | C |
| ATOM | 3406 | O | LEU | A | 754 | 35.066 | −5.140 | −4.862 | 1.00 | 33.82 | O |
| ATOM | 3407 | N | PHE | A | 755 | 33.517 | −5.593 | −6.427 | 1.00 | 35.18 | N |
| ATOM | 3409 | CA | PHE | A | 755 | 32.812 | −4.320 | −6.260 | 1.00 | 34.80 | C |
| ATOM | 3411 | CB | PHE | A | 755 | 31.773 | −4.066 | −7.365 | 1.00 | 35.11 | C |
| ATOM | 3414 | CG | PHE | A | 755 | 30.958 | −2.838 | −7.115 | 1.00 | 36.68 | C |
| ATOM | 3415 | CD1 | PHE | A | 755 | 31.570 | −1.598 | −7.077 | 1.00 | 39.95 | C |
| ATOM | 3417 | CE1 | PHE | A | 755 | 30.861 | −0.476 | −6.794 | 1.00 | 41.31 | C |
| ATOM | 3419 | CZ | PHE | A | 755 | 29.522 | −0.565 | −6.523 | 1.00 | 40.36 | C |
| ATOM | 3421 | CE2 | PHE | A | 755 | 28.899 | −1.780 | −6.553 | 1.00 | 40.16 | C |
| ATOM | 3423 | CD2 | PHE | A | 755 | 29.624 | −2.921 | −6.821 | 1.00 | 39.43 | C |
| ATOM | 3425 | C | PHE | A | 755 | 32.126 | −4.275 | −4.907 | 1.00 | 33.82 | C |
| ATOM | 3426 | O | PHE | A | 755 | 32.186 | −3.268 | −4.222 | 1.00 | 34.54 | O |
| ATOM | 3427 | N | LEU | A | 756 | 31.488 | −5.365 | −4.510 | 1.00 | 33.07 | N |
| ATOM | 3429 | CA | LEU | A | 756 | 30.889 | −5.452 | −3.190 | 1.00 | 33.07 | C |
| ATOM | 3431 | CB | LEU | A | 756 | 30.198 | −6.810 | −2.988 | 1.00 | 33.19 | C |
| ATOM | 3434 | CG | LEU | A | 756 | 28.965 | −7.139 | −3.870 | 1.00 | 35.16 | C |
| ATOM | 3436 | CD1 | LEU | A | 756 | 28.482 | −8.535 | −3.574 | 1.00 | 37.25 | C |
| ATOM | 3440 | CD2 | LEU | A | 756 | 27.831 | −6.214 | −3.702 | 1.00 | 35.98 | C |
| ATOM | 3444 | C | LEU | A | 756 | 31.937 | −5.203 | −2.074 | 1.00 | 33.15 | C |
| ATOM | 3445 | O | LEU | A | 756 | 31.626 | −4.600 | −1.017 | 1.00 | 31.74 | O |
| ATOM | 3446 | N | ALA | A | 757 | 33.166 | −5.682 | −2.309 | 1.00 | 33.12 | N |
| ATOM | 3448 | CA | ALA | A | 757 | 34.238 | −5.537 | −1.323 | 1.00 | 33.06 | C |
| ATOM | 3450 | CB | ALA | A | 757 | 35.395 | −6.482 | −1.623 | 1.00 | 32.92 | C |
| ATOM | 3454 | C | ALA | A | 757 | 34.712 | −4.091 | −1.269 | 1.00 | 33.03 | C |
| ATOM | 3455 | O | ALA | A | 757 | 34.882 | −3.542 | −0.176 | 1.00 | 34.12 | O |
| ATOM | 3456 | N | MET | A | 758 | 34.864 | −3.452 | −2.424 | 1.00 | 32.95 | N |
| ATOM | 3458 | CA | MET | A | 758 | 35.271 | −2.049 | −2.457 | 1.00 | 33.93 | C |
| ATOM | 3460 | CB | MET | A | 758 | 35.527 | −1.574 | −3.881 | 1.00 | 33.91 | C |
| ATOM | 3463 | CG | MET | A | 758 | 36.672 | −2.314 | −4.621 | 1.00 | 35.83 | C |
| ATOM | 3466 | SD | MET | A | 758 | 38.384 | −2.046 | −3.916 | 1.00 | 38.11 | S |
| ATOM | 3467 | CE | MET | A | 758 | 38.783 | −3.635 | −3.324 | 1.00 | 37.96 | C |
| ATOM | 3471 | C | MET | A | 758 | 34.213 | −1.167 | −1.771 | 1.00 | 34.32 | C |
| ATOM | 3472 | O | MET | A | 758 | 34.548 | −0.214 | −1.088 | 1.00 | 33.57 | O |
| ATOM | 3473 | N | LEU | A | 759 | 32.937 | −1.517 | −1.934 | 1.00 | 35.01 | N |
| ATOM | 3475 | CA | LEU | A | 759 | 31.833 | −0.785 | −1.311 | 1.00 | 35.47 | C |
| ATOM | 3477 | CB | LEU | A | 759 | 30.519 | −1.275 | −1.903 | 1.00 | 35.61 | C |
| ATOM | 3480 | CG | LEU | A | 759 | 29.229 | −0.558 | −1.509 | 1.00 | 38.34 | C |
| ATOM | 3482 | CD1 | LEU | A | 759 | 29.323 | 0.977 | −1.635 | 1.00 | 38.93 | C |
| ATOM | 3486 | CD2 | LEU | A | 759 | 28.107 | −1.116 | −2.384 | 1.00 | 38.44 | C |
| ATOM | 3490 | C | LEU | A | 759 | 31.801 | −0.916 | 0.214 | 1.00 | 35.04 | C |
| ATOM | 3491 | O | LEU | A | 759 | 31.481 | 0.038 | 0.926 | 1.00 | 35.94 | O |
| ATOM | 3492 | N | MET | A | 760 | 32.084 | −2.115 | 0.715 | 1.00 | 34.32 | N |
| ATOM | 3494 | CA | MET | A | 760 | 32.197 | −2.345 | 2.140 | 1.00 | 33.02 | C |
| ATOM | 3496 | CB | MET | A | 760 | 32.522 | −3.821 | 2.427 | 1.00 | 33.34 | C |
| ATOM | 3499 | CG | MET | A | 760 | 31.323 | −4.760 | 2.332 | 1.00 | 32.73 | C |
| ATOM | 3502 | SD | MET | A | 760 | 29.985 | −4.481 | 3.492 | 1.00 | 35.30 | S |
| ATOM | 3503 | CE | MET | A | 760 | 30.754 | −4.506 | 5.105 | 1.00 | 33.09 | C |
| ATOM | 3507 | C | MET | A | 760 | 33.293 | −1.448 | 2.691 | 1.00 | 32.13 | C |
| ATOM | 3508 | O | MET | A | 760 | 33.105 | −0.774 | 3.689 | 1.00 | 31.76 | O |
| ATOM | 3509 | N | THR | A | 761 | 34.422 | −1.403 | 2.000 | 1.00 | 31.96 | N |
| ATOM | 3511 | CA | THR | A | 761 | 35.526 | −0.561 | 2.431 | 1.00 | 31.48 | C |
| ATOM | 3513 | CB | THR | A | 761 | 36.770 | −0.755 | 1.568 | 1.00 | 30.73 | C |
| ATOM | 3515 | OG1 | THR | A | 761 | 37.198 | −2.120 | 1.636 | 1.00 | 28.72 | O |
| ATOM | 3517 | CG2 | THR | A | 761 | 37.929 | 0.055 | 2.153 | 1.00 | 30.78 | C |
| ATOM | 3521 | C | THR | A | 761 | 35.127 | 0.897 | 2.445 | 1.00 | 31.80 | C |
| ATOM | 3522 | O | THR | A | 761 | 35.411 | 1.597 | 3.418 | 1.00 | 31.67 | O |
| ATOM | 3523 | N | ALA | A | 762 | 34.438 | 1.338 | 1.395 | 1.00 | 31.82 | N |
| ATOM | 3525 | CA | ALA | A | 762 | 34.028 | 2.740 | 1.254 | 1.00 | 32.31 | C |
| ATOM | 3527 | CB | ALA | A | 762 | 33.340 | 2.968 | −0.094 | 1.00 | 32.30 | C |
| ATOM | 3531 | C | ALA | A | 762 | 33.110 | 3.157 | 2.388 | 1.00 | 32.01 | C |
| ATOM | 3532 | O | ALA | A | 762 | 33.268 | 4.251 | 2.954 | 1.00 | 32.18 | O |
| ATOM | 3533 | N | CYS | A | 763 | 32.198 | 2.256 | 2.772 | 1.00 | 31.80 | N |
| ATOM | 3535 | CA | CYS | A | 763 | 31.290 | 2.513 | 3.913 | 1.00 | 31.62 | C |
| ATOM | 3537 | CB | CYS | A | 763 | 30.127 | 1.541 | 3.891 | 1.00 | 31.53 | C |
| ATOM | 3540 | SG | CYS | A | 763 | 29.097 | 1.770 | 2.386 | 1.00 | 33.88 | S |
| ATOM | 3541 | C | CYS | A | 763 | 31.986 | 2.460 | 5.278 | 1.00 | 31.47 | C |
| ATOM | 3542 | O | CYS | A | 763 | 31.705 | 3.252 | 6.142 | 1.00 | 32.16 | O |
| ATOM | 3543 | N | ASP | A | 764 | 32.915 | 1.529 | 5.435 | 1.00 | 32.05 | N |
| ATOM | 3545 | CA | ASP | A | 764 | 33.661 | 1.338 | 6.657 | 1.00 | 32.31 | C |
| ATOM | 3547 | CB | ASP | A | 764 | 34.548 | 0.107 | 6.478 | 1.00 | 32.85 | C |
| ATOM | 3550 | CG | ASP | A | 764 | 34.997 | −0.484 | 7.793 | 1.00 | 32.98 | C |
| ATOM | 3551 | OD1 | ASP | A | 764 | 34.339 | −0.212 | 8.822 | 1.00 | 35.64 | O |
| ATOM | 3552 | OD2 | ASP | A | 764 | 36.023 | −1.205 | 7.874 | 1.00 | 34.26 | O |
| ATOM | 3553 | C | ASP | A | 764 | 34.536 | 2.559 | 7.006 | 1.00 | 32.69 | C |
| ATOM | 3554 | O | ASP | A | 764 | 34.684 | 2.894 | 8.176 | 1.00 | 32.86 | O |
| ATOM | 3555 | N | LEU | A | 765 | 35.082 | 3.221 | 5.979 | 1.00 | 32.87 | N |
| ATOM | 3557 | CA | LEU | A | 765 | 36.021 | 4.343 | 6.156 | 1.00 | 33.11 | C |
| ATOM | 3559 | CB | LEU | A | 765 | 37.121 | 4.304 | 5.073 | 1.00 | 32.59 | C |
| ATOM | 3562 | CG | LEU | A | 765 | 37.940 | 3.020 | 4.974 | 1.00 | 35.12 | C |
| ATOM | 3564 | CD1 | LEU | A | 765 | 39.050 | 3.150 | 3.927 | 1.00 | 34.93 | C |
| ATOM | 3568 | CD2 | LEU | A | 765 | 38.559 | 2.603 | 6.310 | 1.00 | 36.80 | C |
| ATOM | 3572 | C | LEU | A | 765 | 35.337 | 5.698 | 6.085 | 1.00 | 32.04 | C |
| ATOM | 3573 | O | LEU | A | 765 | 35.994 | 6.720 | 6.171 | 1.00 | 31.39 | O |
| ATOM | 3574 | N | SER | A | 766 | 34.015 | 5.704 | 5.940 | 1.00 | 32.14 | N |
| ATOM | 3576 | CA | SER | A | 766 | 33.288 | 6.902 | 5.555 | 1.00 | 31.82 | C |
| ATOM | 3578 | CB | SER | A | 766 | 31.860 | 6.549 | 5.094 | 1.00 | 32.63 | C |
| ATOM | 3581 | OG | SER | A | 766 | 31.110 | 5.983 | 6.153 | 1.00 | 32.46 | O |
| ATOM | 3583 | C | SER | A | 766 | 33.230 | 8.033 | 6.590 | 1.00 | 31.12 | C |
| ATOM | 3584 | O | SER | A | 766 | 32.846 | 9.120 | 6.226 | 1.00 | 30.64 | O |
| ATOM | 3585 | N | ALA | A | 767 | 33.600 | 7.802 | 7.851 | 1.00 | 30.80 | N |
| ATOM | 3587 | CA | ALA | A | 767 | 33.854 | 8.938 | 8.767 | 1.00 | 30.32 | C |
| ATOM | 3589 | CB | ALA | A | 767 | 34.492 | 8.458 | 10.078 | 1.00 | 30.27 | C |
| ATOM | 3593 | C | ALA | A | 767 | 34.763 | 9.988 | 8.092 | 1.00 | 29.89 | C |
| ATOM | 3594 | O | ALA | A | 767 | 34.628 | 11.170 | 8.343 | 1.00 | 29.21 | O |
| ATOM | 3595 | N | ILE | A | 768 | 35.694 | 9.538 | 7.239 | 1.00 | 30.00 | N |
| ATOM | 3597 | CA | ILE | A | 768 | 36.646 | 10.422 | 6.569 | 1.00 | 30.23 | C |
| ATOM | 3599 | CB | ILE | A | 768 | 37.817 | 9.582 | 5.948 | 1.00 | 30.76 | C |
| ATOM | 3601 | CG1 | ILE | A | 768 | 39.050 | 10.423 | 5.684 | 1.00 | 32.05 | C |
| ATOM | 3604 | CD1 | ILE | A | 768 | 39.698 | 11.003 | 6.933 | 1.00 | 35.25 | C |
| ATOM | 3608 | CG2 | ILE | A | 768 | 37.413 | 9.018 | 4.601 | 1.00 | 32.10 | C |
| ATOM | 3612 | C | ILE | A | 768 | 35.999 | 11.349 | 5.524 | 1.00 | 30.34 | C |
| ATOM | 3613 | O | ILE | A | 768 | 36.625 | 12.337 | 5.070 | 1.00 | 30.09 | O |
| ATOM | 3614 | N | THR | A | 769 | 34.770 | 11.048 | 5.125 | 1.00 | 30.53 | N |
| ATOM | 3616 | CA | THR | A | 769 | 34.037 | 11.910 | 4.186 | 1.00 | 31.36 | C |
| ATOM | 3618 | CB | THR | A | 769 | 33.212 | 11.057 | 3.209 | 1.00 | 31.66 | C |
| ATOM | 3620 | OG1 | THR | A | 769 | 32.133 | 10.411 | 3.905 | 1.00 | 31.66 | O |
| ATOM | 3622 | CG2 | THR | A | 769 | 34.031 | 9.921 | 2.660 | 1.00 | 31.10 | C |
| ATOM | 3626 | C | THR | A | 769 | 33.087 | 12.926 | 4.840 | 1.00 | 31.89 | C |
| ATOM | 3627 | O | THR | A | 769 | 32.474 | 13.732 | 4.144 | 1.00 | 32.05 | O |
| ATOM | 3628 | N | LYS | A | 770 | 32.976 | 12.908 | 6.164 | 1.00 | 31.96 | N |
| ATOM | 3630 | CA | LYS | A | 770 | 31.939 | 13.694 | 6.859 | 1.00 | 31.48 | C |
| ATOM | 3632 | CB | LYS | A | 770 | 31.757 | 13.185 | 8.300 | 1.00 | 31.33 | C |
| ATOM | 3635 | CG | LYS | A | 770 | 31.213 | 11.754 | 8.406 | 1.00 | 31.96 | C |
| ATOM | 3638 | CD | LYS | A | 770 | 29.743 | 11.641 | 8.063 | 1.00 | 33.71 | C |
| ATOM | 3641 | CE | LYS | A | 770 | 29.300 | 10.166 | 8.044 | 1.00 | 35.91 | C |
| ATOM | 3644 | NZ | LYS | A | 770 | 27.964 | 9.987 | 7.459 | 1.00 | 35.13 | N |
| ATOM | 3648 | C | LYS | A | 770 | 32.251 | 15.172 | 6.867 | 1.00 | 30.72 | C |
| ATOM | 3649 | O | LYS | A | 770 | 33.400 | 15.549 | 6.735 | 1.00 | 31.50 | O |
| ATOM | 3650 | N | PRO | A | 771 | 31.243 | 16.034 | 7.036 | 1.00 | 30.88 | N |
| ATOM | 3651 | CA | PRO | A | 771 | 31.515 | 17.472 | 7.157 | 1.00 | 30.34 | C |
| ATOM | 3653 | CB | PRO | A | 771 | 30.162 | 18.066 | 7.577 | 1.00 | 30.70 | C |
| ATOM | 3656 | CG | PRO | A | 771 | 29.144 | 17.093 | 7.075 | 1.00 | 29.87 | C |
| ATOM | 3659 | CD | PRO | A | 771 | 29.801 | 15.730 | 7.154 | 1.00 | 30.49 | C |
| ATOM | 3662 | C | PRO | A | 771 | 32.578 | 17.678 | 8.234 | 1.00 | 30.56 | C |
| ATOM | 3663 | O | PRO | A | 771 | 32.621 | 16.901 | 9.192 | 1.00 | 30.01 | O |
| ATOM | 3664 | N | TRP | A | 772 | 33.415 | 18.690 | 8.067 | 1.00 | 30.74 | N |
| ATOM | 3666 | CA | TRP | A | 772 | 34.580 | 18.907 | 8.922 | 1.00 | 31.54 | C |
| ATOM | 3668 | CB | TRP | A | 772 | 35.215 | 20.260 | 8.605 | 1.00 | 31.15 | C |
| ATOM | 3671 | CG | TRP | A | 772 | 36.310 | 20.628 | 9.554 | 1.00 | 31.74 | C |
| ATOM | 3672 | CD1 | TRP | A | 772 | 36.346 | 21.706 | 10.395 | 1.00 | 30.69 | C |
| ATOM | 3674 | NE1 | TRP | A | 772 | 37.514 | 21.708 | 11.119 | 1.00 | 30.64 | N |
| ATOM | 3676 | CE2 | TRP | A | 772 | 38.252 | 20.610 | 10.766 | 1.00 | 32.70 | C |
| ATOM | 3677 | CD2 | TRP | A | 772 | 37.525 | 19.911 | 9.777 | 1.00 | 31.92 | C |
| ATOM | 3678 | CE3 | TRP | A | 772 | 38.075 | 18.741 | 9.247 | 1.00 | 33.06 | C |
| ATOM | 3680 | CZ3 | TRP | A | 772 | 39.310 | 18.315 | 9.700 | 1.00 | 32.82 | C |
| ATOM | 3682 | CH2 | TRP | A | 772 | 40.017 | 19.042 | 10.664 | 1.00 | 33.20 | C |
| ATOM | 3684 | CZ2 | TRP | A | 772 | 39.505 | 20.191 | 11.210 | 1.00 | 32.99 | C |
| ATOM | 3686 | C | TRP | A | 772 | 34.370 | 18.772 | 10.448 | 1.00 | 32.29 | C |
| ATOM | 3687 | O | TRP | A | 772 | 35.158 | 18.085 | 11.082 | 1.00 | 32.63 | O |
| ATOM | 3688 | N | PRO | A | 773 | 33.353 | 19.406 | 11.045 | 1.00 | 32.84 | N |
| ATOM | 3689 | CA | PRO | A | 773 | 33.174 | 19.318 | 12.505 | 1.00 | 33.39 | C |
| ATOM | 3691 | CB | PRO | A | 773 | 31.938 | 20.202 | 12.785 | 1.00 | 33.59 | C |
| ATOM | 3694 | CG | PRO | A | 773 | 31.831 | 21.109 | 11.595 | 1.00 | 33.72 | C |
| ATOM | 3697 | CD | PRO | A | 773 | 32.313 | 20.241 | 10.420 | 1.00 | 32.94 | C |
| ATOM | 3700 | C | PRO | A | 773 | 32.932 | 17.897 | 13.000 | 1.00 | 33.89 | C |
| ATOM | 3701 | O | PRO | A | 773 | 33.352 | 17.574 | 14.108 | 1.00 | 34.70 | O |
| ATOM | 3702 | N | ILE | A | 774 | 32.275 | 17.069 | 12.200 | 1.00 | 33.06 | N |
| ATOM | 3704 | CA | ILE | A | 774 | 32.049 | 15.689 | 12.561 | 1.00 | 33.95 | C |
| ATOM | 3706 | CB | ILE | A | 774 | 30.906 | 15.131 | 11.696 | 1.00 | 34.28 | C |
| ATOM | 3708 | CG1 | ILE | A | 774 | 29.592 | 15.855 | 12.055 | 1.00 | 36.56 | C |
| ATOM | 3711 | CD1 | ILE | A | 774 | 28.580 | 15.882 | 10.928 | 1.00 | 37.65 | C |
| ATOM | 3715 | CG2 | ILE | A | 774 | 30.780 | 13.606 | 11.848 | 1.00 | 34.99 | C |
| ATOM | 3719 | C | ILE | A | 774 | 33.317 | 14.831 | 12.421 | 1.00 | 34.03 | C |
| ATOM | 3720 | O | ILE | A | 774 | 33.637 | 14.032 | 13.311 | 1.00 | 34.05 | O |
| ATOM | 3721 | N | GLN | A | 775 | 34.029 | 14.991 | 11.305 | 1.00 | 33.53 | N |
| ATOM | 3723 | CA | GLN | A | 775 | 35.276 | 14.259 | 11.075 | 1.00 | 33.26 | C |
| ATOM | 3725 | CB | GLN | A | 775 | 35.781 | 14.483 | 9.630 | 1.00 | 32.88 | C |
| ATOM | 3728 | CG | GLN | A | 775 | 37.294 | 14.320 | 9.411 | 1.00 | 32.21 | C |
| ATOM | 3731 | CD | GLN | A | 775 | 37.819 | 12.966 | 9.789 | 1.00 | 29.57 | C |
| ATOM | 3732 | OE1 | GLN | A | 775 | 37.072 | 11.968 | 9.828 | 1.00 | 32.23 | O |
| ATOM | 3733 | NE2 | GLN | A | 775 | 39.108 | 12.904 | 10.058 | 1.00 | 27.57 | N |
| ATOM | 3736 | C | GLN | A | 775 | 36.334 | 14.620 | 12.138 | 1.00 | 33.48 | C |
| ATOM | 3737 | O | GLN | A | 775 | 36.931 | 13.721 | 12.725 | 1.00 | 33.59 | O |
| ATOM | 3738 | N | GLN | A | 776 | 36.553 | 15.912 | 12.389 | 1.00 | 33.83 | N |
| ATOM | 3740 | CA | GLN | A | 776 | 37.544 | 16.350 | 13.391 | 1.00 | 34.39 | C |
| ATOM | 3742 | CB | GLN | A | 776 | 37.540 | 17.866 | 13.599 | 1.00 | 34.52 | C |
| ATOM | 3745 | CG | GLN | A | 776 | 38.810 | 18.396 | 14.333 | 1.00 | 35.28 | C |
| ATOM | 3748 | CD | GLN | A | 776 | 38.898 | 19.924 | 14.435 | 1.00 | 35.65 | C |
| ATOM | 3749 | OE1 | GLN | A | 776 | 39.982 | 20.490 | 14.355 | 1.00 | 35.83 | O |
| ATOM | 3750 | NE2 | GLN | A | 776 | 37.767 | 20.577 | 14.641 | 1.00 | 37.48 | N |
| ATOM | 3753 | C | GLN | A | 776 | 37.254 | 15.648 | 14.709 | 1.00 | 35.11 | C |
| ATOM | 3754 | O | GLN | A | 776 | 38.138 | 15.040 | 15.306 | 1.00 | 34.23 | O |
| ATOM | 3755 | N | ARG | A | 777 | 35.989 | 15.710 | 15.120 | 1.00 | 35.48 | N |
| ATOM | 3757 | CA | ARG | A | 777 | 35.524 | 15.082 | 16.349 | 1.00 | 36.93 | C |
| ATOM | 3759 | CB | ARG | A | 777 | 34.033 | 15.360 | 16.534 | 1.00 | 37.68 | C |
| ATOM | 3762 | CG | ARG | A | 777 | 33.637 | 15.541 | 17.927 | 1.00 | 42.37 | C |
| ATOM | 3765 | CD | ARG | A | 777 | 33.662 | 14.250 | 18.764 | 1.00 | 47.31 | C |
| ATOM | 3768 | NE | ARG | A | 777 | 33.889 | 14.588 | 20.177 | 1.00 | 50.43 | N |
| ATOM | 3770 | CZ | ARG | A | 777 | 32.935 | 14.780 | 21.062 | 1.00 | 49.65 | C |
| ATOM | 3771 | NH1 | ARG | A | 777 | 31.654 | 14.659 | 20.708 | 1.00 | 51.41 | N |
| ATOM | 3774 | NH2 | ARG | A | 777 | 33.270 | 15.086 | 22.304 | 1.00 | 49.88 | N |
| ATOM | 3777 | C | ARG | A | 777 | 35.747 | 13.568 | 16.411 | 1.00 | 36.41 | C |
| ATOM | 3778 | O | ARG | A | 777 | 36.304 | 13.087 | 17.386 | 1.00 | 36.09 | O |
| ATOM | 3779 | N | ILE | A | 778 | 35.291 | 12.826 | 15.401 | 1.00 | 35.82 | N |
| ATOM | 3781 | CA | ILE | A | 778 | 35.476 | 11.377 | 15.379 | 1.00 | 36.75 | C |
| ATOM | 3783 | CB | ILE | A | 778 | 34.957 | 10.723 | 14.057 | 1.00 | 36.93 | C |
| ATOM | 3785 | CG1 | ILE | A | 778 | 33.437 | 10.792 | 13.978 | 1.00 | 39.16 | C |
| ATOM | 3788 | CD1 | ILE | A | 778 | 32.868 | 10.202 | 12.692 | 1.00 | 40.52 | C |
| ATOM | 3792 | CG2 | ILE | A | 778 | 35.355 | 9.247 | 14.011 | 1.00 | 39.29 | C |
| ATOM | 3796 | C | ILE | A | 778 | 36.944 | 11.027 | 15.497 | 1.00 | 35.85 | C |
| ATOM | 3797 | O | ILE | A | 778 | 37.312 | 10.105 | 16.244 | 1.00 | 35.82 | O |
| ATOM | 3798 | N | ALA | A | 779 | 37.765 | 11.754 | 14.730 | 1.00 | 34.32 | N |
| ATOM | 3800 | CA | ALA | A | 779 | 39.186 | 11.455 | 14.599 | 1.00 | 33.17 | C |
| ATOM | 3802 | CB | ALA | A | 779 | 39.824 | 12.324 | 13.505 | 1.00 | 32.80 | C |
| ATOM | 3806 | C | ALA | A | 779 | 39.854 | 11.697 | 15.947 | 1.00 | 32.84 | C |
| ATOM | 3807 | O | ALA | A | 779 | 40.703 | 10.928 | 16.366 | 1.00 | 31.43 | O |
| ATOM | 3808 | N | GLU | A | 780 | 39.430 | 12.750 | 16.645 | 1.00 | 32.74 | N |
| ATOM | 3810 | CA | GLU | A | 780 | 39.957 | 13.022 | 17.982 | 1.00 | 33.52 | C |
| ATOM | 3812 | CB | GLU | A | 780 | 39.479 | 14.371 | 18.519 | 1.00 | 34.36 | C |
| ATOM | 3815 | CG | GLU | A | 780 | 39.675 | 15.507 | 17.542 | 1.00 | 37.36 | C |
| ATOM | 3818 | CD | GLU | A | 780 | 40.731 | 16.458 | 17.945 | 1.00 | 42.43 | C |
| ATOM | 3819 | OE1 | GLU | A | 780 | 40.376 | 17.546 | 18.475 | 1.00 | 44.44 | O |
| ATOM | 3820 | OE2 | GLU | A | 780 | 41.913 | 16.111 | 17.675 | 1.00 | 47.53 | O |
| ATOM | 3821 | C | GLU | A | 780 | 39.587 | 11.965 | 18.986 | 1.00 | 32.66 | C |
| ATOM | 3822 | O | GLU | A | 780 | 40.396 | 11.621 | 19.829 | 1.00 | 32.54 | O |
| ATOM | 3823 | N | LEU | A | 781 | 38.360 | 11.471 | 18.904 | 1.00 | 32.94 | N |
| ATOM | 3825 | CA | LEU | A | 781 | 37.849 | 10.459 | 19.841 | 1.00 | 33.38 | C |
| ATOM | 3827 | CB | LEU | A | 781 | 36.356 | 10.264 | 19.662 | 1.00 | 33.14 | C |
| ATOM | 3830 | CG | LEU | A | 781 | 35.463 | 11.268 | 20.393 | 1.00 | 37.00 | C |
| ATOM | 3832 | CD1 | LEU | A | 781 | 34.009 | 10.910 | 20.097 | 1.00 | 40.21 | C |
| ATOM | 3836 | CD2 | LEU | A | 781 | 35.731 | 11.316 | 21.899 | 1.00 | 37.56 | C |
| ATOM | 3840 | C | LEU | A | 781 | 38.558 | 9.140 | 19.623 | 1.00 | 33.33 | C |
| ATOM | 3841 | O | LEU | A | 781 | 39.014 | 8.479 | 20.568 | 1.00 | 33.55 | O |
| ATOM | 3842 | N | VAL | A | 782 | 38.689 | 8.810 | 18.354 | 1.00 | 33.23 | N |
| ATOM | 3844 | CA | VAL | A | 782 | 39.315 | 7.586 | 17.916 | 1.00 | 34.70 | C |
| ATOM | 3846 | CB | VAL | A | 782 | 39.143 | 7.435 | 16.382 | 1.00 | 35.07 | C |
| ATOM | 3848 | CG1 | VAL | A | 782 | 40.142 | 6.493 | 15.802 | 1.00 | 37.35 | C |
| ATOM | 3852 | CG2 | VAL | A | 782 | 37.706 | 7.020 | 16.073 | 1.00 | 34.78 | C |
| ATOM | 3856 | C | VAL | A | 782 | 40.758 | 7.578 | 18.352 | 1.00 | 34.45 | C |
| ATOM | 3857 | O | VAL | A | 782 | 41.237 | 6.597 | 18.933 | 1.00 | 35.02 | O |
| ATOM | 3858 | N | ALA | A | 783 | 41.425 | 8.711 | 18.158 | 1.00 | 34.54 | N |
| ATOM | 3860 | CA | ALA | A | 783 | 42.802 | 8.854 | 18.570 | 1.00 | 34.07 | C |
| ATOM | 3862 | CB | ALA | A | 783 | 43.366 | 10.195 | 18.084 | 1.00 | 34.19 | C |
| ATOM | 3866 | C | ALA | A | 783 | 42.940 | 8.715 | 20.077 | 1.00 | 33.96 | C |
| ATOM | 3867 | O | ALA | A | 783 | 43.844 | 8.055 | 20.563 | 1.00 | 34.29 | O |
| ATOM | 3868 | N | THR | A | 784 | 42.061 | 9.362 | 20.829 | 1.00 | 33.54 | N |
| ATOM | 3870 | CA | THR | A | 784 | 42.079 | 9.254 | 22.284 | 1.00 | 33.02 | C |
| ATOM | 3872 | CB | THR | A | 784 | 40.985 | 10.163 | 22.882 | 1.00 | 33.03 | C |
| ATOM | 3874 | OG1 | THR | A | 784 | 41.372 | 11.520 | 22.687 | 1.00 | 33.63 | O |
| ATOM | 3876 | CG2 | THR | A | 784 | 40.873 | 9.995 | 24.372 | 1.00 | 34.14 | C |
| ATOM | 3880 | C | THR | A | 784 | 41.836 | 7.808 | 22.713 | 1.00 | 32.12 | C |
| ATOM | 3881 | O | THR | A | 784 | 42.523 | 7.293 | 23.585 | 1.00 | 30.96 | O |
| ATOM | 3882 | N | GLU | A | 785 | 40.881 | 7.150 | 22.073 | 1.00 | 31.76 | N |
| ATOM | 3884 | CA | GLU | A | 785 | 40.597 | 5.763 | 22.421 | 1.00 | 32.72 | C |
| ATOM | 3886 | CB | GLU | A | 785 | 39.424 | 5.240 | 21.629 | 1.00 | 33.09 | C |
| ATOM | 3889 | CG | GLU | A | 785 | 38.120 | 5.797 | 22.173 | 1.00 | 35.55 | C |
| ATOM | 3892 | CD | GLU | A | 785 | 37.114 | 6.142 | 21.097 | 1.00 | 39.51 | C |
| ATOM | 3893 | OE1 | GLU | A | 785 | 37.160 | 5.569 | 19.956 | 1.00 | 37.48 | O |
| ATOM | 3894 | OE2 | GLU | A | 785 | 36.253 | 6.992 | 21.418 | 1.00 | 40.23 | O |
| ATOM | 3895 | C | GLU | A | 785 | 41.823 | 4.862 | 22.270 | 1.00 | 32.40 | C |
| ATOM | 3896 | O | GLU | A | 785 | 42.063 | 4.000 | 23.121 | 1.00 | 32.01 | O |
| ATOM | 3897 | N | PHE | A | 786 | 42.637 | 5.090 | 21.236 | 1.00 | 31.75 | N |
| ATOM | 3899 | CA | PHE | A | 786 | 43.821 | 4.266 | 21.052 | 1.00 | 31.08 | C |
| ATOM | 3901 | CB | PHE | A | 786 | 44.302 | 4.254 | 19.589 | 1.00 | 30.76 | C |
| ATOM | 3904 | CG | PHE | A | 786 | 45.638 | 3.603 | 19.430 | 1.00 | 28.95 | C |
| ATOM | 3905 | CD1 | PHE | A | 786 | 45.733 | 2.227 | 19.231 | 1.00 | 28.19 | C |
| ATOM | 3907 | CE1 | PHE | A | 786 | 46.966 | 1.598 | 19.126 | 1.00 | 26.34 | C |
| ATOM | 3909 | CZ | PHE | A | 786 | 48.124 | 2.354 | 19.193 | 1.00 | 28.48 | C |
| ATOM | 3911 | CE2 | PHE | A | 786 | 48.046 | 3.737 | 19.396 | 1.00 | 29.04 | C |
| ATOM | 3913 | CD2 | PHE | A | 786 | 46.800 | 4.352 | 19.506 | 1.00 | 27.06 | C |
| ATOM | 3915 | C | PHE | A | 786 | 44.954 | 4.709 | 21.954 | 1.00 | 31.42 | C |
| ATOM | 3916 | O | PHE | A | 786 | 45.556 | 3.899 | 22.664 | 1.00 | 31.10 | O |
| ATOM | 3917 | N | PHE | A | 787 | 45.274 | 5.997 | 21.904 | 1.00 | 31.90 | N |
| ATOM | 3919 | CA | PHE | A | 787 | 46.485 | 6.498 | 22.527 | 1.00 | 33.00 | C |
| ATOM | 3921 | CB | PHE | A | 787 | 46.832 | 7.920 | 22.033 | 1.00 | 32.94 | C |
| ATOM | 3924 | CG | PHE | A | 787 | 47.416 | 7.944 | 20.647 | 1.00 | 33.16 | C |
| ATOM | 3925 | CD1 | PHE | A | 787 | 46.739 | 8.554 | 19.592 | 1.00 | 34.71 | C |
| ATOM | 3927 | CE1 | PHE | A | 787 | 47.260 | 8.551 | 18.300 | 1.00 | 34.34 | C |
| ATOM | 3929 | CZ | PHE | A | 787 | 48.445 | 7.937 | 18.057 | 1.00 | 32.95 | C |
| ATOM | 3931 | CE2 | PHE | A | 787 | 49.141 | 7.319 | 19.119 | 1.00 | 33.74 | C |
| ATOM | 3933 | CD2 | PHE | A | 787 | 48.616 | 7.315 | 20.387 | 1.00 | 31.79 | C |
| ATOM | 3935 | C | PHE | A | 787 | 46.451 | 6.431 | 24.058 | 1.00 | 34.24 | C |
| ATOM | 3936 | O | PHE | A | 787 | 47.504 | 6.333 | 24.660 | 1.00 | 33.67 | O |
| ATOM | 3937 | N | ASP | A | 788 | 45.261 | 6.426 | 24.659 | 1.00 | 35.47 | N |
| ATOM | 3939 | CA | ASP | A | 788 | 45.120 | 6.375 | 26.118 | 1.00 | 37.43 | C |
| ATOM | 3941 | CB | ASP | A | 788 | 44.295 | 7.571 | 26.622 | 1.00 | 37.71 | C |
| ATOM | 3944 | CG | ASP | A | 788 | 44.916 | 8.910 | 26.261 | 1.00 | 39.74 | C |
| ATOM | 3945 | OD1 | ASP | A | 788 | 46.162 | 9.028 | 26.237 | 1.00 | 41.13 | O |
| ATOM | 3946 | OD2 | ASP | A | 788 | 44.226 | 9.908 | 25.985 | 1.00 | 44.69 | O |
| ATOM | 3947 | C | ASP | A | 788 | 44.430 | 5.097 | 26.586 | 1.00 | 38.24 | C |
| ATOM | 3948 | O | ASP | A | 788 | 43.745 | 5.111 | 27.604 | 1.00 | 39.37 | O |
| ATOM | 3949 | N | GLN | A | 789 | 44.604 | 3.998 | 25.856 | 1.00 | 38.19 | N |
| ATOM | 3951 | CA | GLN | A | 789 | 43.942 | 2.744 | 26.211 | 1.00 | 38.18 | C |
| ATOM | 3953 | CB | GLN | A | 789 | 43.888 | 1.806 | 24.985 | 1.00 | 37.38 | C |
| ATOM | 3956 | CG | GLN | A | 789 | 45.238 | 1.289 | 24.523 | 1.00 | 36.46 | C |
| ATOM | 3959 | CD | GLN | A | 789 | 45.095 | 0.409 | 23.304 | 1.00 | 36.18 | C |
| ATOM | 3960 | OE1 | GLN | A | 789 | 45.349 | 0.839 | 22.152 | 1.00 | 34.24 | O |
| ATOM | 3961 | NE2 | GLN | A | 789 | 44.654 | −0.815 | 23.536 | 1.00 | 30.76 | N |
| ATOM | 3964 | C | GLN | A | 789 | 44.563 | 2.029 | 27.439 | 1.00 | 38.14 | C |
| ATOM | 3965 | O | GLN | A | 789 | 45.646 | 2.404 | 27.938 | 1.00 | 39.39 | O |
| ATOM | 3966 | N | LEU | A | 804 | 47.292 | 14.102 | 30.667 | 1.00 | 48.20 | N |
| ATOM | 3968 | CA | LEU | A | 804 | 48.025 | 15.289 | 31.085 | 1.00 | 48.23 | C |
| ATOM | 3970 | CB | LEU | A | 804 | 48.616 | 15.100 | 32.503 | 1.00 | 48.00 | C |
| ATOM | 3973 | CG | LEU | A | 804 | 47.806 | 15.491 | 33.769 | 1.00 | 46.92 | C |
| ATOM | 3975 | CD1 | LEU | A | 804 | 48.696 | 15.513 | 35.023 | 1.00 | 45.99 | C |
| ATOM | 3979 | CD2 | LEU | A | 804 | 47.075 | 16.830 | 33.646 | 1.00 | 46.22 | C |
| ATOM | 3983 | C | LEU | A | 804 | 49.131 | 15.673 | 30.083 | 1.00 | 48.83 | C |
| ATOM | 3984 | O | LEU | A | 804 | 50.011 | 16.473 | 30.414 | 1.00 | 48.85 | O |
| ATOM | 3985 | N | MET | A | 805 | 49.098 | 15.113 | 28.869 | 1.00 | 49.61 | N |
| ATOM | 3987 | CA | MET | A | 805 | 49.982 | 15.606 | 27.787 | 1.00 | 50.21 | C |
| ATOM | 3989 | CB | MET | A | 805 | 50.983 | 14.551 | 27.265 | 1.00 | 50.52 | C |
| ATOM | 3992 | CG | MET | A | 805 | 50.399 | 13.177 | 26.872 | 1.00 | 52.14 | C |
| ATOM | 3995 | SD | MET | A | 805 | 51.017 | 11.863 | 27.954 | 1.00 | 55.69 | S |
| ATOM | 3996 | CE | MET | A | 805 | 52.683 | 11.540 | 27.248 | 1.00 | 53.87 | C |
| ATOM | 4000 | C | MET | A | 805 | 49.149 | 16.213 | 26.649 | 1.00 | 49.81 | C |
| ATOM | 4001 | O | MET | A | 805 | 49.319 | 17.404 | 26.356 | 1.00 | 50.89 | O |
| ATOM | 4002 | N | ASN | A | 806 | 48.271 | 15.410 | 26.030 | 1.00 | 48.96 | N |
| ATOM | 4004 | CA | ASN | A | 806 | 47.228 | 15.890 | 25.111 | 1.00 | 48.12 | C |
| ATOM | 4006 | CB | ASN | A | 806 | 46.341 | 16.948 | 25.794 | 1.00 | 48.40 | C |
| ATOM | 4009 | CG | ASN | A | 806 | 45.306 | 16.328 | 26.687 | 1.00 | 48.99 | C |
| ATOM | 4010 | OD1 | ASN | A | 806 | 44.550 | 15.469 | 26.243 | 1.00 | 48.89 | O |
| ATOM | 4011 | ND2 | ASN | A | 806 | 45.286 | 16.730 | 27.963 | 1.00 | 48.58 | N |
| ATOM | 4014 | C | ASN | A | 806 | 47.739 | 16.454 | 23.804 | 1.00 | 46.98 | C |
| ATOM | 4015 | O | ASN | A | 806 | 47.348 | 15.982 | 22.743 | 1.00 | 46.74 | O |
| ATOM | 4016 | N | ARG | A | 807 | 48.504 | 17.540 | 23.912 | 1.00 | 45.71 | N |
| ATOM | 4018 | CA | ARG | A | 807 | 49.315 | 18.096 | 22.837 | 1.00 | 44.96 | C |
| ATOM | 4020 | CB | ARG | A | 807 | 50.422 | 18.984 | 23.418 | 1.00 | 44.75 | C |
| ATOM | 4023 | CG | ARG | A | 807 | 49.988 | 20.372 | 23.859 | 1.00 | 44.98 | C |
| ATOM | 4026 | CD | ARG | A | 807 | 51.038 | 21.442 | 23.571 | 1.00 | 44.24 | C |
| ATOM | 4029 | NE | ARG | A | 807 | 51.043 | 22.518 | 24.553 | 1.00 | 44.44 | N |
| ATOM | 4031 | CZ | ARG | A | 807 | 51.802 | 23.608 | 24.469 | 1.00 | 44.47 | C |
| ATOM | 4032 | NH1 | ARG | A | 807 | 52.636 | 23.781 | 23.445 | 1.00 | 44.73 | N |
| ATOM | 4035 | NH2 | ARG | A | 807 | 51.736 | 24.535 | 25.419 | 1.00 | 44.86 | N |
| ATOM | 4038 | C | ARG | A | 807 | 49.977 | 17.018 | 22.007 | 1.00 | 44.15 | C |
| ATOM | 4039 | O | ARG | A | 807 | 49.721 | 16.930 | 20.817 | 1.00 | 44.46 | O |
| ATOM | 4040 | N | GLU | A | 808 | 50.823 | 16.206 | 22.640 | 1.00 | 43.47 | N |
| ATOM | 4042 | CA | GLU | A | 808 | 51.549 | 15.127 | 21.952 | 1.00 | 43.32 | C |
| ATOM | 4044 | CB | GLU | A | 808 | 52.339 | 14.273 | 22.962 | 1.00 | 43.39 | C |
| ATOM | 4047 | CG | GLU | A | 808 | 53.743 | 14.811 | 23.263 | 1.00 | 44.43 | C |
| ATOM | 4050 | CD | GLU | A | 808 | 54.087 | 14.859 | 24.752 | 1.00 | 46.76 | C |
| ATOM | 4051 | OE1 | GLU | A | 808 | 53.612 | 13.988 | 25.518 | 1.00 | 49.69 | O |
| ATOM | 4052 | OE2 | GLU | A | 808 | 54.847 | 15.768 | 25.166 | 1.00 | 46.32 | O |
| ATOM | 4053 | C | GLU | A | 808 | 50.621 | 14.252 | 21.100 | 1.00 | 42.71 | C |
| ATOM | 4054 | O | GLU | A | 808 | 50.993 | 13.807 | 20.015 | 1.00 | 43.71 | O |
| ATOM | 4055 | N | LYS | A | 809 | 49.411 | 14.028 | 21.591 | 1.00 | 41.53 | N |
| ATOM | 4057 | CA | LYS | A | 809 | 48.393 | 13.268 | 20.879 | 1.00 | 41.25 | C |
| ATOM | 4059 | CB | LYS | A | 809 | 47.240 | 12.930 | 21.861 | 1.00 | 41.18 | C |
| ATOM | 4062 | CG | LYS | A | 809 | 46.242 | 11.871 | 21.390 | 1.00 | 40.13 | C |
| ATOM | 4065 | CD | LYS | A | 809 | 44.800 | 12.047 | 21.921 | 1.00 | 39.55 | C |
| ATOM | 4068 | CE | LYS | A | 809 | 44.702 | 12.763 | 23.279 | 1.00 | 41.48 | C |
| ATOM | 4071 | NZ | LYS | A | 809 | 43.352 | 12.612 | 23.881 | 1.00 | 42.05 | N |
| ATOM | 4075 | C | LYS | A | 809 | 47.854 | 14.078 | 19.692 | 1.00 | 40.56 | C |
| ATOM | 4076 | O | LYS | A | 809 | 47.896 | 13.638 | 18.547 | 1.00 | 40.59 | O |
| ATOM | 4077 | N | LYS | A | 810 | 47.353 | 15.270 | 19.996 | 1.00 | 39.94 | N |
| ATOM | 4079 | CA | LYS | A | 810 | 46.595 | 16.086 | 19.056 | 1.00 | 39.38 | C |
| ATOM | 4081 | CB | LYS | A | 810 | 46.030 | 17.334 | 19.750 | 1.00 | 39.37 | C |
| ATOM | 4084 | CG | LYS | A | 810 | 44.595 | 17.168 | 20.259 | 1.00 | 41.26 | C |
| ATOM | 4087 | CD | LYS | A | 810 | 44.144 | 18.418 | 21.043 | 1.00 | 43.79 | C |
| ATOM | 4090 | CE | LYS | A | 810 | 42.631 | 18.636 | 20.928 | 1.00 | 44.96 | C |
| ATOM | 4093 | NZ | LYS | A | 810 | 42.290 | 20.033 | 21.357 | 1.00 | 46.60 | N |
| ATOM | 4097 | C | LYS | A | 810 | 47.447 | 16.501 | 17.871 | 1.00 | 38.49 | C |
| ATOM | 4098 | O | LYS | A | 810 | 46.935 | 16.581 | 16.755 | 1.00 | 38.38 | O |
| ATOM | 4099 | N | ASN | A | 811 | 48.728 | 16.756 | 18.138 | 1.00 | 37.29 | N |
| ATOM | 4101 | CA | ASN | A | 811 | 49.707 | 17.123 | 17.120 | 1.00 | 37.08 | C |
| ATOM | 4103 | CB | ASN | A | 811 | 51.015 | 17.581 | 17.771 | 1.00 | 36.70 | C |
| ATOM | 4106 | CG | ASN | A | 811 | 50.949 | 19.013 | 18.267 | 1.00 | 38.21 | C |
| ATOM | 4107 | OD1 | ASN | A | 811 | 50.010 | 19.751 | 17.962 | 1.00 | 40.42 | O |
| ATOM | 4108 | ND2 | ASN | A | 811 | 51.951 | 19.415 | 19.039 | 1.00 | 36.98 | N |
| ATOM | 4111 | C | ASN | A | 811 | 50.029 | 16.029 | 16.094 | 1.00 | 36.40 | C |
| ATOM | 4112 | O | ASN | A | 811 | 50.519 | 16.338 | 15.020 | 1.00 | 36.71 | O |
| ATOM | 4113 | N | LYS | A | 812 | 49.794 | 14.765 | 16.443 | 1.00 | 35.59 | N |
| ATOM | 4115 | CA | LYS | A | 812 | 49.952 | 13.658 | 15.515 | 1.00 | 34.80 | C |
| ATOM | 4117 | CB | LYS | A | 812 | 49.997 | 12.321 | 16.261 | 1.00 | 35.36 | C |
| ATOM | 4120 | CG | LYS | A | 812 | 51.178 | 12.089 | 17.197 | 1.00 | 38.10 | C |
| ATOM | 4123 | CD | LYS | A | 812 | 50.943 | 10.811 | 18.045 | 1.00 | 40.15 | C |
| ATOM | 4126 | CE | LYS | A | 812 | 52.167 | 10.495 | 18.955 | 1.00 | 42.19 | C |
| ATOM | 4129 | NZ | LYS | A | 812 | 51.729 | 10.181 | 20.375 | 1.00 | 43.84 | N |
| ATOM | 4133 | C | LYS | A | 812 | 48.809 | 13.565 | 14.505 | 1.00 | 33.50 | C |
| ATOM | 4134 | O | LYS | A | 812 | 49.002 | 13.066 | 13.399 | 1.00 | 33.42 | O |
| ATOM | 4135 | N | ILE | A | 813 | 47.612 | 13.997 | 14.885 | 1.00 | 31.89 | N |
| ATOM | 4137 | CA | ILE | A | 813 | 46.419 | 13.628 | 14.139 | 1.00 | 31.20 | C |
| ATOM | 4139 | CB | ILE | A | 813 | 45.112 | 13.971 | 14.923 | 1.00 | 31.60 | C |
| ATOM | 4141 | CG1 | ILE | A | 813 | 45.053 | 13.185 | 16.235 | 1.00 | 31.56 | C |
| ATOM | 4144 | CD1 | ILE | A | 813 | 43.972 | 13.688 | 17.154 | 1.00 | 33.08 | C |
| ATOM | 4148 | CG2 | ILE | A | 813 | 43.856 | 13.633 | 14.078 | 1.00 | 31.70 | C |
| ATOM | 4152 | C | ILE | A | 813 | 46.335 | 14.128 | 12.685 | 1.00 | 30.20 | C |
| ATOM | 4153 | O | ILE | A | 813 | 45.916 | 13.360 | 11.822 | 1.00 | 29.75 | O |
| ATOM | 4154 | N | PRO | A | 814 | 46.641 | 15.400 | 12.420 | 1.00 | 29.48 | N |
| ATOM | 4155 | CA | PRO | A | 814 | 46.591 | 15.922 | 11.047 | 1.00 | 29.05 | C |
| ATOM | 4157 | CB | PRO | A | 814 | 47.109 | 17.351 | 11.195 | 1.00 | 29.41 | C |
| ATOM | 4160 | CG | PRO | A | 814 | 46.719 | 17.725 | 12.636 | 1.00 | 28.96 | C |
| ATOM | 4163 | CD | PRO | A | 814 | 46.977 | 16.463 | 13.391 | 1.00 | 29.10 | C |
| ATOM | 4166 | C | PRO | A | 814 | 47.414 | 15.109 | 10.049 | 1.00 | 28.97 | C |
| ATOM | 4167 | O | PRO | A | 814 | 46.860 | 14.740 | 9.019 | 1.00 | 28.23 | O |
| ATOM | 4168 | N | SER | A | 815 | 48.653 | 14.778 | 10.377 | 1.00 | 28.56 | N |
| ATOM | 4170 | CA | SER | A | 815 | 49.506 | 14.002 | 9.462 | 1.00 | 29.09 | C |
| ATOM | 4172 | CB | SER | A | 815 | 50.962 | 13.975 | 9.927 | 1.00 | 28.44 | C |
| ATOM | 4175 | OG | SER | A | 815 | 51.511 | 15.248 | 9.770 | 1.00 | 32.41 | O |
| ATOM | 4177 | C | SER | A | 815 | 49.001 | 12.586 | 9.329 | 1.00 | 28.45 | C |
| ATOM | 4178 | O | SER | A | 815 | 49.115 | 12.000 | 8.246 | 1.00 | 27.40 | O |
| ATOM | 4179 | N | MET | A | 816 | 48.443 | 12.039 | 10.417 | 1.00 | 27.90 | N |
| ATOM | 4181 | CA | MET | A | 816 | 47.820 | 10.719 | 10.346 | 1.00 | 27.88 | C |
| ATOM | 4183 | CB | MET | A | 816 | 47.290 | 10.260 | 11.710 | 1.00 | 27.50 | C |
| ATOM | 4186 | CG | MET | A | 816 | 48.410 | 10.004 | 12.750 | 1.00 | 31.73 | C |
| ATOM | 4189 | SD | MET | A | 816 | 47.733 | 9.786 | 14.449 | 1.00 | 35.38 | S |
| ATOM | 4190 | CE | MET | A | 816 | 47.038 | 8.358 | 14.309 | 1.00 | 33.86 | C |
| ATOM | 4194 | C | MET | A | 816 | 46.685 | 10.684 | 9.332 | 1.00 | 27.46 | C |
| ATOM | 4195 | O | MET | A | 816 | 46.606 | 9.769 | 8.502 | 1.00 | 26.95 | O |
| ATOM | 4196 | N | GLN | A | 817 | 45.791 | 11.654 | 9.418 | 1.00 | 27.39 | N |
| ATOM | 4198 | CA | GLN | A | 817 | 44.628 | 11.668 | 8.558 | 1.00 | 27.92 | C |
| ATOM | 4200 | CB | GLN | A | 817 | 43.554 | 12.624 | 9.076 | 1.00 | 27.63 | C |
| ATOM | 4203 | CG | GLN | A | 817 | 42.950 | 12.260 | 10.414 | 1.00 | 29.16 | C |
| ATOM | 4206 | CD | GLN | A | 817 | 42.262 | 10.929 | 10.457 | 1.00 | 31.41 | C |
| ATOM | 4207 | OE1 | GLN | A | 817 | 41.024 | 10.843 | 10.344 | 1.00 | 36.06 | O |
| ATOM | 4208 | NE2 | GLN | A | 817 | 43.035 | 9.881 | 10.652 | 1.00 | 31.67 | N |
| ATOM | 4211 | C | GLN | A | 817 | 45.011 | 11.996 | 7.108 | 1.00 | 27.83 | C |
| ATOM | 4212 | O | GLN | A | 817 | 44.525 | 11.359 | 6.165 | 1.00 | 27.95 | O |
| ATOM | 4213 | N | VAL | A | 818 | 45.888 | 12.958 | 6.917 | 1.00 | 28.09 | N |
| ATOM | 4215 | CA | VAL | A | 818 | 46.317 | 13.248 | 5.562 | 1.00 | 28.52 | C |
| ATOM | 4217 | CB | VAL | A | 818 | 47.221 | 14.452 | 5.472 | 1.00 | 28.62 | C |
| ATOM | 4219 | CG1 | VAL | A | 818 | 47.834 | 14.545 | 4.071 | 1.00 | 29.96 | C |
| ATOM | 4223 | CG2 | VAL | A | 818 | 46.447 | 15.702 | 5.757 | 1.00 | 29.17 | C |
| ATOM | 4227 | C | VAL | A | 818 | 46.982 | 11.994 | 4.926 | 1.00 | 28.64 | C |
| ATOM | 4228 | O | VAL | A | 818 | 46.704 | 11.661 | 3.763 | 1.00 | 27.81 | O |
| ATOM | 4229 | N | GLY | A | 819 | 47.793 | 11.275 | 5.699 | 1.00 | 27.84 | N |
| ATOM | 4231 | CA | GLY | A | 819 | 48.493 | 10.127 | 5.168 | 1.00 | 27.87 | C |
| ATOM | 4234 | C | GLY | A | 819 | 47.525 | 9.014 | 4.788 | 1.00 | 27.91 | C |
| ATOM | 4235 | O | GLY | A | 819 | 47.700 | 8.314 | 3.792 | 1.00 | 27.09 | O |
| ATOM | 4236 | N | PHE | A | 820 | 46.505 | 8.848 | 5.606 | 1.00 | 27.82 | N |
| ATOM | 4238 | CA | PHE | A | 820 | 45.521 | 7.812 | 5.419 | 1.00 | 28.51 | C |
| ATOM | 4240 | CB | PHE | A | 820 | 44.730 | 7.606 | 6.720 | 1.00 | 29.12 | C |
| ATOM | 4243 | CG | PHE | A | 820 | 43.822 | 6.403 | 6.710 | 1.00 | 30.74 | C |
| ATOM | 4244 | CD1 | PHE | A | 820 | 44.319 | 5.143 | 7.000 | 1.00 | 33.05 | C |
| ATOM | 4246 | CE1 | PHE | A | 820 | 43.486 | 4.028 | 7.004 | 1.00 | 33.70 | C |
| ATOM | 4248 | CZ | PHE | A | 820 | 42.149 | 4.170 | 6.733 | 1.00 | 34.30 | C |
| ATOM | 4250 | CE2 | PHE | A | 820 | 41.622 | 5.438 | 6.498 | 1.00 | 35.70 | C |
| ATOM | 4252 | CD2 | PHE | A | 820 | 42.471 | 6.547 | 6.470 | 1.00 | 34.48 | C |
| ATOM | 4254 | C | PHE | A | 820 | 44.627 | 8.161 | 4.231 | 1.00 | 28.44 | C |
| ATOM | 4255 | O | PHE | A | 820 | 44.289 | 7.300 | 3.437 | 1.00 | 29.38 | O |
| ATOM | 4256 | N | ILE | A | 821 | 44.288 | 9.425 | 4.075 | 1.00 | 28.63 | N |
| ATOM | 4258 | CA | ILE | A | 821 | 43.512 | 9.861 | 2.914 | 1.00 | 29.24 | C |
| ATOM | 4260 | CB | ILE | A | 821 | 43.138 | 11.354 | 3.044 | 1.00 | 29.40 | C |
| ATOM | 4262 | CG1 | ILE | A | 821 | 42.064 | 11.518 | 4.136 | 1.00 | 30.02 | C |
| ATOM | 4265 | CD1 | ILE | A | 821 | 41.748 | 12.975 | 4.560 | 1.00 | 31.19 | C |
| ATOM | 4269 | CG2 | ILE | A | 821 | 42.656 | 11.908 | 1.709 | 1.00 | 29.92 | C |
| ATOM | 4273 | C | ILE | A | 821 | 44.266 | 9.592 | 1.618 | 1.00 | 29.05 | C |
| ATOM | 4274 | O | ILE | A | 821 | 43.742 | 8.990 | 0.711 | 1.00 | 28.50 | O |
| ATOM | 4275 | N | ASP | A | 822 | 45.520 | 10.008 | 1.569 | 1.00 | 29.41 | N |
| ATOM | 4277 | CA | ASP | A | 822 | 46.356 | 9.821 | 0.390 | 1.00 | 29.40 | C |
| ATOM | 4279 | CB | ASP | A | 822 | 47.694 | 10.533 | 0.583 | 1.00 | 30.20 | C |
| ATOM | 4282 | CG | ASP | A | 822 | 47.578 | 12.026 | 0.409 | 1.00 | 30.26 | C |
| ATOM | 4283 | OD1 | ASP | A | 822 | 46.456 | 12.484 | 0.103 | 1.00 | 33.07 | O |
| ATOM | 4284 | OD2 | ASP | A | 822 | 48.535 | 12.817 | 0.581 | 1.00 | 31.23 | O |
| ATOM | 4285 | C | ASP | A | 822 | 46.604 | 8.377 | 0.037 | 1.00 | 29.48 | C |
| ATOM | 4286 | O | ASP | A | 822 | 46.465 | 7.996 | −1.110 | 1.00 | 30.16 | O |
| ATOM | 4287 | N | ALA | A | 823 | 46.947 | 7.557 | 1.021 | 1.00 | 29.03 | N |
| ATOM | 4289 | CA | ALA | A | 823 | 47.291 | 6.173 | 0.780 | 1.00 | 28.80 | C |
| ATOM | 4291 | CB | ALA | A | 823 | 48.068 | 5.616 | 1.972 | 1.00 | 28.84 | C |
| ATOM | 4295 | C | ALA | A | 823 | 46.087 | 5.270 | 0.480 | 1.00 | 29.37 | C |
| ATOM | 4296 | O | ALA | A | 823 | 46.188 | 4.359 | −0.334 | 1.00 | 28.45 | O |
| ATOM | 4297 | N | ILE | A | 824 | 44.968 | 5.514 | 1.157 | 1.00 | 30.57 | N |
| ATOM | 4299 | CA | ILE | A | 824 | 43.870 | 4.549 | 1.248 | 1.00 | 31.06 | C |
| ATOM | 4301 | CB | ILE | A | 824 | 43.657 | 4.173 | 2.744 | 1.00 | 31.11 | C |
| ATOM | 4303 | CG1 | ILE | A | 824 | 44.880 | 3.451 | 3.316 | 1.00 | 32.33 | C |
| ATOM | 4306 | CD1 | ILE | A | 824 | 45.376 | 2.247 | 2.543 | 1.00 | 33.90 | C |
| ATOM | 4310 | CG2 | ILE | A | 824 | 42.377 | 3.424 | 2.948 | 1.00 | 31.53 | C |
| ATOM | 4314 | C | ILE | A | 824 | 42.546 | 5.075 | 0.675 | 1.00 | 31.38 | C |
| ATOM | 4315 | O | ILE | A | 824 | 41.849 | 4.343 | −0.013 | 1.00 | 31.82 | O |
| ATOM | 4316 | N | CYS | A | 825 | 42.188 | 6.319 | 0.977 | 1.00 | 31.69 | N |
| ATOM | 4318 | CA | CYS | A | 825 | 40.791 | 6.784 | 0.810 | 1.00 | 32.07 | C |
| ATOM | 4320 | CB | CYS | A | 825 | 40.430 | 7.766 | 1.923 | 1.00 | 31.91 | C |
| ATOM | 4323 | SG | CYS | A | 825 | 40.509 | 7.071 | 3.573 | 1.00 | 33.39 | S |
| ATOM | 4324 | C | CYS | A | 825 | 40.477 | 7.449 | −0.521 | 1.00 | 32.49 | C |
| ATOM | 4325 | O | CYS | A | 825 | 39.491 | 7.110 | −1.186 | 1.00 | 32.44 | O |
| ATOM | 4326 | N | LEU | A | 826 | 41.305 | 8.411 | −0.887 | 1.00 | 32.14 | N |
| ATOM | 4328 | CA | LEU | A | 826 | 41.068 | 9.228 | −2.060 | 1.00 | 33.82 | C |
| ATOM | 4330 | CB | LEU | A | 826 | 42.279 | 10.141 | −2.258 | 1.00 | 34.35 | C |
| ATOM | 4333 | CG | LEU | A | 826 | 42.094 | 11.541 | −2.771 | 1.00 | 36.34 | C |
| ATOM | 4335 | CD1 | LEU | A | 826 | 41.169 | 12.322 | −1.846 | 1.00 | 37.86 | C |
| ATOM | 4339 | CD2 | LEU | A | 826 | 43.498 | 12.193 | −2.926 | 1.00 | 36.47 | C |
| ATOM | 4343 | C | LEU | A | 826 | 40.796 | 8.398 | −3.330 | 1.00 | 33.83 | C |
| ATOM | 4344 | O | LEU | A | 826 | 39.797 | 8.594 | −4.012 | 1.00 | 33.90 | O |
| ATOM | 4345 | N | GLN | A | 827 | 41.676 | 7.453 | −3.629 | 1.00 | 33.95 | N |
| ATOM | 4347 | CA | GLN | A | 827 | 41.520 | 6.625 | −4.814 | 1.00 | 34.31 | C |
| ATOM | 4349 | CB | GLN | A | 827 | 42.734 | 5.712 | −5.013 | 1.00 | 34.21 | C |
| ATOM | 4352 | CG | GLN | A | 827 | 43.454 | 5.940 | −6.309 | 1.00 | 37.79 | C |
| ATOM | 4355 | CD | GLN | A | 827 | 44.483 | 4.845 | −6.624 | 1.00 | 39.98 | C |
| ATOM | 4356 | OE1 | GLN | A | 827 | 45.145 | 4.330 | −5.722 | 1.00 | 39.97 | O |
| ATOM | 4357 | NE2 | GLN | A | 827 | 44.598 | 4.492 | −7.891 | 1.00 | 37.98 | N |
| ATOM | 4360 | C | GLN | A | 827 | 40.237 | 5.791 | −4.799 | 1.00 | 33.59 | C |
| ATOM | 4361 | O | GLN | A | 827 | 39.624 | 5.592 | −5.826 | 1.00 | 33.68 | O |
| ATOM | 4362 | N | LEU | A | 828 | 39.857 | 5.276 | −3.644 | 1.00 | 33.46 | N |
| ATOM | 4364 | CA | LEU | A | 828 | 38.632 | 4.495 | −3.529 | 1.00 | 33.05 | C |
| ATOM | 4366 | CB | LEU | A | 828 | 38.542 | 3.885 | −2.143 | 1.00 | 33.17 | C |
| ATOM | 4369 | CG | LEU | A | 828 | 37.237 | 3.166 | −1.802 | 1.00 | 35.09 | C |
| ATOM | 4371 | CD1 | LEU | A | 828 | 36.965 | 2.045 | −2.823 | 1.00 | 37.34 | C |
| ATOM | 4375 | CD2 | LEU | A | 828 | 37.321 | 2.625 | −0.429 | 1.00 | 34.66 | C |
| ATOM | 4379 | C | LEU | A | 828 | 37.356 | 5.313 | −3.849 | 1.00 | 32.72 | C |
| ATOM | 4380 | O | LEU | A | 828 | 36.474 | 4.839 | −4.560 | 1.00 | 31.03 | O |
| ATOM | 4381 | N | TYR | A | 829 | 37.250 | 6.521 | −3.292 | 1.00 | 32.51 | N |
| ATOM | 4383 | CA | TYR | A | 829 | 36.064 | 7.347 | −3.485 | 1.00 | 31.96 | C |
| ATOM | 4385 | CB | TYR | A | 829 | 35.979 | 8.420 | −2.386 | 1.00 | 31.66 | C |
| ATOM | 4388 | CG | TYR | A | 829 | 35.683 | 7.843 | −1.009 | 1.00 | 32.31 | C |
| ATOM | 4389 | CD1 | TYR | A | 829 | 36.587 | 7.999 | 0.056 | 1.00 | 30.90 | C |
| ATOM | 4391 | CE1 | TYR | A | 829 | 36.342 | 7.453 | 1.282 | 1.00 | 32.79 | C |
| ATOM | 4393 | CZ | TYR | A | 829 | 35.174 | 6.718 | 1.492 | 1.00 | 33.50 | C |
| ATOM | 4394 | OH | TYR | A | 829 | 34.918 | 6.191 | 2.724 | 1.00 | 29.58 | O |
| ATOM | 4396 | CE2 | TYR | A | 829 | 34.259 | 6.547 | 0.470 | 1.00 | 32.83 | C |
| ATOM | 4398 | CD2 | TYR | A | 829 | 34.526 | 7.101 | −0.780 | 1.00 | 32.09 | C |
| ATOM | 4400 | C | TYR | A | 829 | 36.061 | 7.926 | −4.926 | 1.00 | 32.09 | C |
| ATOM | 4401 | O | TYR | A | 829 | 35.013 | 8.092 | −5.507 | 1.00 | 31.90 | O |
| ATOM | 4402 | N | GLU | A | 830 | 37.237 | 8.178 | −5.505 | 1.00 | 32.32 | N |
| ATOM | 4404 | CA | GLU | A | 830 | 37.357 | 8.521 | −6.938 | 1.00 | 33.53 | C |
| ATOM | 4406 | CB | GLU | A | 830 | 38.813 | 8.832 | −7.374 | 1.00 | 33.92 | C |
| ATOM | 4409 | CG | GLU | A | 830 | 39.422 | 10.137 | −6.848 | 1.00 | 36.91 | C |
| ATOM | 4412 | CD | GLU | A | 830 | 40.952 | 10.292 | −7.084 | 1.00 | 42.60 | C |
| ATOM | 4413 | OE1 | GLU | A | 830 | 41.515 | 11.357 | −6.706 | 1.00 | 46.07 | O |
| ATOM | 4414 | OE2 | GLU | A | 830 | 41.626 | 9.381 | −7.636 | 1.00 | 45.22 | O |
| ATOM | 4415 | C | GLU | A | 830 | 36.817 | 7.382 | −7.788 | 1.00 | 33.38 | C |
| ATOM | 4416 | O | GLU | A | 830 | 35.994 | 7.600 | −8.691 | 1.00 | 32.83 | O |
| ATOM | 4417 | N | ALA | A | 831 | 37.261 | 6.165 | −7.487 | 1.00 | 33.32 | N |
| ATOM | 4419 | CA | ALA | A | 831 | 36.801 | 4.983 | −8.232 | 1.00 | 33.85 | C |
| ATOM | 4421 | CB | ALA | A | 831 | 37.571 | 3.712 | −7.831 | 1.00 | 33.75 | C |
| ATOM | 4425 | C | ALA | A | 831 | 35.301 | 4.760 | −8.066 | 1.00 | 33.80 | C |
| ATOM | 4426 | O | ALA | A | 831 | 34.634 | 4.435 | −9.027 | 1.00 | 34.04 | O |
| ATOM | 4427 | N | LEU | A | 832 | 34.778 | 4.935 | −6.856 | 1.00 | 33.40 | N |
| ATOM | 4429 | CA | LEU | A | 832 | 33.353 | 4.752 | −6.619 | 1.00 | 33.24 | C |
| ATOM | 4431 | CB | LEU | A | 832 | 33.023 | 4.977 | −5.146 | 1.00 | 33.06 | C |
| ATOM | 4434 | CG | LEU | A | 832 | 31.587 | 4.714 | −4.691 | 1.00 | 33.84 | C |
| ATOM | 4436 | CD1 | LEU | A | 832 | 31.165 | 3.309 | −5.086 | 1.00 | 33.85 | C |
| ATOM | 4440 | CD2 | LEU | A | 832 | 31.420 | 4.916 | −3.160 | 1.00 | 36.52 | C |
| ATOM | 4444 | C | LEU | A | 832 | 32.530 | 5.734 | −7.451 | 1.00 | 32.81 | C |
| ATOM | 4445 | O | LEU | A | 832 | 31.456 | 5.410 | −7.942 | 1.00 | 32.89 | O |
| ATOM | 4446 | N | THR | A | 833 | 33.030 | 6.952 | −7.565 | 1.00 | 32.75 | N |
| ATOM | 4448 | CA | THR | A | 833 | 32.333 | 8.020 | −8.272 | 1.00 | 32.75 | C |
| ATOM | 4450 | CB | THR | A | 833 | 33.055 | 9.337 | −7.978 | 1.00 | 33.07 | C |
| ATOM | 4452 | OG1 | THR | A | 833 | 32.827 | 9.686 | −6.599 | 1.00 | 33.82 | O |
| ATOM | 4454 | CG2 | THR | A | 833 | 32.504 | 10.472 | −8.751 | 1.00 | 33.21 | C |
| ATOM | 4458 | C | THR | A | 833 | 32.228 | 7.728 | −9.772 | 1.00 | 32.38 | C |
| ATOM | 4459 | O | THR | A | 833 | 31.234 | 8.077 | −10.405 | 1.00 | 31.86 | O |
| ATOM | 4460 | N | HIS | A | 834 | 33.233 | 7.058 | −10.321 | 1.00 | 32.05 | N |
| ATOM | 4462 | CA | HIS | A | 834 | 33.201 | 6.605 | −11.700 | 1.00 | 32.54 | C |
| ATOM | 4464 | CB | HIS | A | 834 | 34.578 | 6.085 | −12.143 | 1.00 | 32.85 | C |
| ATOM | 4467 | CG | HIS | A | 834 | 35.565 | 7.170 | −12.448 | 1.00 | 34.19 | C |
| ATOM | 4468 | ND1 | HIS | A | 834 | 35.416 | 8.036 | −13.515 | 1.00 | 36.12 | N |
| ATOM | 4470 | CE1 | HIS | A | 834 | 36.436 | 8.876 | −13.540 | 1.00 | 36.63 | C |
| ATOM | 4472 | NE2 | HIS | A | 834 | 37.243 | 8.587 | −12.532 | 1.00 | 36.17 | N |
| ATOM | 4474 | CD2 | HIS | A | 834 | 36.725 | 7.519 | −11.838 | 1.00 | 35.04 | C |
| ATOM | 4476 | C | HIS | A | 834 | 32.128 | 5.529 | −11.913 | 1.00 | 32.37 | C |
| ATOM | 4477 | O | HIS | A | 834 | 31.503 | 5.484 | −12.958 | 1.00 | 30.88 | O |
| ATOM | 4478 | N | VAL | A | 835 | 31.921 | 4.668 | −10.922 | 1.00 | 32.30 | N |
| ATOM | 4480 | CA | VAL | A | 835 | 30.862 | 3.658 | −11.018 | 1.00 | 32.46 | C |
| ATOM | 4482 | CB | VAL | A | 835 | 31.036 | 2.566 | −9.940 | 1.00 | 32.60 | C |
| ATOM | 4484 | CG1 | VAL | A | 835 | 29.819 | 1.639 | −9.907 | 1.00 | 33.06 | C |
| ATOM | 4488 | CG2 | VAL | A | 835 | 32.313 | 1.764 | −10.185 | 1.00 | 32.37 | C |
| ATOM | 4492 | C | VAL | A | 835 | 29.474 | 4.293 | −10.870 | 1.00 | 32.47 | C |
| ATOM | 4493 | O | VAL | A | 835 | 28.527 | 3.897 | −11.548 | 1.00 | 32.48 | O |
| ATOM | 4494 | N | SER | A | 836 | 29.359 | 5.241 | −9.935 | 1.00 | 32.44 | N |
| ATOM | 4496 | CA | SER | A | 836 | 28.134 | 5.995 | −9.720 | 1.00 | 32.19 | C |
| ATOM | 4498 | CB | SER | A | 836 | 27.294 | 5.378 | −8.628 | 1.00 | 32.33 | C |
| ATOM | 4501 | OG | SER | A | 836 | 26.015 | 5.987 | −8.624 | 1.00 | 34.05 | O |
| ATOM | 4503 | C | SER | A | 836 | 28.453 | 7.431 | −9.366 | 1.00 | 31.80 | C |
| ATOM | 4504 | O | SER | A | 836 | 28.979 | 7.724 | −8.286 | 1.00 | 31.10 | O |
| ATOM | 4505 | N | GLU | A | 837 | 28.132 | 8.331 | −10.288 | 1.00 | 31.31 | N |
| ATOM | 4507 | CA | GLU | A | 837 | 28.449 | 9.737 | −10.127 | 1.00 | 31.78 | C |
| ATOM | 4509 | CB | GLU | A | 837 | 28.084 | 10.510 | −11.401 | 1.00 | 32.41 | C |
| ATOM | 4512 | CG | GLU | A | 837 | 28.646 | 11.930 | −11.471 | 1.00 | 36.07 | C |
| ATOM | 4515 | CD | GLU | A | 837 | 30.173 | 11.984 | −11.392 | 1.00 | 41.36 | C |
| ATOM | 4516 | OE1 | GLU | A | 837 | 30.848 | 11.020 | −11.858 | 1.00 | 43.72 | O |
| ATOM | 4517 | OE2 | GLU | A | 837 | 30.700 | 13.002 | −10.869 | 1.00 | 45.04 | O |
| ATOM | 4518 | C | GLU | A | 837 | 27.746 | 10.346 | −8.910 | 1.00 | 30.90 | C |
| ATOM | 4519 | O | GLU | A | 837 | 28.237 | 11.318 | −8.344 | 1.00 | 30.45 | O |
| ATOM | 4520 | N | ASP | A | 838 | 26.615 | 9.771 | −8.509 | 1.00 | 30.19 | N |
| ATOM | 4522 | CA | ASP | A | 838 | 25.896 | 10.213 | −7.307 | 1.00 | 30.79 | C |
| ATOM | 4524 | CB | ASP | A | 838 | 24.494 | 9.626 | −7.312 | 1.00 | 30.45 | C |
| ATOM | 4527 | CG | ASP | A | 838 | 23.733 | 9.988 | −8.566 | 1.00 | 33.30 | C |
| ATOM | 4528 | OD1 | ASP | A | 838 | 23.271 | 11.135 | −8.642 | 1.00 | 30.59 | O |
| ATOM | 4529 | OD2 | ASP | A | 838 | 23.594 | 9.204 | −9.539 | 1.00 | 37.40 | O |
| ATOM | 4530 | C | ASP | A | 838 | 26.595 | 9.856 | −5.964 | 1.00 | 30.92 | C |
| ATOM | 4531 | O | ASP | A | 838 | 26.143 | 10.267 | −4.897 | 1.00 | 30.59 | O |
| ATOM | 4532 | N | CYS | A | 839 | 27.662 | 9.074 | −6.012 | 1.00 | 30.74 | N |
| ATOM | 4534 | CA | CYS | A | 839 | 28.507 | 8.897 | −4.824 | 1.00 | 31.60 | C |
| ATOM | 4536 | CB | CYS | A | 839 | 29.171 | 7.532 | −4.846 | 1.00 | 31.36 | C |
| ATOM | 4539 | SG | CYS | A | 839 | 28.024 | 6.171 | −4.545 | 1.00 | 32.85 | S |
| ATOM | 4540 | C | CYS | A | 839 | 29.562 | 10.003 | −4.724 | 1.00 | 31.85 | C |
| ATOM | 4541 | O | CYS | A | 839 | 30.388 | 9.989 | −3.819 | 1.00 | 31.38 | O |
| ATOM | 4542 | N | PHE | A | 840 | 29.491 | 10.986 | −5.631 | 1.00 | 32.36 | N |
| ATOM | 4544 | CA | PHE | A | 840 | 30.387 | 12.136 | −5.597 | 1.00 | 32.57 | C |
| ATOM | 4546 | CB | PHE | A | 840 | 29.990 | 13.214 | −6.615 | 1.00 | 32.56 | C |
| ATOM | 4549 | CG | PHE | A | 840 | 30.852 | 14.450 | −6.531 | 1.00 | 33.86 | C |
| ATOM | 4550 | CD1 | PHE | A | 840 | 32.178 | 14.413 | −6.946 | 1.00 | 34.98 | C |
| ATOM | 4552 | CE1 | PHE | A | 840 | 32.988 | 15.529 | −6.838 | 1.00 | 34.46 | C |
| ATOM | 4554 | CZ | PHE | A | 840 | 32.480 | 16.693 | −6.291 | 1.00 | 34.57 | C |
| ATOM | 4556 | CE2 | PHE | A | 840 | 31.165 | 16.741 | −5.860 | 1.00 | 34.02 | C |
| ATOM | 4558 | CD2 | PHE | A | 840 | 30.359 | 15.627 | −5.978 | 1.00 | 34.40 | C |
| ATOM | 4560 | C | PHE | A | 840 | 30.548 | 12.796 | −4.211 | 1.00 | 32.63 | C |
| ATOM | 4561 | O | PHE | A | 840 | 31.668 | 13.162 | −3.875 | 1.00 | 33.01 | O |
| ATOM | 4562 | N | PRO | A | 841 | 29.491 | 12.988 | −3.412 | 1.00 | 32.31 | N |
| ATOM | 4563 | CA | PRO | A | 841 | 29.676 | 13.675 | −2.124 | 1.00 | 32.29 | C |
| ATOM | 4565 | CB | PRO | A | 841 | 28.258 | 13.741 | −1.530 | 1.00 | 32.33 | C |
| ATOM | 4568 | CG | PRO | A | 841 | 27.349 | 13.598 | −2.712 | 1.00 | 32.91 | C |
| ATOM | 4571 | CD | PRO | A | 841 | 28.075 | 12.639 | −3.630 | 1.00 | 32.72 | C |
| ATOM | 4574 | C | PRO | A | 841 | 30.657 | 12.981 | −1.180 | 1.00 | 31.80 | C |
| ATOM | 4575 | O | PRO | A | 841 | 31.259 | 13.662 | −0.373 | 1.00 | 32.29 | O |
| ATOM | 4576 | N | LEU | A | 842 | 30.840 | 11.675 | −1.294 | 1.00 | 31.97 | N |
| ATOM | 4578 | CA | LEU | A | 842 | 31.839 | 10.973 | −0.478 | 1.00 | 32.24 | C |
| ATOM | 4580 | CB | LEU | A | 842 | 31.730 | 9.457 | −0.626 | 1.00 | 31.80 | C |
| ATOM | 4583 | CG | LEU | A | 842 | 30.421 | 8.832 | −0.137 | 1.00 | 34.69 | C |
| ATOM | 4585 | CD1 | LEU | A | 842 | 30.347 | 7.408 | −0.600 | 1.00 | 37.43 | C |
| ATOM | 4589 | CD2 | LEU | A | 842 | 30.248 | 8.889 | 1.388 | 1.00 | 36.33 | C |
| ATOM | 4593 | C | LEU | A | 842 | 33.240 | 11.442 | −0.847 | 1.00 | 31.96 | C |
| ATOM | 4594 | O | LEU | A | 842 | 34.037 | 11.751 | 0.023 | 1.00 | 32.60 | O |
| ATOM | 4595 | N | LEU | A | 843 | 33.531 | 11.497 | −2.140 | 1.00 | 31.51 | N |
| ATOM | 4597 | CA | LEU | A | 843 | 34.797 | 12.014 | −2.618 | 1.00 | 31.36 | C |
| ATOM | 4599 | CB | LEU | A | 843 | 34.873 | 11.858 | −4.150 | 1.00 | 31.83 | C |
| ATOM | 4602 | CG | LEU | A | 843 | 36.051 | 12.457 | −4.915 | 1.00 | 31.94 | C |
| ATOM | 4604 | CD1 | LEU | A | 843 | 37.368 | 11.874 | −4.389 | 1.00 | 32.96 | C |
| ATOM | 4608 | CD2 | LEU | A | 843 | 35.880 | 12.174 | −6.413 | 1.00 | 33.16 | C |
| ATOM | 4612 | C | LEU | A | 843 | 34.992 | 13.486 | −2.243 | 1.00 | 30.91 | C |
| ATOM | 4613 | O | LEU | A | 843 | 36.033 | 13.876 | −1.752 | 1.00 | 30.62 | O |
| ATOM | 4614 | N | ASP | A | 844 | 33.989 | 14.306 | −2.501 | 1.00 | 31.32 | N |
| ATOM | 4616 | CA | ASP | A | 844 | 34.071 | 15.736 | −2.204 | 1.00 | 31.76 | C |
| ATOM | 4618 | CB | ASP | A | 844 | 32.764 | 16.414 | −2.608 | 1.00 | 32.08 | C |
| ATOM | 4621 | CG | ASP | A | 844 | 32.871 | 17.923 | −2.647 | 1.00 | 32.63 | C |
| ATOM | 4622 | OD1 | ASP | A | 844 | 33.895 | 18.463 | −3.106 | 1.00 | 35.84 | O |
| ATOM | 4623 | OD2 | ASP | A | 844 | 31.970 | 18.655 | −2.232 | 1.00 | 33.70 | O |
| ATOM | 4624 | C | ASP | A | 844 | 34.385 | 15.959 | −0.708 | 1.00 | 31.46 | C |
| ATOM | 4625 | O | ASP | A | 844 | 35.266 | 16.740 | −0.362 | 1.00 | 31.17 | O |
| ATOM | 4626 | N | GLY | A | 845 | 33.694 | 15.229 | 0.163 | 1.00 | 31.38 | N |
| ATOM | 4628 | CA | GLY | A | 845 | 33.878 | 15.355 | 1.600 | 1.00 | 31.48 | C |
| ATOM | 4631 | C | GLY | A | 845 | 35.266 | 14.898 | 2.028 | 1.00 | 31.50 | C |
| ATOM | 4632 | O | GLY | A | 845 | 35.919 | 15.545 | 2.852 | 1.00 | 31.47 | O |
| ATOM | 4633 | N | CYS | A | 846 | 35.731 | 13.790 | 1.457 | 1.00 | 31.20 | N |
| ATOM | 4635 | CA | CYS | A | 846 | 37.101 | 13.360 | 1.667 | 1.00 | 30.97 | C |
| ATOM | 4637 | CB | CYS | A | 846 | 37.340 | 12.059 | 0.908 | 1.00 | 31.85 | C |
| ATOM | 4640 | SG | CYS | A | 846 | 38.940 | 11.316 | 1.228 | 1.00 | 32.79 | S |
| ATOM | 4641 | C | CYS | A | 846 | 38.132 | 14.435 | 1.253 | 1.00 | 30.87 | C |
| ATOM | 4642 | O | CYS | A | 846 | 39.072 | 14.739 | 1.992 | 1.00 | 29.64 | O |
| ATOM | 4643 | N | ARG | A | 847 | 37.958 | 15.003 | 0.067 | 1.00 | 30.89 | N |
| ATOM | 4645 | CA | ARG | A | 847 | 38.833 | 16.059 | −0.419 | 1.00 | 30.94 | C |
| ATOM | 4647 | CB | ARG | A | 847 | 38.417 | 16.506 | −1.823 | 1.00 | 31.78 | C |
| ATOM | 4650 | CG | ARG | A | 847 | 38.945 | 15.636 | −2.956 | 1.00 | 33.28 | C |
| ATOM | 4653 | CD | ARG | A | 847 | 38.473 | 16.108 | −4.324 | 1.00 | 34.97 | C |
| ATOM | 4656 | NE | ARG | A | 847 | 38.881 | 15.180 | −5.380 | 1.00 | 37.42 | N |
| ATOM | 4658 | CZ | ARG | A | 847 | 38.319 | 15.114 | −6.588 | 1.00 | 38.64 | C |
| ATOM | 4659 | NH1 | ARG | A | 847 | 37.290 | 15.882 | −6.915 | 1.00 | 37.39 | N |
| ATOM | 4662 | NH2 | ARG | A | 847 | 38.782 | 14.241 | −7.472 | 1.00 | 40.75 | N |
| ATOM | 4665 | C | ARG | A | 847 | 38.838 | 17.291 | 0.489 | 1.00 | 30.67 | C |
| ATOM | 4666 | O | ARG | A | 847 | 39.898 | 17.891 | 0.708 | 1.00 | 30.13 | O |
| ATOM | 4667 | N | LYS | A | 848 | 37.669 | 17.672 | 1.010 | 1.00 | 30.00 | N |
| ATOM | 4669 | CA | LYS | A | 848 | 37.572 | 18.873 | 1.840 | 1.00 | 30.06 | C |
| ATOM | 4671 | CB | LYS | A | 848 | 36.119 | 19.316 | 2.035 | 1.00 | 30.40 | C |
| ATOM | 4674 | CG | LYS | A | 848 | 35.493 | 19.974 | 0.810 | 1.00 | 32.67 | C |
| ATOM | 4677 | CD | LYS | A | 848 | 33.969 | 20.151 | 1.034 | 1.00 | 35.48 | C |
| ATOM | 4680 | CE | LYS | A | 848 | 33.273 | 20.910 | −0.099 | 1.00 | 35.58 | C |
| ATOM | 4683 | NZ | LYS | A | 848 | 31.803 | 20.591 | −0.131 | 1.00 | 36.69 | N |
| ATOM | 4687 | C | LYS | A | 848 | 38.244 | 18.652 | 3.185 | 1.00 | 29.16 | C |
| ATOM | 4688 | O | LYS | A | 848 | 38.875 | 19.561 | 3.694 | 1.00 | 28.25 | O |
| ATOM | 4689 | N | ASN | A | 849 | 38.131 | 17.434 | 3.728 | 1.00 | 29.15 | N |
| ATOM | 4691 | CA | ASM | A | 849 | 38.786 | 17.075 | 5.002 | 1.00 | 29.60 | C |
| ATOM | 4693 | CB | ASN | A | 849 | 38.258 | 15.748 | 5.583 | 1.00 | 29.71 | C |
| ATOM | 4696 | CG | ASN | A | 849 | 36.826 | 15.867 | 6.117 | 1.00 | 28.85 | C |
| ATOM | 4697 | OD1 | ASN | A | 849 | 36.402 | 16.925 | 6.530 | 1.00 | 32.39 | O |
| ATOM | 4698 | ND2 | ASN | A | 849 | 36.093 | 14.779 | 6.097 | 1.00 | 28.94 | N |
| ATOM | 4701 | C | ASN | A | 849 | 40.298 | 17.023 | 4.862 | 1.00 | 29.97 | C |
| ATOM | 4702 | O | ASN | A | 849 | 41.024 | 17.445 | 5.777 | 1.00 | 30.10 | O |
| ATOM | 4703 | N | ARG | A | 850 | 40.783 | 16.550 | 3.710 | 1.00 | 30.30 | N |
| ATOM | 4705 | CA | ARG | A | 850 | 42.224 | 16.548 | 3.441 | 1.00 | 30.06 | C |
| ATOM | 4707 | CB | ARG | A | 850 | 42.525 | 15.868 | 2.103 | 1.00 | 30.76 | C |
| ATOM | 4710 | CG | ARG | A | 850 | 43.997 | 15.480 | 1.922 | 1.00 | 30.45 | C |
| ATOM | 4713 | CD | ARG | A | 850 | 44.536 | 15.663 | 0.516 | 1.00 | 31.23 | C |
| ATOM | 4716 | NE | ARG | A | 850 | 45.953 | 15.294 | 0.431 | 1.00 | 31.15 | N |
| ATOM | 4718 | CZ | ARG | A | 850 | 46.981 | 16.122 | 0.656 | 1.00 | 31.68 | C |
| ATOM | 4719 | NH1 | ARG | A | 850 | 48.226 | 15.661 | 0.572 | 1.00 | 31.87 | N |
| ATOM | 4722 | NH2 | ARG | A | 850 | 46.796 | 17.395 | 0.962 | 1.00 | 32.03 | N |
| ATOM | 4725 | C | ARG | A | 850 | 42.765 | 17.979 | 3.457 | 1.00 | 30.37 | C |
| ATOM | 4726 | O | ARG | A | 850 | 43.801 | 18.245 | 4.058 | 1.00 | 29.83 | O |
| ATOM | 4727 | N | GLN | A | 851 | 42.045 | 18.895 | 2.812 | 1.00 | 31.08 | N |
| ATOM | 4729 | CA | GLN | A | 851 | 42.403 | 20.317 | 2.806 | 1.00 | 32.13 | C |
| ATOM | 4731 | CB | GLN | A | 851 | 41.380 | 21.178 | 2.035 | 1.00 | 32.54 | C |
| ATOM | 4734 | CG | GLN | A | 851 | 41.601 | 21.299 | 0.549 | 1.00 | 35.11 | C |
| ATOM | 4737 | CD | GLN | A | 851 | 40.739 | 22.400 | −0.088 | 1.00 | 37.85 | C |
| ATOM | 4738 | OE1 | GLN | A | 851 | 41.250 | 23.451 | −0.507 | 1.00 | 38.21 | O |
| ATOM | 4739 | NE2 | GLN | A | 851 | 39.425 | 22.157 | −0.155 | 1.00 | 39.86 | N |
| ATOM | 4742 | C | GLN | A | 851 | 42.488 | 20.837 | 4.238 | 1.00 | 31.69 | C |
| ATOM | 4743 | O | GLN | A | 851 | 43.473 | 21.471 | 4.608 | 1.00 | 31.56 | O |
| ATOM | 4744 | N | LYS | A | 852 | 41.458 | 20.548 | 5.034 | 1.00 | 31.11 | N |
| ATOM | 4746 | CA | LYS | A | 852 | 41.385 | 21.025 | 6.412 | 1.00 | 31.32 | C |
| ATOM | 4748 | CB | LYS | A | 852 | 39.988 | 20.757 | 7.010 | 1.00 | 31.63 | C |
| ATOM | 4751 | CG | LYS | A | 852 | 38.850 | 21.660 | 6.454 | 1.00 | 32.95 | C |
| ATOM | 4754 | CD | LYS | A | 852 | 38.794 | 23.023 | 1.111 | 1.00 | 35.29 | C |
| ATOM | 4757 | CE | LYS | A | 852 | 37.794 | 24.003 | 6.545 | 1.00 | 36.53 | C |
| ATOM | 4760 | NZ | LYS | A | 852 | 38.394 | 25.348 | 6.227 | 1.00 | 38.20 | N |
| ATOM | 4764 | C | LYS | A | 852 | 42.509 | 20.444 | 7.299 | 1.00 | 30.77 | C |
| ATOM | 4765 | O | LYS | A | 852 | 43.193 | 21.190 | 8.003 | 1.00 | 31.25 | O |
| ATOM | 4766 | N | TRP | A | 853 | 42.732 | 19.135 | 7.238 | 1.00 | 30.53 | N |
| ATOM | 4768 | CA | TRP | A | 853 | 43.836 | 18.505 | 7.981 | 1.00 | 30.28 | C |
| ATOM | 4770 | CB | TRP | A | 853 | 43.772 | 16.973 | 7.904 | 1.00 | 30.25 | C |
| ATOM | 4773 | CG | TRP | A | 853 | 42.685 | 16.327 | 8.734 | 1.00 | 29.17 | C |
| ATOM | 4774 | CD1 | TRP | A | 853 | 41.682 | 15.521 | 8.277 | 1.00 | 29.72 | C |
| ATOM | 4776 | NE1 | TRP | A | 853 | 40.895 | 15.092 | 9.318 | 1.00 | 27.75 | N |
| ATOM | 4778 | CE2 | TRP | A | 853 | 41.394 | 15.608 | 10.489 | 1.00 | 29.96 | C |
| ATOM | 4779 | CD2 | TRP | A | 853 | 42.519 | 16.395 | 10.158 | 1.00 | 29.49 | C |
| ATOM | 4780 | CE3 | TRP | A | 853 | 43.196 | 17.053 | 11.192 | 1.00 | 29.21 | C |
| ATOM | 4782 | CZ3 | TRP | A | 853 | 42.739 | 16.904 | 12.502 | 1.00 | 30.37 | C |
| ATOM | 4784 | CH2 | TRP | A | 853 | 41.628 | 16.120 | 12.798 | 1.00 | 28.68 | C |
| ATOM | 4786 | CZ2 | TRP | A | 853 | 40.930 | 15.475 | 11.810 | 1.00 | 30.57 | C |
| ATOM | 4788 | C | TRP | A | 853 | 45.213 | 18.967 | 7.508 | 1.00 | 30.57 | C |
| ATOM | 4789 | O | TRP | A | 853 | 46.093 | 19.176 | 8.332 | 1.00 | 29.97 | O |
| ATOM | 4790 | N | GLN | A | 854 | 45.405 | 19.133 | 6.195 | 1.00 | 31.37 | N |
| ATOM | 4792 | CA | GLN | A | 854 | 46.696 | 19.613 | 5.660 | 1.00 | 32.06 | C |
| ATOM | 4794 | CB | GLN | A | 854 | 46.722 | 19.603 | 4.131 | 1.00 | 32.60 | C |
| ATOM | 4797 | CG | GLN | A | 854 | 48.091 | 20.008 | 3.493 | 1.00 | 34.51 | C |
| ATOM | 4800 | CD | GLN | A | 854 | 49.224 | 19.077 | 3.896 | 1.00 | 35.78 | C |
| ATOM | 4801 | OE1 | GLN | A | 854 | 48.990 | 17.924 | 4.265 | 1.00 | 35.86 | O |
| ATOM | 4802 | NE2 | GLN | A | 854 | 50.450 | 19.580 | 3.842 | 1.00 | 37.93 | N |
| ATOM | 4805 | C | GLN | A | 854 | 47.025 | 21.017 | 6.144 | 1.00 | 32.21 | C |
| ATOM | 4806 | O | GLN | A | 854 | 48.164 | 21.287 | 6.460 | 1.00 | 31.44 | O |
| ATOM | 4807 | N | ALA | A | 855 | 46.019 | 21.891 | 6.207 | 1.00 | 32.96 | N |
| ATOM | 4809 | CA | ALA | A | 855 | 46.198 | 23.258 | 6.710 | 1.00 | 33.54 | C |
| ATOM | 4811 | CB | ALA | A | 855 | 44.943 | 24.112 | 6.487 | 1.00 | 33.09 | C |
| ATOM | 4815 | C | ALA | A | 855 | 46.553 | 23.222 | 8.185 | 1.00 | 34.08 | C |
| ATOM | 4816 | O | ALA | A | 855 | 47.357 | 24.019 | 8.641 | 1.00 | 34.67 | O |
| ATOM | 4817 | N | LEU | A | 856 | 45.956 | 22.291 | 8.921 | 1.00 | 34.47 | N |
| ATOM | 4819 | CA | LEU | A | 856 | 46.308 | 22.066 | 10.318 | 1.00 | 35.05 | C |
| ATOM | 4821 | CB | LEU | A | 856 | 45.275 | 21.160 | 10.984 | 1.00 | 35.45 | C |
| ATOM | 4824 | CG | LEU | A | 856 | 44.140 | 21.879 | 11.685 | 1.00 | 35.64 | C |
| ATOM | 4826 | CD1 | LEU | A | 856 | 43.111 | 20.854 | 12.128 | 1.00 | 35.60 | C |
| ATOM | 4830 | CD2 | LEU | A | 856 | 44.669 | 22.679 | 12.876 | 1.00 | 36.89 | C |
| ATOM | 4834 | C | LEU | A | 856 | 47.713 | 21.487 | 10.520 | 1.00 | 35.54 | C |
| ATOM | 4835 | O | LEU | A | 856 | 48.405 | 21.871 | 11.469 | 1.00 | 36.00 | O |
| ATOM | 4836 | N | ALA | A | 857 | 48.151 | 20.596 | 9.632 | 1.00 | 36.23 | N |
| ATOM | 4838 | CA | ALA | A | 857 | 49.512 | 20.032 | 9.707 | 1.00 | 36.86 | C |
| ATOM | 4840 | CB | ALA | A | 857 | 49.675 | 18.886 | 8.730 | 1.00 | 36.47 | C |
| ATOM | 4844 | C | ALA | A | 857 | 50.583 | 21.096 | 9.452 | 1.00 | 38.02 | C |
| ATOM | 4845 | O | ALA | A | 857 | 51.708 | 20.990 | 9.947 | 1.00 | 38.57 | O |
| ATOM | 4846 | N | GLU | A | 858 | 50.220 | 22.120 | 8.689 | 1.00 | 39.06 | N |
| ATOM | 4848 | CA | GLU | A | 858 | 51.147 | 23.160 | 8.274 | 1.00 | 39.95 | C |
| ATOM | 4850 | CB | GLU | A | 858 | 50.714 | 23.720 | 6.921 | 1.00 | 40.18 | C |
| ATOM | 4853 | CG | GLU | A | 858 | 51.060 | 22.815 | 5.744 | 1.00 | 40.91 | C |
| ATOM | 4856 | CD | GLU | A | 858 | 50.697 | 23.429 | 4.408 | 1.00 | 42.60 | C |
| ATOM | 4857 | OE1 | GLU | A | 858 | 50.787 | 24.669 | 4.283 | 1.00 | 45.70 | O |
| ATOM | 4858 | OE2 | GLU | A | 858 | 50.337 | 22.685 | 3.475 | 1.00 | 43.19 | O |
| ATOM | 4859 | C | GLU | A | 858 | 51.198 | 24.263 | 9.317 | 1.00 | 40.65 | C |
| ATOM | 4860 | O | GLU | A | 858 | 52.258 | 24.808 | 9.616 | 1.00 | 40.62 | O |
| ATOM | 4861 | N | GLN | A | 859 | 50.034 | 24.584 | 9.870 | 1.00 | 41.69 | N |
| ATOM | 4863 | CA | GLN | A | 859 | 49.931 | 25.557 | 10.945 | 1.00 | 42.43 | C |
| ATOM | 4865 | CB | GLN | A | 859 | 48.473 | 25.911 | 11.212 | 1.00 | 42.25 | C |
| ATOM | 4868 | CG | GLN | A | 859 | 48.283 | 27.204 | 12.027 | 1.00 | 42.92 | C |
| ATOM | 4871 | CD | GLN | A | 859 | 47.100 | 27.140 | 12.973 | 1.00 | 42.72 | C |
| ATOM | 4872 | OE1 | GLN | A | 859 | 46.619 | 26.054 | 13.304 | 1.00 | 42.44 | O |
| ATOM | 4873 | NE2 | GLN | A | 859 | 46.620 | 28.307 | 13.404 | 1.00 | 42.81 | N |
| ATOM | 4876 | C | GLN | A | 859 | 50.582 | 25.026 | 12.218 | 1.00 | 43.48 | C |
| ATOM | 4877 | O | GLN | A | 859 | 51.049 | 25.817 | 13.037 | 1.00 | 44.43 | O |
| ATOM | 4878 | N | GLN | A | 860 | 50.640 | 23.702 | 12.374 | 1.00 | 44.48 | N |
| ATOM | 4880 | CA | GLN | A | 860 | 51.220 | 23.091 | 13.571 | 1.00 | 45.14 | C |
| ATOM | 4882 | CB | GLN | A | 860 | 50.674 | 21.680 | 13.810 | 1.00 | 45.53 | C |
| ATOM | 4885 | CG | GLN | A | 860 | 49.391 | 21.646 | 14.675 | 1.00 | 45.81 | C |
| ATOM | 4888 | CD | GLN | A | 860 | 48.904 | 20.223 | 14.929 | 1.00 | 48.17 | C |
| ATOM | 4889 | OE1 | GLN | A | 860 | 47.774 | 20.011 | 15.408 | 1.00 | 49.37 | O |
| ATOM | 4890 | NE2 | GLN | A | 860 | 49.747 | 19.239 | 14.593 | 1.00 | 48.49 | N |
| ATOM | 4893 | C | GLN | A | 860 | 52.741 | 23.080 | 13.502 | 1.00 | 45.80 | C |
| ATOM | 4894 | O | GLN | A | 860 | 53.399 | 23.165 | 14.541 | 1.00 | 46.30 | O |
| ATOM | 4895 | N | GLU | A | 861 | 53.298 | 23.014 | 12.290 | 1.00 | 46.36 | N |
| ATOM | 4897 | CA | GLU | A | 861 | 54.744 | 23.231 | 12.078 | 1.00 | 46.78 | C |
| ATOM | 4899 | CB | GLU | A | 861 | 55.145 | 22.865 | 10.637 | 1.00 | 46.94 | C |
| ATOM | 4902 | CG | GLU | A | 861 | 55.018 | 21.378 | 10.315 | 1.00 | 48.41 | C |
| ATOM | 4905 | CD | GLU | A | 861 | 54.889 | 21.091 | 8.816 | 1.00 | 51.01 | C |
| ATOM | 4906 | OE1 | GLU | A | 861 | 55.877 | 21.318 | 8.079 | 1.00 | 51.91 | O |
| ATOM | 4907 | OE2 | GLU | A | 861 | 53.799 | 20.630 | 8.368 | 1.00 | 53.39 | O |
| ATOM | 4908 | C | GLU | A | 861 | 55.187 | 24.682 | 12.406 | 1.00 | 46.87 | C |
| ATOM | 4909 | O | GLU | A | 861 | 56.380 | 25.003 | 12.379 | 1.00 | 46.87 | O |
| ATOM | 4910 | N | LYS | A | 862 | 54.213 | 25.542 | 12.722 | 1.00 | 47.14 | N |
| ATOM | 4912 | CA | LYS | A | 862 | 54.433 | 26.943 | 13.075 | 1.00 | 47.01 | C |
| ATOM | 4914 | CB | LYS | A | 862 | 55.288 | 27.099 | 14.355 | 1.00 | 47.27 | C |
| ATOM | 4917 | CG | LYS | A | 862 | 55.193 | 25.943 | 15.392 | 1.00 | 46.80 | C |
| ATOM | 4920 | CD | LYS | A | 862 | 56.571 | 25.256 | 15.615 | 1.00 | 46.48 | C |
| ATOM | 4923 | CE | LYS | A | 862 | 56.457 | 23.740 | 15.812 | 1.00 | 46.64 | C |
| ATOM | 4926 | NZ | LYS | A | 862 | 57.811 | 23.103 | 15.770 | 1.00 | 47.11 | N |
| ATOM | 4930 | C | LYS | A | 862 | 55.078 | 27.670 | 11.893 | 1.00 | 47.28 | C |
| ATOM | 4931 | O | LYS | A | 862 | 54.900 | 27.261 | 10.741 | 1.00 | 47.16 | O |
| ATOM | 4932 | ZN | ZN | A | 1 | 34.525 | −0.993 | 10.630 | 1.00 | 50.38 | ZN |
| ATOM | 4934 | O5 | CIT | L | 101 | 49.023 | 1.293 | −4.093 | 1.00 | 68.10 | O |
| ATOM | 4935 | C6 | CIT | L | 101 | 48.308 | 0.359 | −4.647 | 0.50 | 72.51 | C |
| ATOM | 4936 | O6 | CIT | L | 101 | 47.451 | −0.244 | −3.887 | 1.00 | 72.89 | O |
| ATOM | 4938 | C3 | CIT | L | 101 | 48.403 | −0.153 | −6.141 | 0.50 | 73.65 | C |
| ATOM | 4939 | O7 | CIT | L | 101 | 47.133 | −0.911 | −6.214 | 1.00 | 72.11 | O |
| ATOM | 4941 | C4 | CIT | L | 101 | 48.459 | 0.972 | −7.359 | 1.00 | 77.19 | C |
| ATOM | 4944 | C5 | CIT | L | 101 | 47.271 | 1.292 | −8.386 | 1.00 | 79.59 | C |
| ATOM | 4945 | O4 | CIT | L | 101 | 46.043 | 0.975 | −8.301 | 1.00 | 80.25 | O |
| ATOM | 4947 | O3 | CIT | L | 101 | 47.505 | 1.962 | −9.430 | 1.00 | 81.88 | O |
| ATOM | 4948 | C2 | CIT | L | 101 | 49.610 | −1.212 | −5.995 | 1.00 | 72.14 | C |
| ATOM | 4951 | C1 | CIT | L | 101 | 49.294 | −2.684 | −6.267 | 1.00 | 74.60 | C |
| ATOM | 4952 | O1 | CIT | L | 101 | 49.594 | −3.235 | −7.342 | 1.00 | 77.97 | O |
| ATOM | 4953 | O2 | CIT | L | 101 | 48.717 | −3.440 | −5.450 | 1.00 | 79.07 | O |
| ATOM | 4955 | O | HOH | W | 1 | 48.207 | 0.000 | 9.377 | 0.50 | 26.55 | O |
| ATOM | 4958 | O | HOH | W | 2 | 48.206 | 0.002 | 15.548 | 0.50 | 32.28 | O |
| ATOM | 4961 | O | HOH | W | 3 | 34.289 | 5.273 | 9.154 | 1.00 | 43.88 | O |
| ATOM | 4964 | O | HOH | W | 4 | 49.048 | 8.267 | 7.910 | 1.00 | 45.59 | O |
| ATOM | 4967 | O | HOH | W | 5 | 28.822 | −0.801 | 21.427 | 1.00 | 49.21 | O |
| ATOM | 4970 | O | HOH | W | 6 | 43.739 | −6.442 | 7.412 | 1.00 | 42.96 | O |
| ATOM | 4973 | O | HOH | W | 7 | 18.296 | −16.040 | 10.216 | 1.00 | 47.23 | O |
| ATOM | 4976 | O | HOH | W | 8 | 32.430 | 5.453 | 12.380 | 1.00 | 48.00 | O |
| ATOM | 4979 | O | HOH | W | 9 | 24.183 | −11.250 | 12.977 | 1.00 | 53.40 | O |
| ATOM | 4982 | O | HOH | W | 10 | 33.088 | −13.347 | 14.733 | 1.00 | 50.13 | O |
| ATOM | 4985 | O | HOH | W | 11 | 16.672 | 10.547 | 4.776 | 1.00 | 58.12 | O |
| ATOM | 4988 | O | HOH | W | 12 | 41.666 | −3.613 | 8.704 | 1.00 | 43.31 | O |
| ATOM | 4991 | O | HOH | W | 13 | 50.372 | 16.058 | 12.308 | 1.00 | 55.26 | O |
| ATOM | 4994 | O | HOH | W | 14 | 38.665 | −2.779 | 3.708 | 1.00 | 39.85 | O |
| ATOM | 4997 | O | HOH | W | 15 | 45.675 | −12.816 | 6.127 | 1.00 | 53.14 | O |
| ATOM | 5000 | O | HOH | W | 16 | 34.796 | 5.945 | 18.234 | 1.00 | 65.49 | O |
| ATOM | 5003 | O | HOH | W | 17 | 39.086 | −6.542 | −1.130 | 1.00 | 50.00 | O |
| ATOM | 5006 | O | HOH | W | 18 | 10.303 | −3.731 | 3.425 | 1.00 | 53.09 | O |
| ATOM | 5009 | O | HOH | W | 19 | 31.249 | −7.203 | 22.941 | 1.00 | 66.27 | O |
| ATOM | 5012 | O | HOH | W | 20 | 26.985 | 8.278 | 5.295 | 1.00 | 61.86 | O |
| ATOM | 5015 | O | HOH | W | 21 | 44.919 | −5.862 | −7.194 | 1.00 | 52.06 | O |
| ATOM | 5018 | O | HOH | W | 22 | 32.887 | −9.277 | −1.547 | 1.00 | 51.46 | O |
| ATOM | 5021 | O | HOH | W | 23 | 44.118 | 6.614 | −2.096 | 1.00 | 51.08 | O |
| ATOM | 5024 | O | HOH | W | 24 | 38.266 | 9.274 | 10.146 | 1.00 | 49.52 | O |
| ATOM | 5027 | O | HOH | W | 25 | 42.046 | 4.283 | 16.840 | 1.00 | 64.91 | O |
| ATOM | 5030 | O | HOH | W | 26 | 20.815 | −4.177 | 23.006 | 1.00 | 57.34 | O |
| ATOM | 5033 | O | HOH | W | 27 | 17.407 | −18.805 | 9.260 | 1.00 | 56.41 | O |
| ATOM | 5036 | O | HOH | W | 28 | 50.908 | 17.436 | 1.757 | 1.00 | 71.67 | O |
| ATOM | 5039 | O | HOH | W | 29 | 37.973 | −4.694 | 0.470 | 1.00 | 47.95 | O |
| ATOM | 5042 | O | HOH | W | 30 | 51.895 | 10.582 | 4.609 | 1.00 | 56.86 | O |
| ATOM | 5045 | O | HOH | W | 31 | 46.528 | −3.412 | −5.382 | 1.00 | 46.30 | O |
| ATOM | 5048 | O | HOH | W | 32 | 41.988 | 18.107 | −0.911 | 1.00 | 62.92 | O |
| ATOM | 5051 | O | HOH | W | 33 | 50.668 | 16.518 | 5.962 | 1.00 | 76.18 | O |
| ATOM | 5054 | O | HOH | W | 34 | 26.830 | 8.551 | 21.466 | 1.00 | 67.61 | O |
| ATOM | 5057 | O | HOH | W | 35 | 39.963 | −11.947 | 8.440 | 1.00 | 52.38 | O |
| ATOM | 5060 | O | HOH | W | 36 | 9.206 | −2.461 | 8.722 | 1.00 | 67.69 | O |
| ATOM | 5063 | O | HOH | W | 37 | 27.208 | 7.171 | −13.059 | 1.00 | 66.89 | O |
| ATOM | 5066 | O | HOH | W | 38 | 33.319 | −3.136 | −21.331 | 1.00 | 79.21 | O |
| ATOM | 5069 | O | HOH | W | 39 | 29.807 | 12.113 | 4.210 | 1.00 | 56.70 | O |
| ATOM | 5072 | O | HOH | W | 40 | 26.485 | −2.035 | 22.374 | 1.00 | 59.06 | O |
| ATOM | 5075 | O | HOH | W | 41 | 13.524 | −16.426 | 14.869 | 1.00 | 56.13 | O |
| ATOM | 5078 | O | HOH | W | 42 | 41.242 | 13.929 | −5.574 | 1.00 | 69.30 | O |
| ATOM | 5081 | O | HOH | W | 43 | 44.000 | 3.616 | 30.031 | 1.00 | 79.86 | O |
| ATOM | 5084 | O | HOH | W | 44 | 29.770 | −15.325 | −3.436 | 1.00 | 72.37 | O |
| ATOM | 5087 | O | HOH | W | 45 | 55.046 | −7.275 | 10.420 | 1.00 | 78.57 | O |
| ATOM | 5090 | O | HOH | W | 46 | 42.174 | 23.377 | 8.895 | 1.00 | 59.81 | O |
| ATOM | 5093 | O | HOH | W | 47 | 45.846 | 5.171 | −3.429 | 1.00 | 62.43 | O |
| ATOM | 5096 | O | HOH | W | 48 | 10.721 | 2.483 | −0.428 | 1.00 | 71.72 | O |
| ATOM | 5099 | O | HOH | W | 49 | 38.039 | −8.880 | −0.851 | 1.00 | 59.30 | O |
| ATOM | 5102 | O | HOH | W | 50 | 29.104 | −11.816 | −4.693 | 1.00 | 69.57 | O |
| ATOM | 5105 | O | HOH | W | 51 | 37.981 | −1.356 | 6.040 | 1.00 | 52.06 | O |
| ATOM | 5108 | O | HOH | W | 52 | 38.803 | −5.419 | −9.275 | 1.00 | 74.64 | O |
| ATOM | 5111 | O | HOH | W | 53 | 41.112 | 23.777 | 3.772 | 1.00 | 74.75 | O |
| ATOM | 5114 | O | HOH | W | 54 | 31.043 | 5.736 | 15.133 | 1.00 | 52.37 | O |
| ATOM | 5117 | O | HOH | W | 55 | 25.576 | −9.292 | −16.079 | 1.00 | 78.39 | O |
| ATOM | 5120 | O | HOH | W | 56 | 34.740 | −9.401 | 12.971 | 1.00 | 50.11 | O |
| ATOM | 5123 | O | HOH | W | 57 | 37.034 | −14.827 | 20.525 | 1.00 | 72.86 | O |
| ATOM | 5126 | O | HOH | W | 58 | 48.193 | 0.023 | −15.539 | 0.50 | 75.94 | O |
| ATOM | 5129 | O | HOH | W | 59 | 15.570 | −23.396 | −5.422 | 1.00 | 64.14 | O |
| ATOM | 5132 | O | HOH | W | 60 | 44.168 | −8.356 | −7.513 | 1.00 | 64.26 | O |
| ATOM | 5135 | O | HOH | W | 61 | 28.988 | −3.170 | −14.959 | 1.00 | 77.62 | O |
| ATOM | 5138 | O | HOH | W | 62 | 20.901 | −25.326 | −2.234 | 1.00 | 67.25 | O |
| ATOM | 5141 | O | HOH | W | 63 | 33.982 | 16.977 | 4.436 | 1.00 | 59.29 | O |
| ATOM | 5144 | O | HOH | W | 64 | 37.652 | 17.395 | 18.730 | 1.00 | 70.50 | O |
| ATOM | 5147 | O | HOH | W | 65 | 48.714 | −1.176 | 12.509 | 1.00 | 55.83 | O |
| ATOM | 5150 | O | HOH | W | 66 | 23.648 | 13.228 | 15.636 | 1.00 | 61.83 | O |
| ATOM | 5153 | O | HOH | W | 67 | 37.930 | −5.485 | 14.629 | 1.00 | 62.75 | O |
| ATOM | 5156 | O | HOH | W | 68 | 39.361 | 8.113 | 12.281 | 1.00 | 65.09 | O |
| ATOM | 5159 | O | HOH | W | 69 | 44.416 | 19.041 | 0.025 | 1.00 | 65.31 | O |
| ATOM | 5162 | O | HOH | W | 70 | 31.024 | −11.670 | 12.913 | 1.00 | 51.04 | O |
| ATOM | 5165 | O | HOH | W | 71 | 30.803 | −10.927 | −2.260 | 1.00 | 70.95 | O |
| ATOM | 5168 | O | HOH | W | 72 | 18.394 | 4.353 | 24.131 | 1.00 | 56.96 | O |
| ATOM | 5171 | O | HOH | W | 73 | 40.724 | −9.915 | −6.592 | 1.00 | 71.31 | O |
| ATOM | 5174 | O | HOH | W | 74 | 39.415 | −11.451 | −1.251 | 1.00 | 59.36 | O |
| ATOM | 5177 | O | HOH | W | 75 | 10.729 | 6.380 | 4.422 | 1.00 | 64.07 | O |
| ATOM | 5180 | O | HOH | W | 76 | 32.027 | −16.657 | 1.416 | 1.00 | 71.19 | O |
| ATOM | 5183 | O | HOH | W | 77 | 26.786 | −20.606 | 2.339 | 1.00 | 57.23 | O |
| ATOM | 5186 | O | HOH | W | 78 | 18.583 | 16.228 | 4.823 | 1.00 | 73.42 | O |
| ATOM | 5189 | O | HOH | W | 79 | 27.374 | 7.398 | 8.872 | 1.00 | 62.50 | O |
| ATOM | 5192 | O | HOH | W | 80 | 35.193 | −16.175 | 10.352 | 1.00 | 73.93 | O |
| ATOM | 5195 | O | HOH | W | 81 | 52.965 | 18.374 | 4.826 | 1.00 | 72.09 | O |
| ATOM | 5198 | O | HOH | W | 82 | 11.094 | 0.000 | 0.113 | 1.00 | 85.89 | O |
| ATOM | 5201 | O | HOH | W | 83 | 22.715 | 12.451 | 0.236 | 1.00 | 66.47 | O |
| ATOM | 5204 | O | HOH | W | 84 | 29.996 | 6.584 | 8.368 | 1.00 | 57.82 | O |
| ATOM | 5207 | O | HOH | W | 85 | 42.337 | 13.541 | 20.658 | 1.00 | 71.89 | O |
| ATOM | 5210 | O | HOH | W | 86 | 37.358 | −4.176 | 21.021 | 1.00 | 72.66 | O |
| ATOM | 5213 | O | HOH | W | 87 | 13.285 | −6.976 | −6.448 | 1.00 | 67.69 | O |
| ATOM | 5216 | O | HOH | W | 88 | 43.834 | 23.530 | −1.284 | 1.00 | 77.37 | O |
| ATOM | 5219 | O | HOH | W | 89 | 45.114 | 22.881 | 3.087 | 1.00 | 68.89 | O |
| ATOM | 5222 | O | HOH | W | 90 | 44.237 | −15.595 | 6.458 | 1.00 | 87.26 | O |

TABLE 2

PCR from Human Kidney QUICK-Clone cDNA (Clontech, #7112-1)
Protein in pET15S: 366 aa Mass: 42049.2 pI: 6.68

| | | | | | | |
|---|---|---|---|---|---|---|
| 1 | MGSSHHHHHH | SSGLVPRGSH | MSAAEEETRE | LQSLAAAVVP | SAQTLKITDF | SFSDFELSDL |
| 61 | ETALCTIRMF | TDLNLVQNFQ | MKHEVLCRWI | LSVKKNYRKN | VAYHNWRHAF | NTAQCMFAAL |
| 121 | KAGKIQNKLT | DLEILALLIA | ALSHDLDHRG | VNNSYIQRSE | HPLAQLYCHS | IMEHHHFDQC |
| 181 | LMILNSPGNQ | ILSGLSIEEY | KTTLKIIKQA | ILATDLALYI | KRRGEFFELI | RKNQFNLEDP |
| 241 | HQKELFLAML | MTACDLSAIT | KPWPIQQRIA | ELVATEFFDQ | GDRERKELNI | EPTDLMNREK |
| 301 | KNKIPSMQVG | FIDAICLQLY | EALTHVSEDC | FPLLDGCRKN | RQKWQALAEQ | QEKMLINGES |
| 361 | GQAKRN | | | | | |

| | |
|---|---|
| PDE5A-S: | 5′-GTCGTAT CATATG TCAGCAGCAGAGGAAGAAAC-3′ 33 mer |
| PDE5A-A: | 5′-TCTGCA GTCGAC AGGCCACTCAGTTCCGCTTG-3′ 32 mer |

pET15S sequence (PCR product; 1070 bp)
ATATACCATGGGCAGCAGCCATCATCATCATCATCACAGCAGCGGCCTGGTGCCGCGCGGCAGCCATATG

| | | | | | | |
|---|---|---|---|---|---|---|
| | t | cagcagcaga | | | | |
| 1921 | ggaagaaaca | agagagctac | agtcgttagc | ggctgctgtg | gtgccatctg | cccagaccct |
| 1981 | taaaattact | gactttagct | tcagtgactt | tgagctgtct | gatctggaaa | cagcactgtg |
| 2041 | tacaattcgg | atgtttactg | acctcaacct | tgtgcagaac | ttccagatga | aacatgaggt |
| 2101 | tctttgcaga | tggattttaa | gtgttaagaa | gaattatcgg | aagaatgttg | cctatcataa |
| 2161 | ttggagacat | gcctttaata | cagctcagtg | catgtttgct | gctctaaaag | caggcaaaat |
| 2221 | tcagaacaag | ctgactgacc | tggagatact | tgcattgctg | attgctgcac | taagccacga |
| 2281 | tttggatcac | cgtggtgtga | ataactctta | catacagcga | agtgaacatc | cacttgccca |
| 2341 | gctttactgc | cattcaatca | tggaacacca | tcattttgac | cagtgcctga | tgattcttaa |
| 2401 | tagtccaggc | aatcagattc | tcagtggcct | ctccattgaa | gaatataaga | ccacgttgaa |
| 2461 | aataatcaag | caagctattt | tagctacaga | cctagcactg | tacattaaga | ggcgaggaga |
| 2521 | attttttgaa | cttataagaa | aaaatcaatt | caatttggaa | gatcctcatc | aaaaggagtt |
| 2581 | gtttttggca | atgctgatga | cagcttgtga | tctttctgca | attacaaaac | cctggcctat |
| 2641 | tcaacaacgg | atagcagaac | ttgtagcaac | tgaatttttt | gatcaaggag | acagagagag |
| 2701 | aaaagaactc | aacatagaac | ccactgatct | aatgaacagg | gagaagaaaa | acaaaatccc |
| 2761 | aagtatgcaa | gttgggttca | tagatgccat | ctgcttgcaa | ctgtatgagg | ccctgaccca |
| 2821 | cgtgtcagag | gactgtttcc | ctttgctaga | tggctgcaga | aagaacaggc | agaaatggca |
| 2881 | ggcccttgca | gaacagcagg | agaagatgct | gattaatggg | gaaagcggcc | aggccaagcg |
| 2941 | gaactgagtg | gcct | | | | |

GTCGACTAGAGCCTGCAGTCTCGACCATCATCATCATCATCATTAATAAAAGGGCGAATTCCAGCACACT

[image] TABLE 4

| | |
|---|---|
| LOCUS | PDE5A 3106 bp mRNA linear PRI Nov. 5, 2002 |
| DEFINITION | Homo sapiens phosphodiesterase 5A, cGMP-specific (PDE5A), |
| | transcript variant 1, mRNA. |
| ACCESSION | NM_001083 |
| VERSION | NM_001083.2 GI:15812210 |
| KEYWORDS | . |
| SOURCE | Homo sapiens (human) |
| ORGANISM | Homo sapiens |
| | Eukaryota; Metazoa; Chordata; Craniata; Vertebrate, Euteleostomi; |
| | Mammalia; Eutheria; Primates; Catarrhini; Hominidae; Homo. |
| REFERENCE | 1 (bases 1 to 3106) |
| AUTHORS | Stacey, P., Rulten, S., Dapling, A. and Phillips, S. C. |
| TITLE | Molecular cloning and expression of human cGMP-binding |
| | cGMP-specific phosphodiesterase (PDE5) |
| JOURNAL | Biochem. Biophys. Res. Commun. 247 (2), 249-254 (1998) |
| MEDLINE | 98308101 |
| PUBMED | 9642111 |
| REFERENCE | 2 (bases 1 to 3106) |
| AUTHORS | Yanaka, N., Kotera, J., Ohtsuka, A., Akatsuka, H., Imai, Y., |
| | Michibata, H., Fujishige, K., Kawai, E., Takebayashi, S., Okumura, K. |
| | and Omori, K. |
| TITLE | Expression, structure and chromosomal localization of the human |
| | cGMP-binding cGMP-specific phosphodiesterase PDE5A gene |
| JOURNAL | Eur. J. Biochem. 255 (2), 391-399 (1998) |
| MEDLINE | 98380237 |
| PUBMED | 9716380 |
| REFERENCE | 3 (bases 1 to 3106) |
| AUTHORS | Loughney, K., Hill, T. R., Florio, V. A., Uher, L., Rosman, G. J., |
| | Wolda, S. L., Jones, B. A., Howard, M. L., McAllister-Lucas, L. M. , |
| | Sonnenburg, W. K., Francis, S. H. , Corbin, J. D., Beavo,J. A. and |
| | Ferguson, K. |
| TITLE | Isolation and characterization of cDNAs encoding PDE5A, a human |
| | cGMP-binding, cGMP-specific 3′,5′-cyclic nucleotide |
| | phosphodiesterase |
| JOURNAL | Gene 216 (1), 139-147 (1998) |
| MEDLINE | 98382582 |
| PUBMED | 9714779 |
| REFERENCE | 4 (bases 1 to 3106) |
| AUTHORS | Kotera, J., Fujishige, K. , Imai, Y., Kawai, E., Michibata, H., |
| | Akatsuka, H., Yanaka, N. and Omori, K. |
| TITLE | Genomic origin and transcriptional regulation of two variants of |
| | cGMP-binding cGMP-specif ic phosphodiesterases |
| JOURNAL | Eur. J. Biochem. 262 (3), 866-873 (1999) |
| MEDLINE | 99339957 |
| PUBMED | 10411650 |
| REFERENCE | 5 (bases 1 to 3106) |
| AUTHORS | Lin, C. S., Lau, A., Tu, R. and Lue, T. F. |
| TITLE | Identification of three alternative first exons and an intronic |
| | promoter of human PDE5A gene |
| JOURNAL | Biochem. Biophys. Res. Commun. 268 (2), 596-602 (2000) |
| MEDLINE | 20145478 |
| PUBMED | 10679249 |
| REFERENCE | 6 (bases 1 to 3106) |
| AUTHORS | Lin, C. S., Lau, A., Tu, R. and Lue, T. F. |
| TITLE | Expression of three isoforms of cGMP-binding cGMP-specific |
| | phosphodiesterase (PDE5) in human penile cavernosum |
| JOURNAL | Biochem. Biophys. Res. Commun. 268 (2), 628-635 (2000) |
| MEDLINE | 20145484 |
| PUBMED | 10679255 |
| REFERENCE | 7 (bases 1 to 3106) |
| AUTHORS | Lin, C. S., Chow, S., Lau, A., Tu, R. and Lue, T. F. |
| TITLE | Identification and regulation of human PDE5A gene promoter |
| JOURNAL | Biochem. Biophys. Res. Commun. 280 (3), 684-692 (2001) |
| MEDLINE | 21092663 |
| PUBMED | 11162575 |
| COMMENT | REVIEWED REFSEQ: This record has been curated by NCBI staff. The |
| | reference sequence was derived from AF043731.1. |
| | On Oct. 1, 2001 this sequence version replaced gi:4505666. |
| | Summary: This gene encodes a cGMP-binding, cGMP-specific |
| | phosphodiesterase, a member of the cyclic nucleotide |
| | phosphodiesterase family. This phosphodiesterase specifically |
| | hydrolyzes cGMP to 5--GMP. It is involved in the regulation of |
| | intracellular concentrations of cyclic nucleotides and is important |
| | for smooth muscle relaxation in the cardiovascular system. |
| | Alternative splicing of this gene results in four transcript |
| | variants encoding distinct isoforms. |
| | Transcript Variant: This variant (1) encodes the longest isoform |
| | (1) of this protein. |

| FEATURES | | Location/Qualifiers |
|---|---|---|
| | source | 1..3106 |
| | | /organism = “Homo sapiens” |
| | | /db_xref = “taxon:9606” |
| | | /chromosome = “4” |
| | | /map = “4q25-q27” |
| | gene | 1..3106 |
| | | /gene = “PDE5A” |
| | | /note = “synonyms: CN5A, PDE5, PDE5A1, CGB-PDE” |
| | | /db_xref = “LocusID:8654” |
| | | /db_xref = “MIM:603310” |
| | CDS | 156..2783 |
| | | /gene = “PDE5A” |
| | | /EC_number = “3.1.4.17” |
| | | /note = “cGMP-binding cGMP-specific 3′,5′-cyclic nucleotide |
| | | phosphodiesterase” |
| | | /codon start = 1 |
| | | /product = “phosphodiesterase 5A isoform 1” |
| | | /protein id = “NP 001074.1” |
| | | /db_xref = “GI:4505667” |
| | | /db_xref = “LocusID:8654” |
| | | /db_xref = “MIM:603310” |
| | | /translation = “MERAGPSFGQQRQQQQPQQQKQQQRDQDSVEAWLDDHWDFTFSY |
| | | FVRKATREMVNAWFAERVHTIPVCKEGIRGHTESCSCPLQQSPRADNSVPGTPTRKIS |
| | | ASEFDRPLRPIVVKDSEGTVSFLSDSEKKEQMPLTPPRFDHDEGDQCSRLLELVKDIS |
| | | SHLDVTALCHKIFLHIHGLISADRYSLFLVCEDSSNDKFLISRLFDVAEGSTLEEVSN |
| | | NCIRLEWNKGIVGHVAALGEPLNIKDAYEDPRFNAEVDQITGYKTQSILCMPIKNHRE |
| | | EVVGVAQAINKKSGNGGTFTEKDEKDFAAYLAFCGIVLHNAQLYETSLLENKRNQVLL |
| | | DLASLIFEEQQSLEVILKKIAATIISFMQVQKCTIFIVDEDCSDSFSSVFHMECEELE |
| | | KSSDTLTREHDANKINYMYAQYVKNTMEPLNIPDVSKDKRFPWTTENTGNVNQQCIRS |
| | | LLCTPIKNGKKNKVIGVCQLVNKMEENTGKVKPFNRNDEQFLEAFVIFCGLGIQNTQM |
| | | YEAVERAMAKQMVTLEVLSYHASAAEEETRELQSLAAAVVPSAQTLKITDFSFSDFEL |
| | | SDLETALCTIRMFTDLNLVQNFQMKHEVLCRWILSVKKNYRKNVAYHNWRHAFNTAQC |
| | | MFAALKAGKIQNKLTDLEILALLIAALSHDLDHRGVNNSYIQRSEHPLAQLYCHSIME |
| | | HHHFDQCLMILNSPGNQILSGLSIEEYKTTLKIIKQAILATDLALYIKRRGEFFELIR |
| | | KNQFNLEDPHQKELFLAMLMTACDLSAITKPWPIQQRIAELVATEFFDQGDRERKELN |
| | | IEPTDLMNREKKNKIPSMQVGFIDAICLQLYEALTHVSEDCFPLLDGCRKNRQKWQAL |
| | | AEQQEKMLINGESGQAKRN” |
| | misc feature | 645..1118 |
| | | /gene = “PDE5A” |
| | | /note = “GAF; Region: Domain present in phytochromes and |
| | | cGMP-specific phosphodiesterases” |
| | | /db_xref = “CDD:smart00065” |
| | misc feature | 645..1097 |
| | | /gene = “PDE5A” |
| | | /note = “GAF; Region:GAF domain. Domain present in |
| | | phytochromes and cGMP-specific phosphodiesterases” |
| | | /db_xref = “CDD:pfam01590” |
| | misc feature | 1191..1694 |
| | | /gene = “PDE5A” |
| | | /note = “GAF; Region: Domain present in phytochromes and |
| | | cGMP-specific phosphodieste rases” |
| | | /db_xref = “CDD:smart00065” |
| | misc feature | 1191..1664 |
| | | /gene = “PDE5A” |
| | | /note = “GAF; Region: GAF domain. Domain present in |
| | | phytochromes and cGMP-specific phosphodiesterases” |
| | | /db_xref = “CDD:pfam01590” |
| | raise feature | 1989..2705 |
| | | /gene = “PDE5A” |
| | | /note = “PDEase; Region: 3′5′-cyclic nucleotide |
| | | phosphodiesterase” |
| | | /db xref = “CDD:pfam00233” |
| | variation | complement (433) |
| | | /allele = “G” |
| | | /allele = “A” |
| | | /db xref = “dbSNP: 3733526” |

| BASE COUNT | 916 a 625 c 732 g 833 t |
|---|---|
| ORIGIN | |
| 1 | gcggccgcgc tccggccgct ttgtcgaaag ccggcccgac tggagcagga cgaaggggga |
| 61 | gggtctcgag gccgagtcct gttcttctga gggacggacc ccagctgggg tggaaaagca |
| 121 | gtaccagaga gcctccgagg cgcgcggtgc caaccatgga gcgggccggc cccagcttcg |
| 181 | ggcagcagcg acagcagcag cagccccagc agcagaagca gcagcagagg gatcaggact |
| 241 | cggtcgaagc atggctggac gatcactggg actttacctt ctcatacttt gttagaaaag |
| 301 | ccaccagaga aatggtcaat gcatggtttg ctgagagagt tcacaccatc cctgtgtgca |
| 361 | aggaaggtat cagaggccac accgaatctt gctcttgtcc cttgcagcag agtcctcgtg |
| 421 | cagataacag tgtccctgga acaccaacca ggaaaatctc tgcctctgaa tttgaccggc |
| 481 | ctcttagacc cattgttgtc aaggattctg agggaactgt gagcttcctc tctgactcag |
| 541 | aaaagaagga acagatgcct ctaacccctc caaggtttga tcatgatgaa ggggaccagt |
| 601 | gctcaagact cttggaatta gtgaaggata tttctagtca tttggatgtc acagccttat |
| 661 | gtcacaaaat tttcttgcat atccatggac tgatatctgc tgaccgctat tccctgttcc |
| 721 | ttgtctgtga agacagctcc aatgacaagt ttcttatcag ccgcctcttt gatgttgctg |
| 781 | aaggttcaac actggaagaa gtttcaaata actgtatccg cttagaatgg aacaaaggca |
| 841 | ttgtgggaca tgtggcagcg cttggtgagc ccttgaacat caaagatgca tatgaggatc |
| 901 | ctcggttcaa tgcagaagtt gaccaaatta caggctacaa gacacaaagc attctttgta |
| 961 | tgccaattaa gaatcatagg gaagaggttg ttggtgtagc ccaggccatc aacaagaaat |
| 1021 | caggaaacgg tgggacattt actgaaaaag atgaaaagga ctttgctgct tatttggcat |
| 1081 | tttgtggtat tgttcttcat aatgctcagc tctatgagac ttcactgctg gagaacaaga |
| 1141 | gaaatcaggt gctgcttgac cttgctagtt taatttttga agaacaacaa tcattagaag |
| 1201 | taattttgaa gaaaatagct gccactatta tctctttcat gcaagtgcag aaatgcacca |
| 1261 | ttttcatagt ggatgaagat tgctccgatt ctttttctag tgtgtttcac atggagtgtg |
| 1321 | aggaattaga aaaatcatct gatacattaa caagggaaca tgatgcaaac aaaatcaatt |
| 1381 | acatgtatgc tcagtatgtc aaaaatacta tggaaccact taatatccca gatgtcagta |
| 1441 | aggataaaag atttccctgg acaactgaaa atacaggaaa tgtaaaccag cagtgcatta |
| 1501 | gaagtttgct ttgtacacct ataaaaaatg gaaagaagaa taaagttata ggggtttgcc |
| 1561 | aacttgttaa taagatggag gagaatactg gcaaggttaa gcctttcaac cgaaatgacg |
| 1621 | aacagtttct ggaagctttt gtcatctttt gtggcttggg gatccagaac acgcagatgt |
| 1681 | atgaagcagt ggagagagcc atggccaagc aaatggtcac attggaggtt ctgtcgtatc |
| 1741 | atgcttcagc agcagaggaa gaaacaagag agctacagtc gttagcggct gctgtggtgc |
| 1801 | catctgccca gacccttaaa attactgact ttagcttcag tgactttgag ctgtctgatc |
| 1861 | tggaaacagc actgtgtaca attcggatgt ttactgacct caaccttgtg cagaacttcc |
| 1921 | agatgaaaca tgaggttctt tgcagatgga ttttaagtgt taagaagaat tatcggaaga |
| 1981 | atgttgccta tcataattgg agacatgcct ttaatacagc tcagtgcatg tttgctgctc |
| 2041 | taaaagcagg caaaattcag aacaagctga ctgacctgga gatacttgca ttgctgattg |
| 2101 | ctgcactaag ccacgatttg gatcaccgtg gtgtgaataa ctcttacata cagcgaagtg |
| 2161 | aacatccact tgcccagctt tactgccatt caatcatgga acaccatcat tttgaccagt |
| 2221 | gcctgatgat tcttaatagt ccaggcaatc agattctcag tggcctctcc attgaagaat |
| 2281 | ataagaccac gttgaaaata atcaagcaag ctattttagc tacagaccta gcactgtaca |
| 2341 | ttaagaggcg aggagaattt tttgaactta taagaaaaaa tcaattcaat ttggaagatc |
| 2401 | ctcatcaaaa ggagttgttt ttggcaatgc tgatgacagc ttgtgatctt tctgcaatta |
| 2461 | caaaaccctg gcctattcaa caacggatag cagaacttgt agcaactgaa ttttttgatc |
| 2521 | aaggagacag agagagaaaa gaactcaaca tagaacccac tgatctaatg aacagggaga |
| 2581 | agaaaaacaa aatcccaagt atgcaagttg ggttcataga tgccatctgc ttgcaactgt |
| 2641 | atgaggccct gacccacgtg tcagaggact gtttcccttt gctagatggc tgcagaaaga |
| 2701 | acaggcagaa atggcaggcc cttgcagaac agcaggagaa gatgctgatt aatggggaaa |
| 2761 | gcggccaggc caagcggaac tgagtggcct atttcatgca gagttgaagt ttacagagat |
| 2821 | ggtgtgttct gcaatatgcc tagtttctta cacactgtct gtatagtgtc tgtatttggt |
| 2881 | atatactttg ccactgctgt atttttattt ttgcacaact tttgagagta tagcatgaat |
| 2941 | gtttttagag gactattaca tattttttgt atatttgttt tatgctactg aactgaaagg |
| 3001 | atcaacaaca tccactgtta gcacattgat aaaagcattg tttgtgatat ttcgtgtact |
| 3061 | gcaaagtgta tgcagtattc ttgcactgag gtttttttgc ttgggg |

1. A method for developing ligands binding to PDE5A, comprising
identifying as molecular scaffolds one or more compounds that bind to a binding site of PDE5A;
determining the orientation of at least one molecular scaffold in co-crystals with PDE5A; and
identifying chemical structures of said molecular scaffolds, that, when modified, alter the binding affinity or binding specificity or both between the molecular scaffold and PDE5A; and
synthesizing a ligand wherein one or more of the chemical structures of the molecular scaffold is modified to provide a ligand that binds to PDE5A with altered binding affinity or binding specificity or both.

2. The method of claim 1, wherein said molecular scaffold is a weak binding compound.

3. The method of claim 1, wherein said molecular scaffold binds to a plurality of phosphodiesterases.

4. A method for developing ligands specific for PDE5A, comprising
identifying a compound that binds to a plurality of phosphodiesterases; and
determining whether a derivative of said compound has greater specificity for PDE5A than said compound.

5. The method of claim 4, wherein said compound binds to PDE5A with an affinity at least 10-fold greater than for binding to any of said plurality of phosphodiesterases.

6. The method of claim 5, wherein said compound interacts with at least one conserved PDE5A active site residue.

7. The method of claim 4, wherein said compound binds weakly to said plurality of phosphodiesterases.

8. The method of claim 4, wherein said plurality of phosphodiesterases comprises PDE5A and PDE6.

9. The method of claim 4, wherein said plurality of phosphodiesterases comprises PDE5A and PDE11.

10. A method for identifying potential PDE5A binding compounds, comprising
identifying a molecular scaffold that binds to PDE5A; and
fitting at least one electronic representation of a compound in an electronic representation of a PDE5A binding site, wherein said compound is a derivative of said molecular scaffold.

11. The method of claim 10, wherein said electronic representation of a PDE5A binding site is defined by atomic structural coordinates set forth in Table 1.

12. The method of claim 10, comprising
removing a computer representation of a compound complexed with PDE5A and fitting a computer representation of a compound from a computer database with a computer representation of the active site of PDE5A; and
identifying compounds derived from said molecular scaffold that best fit said active site based on favorable geometric fit and energetically favorable complementary interactions as potential binding compounds.

13. The method of claim 10, comprising
modifying a computer representation of a compound complexed with PDE5A by the deletion or addition or both of one or more chemical groups;
fitting a computer representation of a compound derived from said molecular scaffold from a computer database with a computer representation of the active site of PDE5A; and
identifying compounds derived from aid molecular scaffold that best fit said active site based on favorable geometric fit and energetically favorable complementary interactions as potential binding compounds.

14. The method of claim 10, comprising
removing a computer representation of a molecular scaffold or a derivative compound thereof complexed with PDE5A and; and
searching a database for compounds having structural similarity to said molecular scaffold or derivative compound using a compound searching computer program or replacing portions of said compound with similar chemical structures using a compound construction computer program.

15. The method of claim 10, wherein said compound complexed with PDE5A is non-hydrolyzable cGMP analog.

16. The method of claim 10, wherein said fitting comprises determining whether a said compounds will interact with one or more of conserved PDE5A active site residues.

17. A method for attaching a PDE5A binding compound to an attachment component, comprising
identifying energetically allowed sites for attachment of a said attachment component on a phosphodiesterase binding compound; and
attaching said compound or derivative thereof to said attachment component at said energetically allowed site.

18. The method of claim 17, wherein said attachment component is a linker for attachement to a solid phase medium, and said method further comprises attaching said compound or derivative to a solid phase medium through a linker attached at a said energetically allowed site.

19. The method of claim 17, wherein said phosphodiesterase comprises conserved residues matching at least one conserved PDE5A active site residues.

20. The method of claim 18, wherein said linker is a traceless linker.

21. The method of claim 18, wherein said phosphodiesterase binding compound or derivative thereof is synthesized on a said linker attached to said solid phase medium.

22. The method of claim 21, wherein a plurality of said compounds or derivatives are synthesized in combinatorial synthesis.

23. The method of claim 18, wherein attachment of said compound to said solid phase medium provides an affinity medium.

24. The method of claim 17, wherein said attachment component comprises a label.

25. The method of claim 24, wherein said label comprises a fluorophore.
